(19) United States
(12) Patent Application Publication
(10) Pub. No.: US 2010/0292214 A1
(43) Pub. Date: Nov. 18, 2010

(54) Compounds with Antiparasitic Activity, Applications thereof to the Treatment of Infectious Diseases Caused by Apicomplexans

(75) Inventors: Michael Deligny, Caen (BE); Nadia Saidani, Montpellier (FR); Anne-Laure Bonneau, Paris (FR); Cyrille Botte, Grenoble (FR); Helene Hardre, Chateauvillain (FR); Bernard Rousseau, Levallois-perret (FR); Henri Vial, Montpellier (FR); Corinne Mercier, La Tronche (FR); Roman Lopez, Issy Ies Moulineaux (FR); Eric Marechal, Grenoble (FR)

Correspondence Address: MORGAN LEWIS & BOCKIUS LLP, 1111 PENNSYLVANIA AVENUE NW, WASHINGTON, DC, 20004, US

(73) Assignees: commissariat a l'Energie Atomique, Paris (FR); Centre National De La Recherche Scientifique, Paris (FR)

(21) Appl. No.: 12/602,400

(22) PCT Filed: Jun. 2, 2008
(86) PCT No.: PCT/IB08/02467

§ 371 Date: Jul. 1, 2010

(30) Foreign Application Priority Data
Jun. 1, 2007 (EP) 07290683.7

(51) Int. Cl. A61K 31/55; A61K 31/4545; A61K 31/454; C07D 487/04; C07D 401/14; C07D 401/04; A61P 33/00

(52) U.S. Cl. 514/214.02; 514/316; 514/322; 540/578; 546/187; 546/199

(57) ABSTRACT

The Invention relates to compounds having an antiparasitic activity, and to their use as a drug, in particular as a drug for the prevention and/or treatment of parasitic diseases caused by apicomplexans. The invention also relates to pharmaceutical compositions containing those compounds.

The Invention relates to compounds having an antiparasitic activity, and to their use as a drug, in particular as a drug for the prevention and/or treatment of parasitic diseases caused by apicomplexans. The invention also relates to pharmaceutical compositions containing those compounds.

The Apicomplexa phylum constitutes a particularly diverse group of unicellular protists. Besides members of the Colpodellida sub-group, all apicomplexans are obligate intracellular parasites, infecting virtually all animals from mollusks to mammals. They were originally classified as protozoa (>4000 species). Amongst them, the malarial parasite Plasmodium sp., represents one of the most important threats for humankind, with 300 to 500 million estimated clinical cases and 1.5 to 3 million deaths each year, most of them African children. More benign, but widely spread is Toxoplasma gondii, a parasite causing congenital neurological birth defects and, along with Cryptosporidium, being the most reported opportunistic infection associated with immunosuppressive conditions, including AIDS. Other parasites in this phylum are of veterinary importance including pathogens of cattle and chicken, Theileria and Eimeria.

The Apicomplexa phylum contains several thousands of unicellular parasites among which:
Plasmodium falciparum, the major malaria causative agent, as well as other parasites of the same genus (the three other Plasmodium species that cause human malaria: P. vivax, P. ovale, P. malariae; the Plasmodium species known to infect other vertebrates: P. yoelii, P. berghei, P. chabaudi, P. vivax, P. gallinaceum, P. knowlesi, P. reichenowi, etc.);
Babesia microti, the causative agent of human babesiosis, as well as all other parasites that cause babiesosis in mammals (e.g. B. divergens, B. bigemina, B. major, B. bovis (bovine babesiosis), B. canis, B. gibsoni (canine babesiosis), B. equi, B. caballi (equine babesiosis), etc.);
Toxoplasma gondii, the causative agent of toxoplasmosis in humans and other mammals;
Neospora caninum, the causative agent of neosporosis;
Cryptosporidium hominis, the causative agent of human cryptosporidiosis, as well as other parasites of the same genus (C. parvum, etc.);
Theileria annulata, the causative agent of theileriosis, as well as other parasites of the same genus (T. parva, etc.)
Sarcocystis neurona, Eimeria tenella, Gregarina niphandrodes, as well as any other parasites of the apicomplexa phylum causing infectious diseases in metazoans.

All apicomplexans share a polarized cellular organization schema, with a basal pole and an apical pole. Membrane compartmentalization exhibits some specific features as compared to other eukaryotic cells such as those of yeasts or mammals. The cell is polarized and exhibits a set of subcellular structures called the apical complex that contains a hollow truncated cone, the conoid, and three types of vesicular secretory organelles, namely rhoptries, micronemes and dense granules. During host cell invasion, the apical pole is the first to enter the cell. Membrane compartments include those known to be connected through the endomembrane flow in all eukaryotic cells, i.e. the Golgi, endoplasmic reticulum, nuclear envelope and plasma membrane. A peripheral inner membrane complex is applied to the plasma membrane surface. Semi autonomous organelles include a mitochondria surrounded by two membranes, and the apicoplast (absent in some genera such as Cryptosporidium) surrounded by four membranes.

The apicomplexan parasites are transmitted by different vectors in complex life cycles. For instance, and like all other malaria parasites of the Plasmodium genus, Plasmodium falciparum spreads by infecting successively two types of hosts: humans and female Anopheles mosquitoes. In humans, the parasites grow and multiply first in the liver cells and then in the red cells of the blood. In the blood, successive broods of parasites grow inside the red cells and destroy them, releasing daughter parasites (“merozoites”) that continue the cycle by invading other red cells. The blood stage parasites are those that cause the symptoms of malaria. When certain forms of blood stage parasites (“gametocytes”) are picked up by a female Anopheles mosquito during a blood meal, they start another, different cycle of growth and multiplication in the mosquito. After 10-18 days, the parasites are found (as “sporozoites”) in the mosquito's salivary glands. When the Anopheles mosquito takes a blood meal on another human, the sporozoites are injected with the insect's saliva and start another human infection.

Chemotherapies are needed to fight against apicomplexan-caused diseases. These therapies generally differ with the type of parasite responsible of the infection.

In the case of malaria, this life-threatening disease affects half a billion humans in underdeveloped and developing countries. Its global heartland is Africa, with an appalling death toll of 1 to 2 million people every year (WHO World Malaria Report, Geneva, World Health Organization, WHO/UNICEF; 2005). Endemic malaria ranges from a permanent incidence in sub-Saharan and equatorial Africa, to a seasonal but recently escalating prevalence in Southern Africa Grover-Kopec et al., Malar J. 2006, 5:38). Malaria was eradicated from temperate regions following concerted preventative sanitary actions and after important insecticide campaigns and systematic treatments with available drugs, i.e. quinine and chloroquine. The prophylactic programs of the 1950's and 1960's, essentially based on insecticide and drug treatments, failed to control malaria in subtropical areas. Resistance to chloroquine spread rapidly. Subsequent attempts to achieve progress in malaria prophylaxis have been characterized by the failure of vaccine development, withdrawal of some insecticides because of toxicity and negative environmental impact, the alarming spread of mosquito resistance to insecticides and of resistance of Plasmodium to the very few drugs that have been developed.

In an effort to improve the success rate of drug development, several approaches are being investigated. Most of the strategies are based on re-design of existing drugs, for example artemisinin, an antiplasmodial molecule from Artemisia annua comprising an endoperoxide moiety, and derivatives thereof such as Artesunate, which can be produced efficiently and cheaply (Renslo A. R., McKerrow J. H., Nature Chem. Biol., 2006, 2, 701-710).

Others choose to only keep the endoperoxyde moiety of artemisinin, leading to the discovery of synthetic RBx11160 (OZ277) which mimics the chemical and biological properties of this molecule and is already in clinical trials as antimalaria drug used with piperaquine (Vennerstrom, J. L. et al., Nature, 2004, 430, 900-904).

The novel use of combination of older drugs is also under investigation: Lapdap® (Chloroproguanil-dapsone) or Lapdap®-artesunate are based on combinations of chloroproguanil (CPG) and dapsone (DDS) and was launched in 2003 as an effective and cheap antimalarial (Mutabingwa T, et al., Lancet, 2001, 358, 1218-1223).

Another well known drug is the quinolines family. Work on this old drug eventually succeeded to introduce antiplasmodial compounds such as quinoline and isoquine derivatives (Madrid, P. B. et al., J. J. Med. Chem. 2007, 50, 889-896). Very recently astemizole, a known antihistaminic drug was shown to have a similar activity toward plasmodium than artemisinin and other derivatives (Chong, C. R., et al., Nat. Chem. Biol., 2006, 2, 415-416).

Current efforts focus therefore on chemotherapy using multiple therapies including artemisinin. However, the scientific community is worried that plans for the extensive use of artemisinin might be ruined by emergence of the parasitic resistance it will almost certainly trigger, sooner or later. Given the small number of available drugs and the resistance they have already induced, discovery of new targets and of new drugs remains a key priority.

In the case of toxoplasmosis, Toxoplasma gondii has a broad spectrum of hosts and can virtually infect any mammal. It is well established that the final hosts are cats where sexual reproduction occurs. Transmission to humans occurs most often by infected food, or by direct or indirect contacts with cats. Severity of toxoplasmosis is very variable. In healthy human adults, infection is usually not visible besides serum analyses. Most healthy people don't require any treatment. In infected patients, the parasite remains as cysts that can reactivate a toxoplasmosis in case of immune deficiencies. Treatments are critically needed in the case of pregnant women and babies, people with HIV/AIDS and patient treated for cancer by chemotherapies. Most widely used treatments include pyrimethamine and sulfadiazine

Because these drugs can have serious side effects for both women and babies, they are normally not used during pregnancy. Drug treatment can lessen the severity of congenital toxoplasmosis, but it will not undo any damage that has already occurred. Pyrimethamine is an antimalarial medication also used to treat toxoplasmosis, and acts as a folic acid antagonist. It may prevent the body from absorbing the important B vitamin folate (folic acid, vitamin B-9), especially when taking high doses over a long period of time. Other potential side effects of pyrimethamine include bone marrow suppression and liver toxicity. Sulfadiazine is an antibiotic used in combination with pyrimethamine to treat toxoplasmosis. Possible side effects include nausea, vomiting and diarrhea. Pyrimethamine can also be supplied in conjunction with clindamycin, an antibiotic that can sometimes cause severe diarrhea. Patients suffering from AIDS need to take these medications for life, although in some cases, toxoplasmosis therapies can be stopped if CD4 count remains very high for at least three to six months. Side effects of most drugs can be more severe in people with HIV/AIDS. Toxoplasmosis is also an important veterinary infection in ovines, bovines, caprines and pigs, and is responsible for spontaneous abortion. Efficient drugs with low side effects, acting on cyst forms and usable both for human therapies and preventions in cattle farms are therefore highly needed.

The search for effective therapeutics for toxoplasmosis received much less interest and only a few examples are reported. The main target is dihydrofolate reductase (DHFR) and some inhibitors with high ligand efficiency were reported; for example starting from a QSAR analysis some authors obtained the synthesis of new 6-fluoroquinolones as potential active against T. gondii (Anquetin G., et al., Eur. J. Med. Chem., 2006, 41, 1478-1493.

Very recently others reported the SAR studies based on trimethoprim and methotrexate analogues. Starting from this 1960-drug they obtained high in vitro inhibitory activities toward a homology model of DHFR.

In the case of babesiosis, infection is similar to malaria. Transmission of Babesia parasites is due to bites by ticks. Wild animals (rodents and bovids) are final or intermediary parasitic reservoirs. Some Babesia, such as B. microti and B. divergens can infect humans. Babesiosis is reported in various domestic species, including bovids, equids, and dogs with a large spectrum of symptom, from non-severe cases to lethality. Untreated canine babesiosis is always lethal. Besides a partially efficient vaccine, no drug treatment is currently available to fight against all Babesia species. Human treatments include the use of quinine combined with clindamycin, or hydroxychloroquine. For veterinary purposes, bovine and equine babesiosis caused by only two species can be mainly treated, using imidocarb dipropionate.

In the case of neosporosis, data are fragmentary and poor. Neospora is a parasite closely related to Toxoplasma, capable of infecting virtually any mammal. Dog is the final host were sexual reproduction occurs. Cattles can be infected due to proximity with infected wild animals. Drugs used to treat toxoplasmosis are used to treat neosporosis.

In the case of coccidiosis, tens of species of the Eimeria genus can cause infections of the gastro-intestinal system of birds and mammals. Coccidiosis is a to very frequent plague in bovids, ovids, birds, rodents, etc. Massive poultry breeding requires prophylaxis against avian coccidiosis, particularly against Eimeria necatrix or E. tenella that cause severe infections. Drug treatments include the prescription of antibiotics or non-antibiotic molecules such as decoquinate, a molecule of the hydroxyquinoline family.

New molecules are therefore needed for some specific infections such as malaria or numerous babesioses, or to treat some parasitic stages that are currently resistant to drugs, such as toxoplasmic cysts. Molecules having an effect on numerous apicomplexan species would be benefic for both medical and veterinary purposes.

The inventors have developed the subject of the invention in order to remedy these problems.

A first subject of the Invention is therefore compounds of general formula (I) below:

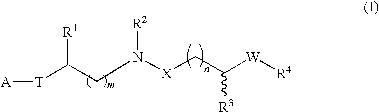

or a pharmaceutically acceptable salt thereof, wherein:
T is selected from the group consisting of —CH₂—, —CH₂CH₂—, —O—, —NH—, a simple bond and a double bond;
m and n, independently, are an integer equal to 0, 1, 2, 3 or 4;
R¹ and R² simultaneously represent an hydrogen atom or R¹ and R² form together an alkyl chain having from 2 or 3 carbon atoms with possibly one insaturation;
R³ represents hydrogen or a group selected among alkyl, alkenyl, cycloalkyl, aryl, arylalkyl, heteroalkyl, heteroaryl and heteroarylalkyl groups wherein said groups defined for R³ are optionally substituted with one or more groups independently selected from halogen, trifluoromethyl, difluoromethyl, azido, alkyl, alkoxy, cyano, nitro and carbethoxy;
R⁴ represents hydrogen or a group selected among alkyl, alkenyl, cycloalkyl, aryl, arylalkyl, heteroaryl, heteroarylalkyl and heteroalkyl, wherein said groups defined for R⁴ are optionally substituted with one or more groups independently selected from halogen, trifluoromethyl, difluoromethyl, azido, alkyl, alkoxy, cyano, nitro, carboxy and carbethoxy;
X represents a single bond, —CH₂— or a functional group chosen among the groups of formulas (X-1) to (X-6) below:

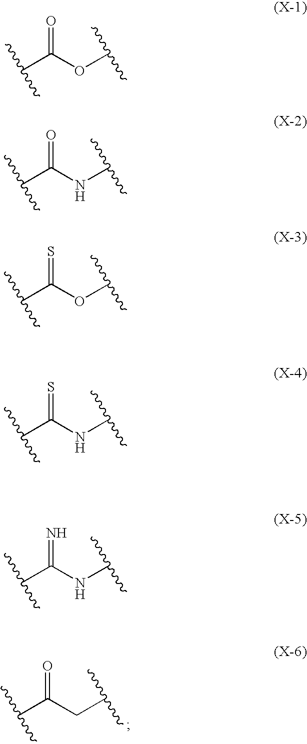

A represents a cyclic moiety selected from the group consisting of:
i) a moiety of formula (A-1) below:

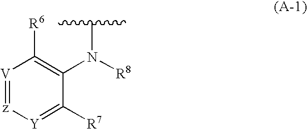

in which:
V, Y and Z, identical or different, represent —CH—, C—R⁶ or N, it being understood that V can also form, together with R⁶ or Z, a fused aryl or heteroaryl ring depending of the nature of R⁶ or Z;
R⁶ represents hydrogen, halogen, a group selected from the group consisting in alkoxy, alkyl, nitro, alkenyl, alkynyl, cycloalkyl, aryl, arylalkyl, heteroaryl and heteroalkyl, wherein said alkoxy, alkyl, alkenyl, alkynyl cycloalkyl, aryl, arylalkyl, heteroaryl and heteroalkyl groups are optionally substituted with one or more groups independently selected from halogen, trifluoromethyl, difluoromethyl, azido, alkyl, alkoxy, amino, cyano and nitro;
R⁷ represents a hydrogen atom, a halogen atom, or a group selected among alkyl, nitro, alkoxy, aminoalkyl, aryl and heteroaryl groups;
R⁸ represents a hydrogen atom, an alkyl group, an alkenyl group, an aryl group, a heteroaryl group or a group selected from the groups having the following formulas —COOR⁹, —C(O)NR⁹R¹⁰, —C(S)NR⁹R¹⁰, —C(O)NHR⁹, —C(O)R⁹, —SO₂R⁹ in which R⁹ and R¹⁰, which may be identical or different, represent a hydrogen atom, an alkoxy group, an amino group, an alkyl group, an alkenyl group, an aryl group and a heteroaryl group;
R⁷ and R⁸, together, can also form a moiety of formula —C(=Het)-N(R¹¹)— in which Het represents an oxygen atom or a sulphur atom and R¹¹ represents a hydrogen atom, a linear or branched alkyl or alkenyl group; a heteroaryl group, a cycloalkyl group, an aryl group and an arylalkyl group, wherein said groups are optionally substituted with one or more groups independently selected from halogen, trifluoromethyl, difluoromethyl, azido, alkyl, alkoxy, cyano, nitro; a SO₂R¹², a COR¹² group or a COOR¹² group wherein R¹² represents a group chosen among alkyl, alkenyl, cycloalkyl, aryl, heteroaryl, arylalkyl and heteroalkyl, wherein said groups mentioned for R¹² are optionally substituted with one or more groups independently selected from halogen, trifluoromethyl, difluoromethyl, azido, alkyl, alkoxy, cyano, nitro, carboxy and carbethoxy; a group of formula-alkylC(O)R¹³ in which R¹³ represents hydrogen or an alkoxy group; a group of formula alkyl-NHR¹⁴ in which R¹⁴ represents an alkoxycarbonyl group or a biotine moiety; R¹¹ may also form, together with Y when Y is a carbon atom, a saturated heterocarbonated ring comprising 3 or 4 carbon atoms in addition to the carbon atom of Y, said ring being optionally substituted with an oxo functional group;
ii) a moiety of formula (A-2) below:

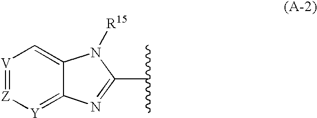

in which:
V, Z, and Y can take the same significations as the moiety of formula (A-1), and
R¹⁵ represents a hydrogen atom, an alkyl or a benzyl radical or a group of formula —NC(O)—OCH₂—CH(CH₃)—N-(Bz)₂ in which Bz stands for benzyl;
W represents C—R¹⁶, N—R¹⁶ or O—R¹⁶ wherein R¹⁶ represents a group selected among alkyl, alkenyl, cycloalkyl, aryl, arylalkyl heteroaryl and heteroalkyl, wherein said groups defined for R¹⁶ are optionally substituted with one or more groups independently selected from halogen, trifluoromethyl, difluoromethyl, azido, alkyl, alkoxy, cyano, nitro, carboxy and carbethoxy; W may also represents a N-containing ring selected among moieties having the formulas W-2, W-3 and W-4 below:

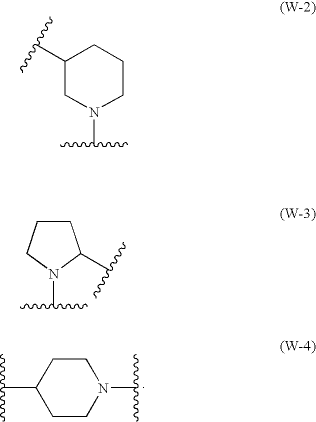

with the proviso that when A represents a moiety of formula (A-1) in which R⁸ is a group of formula —SO₂R¹² in which R¹² stands for 4-chlorophenyl, then R⁷ is different from a fluorine atom; these compounds being already known from international application WO 03/013527 as active principles useful for the treatment of Alzheimer's disease.

According to a preferred embodiment of the present Invention, R¹ and R², together with the carbon atom (for R¹), the nitrogen atom (for R²) and the chain bonding the carbon atom bearing R¹ and the nitrogen atom bearing R² (i.e.: —(CH₂)ₘ—) form a moiety selected from the group consisting of the following moieties:

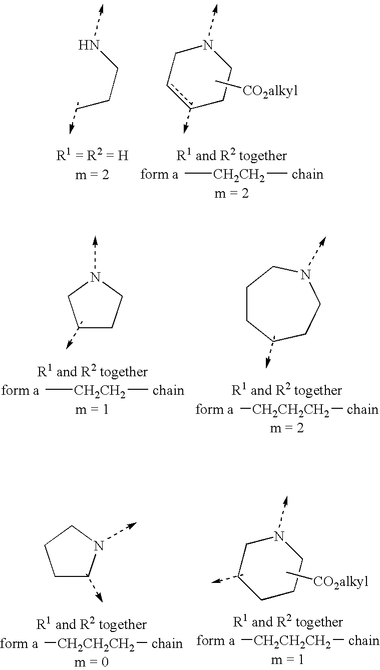

wherein the dashed arrows represent the attachment point of these moieties to T and X respectively via a covalent bond.

According to the present invention, when R⁸ and R⁷ together form a moiety of formula —C(=Het)-N(R¹¹)—, (A-1) has the particular configuration (A-1-a):

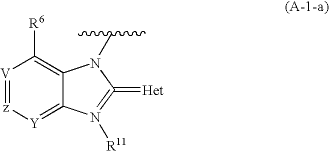

in which V, Z, Y, R⁶, Het and R¹¹ have the same significations as above-mentioned for (A-1).

According to a particular embodiment of the present invention, when (A-1) represents a moiety of configuration (A-1-a), R¹¹ is then preferably different from a hydrogen atom. In this case, R¹¹ is preferably selected from the group consisting of an alkyl radical and a group of formula SO₂R¹² in which R¹² has the same significations as those already mentioned. In this later case, i.e. when R¹¹ is group of formula SO₂R¹², then R¹² preferably represents an aryl or a heteroaryl group.

In the sense of the present Invention, linear and branched alkyl groups (main or auxiliary) mentioned in the present specification are chosen among (C₁-C₄)alkyl groups such as methyl, ethyl, n-propyl, isopropyl, n-butyl, tert-butyl and isobutyl radicals; linear and branched alkoxy groups (main or auxiliary) mentioned in the present specification are chosen among (C₁-C₄)alkoxy groups such as methyloxy, ethyloxy, n-propyloxy, iso-propyloxy, n-butyloxy, tert-butyloxy and isobutyloxy radicals.

Also in the sense of the present Invention, halogen atoms are chosen among bromine, chlorine, fluorine and iodine.

According to the Invention, aryl and heteroaryl groups refer to any functional group or substituent derived from at least one simple aromatic ring; an aromatic ring corresponding to any planar cyclic compound having a delocalized system in which each atom of the ring comprises a p-orbital, said p-orbitals overlapping themselves. Among heteroaryl groups, one can mention furan, pyridine, pyrrole, thiophene, imidazole, pyrazole, oxazole, isoxazole, thiazole, benzene, pyridine, pyrazine, pyrimidine, pyridazine, benzylcyclobutene, pentalene, benzofurane, isobenzofurane, indole, isoindole, benzothiophene, benzo[c]thiophene, benzimidazole, indazole, benzoxazole, benzisoxazole, benzothiazole, naphthalene, quinoline, isoquinoline, quinoxaline, quinazoline, cinnoline, purine, anthracene and acridine.

A large number of the compounds of formula (I) as above-defined possess an asymmetric carbon. In this case, they can have the (R) or the (S) absolute configuration. The present Invention encompasses both configurations and mixtures thereof, in particular racemate mixtures.

As particular compounds of formula (I) above, one can mention:
(S)-2-(dibenzylamino)propyl 4-(1,2-dihydro-2-oxobenzo[d]imidazol-3-yl)piperidine-1-carboxylate;
(S)-2-(dibenzylamino)-3-phenylpropyl 4-(1,2-dihydro-2-oxobenzo[d]imidazol-3-yl)piperidine-1-carboxylate;
2-(dibenzylamino)ethyl 4-(1,2-dihydro-2-oxobenzo[d]imidazol-3-yl)piperidine-1-carboxylate;
3-(dibenzylamino)propyl 4-(1,2-dihydro-2-oxobenzo[d]imidazol-3-yl)piperidine-1-carboxylate;
2-(N-benzyl-N-methylamino)ethyl 4-(1,2-dihydro-2-oxobenzo[d]imidazol-3-yl)piperidine-1-carboxylate;
(S)-2-(dibenzylamino)propyl 5,6-dihydro-4-(1,2-dihydro-2-oxobenzo[d]imidazol-3-yl)pyridine-1(2H)-carboxylate;
2-(benzyloxy)ethyl 4-(1,2-dihydro-2-oxobenzo[d]imidazol-3-yl)piperidine-1-carboxylate;
(S)-2-(dibenzylamino)propyl 4-(1,2-dihydro-2-thioxobenzo[d]imidazol-3-yl)piperidine-1-carboxylate;
(S)-2-(dibenzylamino)propyl 4-(2-oxobenzo[d]oxazol-3(2H)-yl)piperidine-1-carboxylate;
(S)-2-(dibenzylamino)propyl 4-(1H-benzo[d]imidazol-1-yl)piperidine-1-carboxylate;
(S)-2-(dibenzylamino)propyl 4-(1-methyl-1H-benzo[d]imidazol-2-yloxy)piperidine-1-carboxylate;
(S)-2-(dibenzylamino)propyl 3-(1,2-dihydro-2-oxobenzo[d]imidazol-3-yl)pyrrolidine-1-carboxylate;
tert-butyl 3-(1-(((S)-2-(dibenzylamino)propoxy)carbonyl)piperidin-4-yl)-2,3-dihydro-2-oxobenzo[d]imidazole-1-carboxylate;
1-(1-(3-(dibenzylamino)propyl)piperidin-4-yl)-1H-benzo[d]-imidazol-2(3H)-one;
(S)-2-(dibenzylamino)propyl 4-(1,2-dihydro-1-methyl-2-oxobenzo-[d]imidazol-3-yl)piperidine-1-carboxylate;
1-(1-(2-(dibenzylamino)ethyl)piperidin-4-yl)-1H-benzo[d]imidazol-2(3H)-one;
1-(1-((S)-2-(dibenzylamino)-3-phenylpropyl)piperidin-4-yl)-1H-benzo[d]imidazol-2(3H)-one;
1-(1-((S)-2-(dibenzylamino)propyl)piperidin-4-yl)-1H-benzo[d]imidazol-2(3H)-one;
O-2-(dibenzylamino)ethyl 4-(1,2-dihydro-2-oxobenzo[d]imidazol-3-yl)piperidine-1-carbothioate;
O-2-(benzyloxy)ethyl 4-(1,2-dihydro-2-oxobenzo[d]imidazol-3-yl)piperidine-1-carbothioate;
O—(S)-2-(dibenzylamino)propyl 4-(1,2-dihydro-2-oxobenzo[d]imidazol-3-yl)piperidine-1-carbothioate;
(S)-2-(dibenzylamino)propyl 44(1,2-dihydro-2-oxobenzo[d]imidazol-3-yl)methyl)piperidine-1-carboxylate;
(S)-2-(dibenzylamino)propyl 3-(1,2-dihydro-2-oxobenzo[d]imidazol-3-yl)propylcarbamate;
(S)-2-(dibenzylamino)propyl 4-(2,3-dihydro-2-oxoimidazo[4,5-b]pyridin-1-yl)piperidine-1-carboxylate;
1-benzylpiperidin-3-yl 4-(1,2-dihydro-2-oxobenzo[d]imidazol-3-yl)piperidine-1-carboxylate;
(S)-2-(dibenzylamino)propyl 3-(1,2-dihydro-2-oxobenzo[d]imidazol-3-yl)piperidine-1-carboxylate;
(S)-2-(dibenzylamino)propyl 4-(1-benzyl-1,2-dihydro-2-oxobenzo[d]imidazol-3-yl)piperidine-1-carboxylate;
(S)-2-(dibenzylamino)propyl 441-(cyclohexylmethyl)-1,2-dihydro-2-oxobenzo[d]imidazol-3-yl)piperidine-1-carboxylate;
(S)-2-(dibenzylamino)propyl 4-(2-oxo-3-methylsulfonyl-2,3-dihydro-1H-benzo[d]imidazol-1-yl)piperidine-1-carboxylate;
(S)-2-(dibenzylamino)propyl 4-(2-methyl-1H-benzo[d]imidazol-1-yl)piperidine-1-carboxylate;
1-(S)-2-(dibenzylamino)propyl 3-ethyl 4-(1,2-dihydro-2-oxobenzo[d]imidazol-3-yl)piperidine-1,3-dicarboxylate;
(S)-2-(dibenzylamino)propyl 4-(2-oxo-3-phenylsulfonyl-2,3-dihydro-1H-benzo[d]imidazol-1-yl)piperidine-1-carboxylate;
(S)-2-(dibenzylamino)propyl 4-(1,2-dihydro-2-oxo-1-(benzoyl)benzo[d]imidazol-3-yl)piperidine-1-carboxylate;
(S)-2-(dibenzylamino)propyl 4-(6-chloro-1,2-dihydro-2-oxobenzo[d]imidazol-3-yl)piperidine-1-carboxylate;
(S)-2-(dibenzylamino)propyl 4-(3,4-dihydroquinolin-1(2H)-yl)piperidine-1-carboxylate;
(S)-2-(dibenzylamino)propyl 4-(1,2-dihydro-2-oxobenzo[d]imidazol-3-yl)azepane-1-carboxylate;
2-(diethylamino)ethyl 4-(1,2-dihydro-2-oxobenzo[d]imidazol-3-yl)piperidine-1-carboxylate;
(S)-2-(dibenzylamino)propyl 4-(1,2-dihydro-2-oxo-1-(pivaloyl)benzo[d]imidazol-3-yl)piperidine-1-carboxylate;
2-(diisopropylamino)ethyl 4-(1,2-dihydro-2-oxobenzo[d]imidazol-3-yl)piperidine-1-carboxylate;
2-(1-benzylpiperidin-4-yl)ethyl 4-(1,2-dihydro-2-oxobenzo[d]imidazol-3-yl)piperidine-1-carboxylate;
2-(ethyl(phenyl)amino)ethyl 4-(2-oxo-2,3-dihydro-1H-benzo[d]imidazol-1-yl)piperidine-1-carboxylate;
((S)-1-benzylpyrrolidin-2-yl)methyl 4-(1,2-dihydro-2-oxobenzo[d]imidazol-3-yl)piperidine-1-carboxylate;
2-(dimethylamino)ethyl 4-(1,2-dihydro-2-oxobenzo[d]imidazol-3-yl)piperidine-1-carboxylate;
3,3-diphenylpropyl 4-(1,2-dihydro-2-oxobenzo[d]imidazol-3-yl)piperidine-1-carboxylate;
(S)-2-(dibenzylamino)propyl 24(1,2-dihydro-2-oxobenzo[d]imidazol-3-yl)methyl)pyrrolidine-1-carboxylate;
(S)-2-(dibenzylamino)propyl 4-(2-(1,2-dihydro-2-oxobenzo[d]imidazol-3-yl)ethyl)piperidine-1-carboxylate;
(S)-2-(dibenzylamino)propyl 4-(1,2-dihydro-2-oxonaphtho[2,3-d]imidazol-3-yl)piperidine-1-carboxylate;
(S)-2-(dibenzylamino)propyl 4-(1H-indol-3-yl)piperidine-1-carboxy late;
(S)-2-(dibenzylamino)propyl 4-(N-phenylpropionamido)piperidine-1-carboxylate;
(S)-2-(dibenzylamino)propyl 4-(1,2-dihydro-2-oxo-1-phenylbenzo[d]imidazol-3-yl)piperidine-1-carboxylate;
1-(1-(4-(dibenzylamino)butyl)piperidin-4-yl)-1H-benzo[d]imidazol-2(3H)-one;
(S)-2-(dibenzylamino)-3-phenylpropyl 4-(1,2-dihydro-1-methyl-2-oxobenzo[d]imidazol-3-yl)piperidine-1-carboxylate;
(S)-2-(dibenzylamino)-3-phenylpropyl 4-(1,2-dihydro-3-phenylsulfonyl-2-oxobenzo[d]imidazol-1-yl)piperidine-1-carboxylate;
N—((S)-2-(dibenzylamino)propyl)-4-(1,2-dihydro-2-oxobenzo[d]imidazol-3-yl)piperidine-1-carboxamide;
N—((S)-2-(dibenzylamino)propyl)-4-(1,2-dihydro-2-oxobenzo[d]imidazol-3-yl)piperidine-1-carbothioamide;
(S)-2-(dibenzylamino)propyl 4-(3-(4-ethoxy-4-oxobutyl)-2-oxo-2,3-dihydro-1H-benzo[d]imidazol-1-yl)piperidine-1-carboxylate;
(S)-4-(3-(1-((2-(dibenzylamino)propoxy)carbonyl)piperidin-4-yl)-2-oxo-2,3-dihydro-1H-benzo[d]imidazol-1-yl)butanoic acid;
2,2-diphenylethyl 4-(1,2-dihydro-2-oxobenzo[d]imidazol-3-yl)piperidine-1-carboxylate;
(S)-2-(dibenzylamino)propyl 4-(2-oxo-3-(4-propylphenylsulfonyl)-2,3-dihydro-1H-benzo[d]imidazol-1-yl)piperidine-1-carboxylate;
(S)-2-(dibenzylamino)propyl 4-(3-(4-fluorophenylsulfonyl)-2-oxo-2,3-dihydro-1H-benzo[d]imidazol-1-yl)piperidine-1-carboxylate;
(S)-2-(dibenzylamino)propyl 4-(3-(4-methoxyphenylsulfonyl)-2-oxo-2,3-dihydro-1H-benzo[d]imidazol-1-yl)piperidine-1-carboxylate;
(S)-2-(dibenzylamino)propyl 4-(2-oxo-3-(4-(trifluoromethyl)phenylsulfonyl)-2,3-dihydro-1H-benzo[d]imidazol-1-yl)piperidine-1-carboxylate;
(S)-2-(dibenzylamino)propyl 4-(2-oxo-3-(thiophen-2-ylsulfonyl)-2,3-dihydro-1H-benzo[d]imidazol-1-yl)piperidine-1-carboxylate;
(S)-2-(dibenzylamino)propyl 4-(2-oxo-3-(quinolin-8-ylsulfonyl)-2,3-dihydro-1H-benzo[d]imidazol-1-yl)piperidine-1-carboxylate;
(S)-2-(dibenzylamino)propyl 4-(3-(naphthalen-1-ylsulfonyl)-2-oxo-2,3-dihydro-1H-benzo[d]imidazol-1-yl)piperidine-1-carboxylate;
(S)-2-(dibenzylamino)propyl 2-(1-(((S)-2-(dibenzylamino)propoxy)carbonyl)piperidin-4-ylamino)-1H-benzo[d]imidazole-1-carboxylate; ylamino)piperidine-1-carboxylate;
(S)-2-(dibenzylamino)propyl 4-(phenylamino)piperidine-1-carboxylate;
(S)-2-(dibenzylamino)propyl 4-(4-methoxyphenylamino)piperidine-1-carboxylate;
(S)-2-(dibenzylamino)propyl 4-(2,4-dimethoxyphenylamino)piperidine-1-carboxylate;
(S)-2-(dibenzylamino)propyl 4-(3,4,5-trimethoxyphenylamino)piperidine-1-carboxylate;
(S)-2-(dibenzylamino)propyl 4-(4-iodophenylamino)piperidine-1-carboxylate;
2-(N-(4-nitrobenzyl)-N-benzylamino)ethyl 4-(1,2-dihydro-2-oxobenzo[d]imidazol-3-yl)piperidine-1-carboxylate;
(S)-2-(dibenzylamino)propyl 4-(2-oxo-3-(4-acetamidyl-phenylsulfonyl)-2,3-dihydro-1H-benzo[d]imidazol-1-yl)piperidine-1-carboxylate;
(S)-2-(dibenzylamino)-3-phenylpropyl 4-(1-benzyl-1,2-dihydro-2-oxobenzo[d]imidazol-3-yl)piperidine-1-carboxylate;
N—((S)-2-(dibenzylamino)propyl)-4-(1,2-dihydro-2-oxobenzo[d]imidazol-3-yl)piperidine-1-carboxamidine;
(S)-2-(dibenzylamino)propyl 4-(1,3-diphenylureido)piperidine-1-carboxylate;
(S)-2-(dibenzylamino)propyl 4-(N-phenylbenzamido)piperidine-1-carboxylate;
(S)-2-(dibenzylamino)propyl 4-(N-phenylphenylsulfonamido)piperidine-1-carboxylate;
(S)-2-(dibenzylamino)propyl 4-(3-fluorophenylamino)piperidine-1-carboxylate;
(S)-2-(dibenzylamino)propyl 4-(3,4-dichlorophenylamino)piperidine-1-carboxylate;
(S)-2-(dibenzylamino)propyl 4-(2-aminophenylamino)piperidine-1-carboxylate;
(S)-2-(dibenzylamino)propyl 4-(1-acetyl-1,2-dihydro-2-oxobenzo[d]imidazol-3-yl)piperidine-1-carboxylate;
(S)(1-((2-(dibenzylamino)propoxy)carbonyl)piperidin-4-yl)-5,6-dihydro-imidazo[4,5,1,j,k][1]benzazepine-2,7(1H,4H)-dione;
(S)-2-(dibenzylamino)propyl 4-(3-(2-tert-butyloxycarbonylaminoethyl)-2-oxo-2,3-dihydro-1H-benzo[d]imidazol-1-yl)piperidine-1-carboxylate;
(S)-2-(dibenzylamino)-3-phenylpropyl 4-(3-(2-tert-butyloxy carbonylaminoethyl)-2-oxo-2,3-dihydro-1H-benzo[d]imidazol-1-yl)piperidine-1-carboxylate;
(S)-2-(dibenzylamino)-3-phenylpropyl 4-(3-(2 aminoethyl)-2-oxo-2,3-dihydro-1H-benzo[d]imidazol-1-yl)piperidine-1-carboxylate D-biotin amid;
(S)-2-(dibenzylamino)propyl 3-(1,2-dihydro-2-oxobenzo[d]imidazol-3-yl)pyrrolidine-1-carboxylate;
1-[1-(4-dibenzylamino-butyryl)-piperidin-4-yl]-1,3-dihydro-benzoimidazol-2-one;
2-(benzyl(2-(benzyloxy)benzyl)amino)ethyl 4-(2-oxo-2,3-dihydro-1H-benzo[d]imidazol-1-yl)piperidine-1-carboxylate;
2-(benzyl(4-(benzyloxy)benzyl)amino)ethyl 4-(2-oxo-2,3-dihydro-1H-benzo[d]imidazol-1-yl)piperidine-1-carboxylate;
2-(benzyl(2,6-difluorobenzyl)amino)ethyl 4-(2-oxo-2,3-dihydro-1H-benzo[d]imidazol-1-yl)piperidine-1-carboxylate;
(S)-2-(benzyl(furan-2-ylmethyl)amino)propyl 4-(2-oxo-2,3-dihydro-1H-benzo[d]imidazol-1-yl)piperidine-1-carboxylate;
2-(benzyl(3-(benzyloxy)benzyl)amino)ethyl 4-(2-oxo-2,3-dihydro-1H-benzo[d]imidazol-1-yl)piperidine-1-carboxylate;
(E)-2-(benzyl(cinnamyl)amino)ethyl 4-(2-oxo-2,3-dihydro-1H-benzo[d]imidazol-1-yl)piperidine-1-carboxylate;
(S)-2-(benzyl(tert-butoxycarbonyl)amino)-4-methylpentyl 4-(2-oxo-2,3-dihydro-1H-benzo[d]imidazol-1-yl)piperidine-1-carboxylate;
(S)-2-(benzyl(tert-butoxycarbonyl)amino)-3-phenylpropyl 4-(2-oxo-2,3-dihydro-1H-benzo[d]imidazol-1-yl)piperidine-1-carboxylate;
(S)-2-(dibenzylamino)-3-methylbutyl 4-(1,2-dihydro-2-oxobenzo[d]imidazol-3-yl)piperidine-1-carboxylate;
(S)-2-(dibenzylamino)-4-methylpentyl 4-(1,2-dihydro-2-oxobenzo[d]imidazol-3-yl)piperidine-1-carboxylate;
2-(dibenzylamino)-3,3-dimethylbutyl 4-(1,2-dihydro-2-oxobenzo[d]imidazol-3-yl)piperidine-1-carboxylate;
(3-(dibenzylamino)butyl 4-(1,2-dihydro-2-oxobenzo[d]imidazol-3-yl)piperidine-1-carboxylate;
2-(dibenzylamino)-2-methylpropyl 4-(1,2-dihydro-2-oxobenzo[d]imidazol-3-yl)piperidine-1-carboxylate;
(S)-2-(dibenzylamino)-2-phenylethyl 4-(2-oxo-2,3-dihydro-1H-benzo[d]imidazol-1-yl)piperidine-1-carboxylate;
(S)-2-(benzyl(4-nitrobenzyl)amino)propyl 4-(2-oxo-2,3-dihydro-1H-benzo[d]imidazol-1-yl)piperidine-1-carboxylate;
3-(benzyl(4-nitrobenzyl)amino)propyl 4-(2-oxo-2,3-dihydro-1H-benzo[d]imidazol-1-yl)piperidine-1-carboxylate;
2-(benzyl(4-bromobenzyl)amino)ethyl 4-(2-oxo-2,3-dihydro-1H-benzo[d]imidazol-1-yl)piperidine-1-carboxylate;
S)-2-(benzyl(4-bromobenzyl)amino)propyl 4-(2-oxo-2,3-dihydro-1H-benzo[d]imidazol-1-yl)piperidine-1-carboxylate;
3-(benzyl(4-methoxybenzyl)amino)propyl 4-(2-oxo-2,3-dihydro-1H-benzo[d]imidazol-1-yl)piperidine-1-carboxylate;
(R)-2-(dibenzylamino)-3-phenylpropyl 4-(2-oxo-2,3-dihydro-1H-benzo[d]imidazol-1-yl)piperidine-1-carboxylate;
2-(benzyl(4-fluorobenzyl)amino)ethyl 4-(2-oxo-2,3-dihydro-1H-benzo[d]imidazol-1-yl)piperidine-1-carboxylate;
2-(benzyl(3-phenylpropyl)amino)ethyl 4-(2-oxo-2,3-dihydro-1H-benzo[d]imidazol-1-yl)piperidine-1-carboxylate;
2-(benzyl(4-methoxybenzyl)amino)ethyl 4-(2-oxo-2,3-dihydro-1H-benzo[d]imidazol-1-yl)piperidine-1-carboxylate;
2-(benzyl(3-methoxybenzyl)amino)ethyl 4-(2-oxo-2,3-dihydro-1H-benzo[d]imidazol-1-yl)piperidine-1-carboxylate;
2-(benzyl(3-phenoxybenzyl)amino)ethyl 4-(2-oxo-2,3-dihydro-1H-benzo[d]imidazol-1-yl)piperidine-1-carboxylate;
2-(benzyl(3-chlorobenzyl)amino)ethyl 4-(2-oxo-2,3-dihydro-1H-benzo[d]imidazol-1-yl)piperidine-1-carboxylate;
(S)-2-(benzyl(3-phenylpropyl)amino)propyl 4-(2-oxo-2,3-dihydro-1H-benzo[d]imidazol-1-yl)piperidine-1-carboxylate;
(S)-2-(benzyl(3-methoxybenzyl)amino)propyl 4-(2-oxo-2,3-dihydro-1H-benzo[d]imidazol-1-yl)piperidine-1-carboxylate;
(S)-2-(benzyl(3-phenoxybenzyl)amino)propyl 4-(2-oxo-2,3-dihydro-1H-benzo[d]imidazol-1-yl)piperidine-1-carboxylate;
(S)-2-(benzyl(3-chlorobenzyl)amino)propyl 4-(2-oxo-2,3-dihydro-1H-benzo[d]imidazol-1-yl)piperidine-1-carboxylate;
(S)-2-(benzyl(4-chlorobenzyl)amino)propyl 4-(2-oxo-2,3-dihydro-1H-benzo[d]imidazol-1-yl)piperidine-1-carboxylate;
2-(benzyl(naphthalen-1-ylmethyl)amino)ethyl 4-(2-oxo-2,3-dihydro-1H-benzo[d]imidazol-1-yl)piperidine-1-carboxylate;
2-(benzyl(quinolin-4-ylmethyl)amino)ethyl 4-(2-oxo-2,3-dihydro-1H-benzo[d]imidazol-1-yl)piperidine-1-carboxylate;
2-(benzyl((1-methyl-1H-indol-2-yl)methyl)amino)ethyl 4-(2-oxo-2,3-dihydro-1H-benzo[d]imidazol-1-yl)piperidine-1-carboxylate;
(S)-2-(benzyl(quinolin-4-ylmethyl)amino)propyl 4-(2-oxo-2,3-dihydro-1H-benzo[d]imidazol-1-yl)piperidine-1-carboxylate;
3-(benzyl(pyridin-3-ylmethyl)amino)propyl 4-(2-oxo-2,3-dihydro-1H-benzo[d]imidazol-1-yl)piperidine-1-carboxylate;
3-(benzyl(pyridin-2-ylmethyl)amino)propyl 4-(2-oxo-2,3-dihydro-1H-benzo[d]imidazol-1-yl)piperidine-1-carboxylate;
(S)-2-(benzyl(3-(benzyloxy)benzyl)amino)propyl 4-(2-oxo-2,3-dihydro-1H-benzo[d]imidazol-1-yl)piperidine-1-carboxylate;
(S)-2-(benzyl(phenanthren-9-ylmethyl)amino)propyl 4-(2-oxo-2,3-dihydro-1H-benzo[d]imidazol-1-yl)piperidine-1-carboxylate;
(S)-2-(benzyl(naphthalen-1-ylmethyl)amino)propyl 4-(2-oxo-2,3-dihydro-1H-benzo[d]imidazol-1-yl)piperidine-1-carboxylate;
(S)-2-(benzyl(thiophen-2-ylmethyl)amino)propyl 4-(2-oxo-2,3-dihydro-1H-benzo[d]imidazol-1-yl)piperidine-1-carboxylate;
(S)-2-(benzyl(pyridin-2-ylmethyl)amino)propyl 4-(2-oxo-2,3-dihydro-1H-benzo[d]imidazol-1-yl)piperidine-1-carboxylate;
3-(benzyl(thiophen-2-ylmethyl)amino)propyl 4-(2-oxo-2,3-dihydro-1H-benzo[d]imidazol-1-yl)piperidine-1-carboxylate;
3-(benzyl(3-chlorobenzyl)amino)propyl 4-(2-oxo-2,3-dihydro-1H-benzo[d]imidazol-1-yl)piperidine-1-carboxylate;
3-(benzyl(2-(benzyloxy)ethyl)amino)propyl 4-(2-oxo-2,3-dihydro-1H-benzo[d]imidazol-1-yl)piperidine-1-carboxylate;
3-(benzyl(naphthalen-1-ylmethyl)amino)propyl 4-(2-oxo-2,3-dihydro-1H-benzo[d]imidazol-1-yl)piperidine-1-carboxylate;
3-(benzyl(quinolin-4-ylmethyl)amino)propyl 4-(2-oxo-2,3-dihydro-1H-benzo[d]imidazol-1-yl)piperidine-1-carboxylate;
3-(benzyl(3-phenoxybenzyl)amino)propyl 4-(2-oxo-2,3-dihydro-1H-benzo[d]imidazol-1-yl)piperidine-1-carboxylate;
3-(benzyl(4-fluorobenzyl)amino)propyl 4-(2-oxo-2,3-dihydro-1H-benzo[d]imidazol-1-yl)piperidine-1-carboxylate;
3-(benzyl(3-phenylpropyl)amino)propyl 4-(2-oxo-2,3-dihydro-1H-benzo[d]imidazol-1-yl)piperidine-1-carboxylate;
3-(benzyl(3-methoxybenzyl)amino)propyl 4-(2-oxo-2,3-dihydro-1H-benzo[d]imidazol-1-yl)piperidine-1-carboxylate;
2-(benzyl(4-chlorobenzyl)amino)ethyl 4-(2-oxo-2,3-dihydro-1H-benzo[d]imidazol-1-yl)piperidine-1-carboxylate;
2-(benzyl(2-methoxybenzyl)amino)ethyl 4-(2-oxo-2,3-dihydro-1H-benzo[d]imidazol-1-yl)piperidine-1-carboxylate;
(S)-2-(dibenzylamino)-3-phenylpropyl 4-(phenylamino)piperidine-1-carboxylate;
(S)-2-(dibenzylamino)propyl 4-(2-phenyl-1H-benzo[d]imidazol-1-yl)piperidine-1-carboxylate;
(S)-2-(dibenzylamino)propyl 4-(N-phenyl-4-propylphenylsulfonamido)piperidine-1-carboxylate;
(S)-2-(dibenzylamino)propyl 4-(N-phenyl-4-fluorophenylsulfonamido)piperidine-1-carboxylate;
(S)-2-(dibenzylamino)propyl 4-(N-phenyl-4-methoxyphenylsulfonamido)piperidine-1-carboxylate;
(S)-2-(dibenzylamino)-3-phenylpropyl 4-(N-phenyl-4-methoxyphenylsulfonamido)piperidine-1-carboxylate;
(S)-2-(dibenzylamino)propyl 4-(N-phenyl-4-(trifluoromethyl)phenylsulfonamido)piperidine-1-carboxylate;
(S)-2-(dibenzylamino)propyl 4-(N-phenylthiophen-2-ylsulfonamido)piperidine-1-carboxylate;
(S)-2-(dibenzylamino)propyl 4-(N-phenylquinolin-8-ylsulfonamido)piperidine-1-carboxylate;
(S)-2-(dibenzylamino)propyl 4-(N-phenyl-naphthalen-1-ylsulfonamido)piperidine-1-carboxylate;
(S)-2-(dibenzylamino)-3-phenylpropyl 4-(N-phenyl-naphthalen-1-ylsulfonamido)piperidine-1-carboxylate;
(S)-2-(dibenzylamino)propyl 4-(N-phenylpivalamido)piperidine-1-carboxylate;
(S)-2-(dibenzylamino)propyl 4-(2-(phenylsulfonyl)phenylamino)piperidine-1-carboxylate;
(S)-2-(dibenzylamino)propyl 4-(1,3-diphenylthioureido)piperidine-1-carboxylate;
(S)-2-(dibenzylamino)propyl 4-(N-methyl-N-phenylamino)piperidine-1-carboxylate;
(S)-2-(dibenzylamino)propyl 4-(N-phenyl-2-methylphenylsulfonamido)piperidine-1-carboxylate;
(S)-2-(dibenzylamino)propyl 4-(N-phenyl-3-methylphenylsulfonamido)piperidine-1-carboxylate;
(S)-2-(dibenzylamino)propyl 4-(N-phenyl-3-(trifluoromethyl)phenylsulfonamido)piperidine-1-carboxylate;
(S)-2-(dibenzylamino)propyl 4-(N-phenyl-biphenyl-4-sulfonamido)piperidine-1-carboxylate;
(S)-2-(dibenzylamino)propyl 4-(N-phenyl-naphthalen-2-ylsulfonamido)piperidine-1-carboxylate;
(S)-2-(dibenzylamino)propyl 4-(N-phenyl-(5-dimethylamino)naphthalen-1-ylsulfonamido)piperidine-1-carboxylate;
(S)-2-(dibenzylamino)propyl 4-(2-(4-chlorophenylthio)phenylamino)piperidine-1-carboxylate;
(S)-2-(dibenzylamino)-3-phenylpropyl 4-(2,6-dimethylphenylamino)piperidine-1-carboxylate;
(S)-2-(dibenzylamino)-3-phenylpropyl diethylphenylamino)piperidine-1-carboxylate;
(S)-2-(dibenzylamino)-3-phenylpropyl 4-(2-(methylthio)phenylamino)piperidine-1-carboxylate;
(S)-2-(dibenzylamino)propyl 4-(2-amino-1H-benzo[d]imidazol-1-yl)piperidine-1-carboxylate;
4-(dibenzylamino)phenyl 4-(1,2-dihydro-2-oxobenzo[d]imidazol-3-yl)piperidine-1-carboxylate;
(S)-2-(dibenzylamino)propyl 4-(2-oxo-2,3-dihydro-1H-benzo[d]imidazol-4-yl)piperidine-1-carboxylate;
6-(dibenzylamino)hexyl 4-(1,2-dihydro-2-oxobenzo[d]imidazol-3-yl)piperidine-1-carboxylate;
Benzyl 4-(1,2-dihydro-2-oxobenzo[d]imidazol-3-yl)piperidine-1-carboxylate;
(1-benzylpiperidin-2-yl)methyl 4-(1,2-dihydro-2-oxobenzo[d]imidazol-3-yl)piperidine-1-carboxylate;
(S)-2-(dibenzylamino)-3-phenylpropyl 4-(N-phenyl-4-(trifluoromethyl)phenylsulfonamido)piperidine-1-carboxylate;
(S)-2-(benzyl(4-fluorobenzyl)amino)propyl 4-(2-oxo-2,3-dihydro-1H-benzo[d]imidazol-1-yl)piperidine-1-carboxylate;
2-(benzyl(tert-butoxycarbonyl)amino)ethyl 4-(2-oxo-2,3-dihydro-1H-benzo[d]imidazol-1-yl)piperidine-1-carboxylate;
3-(benzyl((1-methyl-1H-indol-2-yl)methyl)amino)propyl 4-(2-oxo-2,3-dihydro-1H-benzo[d]imidazol-1-yl)piperidine-1-carboxylate hydrochloride;
2-(benzyl(4-(methoxycarbonyl)benzyl)amino)ethyl 4-(2-oxo-2,3-dihydro-1H-benzo[d]imidazol-1-yl)piperidine-1-carboxylate;
3-(benzyl(3-(benzyloxy)benzyl)amino)propyl 4-(2-oxo-2,3-dihydro-1H-benzo[d]imidazol-1-yl)piperidine-1-carboxylate;
(S)-2-(benzylamino)-4-methylpentyl 4-(2-oxo-2,3-dihydro-1H-benzo[d]imidazol-1-yl)piperidine-1-carboxylate;
2-(benzylamino)-3-phenylpropyl 4-(2-oxo-2,3-dihydro-1H-benzo[d]imidazol-1-yl)piperidine-1-carboxylate and pharmaceutically acceptable salts thereof.

The pharmaceutically acceptable salts of the compounds of formula (I) can be acid addition salts formed with pharmaceutically acceptable acids. Such a definition encompasses hydrochloride, hydrobromide, sulphate or bisulphate, phosphate or hydrogenophosphate, acetate, benzoate, succinate, fumarate, maleate, lactate, citrate, tartrate, gluconate, methanesulphonate, benzene-sulphonate, paratoluene-sulphonate salts of compounds of formula (I).

Two different protocols can be employed for the preparation of all compounds of the above defined formula (I):
a classical solution phase synthesis and
a solid phase synthesis on solid support.

Firstly, in the case of classical solution phase synthesis, all compounds of formula (I) according to the present Invention can be prepared using state of the art methodologies which are briefly described here. Final compounds belong to general structure I which was decomposed in 3 substructures A, B, and C with a link (“X”) between the substructures B and C as depicted in the following formula:

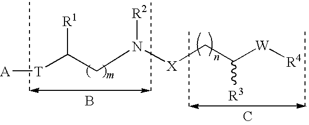

in which A, T, R¹, R², R³, R⁴, m, n, X and W can have the same significations than in formula (I) above.

Several strategies can be employed which all include a link procedure. The linkage takes place using either synthetic A-B or some commercially available A-B as building blocks but also B blocks for a later introduction of the A aryl/heteroaryl moiety. Then, scheme 1 bellow illustrates the linking procedures between AB and C or between B [(II-a) or (II-b)] and C:

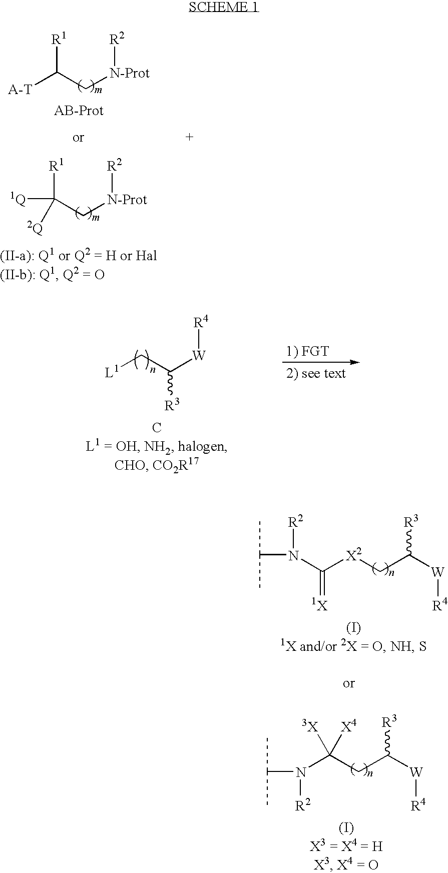

in which A, T, R¹, R², R³, R⁴, m, n, W can have the same significations than in formula (I) as defined above; Prot represents a classical protective group, L¹ represents OH, NH₂, halogen, CHO or COOR¹⁷ whereas R¹⁷ represents hydrogen, halogen or alkyl, X¹ and X², which may be identical or different, represent O, NH or S and X³ and X⁴, which are identical, represent H or O. Protecting groups are well known from the one skilled in the art and are described for example in “Protective groups in organic synthesis”, T. W. GREENE et al., 2^{nd} edition, Wiley Interscience, 1991. As examples of these protecting groups, one can mention benzyl; benzyloxycarbonyle (Z); trifluoroacetyle (TFA); tert-butyloxycarbonyle (Boc); trimethylsilylethoxycarbonyle (Teoc) and fluorenylmethyloxycarbonyle (Fmoc) groups. These protecting groups can be removed via hydrogenation or acidic treatment (for example with trifluoroacetic acid: TFA) using the standard corresponding protocols.

Intermediary compounds of formula C in which L¹=NH₂ are either commercially available or prepared from the corresponding alcohol using phtalimide followed by hydrazine deprotection as described for example in the article by Berger, Y., et al., J. Med. Chem., 2005, 48, 483-498. In the case where compounds of formula C are non commercial aminoalcohols (L¹=OH, W═N—R¹⁶), they can be prepared by amine alkylation using the convenient halide in the presence of potassium carbonate as described for example by Le Bihan, G. et al., J. Med. Chem., 1999, 42, 1587-1603, or functional group transformation (FGT) starting from commercially available aminoacids. FGT also means for protection/deprotection sequences.

For alkyl chain length (n) variations, compounds can be obtained after addition of dialkylamines on different cyclic anhydrides followed by standard hydride reduction or alkylations using the convenient halogeno alcohols. Aldehydes can be obtained by diisobutylaluminium hydride reduction of the corresponding methylesters. Acids and derivatives may be obtained after esters hydrolysis using hydrogen chloride in methanol for hydrolysis. The esters precursors are prepared by nucleophilic displacement with a corresponding reactive H—WR⁴. When L¹=halogen, compounds can be prepared from the corresponding alcohols by halogenation for example using triphenylphosphin with carbon tetrabromide according to the methods classically used by the one skilled in the art.

The link procedure between A-B and C or between intermediate of formula (II) and C depends on the nature of L¹. When L¹=OH or NH₂, carbamates or ureas can be classically prepared using treatment of alcohol or amines with carbonyldiimidazol (CDI) as described for example by Matsuno, K. et al., J. Med. Chem., 2002, 45, 3057-3066, followed by nucleophilic displacement with the corresponding amines. For thiocarbamates preparation, sodium salt of the corresponding alcohols (L¹=OH) are first treated with carbondisulfide in hot methanol followed by methylation with methyl iodide. The corresponding dithiocarbonates obtained are then added to the different (II-a)/(II-b) or A-B moieties. For thioureas preparation (L¹=NH₂), amines are added to carbondisulfide in acetonitrile generating the corresponding thiols which are activated by 1-(3-dimethylaminopropyl)-3-ethylcarbodiimide hydrochloride (EDCI) before addition of the (II-a)/(II-b) or AB moieties in order to afford thioureas.

When L¹=NH₂, guanidines can be obtained by successive addition of the two corresponding amines on diimidazolmethanimine prepared in a mixture tetrahydrofurane/dimethylformamide (THF/DMF) as described for example in the article by Wu, Y.-Q. et al., J. Org. Chem., 2002, 67, 7553-7556.

Other compounds can be obtained either by amines alkylation of the corresponding halides using potassium carbonate with catalytic amounts of potassium iodide in hot DMF or reductive amination of the corresponding aldehydes using sodium triacetoxy borohydride in dichloroethane (DCE).

Amides are obtained by nucleophilic addition of the (II-a)/(II-b) or A-B moieties on acyl chlorides using triethylamine (TEA) in DCM (dichloromethane). Acyl chlorides are conveniently prepared from the corresponding acids by thionyl chloride treatment.

The linking method between A and BC and A and B are represented on the following Schemes 2 and 3:

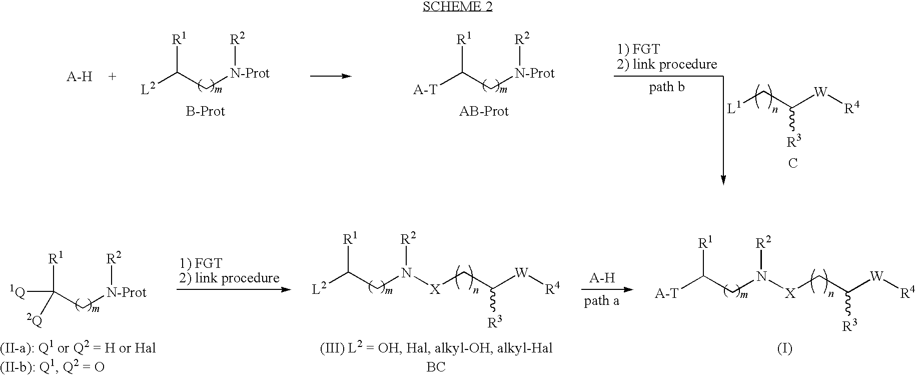

in which in which A, T, R¹, R², R³, R⁴, m, n, X and W have the same significations as in formula (I) as defined above; Prot represents a classical protective group as defined in scheme 1, L² represents OH, halogen, hydroxyalkyl or halogenoalkyl.

Different aryl/heteroaryl moieties A-H can be attached to the corresponding alcohols (B-C type, path a) using the method according to Mitsunobu O. (Synthesis, 1981, 1-28) which is a well established method for the preparation of amines from alcohols, or using standard alkylation procedures. In the case when part C is further connected (path b), protective groups, such a those mentioned before for scheme 1, are used. Deprotection and link procedures are employed as previously described affording final compounds.

In some cases alcohols can be obtained from the corresponding ketones using sodium borohydride reduction.

Connexions between A and BC or between A and B can also be performed by a reductive amination process as described on Scheme 3 below:

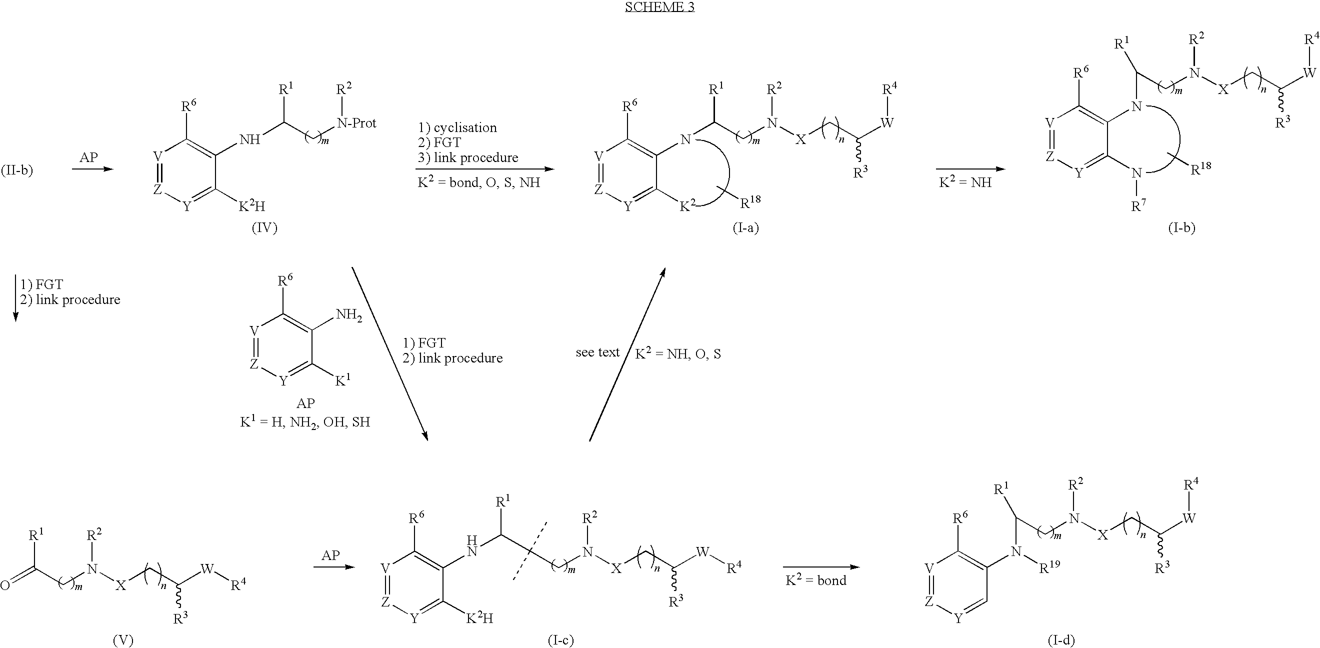

in which V, Z, Y, R¹, R², R³, R⁴, R⁶, R⁷, m, n, X, W can have the same significations than those recited for formula (I) and sub-formula (A-1) as defined above; Prot represents a classical protective group as defined in scheme 1, K¹ represents H, OH, SH or NH₂, K² represents a bond or O, S atoms or a NH group, R¹⁸ represents C(=Het)-, C(=Het)CH₂—, C(=Het)C(=Het), C(=Het)CH₂CH₂—; Het having the same signification as previously mentioned for (I).

According to this scheme, final compounds can be obtained upon two interconnected pathways starting from ketone of formula (II-b):
by connecting the A ring precursor (AP) to (II-b) by reductive amination, leading to compound (IV) which undergoes cyclisation and link procedure; or
by performing first the link procedure leading to compound of formula (V) following by (AP) connection and further transformations.

In the case of aniline derivatives, the secondary amines obtained (I-c) are converted into compounds (I-d) where R¹⁹ represents a hydrogen atom, an alkyl group, an alkenyl group, an aryl group, a heteroaryl group or a group selected from the groups of the following formulas —COOR²⁰, —C(O)NHR²⁰, —C(O)R²⁰, —C(S)R²⁰, —SO₂R²⁰ in which R²⁰ represents a hydrogen atom, an alkyl group, an alkenyl group, an aryl or heteroaryl groups; wherein said aryl, heteroaryl are optionally substituted with one or more groups independently selected from halogen, trifluoromethyl, difluoromethyl, azido, alkyl, alkoxy, cyano, nitro and carboalkoxy.

The conversion can be performed using different electrophiles such as isocyanates, acyls chlorides, sulfonyl chlorides in the presence of triethylamine in dichloromethane (DCM).

In the case of compounds of formula (I-c) and (IV), the corresponding mono alkylated compounds are cycled using carbonyldimidazol (CDI) or others reagents such as glyoxal, glyoxylates, pyruvates, halogenoalkyl acyl halides or carboxylic acids in order to obtain the fused new heterocyclic structures bearing R¹⁸ as defined before.

In the case of some dihydro aromatic heterocyclic moieties, oxidation in order to get the full aromatic structure can be performed using 2,3-dichloro-5,6-dicyano-1,4-benzoquinone (DDQ) as for example described in the article by Zhang, X. et al., Bioorg. Med. Chem. Lett., 2004, 14, 6011-6016.

The precedent methodology is also used with different cyclic amino-ketones (V) already linked to part C as previously described. In some cases, when a carbonyl group adjacent to the keto function (CO₂alkyl) is present, supported cyanoborohydride can be used with acetic acid in methanol instead of the sodium triacetoxyborydride procedure as described for example in the article by Ley, S. V et al., J. Chem. Soc., Perkin Trans 1, 1998, 1, 2239-2241. In some cases when bifunctional reagents are used for alkylation (vide infra) of molecules of type (I-c), the corresponding adducts are cycled affording new heterocyclic structures A.

Sodium salts of molecules of formula (I-a) in which K²═NH, obtained upon NaH treatment are treated with different electrophiles such as acyl chlorides, sulfonyl chlorides, alkyl halides according to methods well known in the art. Aryl substitution was also performed using copper catalysed coupling reactions according to a method as described for example in the article by Lam, P. Y. S. et al., Tetrahedron Lett., 2001, 42, 3415-3418.

In the case of alkylations with alkyl halides reactions take usually place in DMF or THF with a catalytic amount of potassium iodide.

In the case of alkyl group with carbonyl function, acyl activated compounds are obtained by the corresponding esters hydrolysis with sodium hydroxide and are treated upon Friedel et Crafts conditions in order to obtain cyclic compounds (Salaski, E. J., Tetrahedron Lett., 1995, 36, 1387-1390).

Compounds of type (I-a) are functionalized via alkylamino chain linker and eventually connected to another carboxylic acid moiety via classical peptide coupling using O-benzotriazole-N,N,N′,N′-tetramethyl-uronium-hexafluoro-phosphate (HBTU). In the case of benzimidazolones, sodium salts generated with NaH are used in DMF for the last stage alkylation or acylation reactions.

The corresponding thioureas are also obtained exclusively from some A rings using Lawesson reagent as described for example in the article by Zhang, P. et al., Bioorg. Med. Chem. Lett., 2001, 11, 2747.

Other AB fragments can be obtained using diverse methods: for example condensation of indolinone sodium salt with N-protected amino ketones of the formula (II-b) type afforded insaturated compounds which can also be reduced by hydrogenation in acetic acid dichloromethane. After deprotection, the same procedure as those detailed above can be used to link the different moieties.

Benzimidazoles (compounds of formula (I) in which A is a moiety of formula (A-2) as above-defined and T is —O— or —NH—) can be prepared using classical methods starting either from the corresponding diaminoarylic precursors AP which reacted with a carbonyl derivative of formula (II-b) as above-defined, or starting from chlorobenzimidazole by nucleophilic substitution with different amines according to the well known in the art methods.

In the case of protected amines, deprotection was followed by link procedure in order to connect to C fragment. Cyclisations were performed heating with carboxylic acids according to well know methods.

Secondly, it is also possible to synthesise compounds of formula (I) according to the present invention, using solid support library synthesis

Solid support parallel synthesis uses an adaptation to library production of the triazene linker strategy developed by Brase, S. et al., Angew. Chem. Int. Ed., 1998, 37, 3413-3415 and Brase, S., Acc. Chem. Res., 2004, 37, 8005-8016. This methodology allows the production of diazonium salts upon cleavage. The known properties of theses salts allow the possibility of a traceless synthesis via reduction or functionalisation. This methodology is used for the complete construction of the final compounds of formula (I) as previously defined and can be schematically represented on the following schemes 4 and 5:

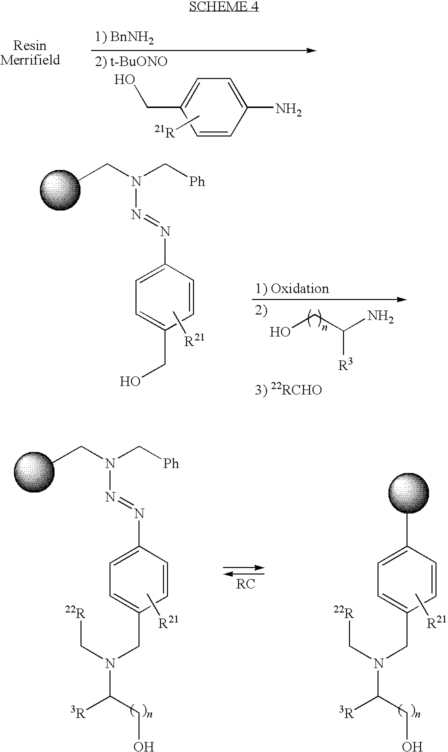

in which R³ and n have the same definition as in general formula (I), R²¹ represents hydrogen, halogen, or an alkoxy or alkyl group, R²² represents a group selected among alkyl, alkenyl, cycloalkyl, aryl, heteroaryl, arylalkyl and heteroarylalkyl groups wherein said groups defined for R²² are optionally substituted with one or more groups independently selected from halogen, trifluoromethyl, difluoromethyl, azido, alkyl, alkoxy, cyano, nitro and carbethoxy.

Starting from Merrifield resin, benzylamin resin can be obtained using the reported procedure and triazene are formed by action of sodium tert-butyl nitrite, Lewis acid and the corresponding anilines such as 4-hydroxymethyl aniline (R²¹═H). Oxidation to aldehyde takes place using ioded-based reagents such as 1-hydroxy-1,2-benziodoxol-3(1H)-one 1-oxide (IBX) or 1,1,1-triacetoxy-1,1-dihydro-1,2-benziodoxol-3(1H)-one (Dess Martin periodinane). First element of diversity is introduced via the reductive amination using the selected aminoalcohols bearing R³ group. Second element of diversity is introduced via a second reductive amination using the selected aldehydes.

Starting from those supported aminoalcohols, two synthetic pathways (Paths a and b) can be used depending of linker stability toward further reactions employed according to Scheme 5 below:

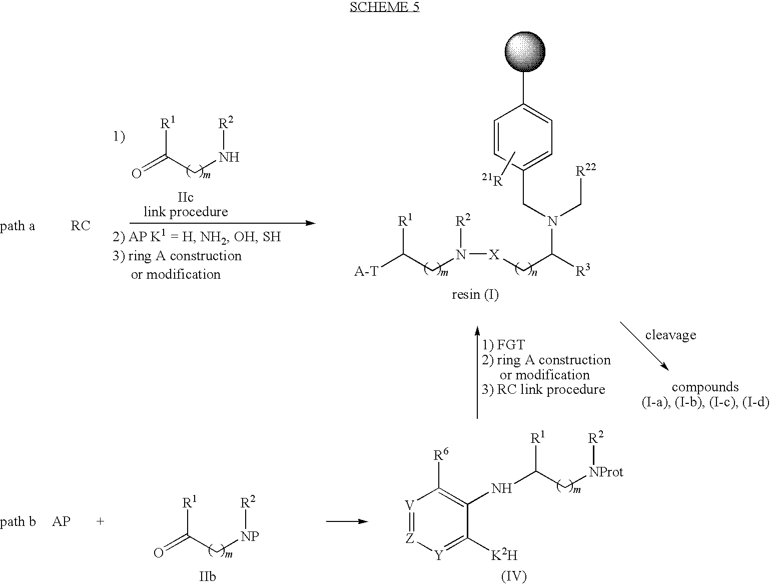

in which A, T, R¹, R², R⁶, m, n, V, Z, Y and X have the same meanings as those defined for formula (I), K¹ and K² have the same meanings as those defined in Scheme 3 and Prot has the same signification as those defined for scheme 2.

According to Path a, (see Scheme 5), compounds of formula (I) are obtained in a linear <<all supported>> way: i.e. starting from the aminoalcohols. Resins R—C are first connected using the link procedure as described above with aminoketones (II-c), followed by reductive amination with precursor AP and A ring functionalisation/building using adapted versions of already discussed methodologies afforded supported final compounds Resin (I).

According to Path b, compounds of formula (I) are obtained using a <<catch & release>> methodology allowing a convergent synthesis: first molecules of formula (IV) are prepared without any purification in solution phase as described before and functionnalized or cyclized prior to the connection to the resin R—C with the link procedure affording Resin (I).

Cleavages of compounds (I) from the resin were performed on Resin (I) with treatments using an acid reagent such as TFA or trichlorosilane (HSiCl₃) with an adapted procedure of the literature and afforded compounds of formula (I), i.e. of formulas (I-a) to (I-d) as above described.

All the reactions used to obtain compounds of formula (I) are classically used in organic chemistry and are well known from the one skilled in the art.

After the synthesis, compounds of formula (I) according to the present invention can be recovered and purified according to methods also classically used in the art.

As it will be demonstrated in the examples illustrating the Invention, compounds of formula (I) according to the present Invention, (including pharmaceutically acceptable salts thereof), have a potent activity on the inhibition of the proliferation of apicomplexan parasites. Compounds of formula (I) then make possible to prevent and/or treat parasitic diseases involving apicomplexan parasites, when they are administered alone or in combination with usual antiparasitic drugs.

Another subject matter of the invention is therefore the compounds of formula (I) for a use as a drug, in particular as a drug for the prevention and/or the treatment of parasitic diseases involving apicomplexan parasites. According to a very preferred embodiment of the invention, the drug is for the prevention and/or the treatment of malaria or toxoplasmosis.

When the drug is intended for the prevention and/or treatment of malaria, compounds of formula (I) are preferably selected in the group consisting of:
(S)-2-(dibenzylamino)propyl 4-(1H-benzo[d]imidazol-2-ylamino)piperidine-1-carboxylate;
(S)-2-(dibenzylamino)propyl 4-(2-oxo-3-phenylsulfonyl-2,3-dihydro-1H-benzo[d]imidazol-1-yl)piperidine-1-carboxylate;
(S)-2-(dibenzylamino)propyl 4-(3-(4-fluorophenylsulfonyl)-2-oxo-2,3-dihydro-1H-benzo[d]imidazol-1-yl)piperidine-1-carboxylate;
(S)-2-(dibenzylamino)propyl 4-(3-(4-methoxyphenylsulfonyl)-2-oxo-2,3-dihydro-1H-benzo[d]imidazol-1-yl)piperidine-1-carboxylate;
(S)-2-(dibenzylamino)propyl 4-(2-oxo-3-(4-(trifluoromethyl)phenylsulfonyl)-2,3-dihydro-1H-benzo[d]imidazol-1-yl)piperidine-1-carboxylate;
(S)-2-(dibenzylamino)-3-phenylpropyl 4-(1,2-dihydro-1-methyl-2-oxobenzo[d]imidazol-3-yl)piperidine-1-carboxylate;
(S)-2-(dibenzylamino)propyl 4-(2-oxo-3-(thiophen-2-ylsulfonyl)-2,3-dihydro-1H-benzo[d]imidazol-1-yl)piperidine-1-carboxylate;
N—((S)-2-(dibenzylamino)propyl)-4-(1,2-dihydro-2-oxobenzo[d]imidazol-3-yl)piperidine-1-carboxamidine;
(S)-2-(dibenzylamino)propyl 4-(N-phenylphenylsulfonamido)piperidine-1-carboxylate;
(S)-2-(dibenzylamino)propyl 4-(3-(4-ethoxy-4-oxobutyl)-2-oxo-2,3-dihydro-1H-benzo[d]imidazol-1-yl)piperidine-1-carboxylate;
(S)-2-(dibenzylamino)propyl 4-(1-benzyl-1,2-dihydro-2-oxobenzo[d]imidazol-3-yl)piperidine-1-carboxylate;
(S)-2-(dibenzylamino)propyl 4-(2-oxo-3-(quinolin-8-ylsulfonyl)-2,3-dihydro-1H-benzo[d]imidazol-1-yl)piperidine-1-carboxylate;
tert-butyl 3-(1-(((S)-2-(dibenzylamino)propoxy)carbonyl)piperidin-4-yl)-2,3-dihydro-2-oxobenzo[d]imidazole-1-carboxylate;
(S)-2-(dibenzylamino)-3-phenylpropyl 4-(1-benzyl-1,2-dihydro-2-oxobenzo[d]imidazol-3-yl)piperidine-1-carboxylate;
(S)-2-(dibenzylamino)propyl 4-(3-(naphthalen-1-ylsulfonyl)-2-oxo-2,3-dihydro-1H-benzo[d]imidazol-1-yl)piperidine-1-carboxylate;
(S)-2-(dibenzylamino)propyl 4-(1-(cyclohexylmethyl)-1,2-dihydro-2-oxobenzo[d]imidazol-3-yl)piperidine-1-carboxylate;
(S)-2-(dibenzylamino)propyl 4-(1,2-dihydro-2-thioxobenzo[d]imidazol-3-yl)piperidine-1-carboxylate;
1-(1-(3-(dibenzylamino)propyl)piperidin-4-yl)-1H-benzo[d]imidazol-2(3H)-one;
(S)-2-(dibenzylamino)-3-phenylpropyl 4-(1,2-dihydro-2-oxobenzo[d]imidazol-3-yl)piperidine-1-carboxylate;
(S)-2-(dibenzylamino)propyl 4-(N-phenylbenzamido)piperidine-1-carboxylate;
1-(S)-2-(dibenzylamino)propyl 3-ethyl 4-(1,2-dihydro-2-oxobenzo[d]imidazol-3-yl)piperidine-1,3-dicarboxylate;
(S)-2-(dibenzylamino)propyl 4-(1,2-dihydro-1-methyl-2-oxobenzo[d]imidazol-3-yl)piperidine-1-carboxylate;
(S)-2-(dibenzylamino)propyl 4-(2-oxo-3-(4-acetamidyl phenylsulfonyl)-2,3-dihydro-1H-benzo[d]imidazol-1-yl)piperidine-1-carboxylate;
1-(1-(2-(dibenzylamino)ethyl)piperidin-4-yl)-1H-benzo[d]imidazol-2(3H)-one;
(S)-2-(dibenzylamino)propyl 4-(2-oxo-3-methylsulfonyl-2,3-dihydro-1H-benzo[d]imidazol-1-yl)piperidine-1-carboxylate;
(S)-2-(dibenzylamino)propyl 4-(1H-benzo[d]imidazol-1-yl)piperidine-1-carboxylate;
(S)-2-(dibenzylamino)-3-phenylpropyl 4-(1,2-dihydro-3-phenylsulfonyl-2-oxobenzo[d]imidazol-1-yl)piperidine-1-carboxylate;
(S)-2-(dibenzylamino)propyl 4-(3-(2-tert-butyloxy carbonylaminoethyl)-2-oxo-2,3-dihydro-1H-benzo[d]imidazol-1-yl)piperidine-1-carboxylate;
(S)-2-(dibenzylamino)-3-phenylpropyl 4-(3-(2-tert-butyloxy carbonylaminoethyl)-2-oxo-2,3-dihydro-1H-benzo[d]imidazol-1-yl)piperidine-1-carboxylate;
(S)-2-(dibenzylamino)-3-phenylpropyl 4-(3-(2 aminoethyl)-2-oxo-2,3-dihydro-1H-benzo[d]imidazol-1-yl)piperidine-1-carboxylate D-biotin amid;
2-(benzyl(2-(benzyloxy)benzyl)amino)ethyl 4-(2-oxo-2,3-dihydro-1H-benzo[d]imidazol-1-yl)piperidine-1-carboxylate;
2-(benzyl(4-(benzyloxy)benzyl)amino)ethyl 4-(2-oxo-2,3-dihydro-1H-benzo[d]imidazol-1-yl)piperidine-1-carboxylate;
2-(benzyl(2,6-difluorobenzyl)amino)ethyl 4-(2-oxo-2,3-dihydro-1H-benzo[d]imidazol-1-yl)piperidine-1-carboxylate;
(S)-2-(benzyl(furan-2-ylmethyl)amino)propyl 4-(2-oxo-2,3-dihydro-1H-benzo[d]imidazol-1-yl)piperidine-1-carboxylate;
2-(benzyl(3-(benzyloxy)benzyl)amino)ethyl 4-(2-oxo-2,3-dihydro-1H-benzo[d]imidazol-1-yl)piperidine-1-carboxylate;
(E)-2-(benzyl(cinnamyl)amino)ethyl 4-(2-oxo-2,3-dihydro-1H-benzo[d]imidazol-1-yl)piperidine-1-carboxylate;
(S)-2-(benzyl(tert-butoxycarbonyl)amino)-4-methylpentyl 4-(2-oxo-2,3-dihydro-1H-benzo[d]imidazol-1-yl)piperidine-1-carboxylate;
(S)-2-(benzyl(tert-butoxycarbonyl)amino)-3-phenylpropyl 4-(2-oxo-2,3-dihydro-1H-benzo[d]imidazol-1-yl)piperidine-1-carboxylate;
(S)-2-(dibenzylamino)-3-methylbutyl 4-(1,2-dihydro-2-oxobenzo[d]imidazol-3-yl)piperidine-1-carboxylate;
(S)-2-(dibenzylamino)-4-methylpentyl 4-(1,2-dihydro-2-oxobenzo[d]imidazol-3-yl)piperidine-1-carboxylate;
2-(dibenzylamino)-3,3-dimethylbutyl 4-(1,2-dihydro-2-oxobenzo[d]imidazol-3-yl)piperidine-1-carboxylate;
(3-(dibenzylamino)butyl 4-(1,2-dihydro-2-oxobenzo[d]imidazol-3-yl)piperidine-1-carboxylate;
2-(dibenzylamino)-2-methylpropyl 4-(1,2-dihydro-2-oxobenzo[d]imidazol-3-yl)piperidine-1-carboxylate;
(S)-2-(dibenzylamino)-2-phenylethyl 4-(2-oxo-2,3-dihydro-1H-benzo[d]imidazol-1-yl)piperidine-1-carboxylate;
(S)-2-(benzyl(4-nitrobenzyl)amino)propyl 4-(2-oxo-2,3-dihydro-1H-benzo[d]imidazol-1-yl)piperidine-1-carboxylate;
3-(benzyl(4-nitrobenzyl)amino)propyl 4-(2-oxo-2,3-dihydro-1H-benzo[d]imidazol-1-yl)piperidine-1-carboxylate;
2-(benzyl(4-bromobenzyl)amino)ethyl 4-(2-oxo-2,3-dihydro-1H-benzo[d]imidazol-1-yl)piperidine-1-carboxylate;
S)-2-(benzyl(4-bromobenzyl)amino)propyl 4-(2-oxo-2,3-dihydro-1H-benzo[d]imidazol-1-yl)piperidine-1-carboxylate;
3-(benzyl(4-methoxybenzyl)amino)propyl 4-(2-oxo-2,3-dihydro-1H-benzo[d]imidazol-1-yl)piperidine-1-carboxylate;
(R)-2-(dibenzylamino)-3-phenylpropyl 4-(2-oxo-2,3-dihydro-1H-benzo[d]imidazol-1-yl)piperidine-1-carboxylate;
2-(benzyl(4-fluorobenzyl)amino)ethyl 4-(2-oxo-2,3-dihydro-1H-benzo[d]imidazol-1-yl)piperidine-1-carboxylate;
2-(benzyl(3-phenylpropyl)amino)ethyl 4-(2-oxo-2,3-dihydro-1H-benzo[d]imidazol-1-yl)piperidine-1-carboxylate;
2-(benzyl(4-methoxybenzyl)amino)ethyl 4-(2-oxo-2,3-dihydro-1H-benzo[d]imidazol-1-yl)piperidine-1-carboxylate;
2-(benzyl(3-methoxybenzyl)amino)ethyl 4-(2-oxo-2,3-dihydro-1H-benzo[d]imidazol-1-yl)piperidine-1-carboxylate;
2-(benzyl(3-phenoxybenzyl)amino)ethyl 4-(2-oxo-2,3-dihydro-1H-benzo[d]imidazol-1-yl)piperidine-1-carboxylate;
2-(benzyl(3-chlorobenzyl)amino)ethyl 4-(2-oxo-2,3-dihydro-1H-benzo[d]imidazol-1-yl)piperidine-1-carboxylate;
(S)-2-(benzyl(3-phenylpropyl)amino)propyl 4-(2-oxo-2,3-dihydro-1H-benzo[d]imidazol-1-yl)piperidine-1-carboxylate;
(S)-2-(benzyl(3-methoxybenzyl)amino)propyl 4-(2-oxo-2,3-dihydro-1H-benzo[d]imidazol-1-yl)piperidine-1-carboxylate;
(S)-2-(benzyl(3-phenoxybenzyl)amino)propyl 4-(2-oxo-2,3-dihydro-1H-benzo[d]imidazol-1-yl)piperidine-1-carboxylate;
(S)-2-(benzyl(3-chlorobenzyl)amino)propyl 4-(2-oxo-2,3-dihydro-1H-benzo[d]imidazol-1-yl)piperidine-1-carboxylate;
(S)-2-(benzyl(4-chlorobenzyl)amino)propyl 4-(2-oxo-2,3-dihydro-1H-benzo[d]imidazol-1-yl)piperidine-1-carboxylate;
2-(benzyl(naphthalen-1-ylmethyl)amino)ethyl 4-(2-oxo-2,3-dihydro-1H-benzo[d]imidazol-1-yl)piperidine-1-carboxylate;
2-(benzyl(quinolin-4-ylmethyl)amino)ethyl 4-(2-oxo-2,3-dihydro-1H-benzo[d]imidazol-1-yl)piperidine-1-carboxylate;
2-(benzyl((1-methyl-1H-indol-2-yl)methyl)amino)ethyl 4-(2-oxo-2,3-dihydro-1H-benzo[d]imidazol-1-yl)piperidine-1-carboxylate;
(S)-2-(benzyl(quinolin-4-ylmethyl)amino)propyl 4-(2-oxo-2,3-dihydro-1H-benzo[d]imidazol-1-yl)piperidine-1-carboxylate;
3-(benzyl(pyridin-3-ylmethyl)amino)propyl 4-(2-oxo-2,3-dihydro-1H-benzo[d]imidazol-1-yl)piperidine-1-carboxylate;
3-(benzyl(pyridin-2-ylmethyl)amino)propyl 4-(2-oxo-2,3-dihydro-1H-benzo[d]imidazol-1-yl)piperidine-1-carboxylate;
(S)-2-(benzyl(3-(benzyloxy)benzyl)amino)propyl 4-(2-oxo-2,3-dihydro-1H-benzo[d]imidazol-1-yl)piperidine-1-carboxylate;
(S)-2-(benzyl(phenanthren-9-ylmethyl)amino)propyl 4-(2-oxo-2,3-dihydro-1H-benzo[d]imidazol-1-yl)piperidine-1-carboxylate;
(S)-2-(benzyl(naphthalen-1-ylmethyl)amino)propyl 4-(2-oxo-2,3-dihydro-1H-benzo[d]imidazol-1-yl)piperidine-1-carboxylate;
(S)-2-(benzyl(thiophen-2-ylmethyl)amino)propyl 4-(2-oxo-2,3-dihydro-1H-benzo[d]imidazol-1-yl)piperidine-1-carboxylate;
(S)-2-(benzyl(pyridin-2-ylmethyl)amino)propyl 4-(2-oxo-2,3-dihydro-1H-benzo[d]imidazol-1-yl)piperidine-1-carboxylate;
3-(benzyl(thiophen-2-ylmethyl)amino)propyl 4-(2-oxo-2,3-dihydro-1H-benzo[d]imidazol-1-yl)piperidine-1-carboxylate;
3-(benzyl(3-chlorobenzyl)amino)propyl 4-(2-oxo-2,3-dihydro-1H-benzo[d]imidazol-1-yl)piperidine-1-carboxylate;
3-(benzyl(2-(benzyloxy)ethyl)amino)propyl 4-(2-oxo-2,3-dihydro-1H-benzo[d]imidazol-1-yl)piperidine-1-carboxylate;
3-(benzyl(naphthalen-1-ylmethyl)amino)propyl 4-(2-oxo-2,3-dihydro-1H-benzo[d]imidazol-1-yl)piperidine-1-carboxylate;
3-(benzyl(quinolin-4-ylmethyl)amino)propyl 4-(2-oxo-2,3-dihydro-1H-benzo[d]imidazol-1-yl)piperidine-1-carboxylate;
3-(benzyl(3-phenoxybenzyl)amino)propyl 4-(2-oxo-2,3-dihydro-1H-benzo[d]imidazol-1-yl)piperidine-1-carboxylate;
3-(benzyl(4-fluorobenzyl)amino)propyl 4-(2-oxo-2,3-dihydro-1H-benzo[d]imidazol-1-yl)piperidine-1-carboxylate;
3-(benzyl(3-phenylpropyl)amino)propyl 4-(2-oxo-2,3-dihydro-1H-benzo[d]imidazol-1-yl)piperidine-1-carboxylate;
3-(benzyl(3-methoxybenzyl)amino)propyl 4-(2-oxo-2,3-dihydro-1H-benzo[d]imidazol-1-yl)piperidine-1-carboxylate;
2-(benzyl(4-chlorobenzyl)amino)ethyl 4-(2-oxo-2,3-dihydro-1H-benzo[d]imidazol-1-yl)piperidine-1-carboxylate;
2-(benzyl(2-methoxybenzyl)amino)ethyl 4-(2-oxo-2,3-dihydro-1H-benzo[d]imidazol-1-yl)piperidine-1-carboxylate;
(S)-2-(dibenzylamino)-3-phenylpropyl 4-(phenylamino)piperidine-1-carboxylate;
(S)-2-(dibenzylamino)propyl 4-(2-phenyl-1H-benzo[d]imidazol-1-yl)piperidine-1-carboxylate;
(S)-2-(dibenzylamino)propyl 4-(N-phenyl-4-propylphenylsulfonamido)piperidine-1-carboxylate;
(S)-2-(dibenzylamino)propyl 4-(N-phenyl-4-fluorophenylsulfonamido)piperidine-1-carboxylate;
(S)-2-(dibenzylamino)propyl 4-(N-phenyl-4-methoxyphenylsulfonamido)piperidine-1-carboxylate;
(S)-2-(dibenzylamino)-3-phenylpropyl 4-(N-phenyl-4-methoxyphenylsulfonamido)piperidine-1-carboxylate;
(S)-2-(dibenzylamino)propyl 4-(N-phenyl-4-(trifluoromethyl)phenylsulfonamido)piperidine-1-carboxylate;
(S)-2-(dibenzylamino)propyl 4-(N-phenylthiophen-2-ylsulfonamido)piperidine-1-carboxylate;
(S)-2-(dibenzylamino)propyl 4-(N-phenylquinolin-8-ylsulfonamido)piperidine-1-carboxylate;
(S)-2-(dibenzylamino)propyl 4-(N-phenyl-naphthalen-1-ylsulfonamido)piperidine-1-carboxylate;
(S)-2-(dibenzylamino)-3-phenylpropyl 4-(N-phenyl-naphthalen-1-ylsulfonamido)piperidine-1-carboxylate;
(S)-2-(dibenzylamino)propyl 4-(N-phenylpivalamido)piperidine-1-carboxylate;
(S)-2-(dibenzylamino)propyl 4-(2-(phenylsulfonyl)phenylamino)piperidine-1-carboxylate;
(S)-2-(dibenzylamino)propyl 4-(1,3-diphenylthioureido)piperidine-1-carboxylate;
(S)-2-(dibenzylamino)propyl 4-(N-methyl-N-phenylamino)piperidine-1-carboxylate;
(S)-2-(dibenzylamino)propyl 4-(N-phenyl-2-methylphenylsulfonamido)piperidine-1-carboxy late;
(S)-2-(dibenzylamino)propyl 4-(N-phenyl-3-methylphenylsulfonamido)piperidine-1-carboxylate;
(S)-2-(dibenzylamino)propyl 4-(N-phenyl-3-(trifluoromethyl)phenylsulfonamido)piperidine-1-carboxylate;
(S)-2-(dibenzylamino)propyl 4-(N-phenyl-biphenyl-4-sulfonamido)piperidine-1-carboxylate;
(S)-2-(dibenzylamino)propyl 4-(N-phenyl-naphthalen-2-ylsulfonamido)piperidine-1-carboxylate;
(S)-2-(dibenzylamino)propyl 4-(N-phenyl-(5-dimethylamino)naphthalen-1-ylsulfonamido)piperidine-1-carboxylate;
(S)-2-(dibenzylamino)propyl 4-(2-(4-chlorophenylthio)phenylamino)piperidine-1-carboxylate;
(S)-2-(dibenzylamino)-3-phenylpropyl dimethylphenylamino)piperidine-1-carboxylate;
(S)-2-(dibenzylamino)-3-phenylpropyl 4-(2,6-diethylphenylamino)piperidine-1-carboxylate;
(S)-2-(dibenzylamino)-3-phenylpropyl 4-(2-(methylthio)phenylamino)piperidine-1-carboxylate; and pharmaceutically acceptable salts thereof.

When the drug is intended for the prevention and/or treatment of toxoplasmose, compounds of formula (I) are preferably selected in the group consisting of:
O-2-(dibenzylamino)ethyl 4-(1,2-dihydro-2-oxobenzo[d]imidazol-3-yl)piperidine-1-carbothioate;
(S)-2-(dibenzylamino)propyl 4-(1,2-dihydro-1-methyl-2-oxobenzo[d]imidazol-3-yl)piperidine-1-carboxylate;
(S)-2-(dibenzylamino)propyl 3-(1,2-dihydro-2-oxobenzo[d]imidazol-3-yl)propylcarbamate;
1-benzylpiperidin-3-yl 4-(1,2-dihydro-2-oxobenzo[d]imidazol-3-yl)piperidine-1-carboxylate;
2-(benzyloxy)ethyl 4-(1,2-dihydro-2-oxobenzo[d]imidazol-3-yl)piperidine-1-carboxylate;
(S)-2-(dibenzylamino)propyl 4-(1,2-dihydro-2-oxonaphtho[2,3-d]imidazol-3-yl)piperidine-1-carboxylate;
1-(1-(3-(dibenzylamino)propyl)piperidin-4-yl)-1H-benzo[d]imidazol-2(3H)-one;
(S)-2-(dibenzylamino)propyl 4-(2-oxobenzo[d]oxazol-3(2H)-yl)piperidine-1-carboxylate;
(S)-2-(dibenzylamino)-3-phenylpropyl 4-(1,2-dihydro-1-methyl-2-oxobenzo[d]imidazol-3-yl)piperidine-1-carboxylate;
(S)-2-(dibenzylamino)propyl 4-(1,2-dihydro-2-oxo-1-(benzoyl)benzo[d]imidazol-3-yl)piperidine-1-carboxylate;
2-(1-benzylpiperidin-4-yl)ethyl 4-(1,2-dihydro-2-oxobenzo[d]imidazol-3-yl)piperidine-1-carboxylate;
(S)-2-(dibenzylamino)propyl 4-(1H-benzo[d]imidazol-2-ylamino)piperidine-1-carboxylate;
(S)-2-(dibenzylamino)propyl 4-(1-acetyl-1,2-dihydro-2-oxobenzo[d]imidazol-3-yl)piperidine-1-carboxylate;
(S)-2-(dibenzylamino)propyl 4-(6-chloro-1,2-dihydro-2-oxobenzo[d]imidazol-3-yl)piperidine-1-carboxylate;
O-2-(benzyloxy)ethyl 4-(1,2-dihydro-2-oxobenzo[d]imidazol-3-yl)piperidine-1-carbothioate;
O—(S)-2-(dibenzylamino)propyl 4-(1,2-dihydro-2-oxobenzo[d]imidazol-3-yl)piperidine-1-carbothioate;
3,3-diphenylpropyl 4-(1,2-dihydro-2-oxobenzo[d]imidazol-3-yl)piperidine-1-carboxylate;
(S)-2-(dibenzylamino)propyl 4-(N-phenylpropionamido)piperidine-1-carboxylate;
1-(1-(4-(dibenzylamino)butyl)piperidin-4-yl)-1H-benzo[d]imidazol-2(3H)-one;
(S)-2-(dibenzylamino)propyl 4-(3-(4-fluorophenylsulfonyl)-2-oxo-2,3-dihydro-1H-benzo[d]imidazol-1-yl)piperidine-1-carboxylate;
(S)-2-(dibenzylamino)propyl 4-(1-(cyclohexylmethyl)-1,2-dihydro-2-oxobenzo[d]imidazol-3-yl)piperidine-1-carboxylate;
(S)-2-(dibenzylamino)propyl 4-(2-oxo-3-phenylsulfonyl-2,3-dihydro-1H-benzo[d]imidazol-1-yl)piperidine-1-carboxylate;
(S)-4-(3-(1-((2-(dibenzylamino)propoxy)carbonyl)piperidin-4-yl)-2-oxo-2,3-dihydro-1H-benzo[d]imidazol-1-yl)butanoic acid;
(S)-2-(dibenzylamino)propyl 4-(3-(4-methoxyphenylsulfonyl)-2-oxo-2,3-dihydro-1H-benzo[d]imidazol-1-yl)piperidine-1-carboxylate;
(S)-2-(dibenzylamino)propyl 4-(N-phenylphenylsulfonamido)piperidine-1-carboxylate;
(S)-2-(dibenzylamino)propyl 4-(1-benzyl-1,2-dihydro-2-oxobenzo[d]imidazol-3-yl)piperidine-1-carboxylate;
2-(ethyl(phenyl)amino)ethyl 4-(2-oxo-2,3-dihydro-1H-benzo[d]imidazol-1-yl)piperidine-1-carboxylate;
(S)-2-(dibenzylamino)-3-phenylpropyl 4-(1-benzyl-1,2-dihydro-2-oxobenzo[d]imidazol-3-yl)piperidine-1-carboxylate;
(S)-2-(dibenzylamino)propyl 4-(1H-indol-3-yl)piperidine-1-carboxylate;
(S)-2-(dibenzylamino)propyl 4-(2-oxo-3-methylsulfonyl-2,3-dihydro-1H-benzo[d]imidazol-1-yl)piperidine-1-carboxylate;
(S)-2-(dibenzylamino)propyl 4-(1H-benzo[d]imidazol-2-ylamino)piperidine-1-carboxylate;
(S)-2-(dibenzylamino)propyl 4-(2-oxo-3-phenylsulfonyl-2,3-dihydro-1H-benzo[d]imidazol-1-yl)piperidine-1-carboxylate;
(S)-2-(dibenzylamino)propyl 4-(3-(4-fluorophenylsulfonyl)-2-oxo-2,3-dihydro-1H-benzo[d]imidazol-1-yl)piperidine-1-carboxylate;
(S)-2-(dibenzylamino)propyl 4-(3-(4-methoxyphenylsulfonyl)-2-oxo-2,3-dihydro-1H-benzo[d]imidazol-1-yl)piperidine-1-carboxylate;
(S)-2-(dibenzylamino)propyl 4-(2-oxo-3-(4-(trifluoromethyl)phenylsulfonyl)-2,3-dihydro-1H-benzo[d]imidazol-1-yl)piperidine-1-carboxylate;
(S)-2-(dibenzylamino)-3-phenylpropyl 4-(1,2-dihydro-1-methyl-2-oxobenzo[d]imidazol-3-yl)piperidine-1-carboxylate;
(S)-2-(dibenzylamino)propyl 4-(2-oxo-3-(thiophen-2-ylsulfonyl)-2,3-dihydro-1H-benzo[d]imidazol-1-yl)piperidine-1-carboxylate;
N—((S)-2-(dibenzylamino)propyl)-4-(1,2-dihydro-2-oxobenzo[d]imidazol-3-yl)piperidine-1-carboxamidine;
(S)-2-(dibenzylamino)propyl 4-(N-phenylphenylsulfonamido)piperidine-1-carboxylate;
(S)-2-(dibenzylamino)propyl 4-(3-(4-ethoxy-4-oxobutyl)-2-oxo-2,3-dihydro-1H-benzo[d]imidazol-1-yl)piperidine-1-carboxylate;
(S)-2-(dibenzylamino)propyl 4-(1-benzyl-1,2-dihydro-2-oxobenzo[d]imidazol-3-yl)piperidine-1-carboxylate;
(S)-2-(dibenzylamino)propyl 4-(2-oxo-3-(quinolin-8-ylsulfonyl)-2,3-dihydro-1H-benzo[d]imidazol-1-yl)piperidine-1-carboxylate;
tert-butyl 3-(1-4(S)-2-(dibenzylamino)propoxy)carbonyl)piperidin-4-yl)-2,3-dihydro-2-oxobenzo[d]imidazole-1-carboxylate;
(S)-2-(dibenzylamino)-3-phenylpropyl 4-(1-benzyl-1,2-dihydro-2-oxobenzo[d]imidazol-3-yl)piperidine-1-carboxylate;
(S)-2-(dibenzylamino)propyl 4-(3-(naphthalen-1-ylsulfonyl)-2-oxo-2,3-dihydro-1H-benzo[d]imidazol-1-yl)piperidine-1-carboxylate;
(S)-2-(dibenzylamino)propyl 4-(1-(cyclohexylmethyl)-1,2-dihydro-2-oxobenzo[d]imidazol-3-yl)piperidine-1-carboxylate;
(S)-2-(dibenzylamino)propyl 4-(1,2-dihydro-2-thioxobenzo[d]imidazol-3-yl)piperidine-1-carboxylate;
1-(1-(3-(dibenzylamino)propyl)piperidin-4-yl)-1H-benzo[d]imidazol-2(3H)-one;
(S)-2-(dibenzylamino)-3-phenylpropyl 4-(1,2-dihydro-2-oxobenzo[d]imidazol-3-yl)piperidine-1-carboxylate;
(S)-2-(dibenzylamino)propyl 4-(N-phenylbenzamido)piperidine-1-carboxylate;
1-(S)-2-(dibenzylamino)propyl 3-ethyl 4-(1,2-dihydro-2-oxobenzo[d]imidazol-3-yl)piperidine-1,3-dicarboxylate;
(S)-2-(dibenzylamino)propyl 4-(1,2-dihydro-1-methyl-2-oxobenzo[d]imidazol-3-yl)piperidine-1-carboxylate;
(S)-2-(dibenzylamino)propyl 4-(2-oxo-3-(4-acetamidyl phenylsulfonyl)-2,3-dihydro-1H-benzo[d]imidazol-1-yl)piperidine-1-carboxylate;
1-(1-(2-(dibenzylamino)ethyl)piperidin-4-yl)-1H-benzo[d]imidazol-2(3H)-one;
(S)-2-(dibenzylamino)propyl 4-(2-oxo-3-methylsulfonyl-2,3-dihydro-1H-benzo[d]imidazol-1-yl)piperidine-1-carboxylate;
(S)-2-(dibenzylamino)propyl 4-(1H-benzo[d]imidazol-1-yl)piperidine-1-carboxylate;
(S)-2-(dibenzylamino)-3-phenylpropyl 4-(1,2-dihydro-3-phenylsulfonyl-2-oxobenzo[d]imidazol-1-yl)piperidine-1-carboxylate;
(S)-2-(dibenzylamino)propyl 4-(3-(2-tert-butyloxy carbonylaminoethyl)-2-oxo-2,3-dihydro-1H-benzo[d]imidazol-1-yl)piperidine-1-carboxylate;
(S)-2-(dibenzylamino)-3-phenylpropyl 4-(3-(2-tert-butyloxy carbonylaminoethyl)-2-oxo-2,3-dihydro-1H-benzo[d]imidazol-1-yl)piperidine-1-carboxylate;
(S)-2-(dibenzylamino)-3-phenylpropyl 4-(3-(2 aminoethyl)-2-oxo-2,3-dihydro-1H-benzo[d]imidazol-1-yl)piperidine-1-carboxylate D-biotin amid;
(S)-2-(dibenzylamino)propyl 4-(2-phenyl-1H-benzo[d]imidazol-1-yl)piperidine-1-carboxylate;
(S)-2-(dibenzylamino)propyl 4-(2-amino-1H-benzo[d]imidazol-1-yl)piperidine-1-carboxylate;
(S)-2-(dibenzylamino)propyl 4-(4-fluoro-N-phenylphenylsulfonamido)piperidine-1-carboxylate;
(S)-2-(dibenzylamino)propyl 4-(4-methoxy-N-phenylphenylsulfonamido)piperidine-1-carboxylate;
(S)-2-(dibenzylamino)propyl 4-(N-phenylthiophene-2-sulfonamido)piperidine-1-carboxylate;
(S)-2-(dibenzylamino)propyl 4-(N-phenylquinoline-8-sulfonamido)piperidine-1-carboxylate;
4-(dibenzylamino)phenyl 4-(1,2-dihydro-2-oxobenzo[d]imidazol-3-yl)piperidine-1-carboxylate;
(S)-2-(dibenzylamino)propyl 4-(N-phenylpivalamido)piperidine-1-carboxylate;
(S)-2-(dibenzylamino)propyl 4-(2-oxo-2,3-dihydro-1H-benzo[d]imidazol-4-yl)piperidine-1-carboxylate;
6-(dibenzylamino)hexyl 4-(1,2-dihydro-2-oxobenzo[d]imidazol-3-yl)piperidine-1-carboxylate;
(S)-2-(dibenzylamino)-4-methylpentyl 4-(2-oxo-2,3-dihydro-1H-benzo[d]imidazol-1-yl)piperidine-1-carboxylate;
(S)-3-(dibenzylamino)butyl 4-(2-oxo-2,3-dihydro-1H-benzo[d]imidazol-1-yl)piperidine-1-carboxylate;
(S)-2-(dibenzylamino)-2-phenylethyl 4-(1,2-dihydro-2-oxobenzo[d]imidazol-3-yl)piperidine-1-carboxylate;
Benzyl 4-(1,2-dihydro-2-oxobenzo[d]imidazol-3-yl)piperidine-1-carboxylate;
(1-benzylpiperidin-2-yl)methyl 4-(1,2-dihydro-2-oxobenzo[d]imidazol-3-yl)piperidine-1-carboxylate;
(S)-2-(dibenzylamino)propyl 4-(1,3-diphenylthioureido)piperidine-1-carboxylate;
3-(benzyl(4-nitrobenzyl)amino)propyl 4-(2-oxo-2,3-dihydro-1H-benzo[d]imidazol-1-yl)piperidine-1-carboxylate;
2-(benzyl(4-bromobenzyl)amino)ethyl 4-(2-oxo-2,3-dihydro-1H-benzo[d]imidazol-1-yl)piperidine-1-carboxylate;
(S)-2-(dibenzylamino)-3-phenylpropyl 4-(phenylamino)piperidine-1-carboxylate;
(S)-2-(dibenzylamino)propyl 4-(2-methyl-N-phenylphenylsulfonamido)piperidine-1-carboxylate;
(S)-2-(dibenzylamino)propyl 4-(3-methyl-N-phenylphenylsulfonamido)piperidine-1-carboxylate;
(S)-2-(dibenzylamino)propyl 4-(N-phenyl-3-(trifluoromethyl)phenylsulfonamido)piperidine-1-carboxylate;
(S)-2-(dibenzylamino)propyl 4-(5-(dimethylamino)-N-phenylnaphthalene-1-sulfonamido)piperidine-1-carboxylate;
(R)-2-(dibenzylamino)-3-phenylpropyl 4-(1,2-dihydro-2-oxobenzo[d]imidazol-3-yl)piperidine-1-carboxylate;
2-(benzyl(4-fluorobenzyl)amino)ethyl 4-(2-oxo-2,3-dihydro-1H-benzo[d]imidazol-1-yl)piperidine-1-carboxylate;
2-(benzyl(3-phenylpropyl)amino)ethyl 4-(2-oxo-2,3-dihydro-1H-benzo[d]imidazol-1-yl)piperidine-1-carboxylate;
2-(benzyl(4-methoxybenzyl)amino)ethyl 4-(2-oxo-2,3-dihydro-1H-benzo[d]imidazol-1-yl)piperidine-1-carboxylate;
2-(benzyl(3-methoxybenzyl)amino)ethyl 4-(2-oxo-2,3-dihydro-1H-benzo[d]imidazol-1-yl)piperidine-1-carboxylate;
2-(benzyl(3-phenoxybenzyl)amino)ethyl 4-(2-oxo-2,3-dihydro-1H-benzo[d]imidazol-1-yl)piperidine-1-carboxylate;
(S)-2-(dibenzylamino)-3-phenylpropyl 4-(N-phenyl-4-(trifluoromethyl)phenylsulfonamido)piperidine-1-carboxylate;
(S)-2-(benzyl(4-fluorobenzyl)amino)propyl 4-(2-oxo-2,3-dihydro-1H-benzo[d]imidazol-1-yl)piperidine-1-carboxylate;
(S)-2-(benzyl(3-phenylpropyl)amino)propyl 4-(2-oxo-2,3-dihydro-1H-benzo[d]imidazol-1-yl)piperidine-1-carboxylate;
(S)-2-(benzyl(quinolin-4-ylmethyl)amino)propyl 4-(2-oxo-2,3-dihydro-1H-benzo[d]imidazol-1-yl)piperidine-1-carboxylate;
(S)-2-(benzyl(3-methoxybenzyl)amino)propyl 4-(2-oxo-2,3-dihydro-1H-benzo[d]imidazol-1-yl)piperidine-1-carboxylate;
(S)-2-(benzyl(3-chlorobenzyl)amino)propyl 4-(2-oxo-2,3-dihydro-1H-benzo[d]imidazol-1-yl)piperidine-1-carboxylate;
2-(benzyl(quinolin-4-ylmethyl)amino)ethyl 4-(2-oxo-2,3-dihydro-1H-benzo[d]imidazol-1-yl)piperidine-1-carboxylate;
2-(benzyl((1-methyl-1H-indol-2-yl)methyl)amino)ethyl 4-(2-oxo-2,3-dihydro-1H-benzo[d]imidazol-1-yl)piperidine-1-carboxylate;
3-(benzyl(pyridin-2-ylmethyl)amino)propyl 4-(2-oxo-2,3-dihydro-1H-benzo[d]imidazol-1-yl)piperidine-1-carboxylate;
(S)-2-(benzyl(3-(benzyloxy)benzyl)amino)propyl 4-(2-oxo-2,3-dihydro-1H-benzo[d]imidazol-1-yl)piperidine-1-carboxylate;
(S)-2-(benzyl(phenanthren-9-ylmethyl)amino)propyl 4-(2-oxo-2,3-dihydro-1H-benzo[d]imidazol-1-yl)piperidine-1-carboxylate;
(S)-2-(benzyl(naphthalen-1-ylmethyl)amino)propyl 4-(2-oxo-2,3-dihydro-1H-benzo[d]imidazol-1-yl)piperidine-1-carboxylate;
(S)-2-(benzyl(thiophen-2-ylmethyl)amino)propyl 4-(2-oxo-2,3-dihydro-1H-benzo[d]imidazol-1-yl)piperidine-1-carboxylate;
(S)-2-(benzyl(pyridin-2-ylmethyl)amino)propyl 4-(2-oxo-2,3-dihydro-1H-benzo[d]imidazol-1-yl)piperidine-1-carboxylate;
2-(benzyl(tert-butoxycarbonyl)amino)ethyl 4-(2-oxo-2,3-dihydro-1H-benzo[d]imidazol-1-yl)piperidine-1-carboxylate;
3-(benzyl(thiophen-2-ylmethyl)amino)propyl 4-(2-oxo-2,3-dihydro-1H-benzo[d]imidazol-1-yl)piperidine-1-carboxylate;
3-(benzyl(3-methoxybenzyl)amino)propyl 4-(2-oxo-2,3-dihydro-1H-benzo[d]imidazol-1-yl)piperidine-1-carboxylate;
3-(benzyl(3-chlorobenzyl)amino)propyl 4-(2-oxo-2,3-dihydro-1H-benzo[d]imidazol-1-yl)piperidine-1-carboxylate;
3-(benzyl(naphthalen-1-ylmethyl)amino)propyl 4-(2-oxo-2,3-dihydro-1H-benzo[d]imidazol-1-yl)piperidine-1-carboxylate;
3-(benzyl(quinolin-4-ylmethyl)amino)propyl 4-(2-oxo-2,3-dihydro-1H-benzo[d]imidazol-1-yl)piperidine-1-carboxylate;
3-(benzyl((1-methyl-1H-indol-2-yl)methyl)amino)propyl 4-(2-oxo-2,3-dihydro-1H-benzo[d]imidazol-1-yl)piperidine-1-carboxylate;
3-(benzyl(3-phenoxybenzyl)amino)propyl 4-(2-oxo-2,3-dihydro-1H-benzo[d]imidazol-1-yl)piperidine-1-carboxylate;
2-(benzyl(4-(methoxycarbonyl)benzyl)amino)ethyl 4-(2-oxo-2,3-dihydro-1H-benzo[d]imidazol-1-yl)piperidine-1-carboxylate;
2-(benzyl(4-chlorobenzyl)amino)ethyl 4-(2-oxo-2,3-dihydro-1H-benzo[d]imidazol-1-yl)piperidine-1-carboxylate;
2-(benzyl(2-(benzyloxy)benzyl)amino)ethyl 4-(2-oxo-2,3-dihydro-1H-benzo[d]imidazol-1-yl)piperidine-1-carboxylate;
3-(benzyl(3-(benzyloxy)benzyl)amino)propyl 4-(2-oxo-2,3-dihydro-1H-benzo[d]imidazol-1-yl)piperidine-1-carboxylate;
(S)-2-(benzyl(tert-butoxycarbonyl)amino)-4-methylpentyl 4-(2-oxo-2,3-dihydro-1H-benzo[d]imidazol-1-yl)piperidine-1-carboxylate;
(S)-2-(benzylamino)-4-methylpentyl 4-(2-oxo-2,3-dihydro-1H-benzo[d]imidazol-1-yl)piperidine-1-carboxylate;
2-(benzylamino)-3-phenylpropyl 4-(2-oxo-2,3-dihydro-1H-benzo[d]imidazol-1-yl)piperidine-1-carboxylate and pharmaceutically acceptable salts thereof.

Finally, a subject matter of the invention is also a pharmaceutical composition comprising, as an active principle, at least one compound of formula (I) (as a free base or in the form of a pharmaceutically acceptable salt), and at least one pharmaceutically acceptable excipient.

The pharmaceutical composition of the present invention may be administered by any suitable route, for example, by oral, buccal, inhalation, sublingual, nasal, percutaneous, i.e., transdermal, or parenteral (including intravenous, intramuscular, subcutaneous and intracoronary) administration. Therefore, the pharmaceutical composition of the invention can be provided in various forms, such as in the form of hard gelatin capsules, of capsules, of compressed tablets, of suspensions to be taken orally, of lozenges or of injectable solutions or in any other form appropriate to the method of administration.

According to a preferred embodiment of the invention, the pharmaceutical composition is for an oral administration.

The pharmaceutical composition according to the invention includes those wherein the at least one compound of the above-defined formula (I) is administered in an effective amount to achieve its intended purpose. Determination of the effective amounts is well within the capability of those skilled in the art.

A “therapeutically effective dose” refers to that amount of the compounds of formula (I) that results in achieving the desired effect. It has to be noted that when the compound of formula (I) is in the form of a pharmaceutically acceptable salt, this amount is calculated on the basis of the mass of said compound of formula (I) in the form of its free base. Toxicity and therapeutic efficacy of such compounds can be easily determined by standard pharmaceutical procedures in cell cultures or experimental animals, e.g., for determining the LD₅₀ (the dose lethal to 50% of the population) end the ED₅₀ (the dose therapeutically effective in 50% of the population). The dose ratio between toxic and therapeutic effects is the therapeutic index, which is expressed as the ratio between LD₅₀ and ED₅₀ compounds of formula (I) which exhibit high therapeutic indices are of course preferred. The data obtained from such data can be used in formulating range of dosage for use in humans. The dosage of compounds of formula (I) preferably lies within a range of circulating concentrations that include the ED₅₀ with little or no toxicity. The dosage can vary within this range depending upon the dosage form employed, and the route of administration.

The exact formulation, route of administration, and dosage can be chosen by the individual physician in view of the patient's conditions. Dosage amount and interval of administration can be adjusted individually to provide plasma levels of the compound of formula (I) which are sufficient to maintain the preventive or therapeutic effects.

The amount of pharmaceutical composition administered will therefore depend on the subject being treated, on the subject's weight, the severity of the affliction and the manner of administration.

For human use, the compounds of formula (I) can be administered alone, but they are preferably administered in admixture with at least one pharmaceutically acceptable carrier, the nature of which will depend on the intended route of administration and the presentation form. Pharmaceutical composition for use according to the present invention thus can be formulated in a conventional manner using one or more physiologically acceptable carriers comprising one or more excipients and auxiliaries that facilitate processing of the compounds of formula (I) into preparations which can be used pharmaceutically. Amongst the excipients and auxiliaries which can be used in the pharmaceutical composition according to the invention, one can mention antiagglomerating agents, antioxidants, preservatives agents, dyes, vitamins, inorganic salts, taste-modifying agents, smoothing agents, coating agents, isolating agents, stabilizing agents, wetting agents, anti-caking agents, dispersing agents, emulsifying agents, aromas, penetrating agents, solubilizing agents, etc. . . . , mixtures thereof and generally any excipient conventionally used in the pharmaceutical industry.

By way of example, when the pharmaceutical composition is administered orally, the carrier may comprise one or several excipients such as talc, lactose, starch or modified starches, cellulose or cellulose derivatives, polyethylene glycols, acrylic acid polymers, gelatin, magnesium stearate, animal or vegetal fats of natural or synthetic origin, paraffin derivatives, glycols, etc. . . . .

In addition to the at least one compound of formula (I), the pharmaceutical composition may also comprises one or more additional usually used antiparasitic active principle, such as for example an anti-malarial drugs (as example chloroquine, quinacrine, primaquine and artemisinine).

For general information about the formulation and administration of pharmaceutical compositions, one can obviously refer to the book “Remington's Pharmaceutical Sciences”, last edition. Of course, a person skilled in the art will take care on this occasion that the excipient(s) and/or auxiliary(ies) optionally used are compatible with the intrinsic properties attached to the pharmaceutical composition in accordance with the invention.

These pharmaceutical compositions can be manufactured in a conventional manner, e.g., by conventional mixing, dissolving, granulating, dragee-making, emulsifying, encapsulating, entrapping, or lyophilizing processes. Proper formulation is dependent upon the route of administration chosen.

Besides the arrangements above, the invention also comprises other arrangements which will emerge from the following description, which refers to examples of preparation of compounds of formula (I) according to the invention and to in vitro demonstration of the antiparasitic activity of compounds of formula (I).

It should be clearly understood, however, that these examples are given only by way of illustration of the subject of the invention, of which they in no way constitute a limitation.

### EXAMPLES

### Example 1

### General Protocol and Synthesis of Different Compounds of Formula (I)

### General Procedure

Commercial reagents were purchased from Aldrich and were used as received without additional purification. All reactions were carried out under nitrogen with dry, freshly distilled solvents and oven- or flame-dried glassware.

For solid phase synthesis, the moderate-scale reactions (100-500 mg of resin) were carried out using flasks fitted with a frit at the bottom and a stopper. All solid phase reactions were agitated by variable speed orbital mixer. Merrifield resin was obtained from Aldrich (100-200 mesh; 1% cross-linked; loading: 1.97 mmol/g).

Chromatography was carried out on Merck silica gel 60 (particle size 230-400 mesh). Thin layer chromatography (TLC) was performed on a 0.2 mm precoated plates of silica gel 60F-264 (Merck). Visualization was made with ultraviolet light or iodide or phosphomolibdic acid spray. Preparative TLC was performed on a 1.0 mm precoated plates of silica gel 60F-264 (Whatman). Visualization was made with ultraviolet light.

¹H and ¹³C NMR spectra were recorded on a Brucker Avance 400 with a BBO probe.

IR spectra of resin materials in KBr tablets were taken with a Perkin Elmer 2000 FTIR.

Analytical Method for LC/MS:

Column: Xbridge C18 3-5 μM, 4.6 mm×100 mm

Flow rate: 1.0 mL/min

Detector: Photodiode Array Detector Waters 2996: UV (200-400 nm), PL-ELS 1000, MS ZQ 2000

Injection volume: 1 μl, using Autosampler: Waters 2767

Method: 95% A, 5% B to 0% A, 100% B with 8 minutes gradient then 5 minutes hold

A: 100% water, 0.1% formic acid

B: 100% acetonitrile

### 1) Mitsunobu Procedure

a. General Procedure for Primary Alcohol:

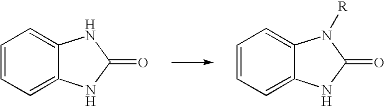

To a stirred suspension of 1H-benzo[d]imidazol-2(3H)-one (1 equiv.), appropriate alcohol (2 equiv.) and PPh₃ (1.5 equiv.) in THF (22 mL/mmol) cooled to 0° C. under N₂ was added dropwise DEAD (1.5 equiv.). The resulting mixture was allowed to warm to room temperature and stirred for 16 h. The solvent was evaporated. The residue was purified by appropriate method.

### Preparation of tert-butyl 4-((1,2-dihydro-2-oxobenzo[d]imidazol-3-yl)methyl)piperidine-1-carboxylate (Intermediate)

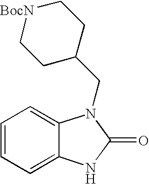

From tert-butyl 4-(hydroxymethyl)piperidine-1-carboxylate

Flash chromatography on silica gel (methylene chloride/acetone 8/2)

Colorless oil (60%).

LC/MS (ES⁺) m/z 332.1 (M+H)⁺

### Preparation of tert-butyl 4-(2-(1,2-dihydro-2-oxobenzo[d]imidazol-3-yl)ethyl)piperidine-1-carboxylate (Intermediate)

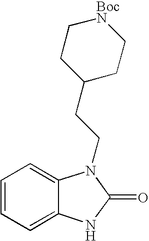

From tert-butyl 4-(2-hydroxyethyl)piperidine-1-carboxylate

Flash chromatography on silica gel (cyclohexane/ethyl acetate 5/5)

Colorless oil (50%).

LC/MS (ES⁺) m/z 246.1 ((M-Boc)+H)⁺

### Preparation of 1-((1-benzylpyrrolidin-2-yl)methyl)-1H-benzo[d]imidazol-2(3H)-one (Intermediate)

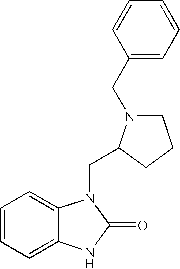

From S-(1-benzylpyrrolidin-2-yl)methanol

Flash chromatography on silica gel (cyclohexane/ethyl acetate 5/5)

Colorless oil (35%).

LC/MS (ES⁺) m/z 308.2 (M+H)⁺

b. General Procedure for Secondary Alcohol:

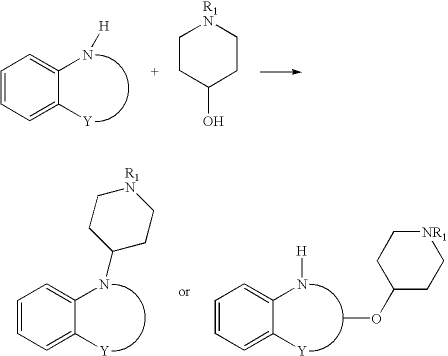

To a stirred suspension of appropriate heterocycle (0.37 mmol, 2 equiv.), (S)-2-(dibenzylamino)propyl 4-hydroxypiperidine-1-carboxylate (70 mg, 0.18 mmol) and PPh₃ (72 mg, 0.27 mmol) in THF (4 mL) cooled to 0° C. under N₂ was added dropwise DEAD (43 μl, 0.27 mmol). The resulting mixture was allowed to warm to room temperature and stirred overnight. The solvent was evaporated. The residue was purified by appropriate method.

### Preparation of (S)-2-(dibenzylamino)propyl 4-(1H-benzo[d]imidazol-1-yl)piperidine-1-carboxylate (Compound no 3)

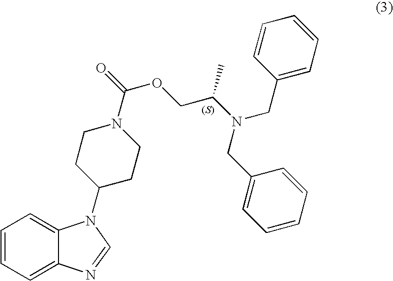

From benzimidazole

Colorless oil (65%).

preparative LCMS

LC/MS (ES+) m/z 483.1 (M+H)+

### 2) Seven-Member Cycle Preparation

### Preparation of azepan-4-one hydrochloride (Intermediate)

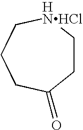

To a stirred solution of N-carbethoxy-4-piperidone (600 mg, 3.00 mmol) in anhydrous Et₂O (1.5 mL) were simultaneously added solutions of BF₃.Et₂O (380 μL, 3.00 mmol) and ethyl diazoacetate (412 μL, 3.92 mmol), each in anhydrous Et₂O (0.4 mL) at −35° C. under a nitrogen atmosphere. The reaction mixture was stirred for 1 h30 at −35° C. and allowed to warm to room temperature. The solution was washed with 30% aqueous K₂CO₃ solution (3 mL) and the organic phase was extracted with ethyl acetate (3×5 mL). The organic layers were dried over anhydrous Na₂SO₄, filtered and concentrated under vacuum. The crude material was refluxed in 4N aqueous HCl (13 mL) for 6 h and the solvent was removed on vacuo to afford a pale yellow oil (400 mg, 88%):

¹H NMR (400 MHz, CDCl₃) δ 3.04 (m, 4H, CH₂NH), 2.59 (m, 4H, CH₂CO), 1.86 (m, 1H, NH), 1.75 (m, 2H, CH₂CH₂NH).

### Preparation of (S)-2-(dibenzylamino)propyl 4-oxoazepane-1-carboxylate (Intermediate)

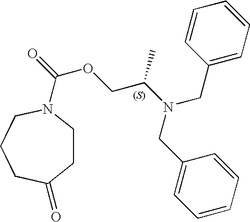

To a stirred solution of the (S)-2-(dibenzylamino)propan-1-ol (285 mg, 1.20 mmol) in THF (6 mL) at 0° C. was added CDI (213 mg, 1.30 mmol) under N₂ atmosphere. The reaction mixture was stirred for 2 h at room temperature. The azepan-4-one hydrochloride (140 mg, 1.12 mmol) in DMF (4 mL) and triethylamine (568 μL, 4.00 mmol) were added and the reaction mixture was heated at 50° C. for 72 h. The solvent was removed on vacuo and the crude material was purified by flash chromatography (silica gel, cyclohexane/ethyl acetate 5/5) to afford a pale yellow oil (20 mg, 4%).

¹H NMR (400 MHz, CDCl₃) δ 7.35 (d, J=7.2 Hz, 4H, Bn), 7.28 (t, J=7.2 Hz, 4H, Bn), 7.22 (t, J=7.2 Hz, 2H, Bn), 4.22 (dd, J=11.2, 7.2 Hz, 1H, CH₂O), 4.02 (dd, J=11.2, 5.6 Hz, 1H, CH₂O), 3.73 (d, J=14.0 Hz, 2H, NCH₂Ph), 3.64 (m, 4H, CH₂CH₂N, CH₂CH₂CH₂N), 3.54 (d, J=14.0 Hz, 2H, NCH₂Ph), 3.12 (m, 1H, CHMe), 2.67 (m, 4H, CH₂CH₂N, CH₂CH₂CH₂N), 1.80 (m, 2H, CH₂CH₂CH₂N), 1.08 (d, J=4.4 Hz, 3H, Me)

LC/MS (ES⁺) m/z 395.2 (M+H)⁺

### 3) Alkyl Derivative Preparation

### Preparation of (S)-2-(dibenzylamino)propyl 3-bromopropylcarbamate (Intermediate)

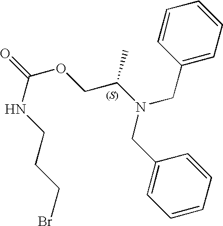

To a stirred solution of (S)-2-(dibenzylamino)propan-1-ol (280 mg, 1.10 mmol) in THF (2.5 mL) at 0° C. was added CDI (194 mg, 1.20 mmol) under N₂ atmosphere. The reaction mixture was stirred for 2 h at room temperature. The 3-bromopropan-1-amine hydrobromide (219 mg, 1.0 mmol) and triethylamine (209 μL, 1.50 mmol) were added and the reaction mixture was stirred for 72 h at room temperature. The solvent was removed on vacuo and the crude material was purified by flash chromatography (silica gel, cyclohexane/ethyl acetate 5/5) to afford a pale yellow oil (20 mg, 4%).

¹H NMR (400 MHz, CDCl₃) δ 7.36 (d, J=7.2 Hz, 4H, Bn), 7.29 (t, J=7.2 Hz, 4H, Bn), 7.22 (t, J=7.2 Hz, 2H, Bn), 4.77 (s, 1H, NH), 4.21 (dd, J=11.2, 7.2 Hz, 1H, CH₂O), 4.02 (dd, J=11.2, 6.0 Hz, 1H, CH₂O), 3.72 (d, J=14.0 Hz, 2H, NCH₂Ph), 3.54 (d, J=14.0 Hz, 2H, NCH₂Ph), 3.45 (t, J=6.4 Hz, 2H, CH₂CH₂N), 3.34 (q, J=6.4 Hz, 2H, CH₂CH₂N), 3.06 (m, 1H, CHMe), 2.08 (t, J=6.4 Hz, 2H, CH₂Br), 1.07 (d, J=6.8 Hz, 3H, Me)

LC/MS (ES⁺) m/z 419.1-421.0 (M+H)⁺

### Preparation of (S)-2-(dibenzylamino)propyl 3-(1,2-dihydro-2-oxobenzo[d]imidazol-3-yl)propylcarbamate (Compound no 6)

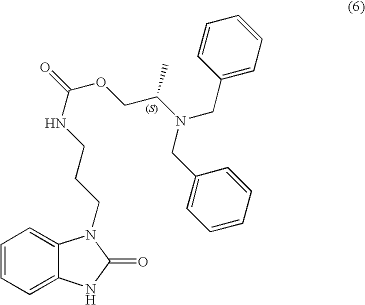

To a stirred solution of benzimidazolone (11 mg, 0.08 mmol) in DMF (0.1 mL) at 0° C. was added NaH (60% in mineral oil) (3.2 mg, 0.08 mmol) under N₂ atmosphere. The reaction mixture was stirred for 30 min at 0° C. The brominated derivative (30 mg, 0.07 mmol) in DMF (0.1 mL) was added and the reaction mixture was stirred overnight at room temperature. Water (2 mL) was introduced and the mixture was extracted with ethyl acetate (3×5 mL). The combined organic layers were dried over anhydrous Na₂SO₄, filtered and concentrated under vacuum. The crude material was purified by HPLC preparative to afford a white solid (2 mg, 6%).

¹H NMR (400 MHz, CDCl₃) δ 8.45 (s, 1H, NH), 7.36-7.12 (m, 10H, Ar), 7.02 (m, 4H, Ar), 5.72 (s, 1H, NH), 4.31 (m, 1H, CH₂O), 4.12 (m, 1H, CH₂O), 3.95 (m, 4H, CH₂CH₂CH₂NH, CH₂CH₂CH₂NH), 3.72 (m, 2H, NCH₂Ph), 3.58 (m, 2H, NCH₂Ph), 3.18 (m, 1H, CHMe), 1.70 (m, 2H, CH₂CH₂CH₂NH), 1.09 (m, 3H, Me)

LC/MS (ES⁺) m/z 473.2 (M+H)⁺

### 4) Reductive Amination

a. Reductive Amination (Primary Amine):

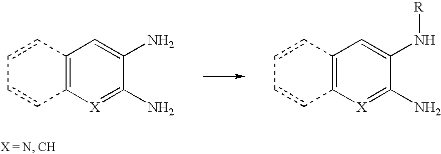

General Procedure:

To a stirred solution of appropriate diamine (3 equiv.) in 1,2-dichloroethane (1 mL/mmol) were added appropriate ketone (1 equiv.), acetic acid to (1.7 equiv.) followed by sodium triacetoxyborohydride (1.9 equiv.) under a nitrogen atmosphere. The reaction mixture was stirred for 16 h at room temperature. The solvent was removed on vacuo and the residue was partitioned between ethyl acetate and 2M aqueous Na₂CO₃ solution (5 mL/mmol). The organic phase was separated and washed by 2M aqueous Na₂CO₃ solution (2×5 mL/mmol). The organic layer was dried over anhydrous Na₂SO₄, filtered and concentrated under vacuum. The crude material was purified by flash chromatography on silica gel.

### Preparation of tert-butyl 4-(2-aminophenylamino)piperidine-1-carboxylate (Intermediate)

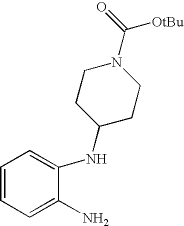

From benzene-1,2-diamine and tert-butyl 4-oxopiperidine-1-carboxylate

Flash chromatography on silica gel (cyclohexane/ethyl acetate 5/5)

Brown solid (88%).

¹H NMR (400 MHz, CDCl₃) δ 6.80 (m, 1H, Ar), 6.75 (m, 3H, Ar), 4.03 (bs, 2H, CHCH₂CH₂Nₚᵢₚ), 3.40 (m, 1H, CHCH₂CH₂Nₚᵢₚ), 2.92 (bt, 2H, CHCH₂CH₂Nₚᵢₚ), 2.04 (m, 2H, CHCH₂CH₂Nₚᵢₚ), 1.46 (s, 9H, OC(CH₃)₃), 1.14 (m, 2H, CHCH₂CH₂Nₚᵢₚ)

LC/MS (ES⁺) m/z 292.1 (M+H)⁺

### Preparation of (S)-2-(dibenzylamino)propyl 4-(2-aminopyridin-3-ylamino)piperidine-1-carboxylate (Intermediate)

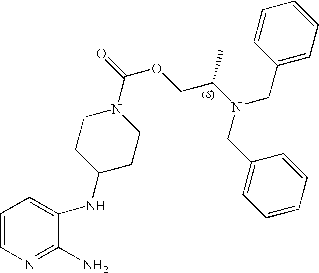

From pyridine-2,3-diamine and (S)-2-(dibenzylamino)propyl 4-oxopiperidine-1-carboxylate

Flash chromatography on silica gel (methylene chloride/ethanol 95/5)

Yellow solid (40%).

LC/MS (ES⁺) m/z 474.2 (M+H)⁺

### Preparation of tert-butyl 3-(2-aminophenylamino)piperidine-1-carboxylate (Intermediate)

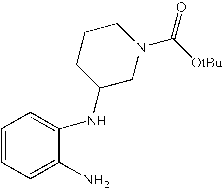

From benzene-1,2-diamine and tert-butyl 3-oxopiperidine-1-carboxylate

Flash chromatography on silica gel (cyclohexane/ethyl acetate 6/4)

Brown oil (50%).

¹H NMR (400 MHz, CDCl₃) δ 6.82-6.70 (m, 4H, Ar), 4.03 (m, 1H, CHCH₂N), 3.72 (bd, J=10.8 Hz, 1H, CHCH₂N), 3.36 (m, 1H, CHCH₂N), 3.20 (bs, 2H, NH₂), 3.08 (m, 1H, CHCH₂CH₂N), 2.91 (m, 1H, CHCH₂CH₂N), 2.02 (m, 1H, CHCH₂CH₂CH₂N), 1.76 (m, 1H, CHCH₂CH₂CH₂N), 1.57 (m, 2H, CHCH₂CH₂CH₂N), 1.45 (s, 9H, C(CH₃)₃)

LC/MS (ES⁺) m/z 292.1 (M+H)⁺

### Preparation of N-(1-benzylpyrrolidin-3-yl)benzene-1,2-diamine (Intermediate)

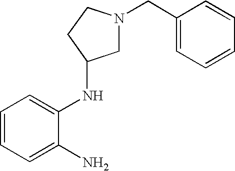

From benzene-1,2-diamine and 1-benzylpyrrolidin-3-one

Flash chromatography on silica gel (ethyl acetate)

Brown oil (40%).

LC/MS (ES⁺) m/z 268.2 (M+H)⁺

### Preparation of (S)-2-(dibenzylamino)propyl 4-(2-aminonaphthalen-3-ylamino)piperidine-1-carboxylate (Intermediate)

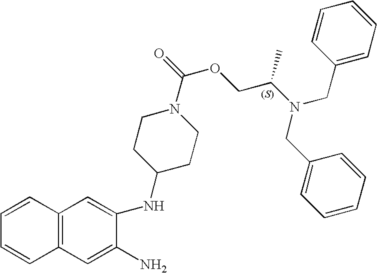

From naphthalene-2,3-diamine and (S)-2-(dibenzylamino)propyl 4-oxopiperidine-1-carboxylate

Flash chromatography on silica gel (cyclohexane/ethyl acetate 7/3)

Brown oil (20%).

LC/MS (ES⁺) m/z 523.2 (M+H)⁺

### Preparation of (S)-2-(dibenzylamino)propyl 4-(2-aminophenylamino)azepane-1-carboxylate (Intermediate)

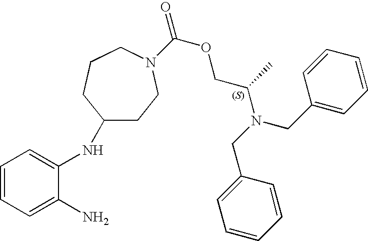

From benzene-1,2-diamine and (S)-2-(dibenzylamino)propyl 4-oxoazepane-1-carboxylate.

Flash chromatography on silica gel (cyclohexane/ethyl acetate 6/4)

Orange oil (58%).

LC/MS (ES⁺) m/z 487.2 (M+H)⁺

b. Reductive Amination (Primary Amine with Electrowithdrawing Group on the α Position of C═O)

To a stirred solution of appropriate diamine (2 equiv.) in methanol (1.5 mL/mmol) were added appropriate ketone (1 equiv.), acetic acid (1.3 equiv.) followed by supported sodium cyanoborohydride (1 equiv.) under a nitrogen atmosphere. The reaction mixture was stirred for 48 h at room temperature and was filtered. The filtrate was concentrated under vacuo and the residue was partitioned between ethyl acetate and saturated aqueous Na₂CO₃ solution (5 mL/mmol). The organic phase was separated and washed by saturated aqueous Na₂CO₃ solution (2×5 mL/mmol). The organic layer was dried over anhydrous Na₂SO₄, filtered and concentrated under vacuum. The crude material was purified by flash chromatography on silica gel.

### Preparation of ethyl 4-(2-aminophenylamino)-1-benzylpiperidine-3-carboxylate (Intermediate)

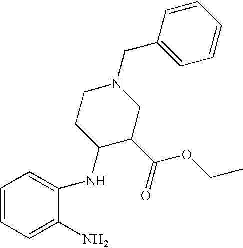

From benzene-1,2-diamine and ethyl 1-benzyl-4-oxopiperidine-3-carboxylate

Flash chromatography on silica gel (cyclohexane/ethyl acetate 5/5)

Brown oil (36%).

LC/MS (ES⁺) m/z 354.2 (M+H)⁺

c. Reductive Amination (Secondary Amine):

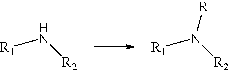

General Procedure:

To a mixture of appropriate amine (1 equiv.) and appropriate ketone or aldehyde (1.5 equiv.) were added acetic acid (1.7 mL/mmol) followed by sodium triacetoxyborohydride (1.5 equiv.) under a nitrogen atmosphere. The reaction mixture was stirred for 1 h at room temperature. The solvent was removed on vacuo and the residue was dissolved in ethyl acetate. Aqueous 4M NaOH solution was added dropwise to adjust pH to 10. The mixture was extracted with ethyl acetate (3×14 mL/mmol). The combined organic layers were dried over anhydrous Na₂SO₄, filtered and concentrated under vacuum. The crude material was purified by flash chromatography on silica gel.

### Preparation of 1-(1-(4-(dibenzylamino)butyl)piperidin-4-yl)-1H-benzo[d]imidazol-2(3H)-one (Compound no 9)

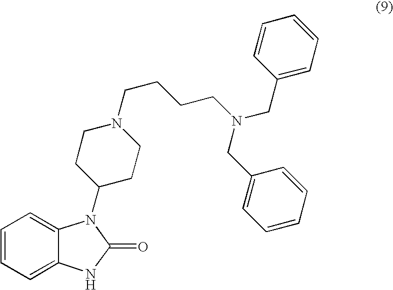

From 4-(2-keto-1-benzimidazolinyl)-piperidine and 4-dibenzylaminobutyraldehyde

Colorless oil (50%).

Flash chromatography on silica gel (cyclohexane/ethyl acetate 2/8)

¹H NMR (400 MHz, CDCl₃) δ 9.10 (s, 1H), 7.38 (d, J=7.3 Hz, 4H, Ph), 7.32 (t, J=7.4 Hz, 4H, Ph), 7.24 (m, 3H, Ph), 7.07 (m, 3H, Ph), 4.38 (m, 1H, CHCH₂CH₂N), 3.57 (s, 4H, NCH₂Ph), 3.04 (m, 2H, CHCH₂CH₂N), 2.45 (t, J=6.2 Hz, 2H, NCH₂(CH₂)₂CH₂NBn₂), 2.32 (m, 2H, CHCH₂CH₂N), 2.11 (m, 2H, CHCH₂CH₂N), 1.82 (m, 2H, CHCH₂CH₂N), 1.68 (m, 2H, NCH₂(CH₂)₂CH₂NBn₂), 1.55 (m, 2H, NCH₂(CH₂)₂CH₂NBn₂), 1.26 (m, 2H, NCH₂(CH₂)₂CH₂NBn₂)

LC/MS (ES⁺) m/z 469.3 (M+H)⁺

### 5) Cyclisation (with Carbonyldiimidazole)

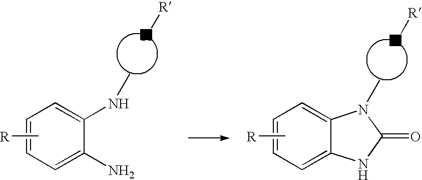

General Procedure:

To a stirred solution of appropriate diamine (1 equiv.) in THF (10 mL/mmol) was added CDI (1.5 equiv) under N₂ atmosphere. The reaction mixture was stirred overnight at room temperature. The solvent was removed on vacuo and the residue was purified by flash chromatography on silica gel.

### Preparation of (S)-2-(dibenzylamino)propyl 4-(2,3-dihydro-2-oxoimidazo[4,5-b]pyridin-1-yl)piperidine-1-carboxylate (Compound no 11)

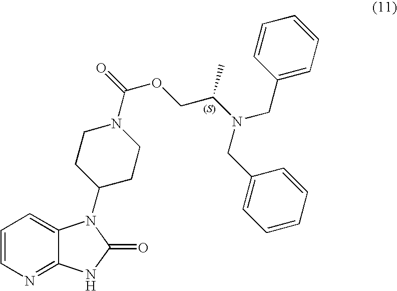

From (S)-2-(dibenzylamino)propyl 4-(2-aminopyridin-3-ylamino)piperidine-1-carboxylate

Stirred for 2 days at room temperature.

Flash chromatography on silica gel (methylene chloride/acetone 7/3)

Colorless oil (57%).

¹H NMR (400 MHz, CDCl₃) δ 10.34 (bs, 1H, NH), 8.07 (d, J=5.2 Hz, 1H, Ar), 7.37 (m, 4H, Ar), 7.28-7.21 (m, 7H, Ar), 6.95 (t, J=7.6 Hz, 1H, Ar), 4.60 (m, 1H, CHCH₂CH₂Nₚᵢₚ), 4.40 (m, 2H, CHCH₂CH₂Nₚᵢₚ), 4.27 (m, 1H, CH₂O), 4.08 (dd, J=5.2, 10.4 Hz, 1H, CH₂O), 3.76 (d, J=13.8 Hz, 2H, NCH₂Ph), 3.56 (d, J=13.8 Hz, 2H, NCH₂Ph), 3.14 (m, 1H, CHMe), 2.94 (m, 2H, CHCH₂CH₂Nₚᵢₚ), 2.24 (m, 2H, CHCH₂CH₂Nₚᵢₚ), 1.92 (m, 2H, CHCH₂CH₂Nₚᵢₚ), 1.11 (d, J=6.4 Hz, 3H, Me).

¹³C NMR (100 MHz, CDCl₃) δ 155.1 (C═O), 153.3 (C═O), 143.1 (C_{Ar}), 140.2 (C_{Ar}), 140.1 (CH_{Ar}), 133.1 (C_{Ar}), 128.6 (CH_{Ar}), 128.2 (CH_{Ar}), 126.8 (CH_{Ar}), 116.8 (CH_{Ar}), 115.4 (CH_{Ar}), 66.9 (CH₂O), 53.4 (NCH₂Ph), 51.6 (CHMe), 50.1 (CHCH₂CH₂Nₚᵢₚ), 43.1 (CHCH₂CH₂Nₚᵢₚ), 28.9 (CHCH₂CH₂Nₚᵢₚ), 11.9 (Me).

LC/MS (ES⁺) m/z 500.2 (M+H)⁺

### Preparation of (S)-2-(dibenzylamino)propyl 4-(1,2-dihydro-2-oxonaphtho[2,3-d]imidazol-3-yl)piperidine-1-carboxylate (Compound no 12)

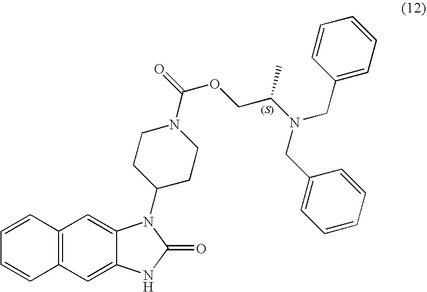

From (S)-2-(dibenzylamino)propyl 4-(2-aminonaphthalen-3-ylamino)piperidine-1-carboxylate

Preparative TLC (silica gel) (cyclohexane/ethyl acetate 5/5)

Green oil (38%).

¹H NMR (400 MHz, CDCl₃) δ 9.34 (bs, 1H, NH), 7.78 (m, 2H, Ar), 7.40 (m, 8H, Ar), 7.37 (m, 4H, Ar), 7.20 (m, 2H, Ar), 4.58 (m, 1H, CHCH₂CH₂Nₚᵢₚ), 4.46 (m, 2H, CHCH₂CH₂Nₚᵢₚ), 4.31 (dd, J=7.6, 10.8 Hz, 1H, CH₂O), 4.11 (dd, J=5.6, 10.8 Hz, 1H, CH₂O), 3.77 (d, J=13.8 Hz, 2H, NCH₂Ph), 3.60 (d, J=13.8 Hz, 2H, NCH₂Ph), 3.18 (m, 1H, CHMe), 3.00 (m, 2H, CHCH₂CH₂Nₚᵢₚ), 2.54 (m, 2H, CHCH₂CH₂Nₚᵢₚ), 1.92 (m, 2H, CHCH₂CH₂Nₚᵢₚ), 1.13 (d, J=6.4 Hz, 3H, Me).

¹³C NMR (100 MHz, CDCl₃) δ 155.6 (C═O), 155.3 (C═O), 140.2 (C_{Ar}), 129.9 (C_{Ar}), 129.4 (C_{Ar}), 128.5 (CH_{Ar}), 128.4 (C_{Ar}), 128.2 (CH_{Ar}), 127.2 (CH_{Ar}), 126.9 (C_{Ar}), 126.8 (2×CH_{Ar}), 124.3 (CH_{Ar}), 124.3 (CH_{Ar}), 105.4 (CH_{Ar}), 105.1 (CH_{Ar}), 66.8 (CH₂O), 53.6 (NCH₂Ph), 51.9 (CHMe), 50.9 (CHCH₂CH₂Nₚᵢₚ), 43.6 (CHCH₂CH₂Nₚᵢₚ), 28.9 (CHCH₂CH₂Nₚᵢₚ).

LC/MS (ES⁺) m/z 549.2 (M+H)⁺

### Preparation of (S)-2-(dibenzylamino)propyl 4-(1,2-dihydro-2-oxobenzo[d]imidazol-3-yl)azepane-1-carboxylate (Compound no 13)

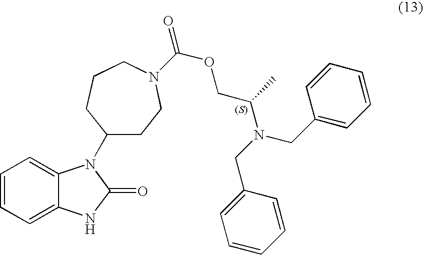

From (S)-2-(dibenzylamino)propyl 4-(2-aminophenylamino)azepane-1-carboxylate.

Flash chromatography on silica gel (cyclohexane/ethyl acetate 5/5)

Colorless oil (66%).

¹H NMR (400 MHz, CDCl₃) δ 9.37 (bd, 1H, NH), 7.39-7.21 (m, 10H, Ar), 7.04 (m, 4H, Ar), 4.46 (m, 1H, CHCH₂CH₂N), 4.28 (m, 1H, CH₂O), 4.12 (m, 1H, CH₂O), 3.72 (m, 3H, NCH₂Ph, CH₂CH₂CH₂N), 3.61 (m, 4H, NCH₂Ph, CHCH₂CH₂N), 3.33 (m, 1H, CH₂CH₂CH₂N), 3.14 (m, 1H, CHMe), 2.36 (m, 2H, CHCH₂CH₂N), 2.05 (m, 3H, CH₂CH₂CH₂N, CH₂CH₂CH₂N), 1.72 (m, 1H, CH₂CH₂CH₂N), 1.10 (m, 3H, Me).

¹³C NMR (100 MHz, CDCl₃) δ 156.2 (C═O), 154.4 (C═O), 140.3 (140.2) (C_{Ar}), 128.9 (128.8) (C_{Ar}), 128.6 (128.5) (CH_{Ar}), 128.1 (CH_{Ar}), 127.9 (127.8) (C_{Ar}), 126.8 (126.7) (CH_{Ar}), 121.2 (CH_{Ar}), 121.1 (121.1) (CH_{Ar}), 109.7 (109.6) (CH_{Ar}), 109.3 (109.2) (CH_{Ar}), 66.8 (CH₂O), 53.8 (NCH₂Ph), 53.7 (CHCH₂CH₂N), 51.9 (CHMe), 46.5 (46.6) (CHCH₂CH₂N), 43.0 (CH₂CH₂CH₂N), 33.0 (32.9) (CHCH₂CH₂N), 30.1 (CH₂CH₂CH₂N), 26.8 (CH₂CH₂CH₂N), 11.6 (Me).

LC/MS (ES⁺) m/z 513.3 (M+H)⁺

### Preparation of (S)-2-(dibenzylamino)propyl 4-(2-phenyl-1H-benzo[d]imidazol-1-yl)piperidine-1-carboxylate (Compound no 153)

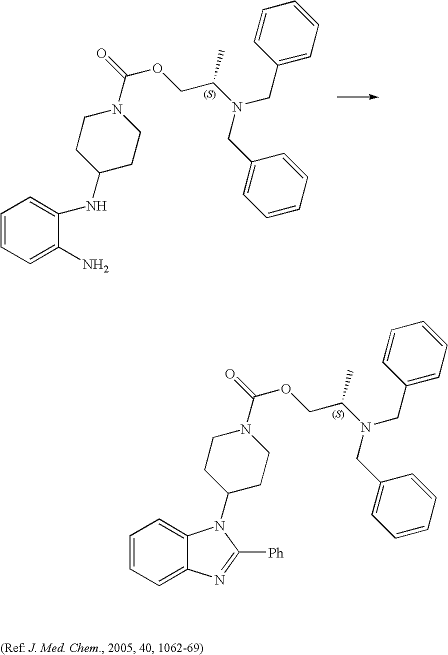

To a stirred solution of benzaldehyde (12 μL, 0.117 mmol) in EtOH (0.4 mL) was added Na₂S₂O₃ (20 mg, 0.117 mmol) in water (0.1 mL). The reaction mixture was stirred at room temperature for 20 min and cooled at 0° C. for 3 h. The white precipitate was filtered and dried on vacuum. To the crude precipitate was added a solution of (S)-2-(dibenzylamino)propyl 4-(2-aminophenylamino)piperidine-1-carboxylate (50 mg, 0.106 mmol) in DMF (3 mL) under N₂ atmosphere. The reaction mixture was stirred at room temperature for 4 h. Water (3 mL) was introduced and the mixture was extracted with ethyl acetate (3×5 mL). The combined organic layers were dried over anhydrous Na₂SO₄, filtered and concentrated under vacuum. The residue was purified by flash chromatography on silica gel (dichloromethane/Et₂O 95/5) to obtain a brown oil (40 mg, 68%).

¹HNMR (400 MHz, CDCl₃) δ 7.82 (d, J=7.6 Hz, 1H, Ar), 7.64 (m, 2H, Ar), 7.56 (m, 4H, Ar), 7.38 (d, J=7.2 Hz, 4H, Ar), 7.20 (m, 8H, Ar), 4.55 (m, 1H, CHCH₂CH₂Nₚᵢₚ), 4.42 (m, 2H, CHCH₂CH₂Nₚᵢₚ), 4.29 (dd, J=7.6, 11.2 Hz, 1H, CH₂O), 4.08 (m, 1H, CH₂O), 3.77 (d, J=13.8 Hz, 2H, NCH₂Ph), 3.56 (d, J=13.8 Hz, 2H, NCH₂Ph), 3.14 (m, 1H, CHMe), 2.82 (m, 2H, CHCH₂CH₂Nₚᵢₚ), 2.55 (m, 2H, CHCH₂CH₂Nₚᵢₚ), 1.95 (bd, J=10.4 Hz, 2H, CHCH₂CH₂Nₚᵢₚ), 1.11 (d, J=6.8 Hz, 3H, Me).

¹³C NMR (100 MHz, CDCl₃) δ 155.1 (C═O), 153.6 (C═N), 142.9 (Ar), 140.2 (Ar), 133.5 (Ar), 130.8 (Ar), 129.8 (Ar), 129.3 (Ar), 128.8 (Ar), 128.5 (Ar), 128.1 (Ar), 126.8 (Ar), 122.6 (Ar), 122.3 (Ar), 120.4 (Ar), 112.2 (Ar), 66.9 (CH₂O), 55.0 (CHCH₂CH₂Nₚᵢₚ), 53.6 (NCH₂Ph), 51.9 (CHMe), 43.4 (CHCH₂CH₂Nₚᵢₚ), 30.4 (CHCH₂CH₂Nₚᵢₚ), 11.2 (Me).

LC/MS (ES⁺) m/z 559.2 (M+H)⁺

### Preparation of (S)-2-(dibenzylamino)propyl 4-(2-amino-1H-benzo[d]imidazol-1-yl)piperidine-1-carboxylate (Compound no 177)

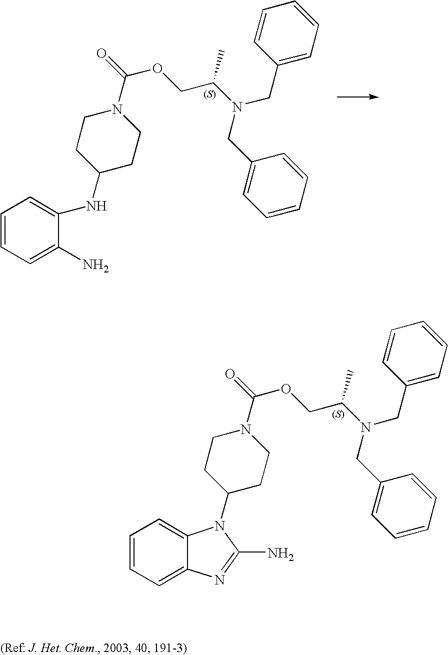

To a stirred solution of (S)-2-(dibenzylamino)propyl 4-(2-aminophenylamino)piperidine-1-carboxylate (71 mg, 0.15 mmol) in THF (5 mL) was added di(1H-imidazol-1-yl)methanimine (29 mg, 0.18 mmol) under N₂ atmosphere. The reaction mixture was refluxed overnight and concentrated under vacuum. The residue was purified by flash chromatography on silica gel (cyclohexane/ethyl acetate 4/6) to obtain a brown solid (15 mg, 20%).

¹H NMR (400 MHz, CDCl₃) δ 8.11 (s, 1H, NH), 7.38 (m, 4H, Ar), 7.29 (m, 6H, Ar), 7.20 (m, 2H, Ar), 7.06 (m, 2H, Ar), 4.50 (m, 1H, CHCH₂CH₂Nₚᵢₚ), 4.41 (m, 2H, CHCH₂CH₂Nₚᵢₚ), 4.29 (dd, J=7.6, 11.2 Hz, 1H, CH₂O), 4.07 (dd, J=5.6, 11.2 Hz, 1H, CH₂O), 3.76 (d, J=13.6 Hz, 2H, NCH₂Ph), 3.56 (d, J=13.6 Hz, 2H, NCH₂Ph), 3.14 (m, 1H, CHMe), 2.92 (m, 2H, CHCH₂CH₂Nₚᵢₚ), 2.38 (m, 2H, CHCH₂CH₂Nₚᵢₚ), 1.88 (m, 2H, CHCH₂CH₂Nₚᵢₚ), 1.11 (d, J=6.8 Hz, 3H, Me).

LC/MS (ES⁺) m/z 498.3 (M+H)⁺

### 6) Cyclisation (CDI)/NBoc Deprotection/Carbamate Preparation

General Procedure:

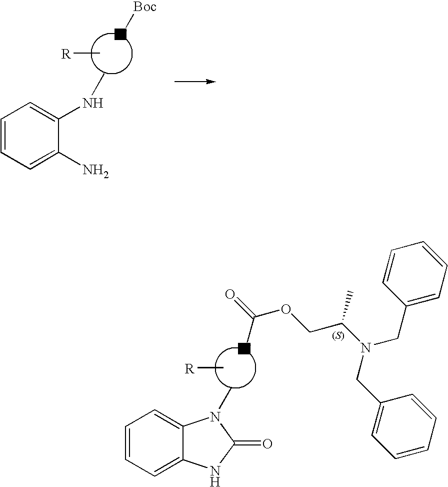

To a stirred solution of appropriate diamine (1 equiv.) in THF (10 mL/mmol) was added CDI (1.5 equiv) under N₂ atmosphere. The reaction mixture was stirred overnight at room temperature. The solvent was removed on vacuo. Ethyl acetate was added and the organic layer was washed with water, dried over Na₂SO₄ and concentrated in vacuo. To a solution of the crude material (1 equiv.) in CH₂Cl₂ (2.5 mL/mmol) cooled to 0° C. under N₂ was added TFA (8 equiv.). The reaction mixture was stirred for 2 h at room temperature. The solvent was removed on vacuo and the residue was triturated with Et₂O to give a solid. To a stirred solution of (S)-2-(dibenzylamino)propan-1-ol (1 equiv.) in THF cooled to 0° C. was added CDI (1.1 equiv.) under N₂ atmosphere. The reaction mixture was stirred for 2 h at room temperature. Triethylamine (1.6 equiv.) and the crude salt (1 equiv.) were added and the reaction mixture was stirred for 72 h at room temperature. The solvent was removed on vacuo and the residue was purified by flash chromatography on silica gel.

### Preparation of (S)-2-(dibenzylamino)propyl 3-(1,2-dihydro-2-oxobenzo[d]imidazol-3-yl)piperidine-1-carboxylate (Compound no 16)

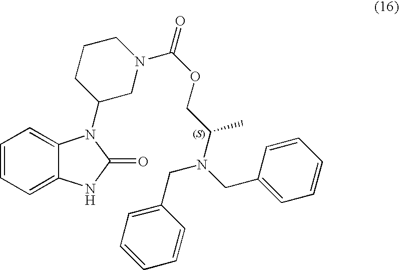

From tert-butyl 3-(2-aminophenylamino)piperidine-1-carboxylate

Flash chromatography on silica gel (cyclohexane/ethyl acetate 7/3)

Pale yellow solid (77%).

¹HNMR (400 MHz, CDCl₃) δ 10.42-10.31 (bs, 1H, NH), 7.40-7.26 (m, 8H, Ar), 7.22-7.07 (m, 6H, Ar), 4.31 (m, 4H, CHCH₂N, CHCH₂N, CH₂O), 4.07 (m, 2H, CH₂O, CH₂CH₂CH₂N), 3.74 (m, 2H, NCH₂Ph), 3.56 (m, 3H, NCH₂Ph, CH₂CH₂CH₂N), 3.13 (m, 1H, CHMe), 2.86 (bs, 1H, CH₂CH₂CH₂N), 2.51 (bs, 1H, CH₂CH₂CH₂N), 1.92 (bs, 1H, CH₂CH₂CH₂N), 1.86 (m, 1H, CH₂CH₂CH₂N), 1.10 (bs, 3H, Me).

¹³C NMR (100 MHz, CDCl₃) δ 155.4 (C═O), 153.2 (C═O), 140.2 (C_{Ar}), 129.1 (C_{Ar}), 128.5 (CH_{Ar}), 128.1 (CH_{Ar}), 128.0 (C_{Ar}), 126.7 (CH_{Ar}), 121.4 (CH_{Ar}), 121.1 (CH_{Ar}), 109.9 (CH_{Ar}), 108.5 (CH_{Ar}), 66.9 (CH₂O), 53.7 (NCH₂Ph), 51.8 (CHMe), 50.2 (CHCH₂N), 45.8 (CHCH₂N), 43.9 (CH₂CH₂CH₂N), 28.0 (CH₂CH₂CH₂N), 27.8 (CH₂CH₂CH₂N), 11.3 (Me).

LC/MS (ES⁺) m/z 499.1 (M+H)⁺

### 7) Cyclisation (CDI)/NBn Deprotection/Carbamate Preparation

General Procedure

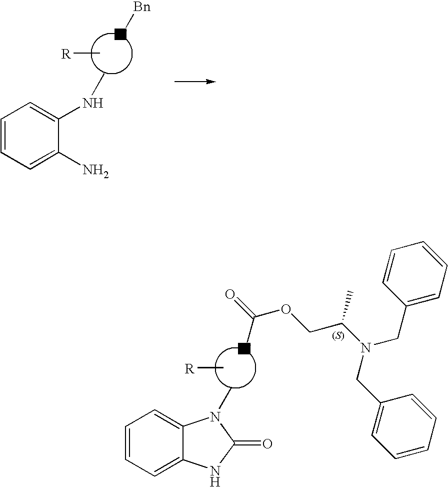

To a stirred solution of appropriate diamine (1 equiv.) in THF (10 mL/mmol) was added CDI (1.5 equiv) under N₂ atmosphere. The reaction mixture was stirred overnight at room temperature. The solvent was removed on vacuo. Ethyl acetate was added and the organic layer was washed with water, dried over Na₂SO₄ and concentrated in vacuo. A solution of the crude material (1 equiv.) in EtOH (10 mL/mmol) was submitted to hydrogenation in the presence of acetic acid (6 equiv.) and 10% Pd/C (100 mg/mmol) at room pressure and temperature for 24 h. The reaction mixture was filtered through a pad of Celite. The filtrate was concentrated under vacuo. To a stirred solution of (S)-2-(dibenzylamino)propan-1-ol (1 equiv.) in THF cooled to 0° C. was added CDI (1.1 equiv.) under N₂ atmosphere. The reaction mixture was stirred for 2 h at room temperature. Triethylamine (1.6 equiv.) and the crude salt (1 equiv.) were added and the reaction mixture was stirred for 72 h at room temperature. The solvent was removed on vacuo and the residue was purified by flash chromatography on silica gel.

### Preparation of (S)-2-(dibenzylamino)propyl 3-(1,2-dihydro-2-oxobenzo[d]imidazol-3-yl)pyrrolidine-1-carboxylate (Compound no 17)

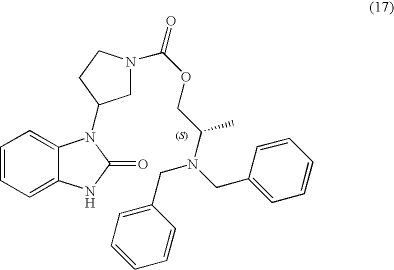

From N-1-(1-benzylpyrrolidin-3-yl)benzene-1,2-diamine

Flash chromatography on silica gel (cyclohexane/ethyl acetate 5/5)

Yellow oil (12%).

¹H NMR (400 MHz, CDCl₃) δ 9.07 (bd, 1H, NH), 7.38-7.16 (m, 10H, Ar), 7.09 (m, 4H, Ar), 5.15 (m, 1H, CHCH₂N), 4.27 (m, 1H, CH₂O), 4.10 (dd, J=5.6, 10.8 Hz, 1H, CH₂O), 3.79 (m, 5H, NCH₂Ph, CHCH₂N, CH₂CH₂N), 3.56 (m, 3H, NCH₂Ph, CH₂CH₂N), 3.10 (m, 1H, CHMe), 2.63 (m, 1H, CH₂CH₂N), 2.29 (m, 1H, CH₂CH₂N), 0.93 (bd, 3H, Me).

LC/MS (ES⁺) m/z 485.2 (M+H)⁺

### Preparation of 1-(S)-2-(dibenzylamino)propyl 3-ethyl 4-(1,2-dihydro-2-oxobenzo[d]imidazol-3-yl)piperidine-1,3-dicarboxylate (Compound no 18)

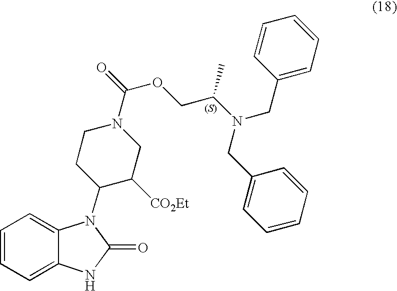

From ethyl 4-(2-aminophenylamino)-1-benzylpiperidine-3-carboxylate.

Flash chromatography on silica gel (cyclohexane/ethyl acetate 5/5)

Yellow solid (19%).

¹H NMR (400 MHz, CDCl₃) δ 9.51 (bs, 1H, NH), 7.47 (m, 4H, Ar), 7.21 (m, 7H, Ar), 7.04 (m, 3H, Ar), 4.65 (m, 1H, CHCH₂CH₂N), 4.47 (m, 2H, CH₂O, CHCHCH₂N), 4.31 (m, 2H, CH₂O, CHCH₂CH₂N), 3.87 (m, 3H, NCH₂Ph, OCH₂CH₃), 3.74 (m, 3H, NCH₂Ph, OCH₂CH₃), 3.26 (m, 2H, CHMe, CHCHCH₂N), 3.06 (m, 3H, CHCH₂CH₂N, CHCH₂CH₂N), 1.94 (m, 1H, CHCHCH₂N), 1.22 (m, 3H, Me), 0.89 (bq, 1.5H, OCH₂CH₃), 0.74 (m, 1.5H, OCH₂CH₃).

LC/MS (ES⁺) m/z 571.3 (M+H)⁺

### 8) NBoc Deprotection/Carbamate Preparation

General Procedure

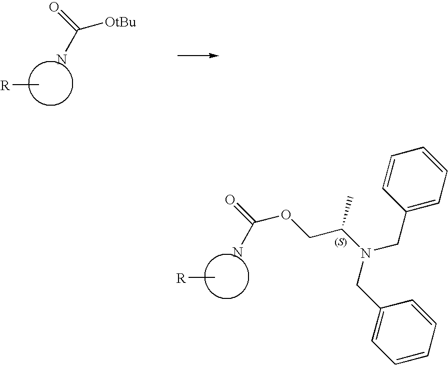

To a stirred solution of appropriate N-tert-butyloxycarbonyl derivative (1 equiv.) in CH₂Cl₂ (2.5 mL/mmol) cooled to 0° C. under N₂ was added TFA (8 equiv.). The reaction mixture was stirred for 2 h at room temperature. The solvent was removed on vacuo and the residue was triturated with Et₂O to give a solid. To a stirred solution of (S)-2-(dibenzylamino)propan-1-ol (1 equiv.) in THF (2 mL/mmol) at 0° C. was added CDI (1.1 equiv.) under N₂ atmosphere. The reaction mixture was stirred for 2 h at room temperature. Triethylamine (1.8 equiv.) and the crude salt (1.3 equiv.) were added and the reaction mixture was stirred for 72 h at room temperature. The solvent was removed on vacuo and the residue was purified by flash chromatography on silica gel.

### Preparation of (S)-2-(dibenzylamino)propyl 4-oxopiperidine-1-carboxylate (Intermediate)

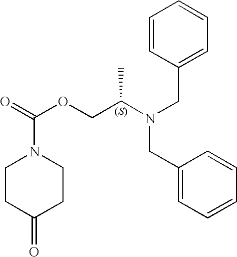

From N-tert-butyloxycarbonylpiperid-4-one and (S)-2-(dibenzylamino)propan-1-ol.

Flash chromatography on silica gel (methylene chloride/acetone 97/3).

White solid (31%).

¹H NMR (400 MHz, CDCl₃) δ 7.35 (d, J=7.2 Hz, 4H, Ar), 7.28 (t, J=7.2 Hz, 4H, Ar), 7.18 (t, J=7.2 Hz, 2H, Ar), 4.27 (dd, J=7.6, 11.2 Hz, 1H, CH₂O), 4.06 (dd, J=5.6, 11.2 Hz, 1H, CH₂O), 3.75 (m, 6H, NCH₂Ph, CH₂CH₂Nₚᵢₚ), 3.54 (d, J=13.6 Hz, 2H, NCH₂Ph), 3.14 (m, 1H, CHMe), 2.45 (m, 4H, CH₂CH₂Nₚᵢₚ), 1.10 (d, J=6.8 Hz, 3H, Me).

LC/MS (ES⁺) m/z 381.1 (M+H)⁺

### Preparation of (S)-2-(dibenzylamino)propyl 4-hydroxypiperidine-1-carboxylate (Intermediate)

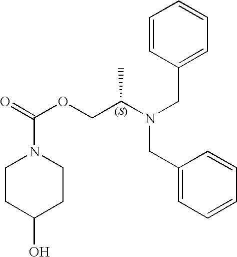

From N-tert-butyloxy-carbonyl-4-hydroxypiperide and (S)-2-(dibenzylamino)propan-1-ol

Flash chromatography on silica gel (cyclohexane/ethyl acetate: 7/3)

Colorless oil (95%).

¹H NMR (400 MHz, CDCl₃) δ 7.37 (d, J=7.2 Hz, 4H), 7.29 (t, J=7.4 Hz, 4H), 7.23 (m, 2H), 4.22 (dd, J=10.9, 7.5 Hz, 1H, CHCH₂CH₂N), 4.03 (dd, J=10.9, 5.5 Hz, 1H, OCH₂), 3.90 (m, 3H, OCH₂, CHCH₂CH₂N), 3.74 (d, J=13.9 Hz, 2H, NCH₂Ph), 3.56 (d, J=13.9 Hz, 2H, NCH₂Ph), 3.15 (m, 3H, CHCH₃, CHCH₂CH₂N), 1.89 (d, J=9.0 Hz, 2H, CHCH₂CH₂N), 1.61-1.44 (m, 2H, CHCH₂CH₂N), 1.08 (d, J=6.8 Hz, 3H, CHCH₃)

LC/MS (ES⁺) m/z 383.0 (M+H)⁺

### Preparation of (S)-2-(dibenzylamino)propyl 4-((1,2-dihydro-2-oxobenzo[d]imidazol-3-yl)methyl)piperidine-1-carboxylate (Compound no 19)

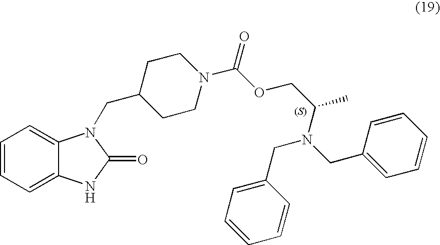

From tert-butyl 44(1,2-dihydro-2-oxobenzo[d]imidazol-3-yl)methyl)piperidine-1-carboxylate.

THF (10 mL/mmol) was added for the second step to improve solubility.

Flash chromatography on silica gel (cyclohexane/ethyl acetate 4/6)

Pale yellow oil (10%).

¹H NMR (400 MHz, CDCl₃) δ 9.05 (bs, 1H, NH), 7.36 (d, J=7.2 Hz, 4H, Bn), 7.27 (m, 4H, Bn), 7.18 (m, 2H, Bn), 7.08 (m, 3H, Ar), 6.98 (m, 1H, Ar), 4.21 (m, 3H, CHCH₂CH₂Nₚᵢₚ, CH₂O), 4.02 (dd, J=5.6, 10.8 Hz, 1H, CH₂O), 3.77 (m, 4H, NCH₂CH, NCH₂Ph), 3.55 (d, J=14.0 Hz, 2H, NCH₂Ph), 3.10 (m, 1H, CHMe), 2.74 (m, 2H, CHCH₂CH₂Nₚᵢₚ), 2.12 (m, 1H, CHCH₂CH₂Nₚᵢₚ), 1.74 (m, 2H, CHCH₂CH₂Nₚᵢₚ), 1.25 (m, 2H, CHCH₂CH₂Nₚᵢₚ), 1.07 (d, J=6.9 Hz, 3H, Me).

LC/MS (ES⁺) m/z 513.2 (M+H)⁺

### Preparation of (S)-2-(dibenzylamino)propyl 4-(2-(1,2-dihydro-2-oxobenzo[d]imidazol-3-yl)ethyl)piperidine-1-carboxylate (Compound no 20)

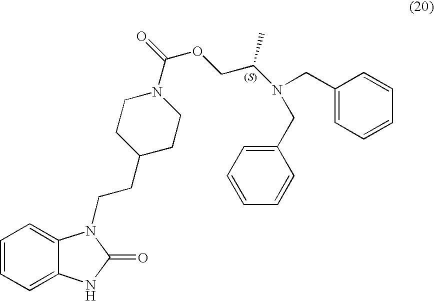

From tert-butyl 4-(2-(1,2-dihydro-2-oxobenzo[d]imidazol-3-yl)ethyl)piperidine-1-carboxylate.

THF (10 mL/mmol) was added for the second step to improve solubility.

Flash chromatography on silica gel (cyclohexane/ethyl acetate 5/5)

Pale yellow oil (50%).

¹H NMR (400 MHz, CDCl₃) δ 9.79 (bs, 1H, NH), 7.37 (d, J=7.2 Hz, 4H, Bn), 7.36 (t, J=7.2 Hz, 4H, Bn), 7.21 (t, J=7.2 Hz, 2H, Bn), 7.07 (m, 3H, Ar), 6.97 (d, J=7.2 Hz, 1H, Ar), 4.22 (m, 3H, CHCH₂CH₂Nₚᵢₚ, CH₂O), 4.03 (m, 1H, CH₂O), 3.96 (t, J=7.2 Hz, 2H, NCH₂CH₂CH), 3.74 (d, J=14.0 Hz, 2H, NCH₂Ph), 3.57 (d, J=14.0 Hz, 2H, NCH₂Ph), 3.11 (m, 1H, CHMe), 2.77 (m, 2H, CHCH₂CH₂Nₚᵢₚ), 1.84 (m, 2H, CHCH₂CH₂Nₚᵢₚ), 1.75 (m, 2H, NCH₂CH₂CH), 1.55 (m, 1H, CHCH₂CH₂Nₚᵢₚ), 1.28 (m, 2H, CHCH₂CH₂Nₚᵢₚ), 1.08 (d, J=6.8 Hz, 3H).

¹³C NMR (100 MHz, CDCl₃) δ 155.4 (C═O), 155.3 (C═O), 140.3 (C_{Ar}), 130.1 (C_{Ar}), 128.5 (CH_{Ar}), 128.1 (CH_{Ar}), 128.0 (C_{Ar}), 126.8 (CH_{Ar}), 121.6 (CH_{Ar}), 121.3 (CH_{Ar}), 109.6 (CH_{A}), 107.7 (CH_{Ar}), 66.5 (CH₂O), 53.7 (NCH₂Ph), 51.9 (CHMe), 44.0 (CHCH₂CH₂Nₚᵢₚ), 38.3 (NCH₂CH₂CH), 34.8 (NCH₂CH₂CH), 33.5 (CHCH₂CH₂Nₚᵢₚ), 27.0 (CHCH₂CH₂Nₚᵢₚ), 11.3 (Me).

LC/MS (ES⁴) m/z 527.2 (M+H)⁺

### 9) NBn Deprotection/Carbamate Preparation

General Procedure

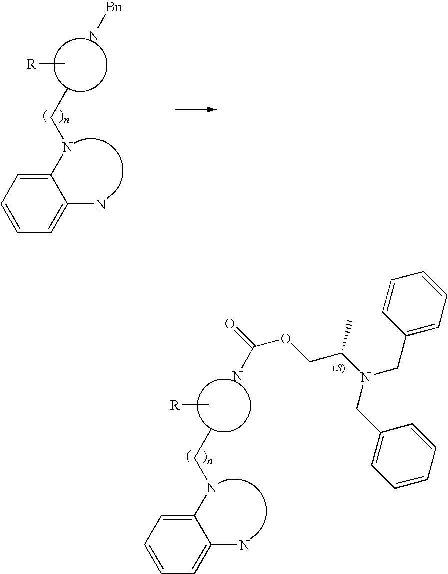

A solution of the N-benzyl derivative (1 equiv.) in EtOH (10 mL/mmol) was submitted to hydrogenation in the presence of acetic acid (6 equiv.) and 10% Pd/C (100 mg/mmol) at room pressure and temperature for 24 h. The reaction mixture was filtered through a pad of Celite. The filtrate was concentrated under vacuo. To a stirred solution of (S)-2-(dibenzylamino)propan-1-ol (1 equiv.) in THF (4 mL/mmol) was added CDI (1.1 equiv.) at 0° C. under N₂ atmosphere. The reaction mixture was stirred for 2 h at room temperature. Triethylamine (1.6 equiv.) and the crude salt (1.3 equiv.) in THF (4 mL/mmol) were added and the reaction mixture was stirred for 72 h at room temperature. The solvent was removed on vacuo and the residue was purified by flash chromatography on silica gel.

### Preparation of (S)-2-(dibenzylamino)propyl 24(1,2-dihydro-2-oxobenzoldiimidazol-3-yl)methyl)pyrrolidine-1-carboxylate (Compound no 23)

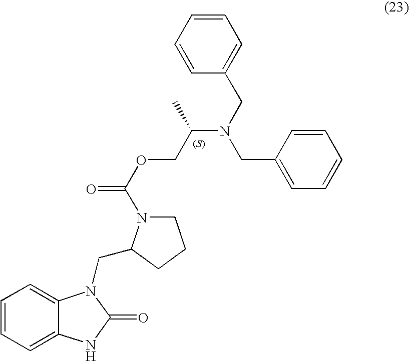

From 1-((1-benzylpyrrolidin-2-yl)methyl)-1H-benzo[d]imidazol-2(3H)-one

Colorless oil (24%).

Flash chromatography on silica gel (cyclohexane/ethyl acetate 5/5)

¹H NMR (400 MHz, CDCl₃) δ 9.67 (bs, 1H, NH), 7.39 (m, 5H, Ar), 7.29 (m, 6H, Ar), 7.22 (m, 3H, Ar), 4.25 (m, 2H, CH₂O, CH₂CHN), 4.12 (m, 1H, CH₂O), 4.02 (m, 2H, CH₂CH₂N), 3.78 (d, J=13.8 Hz, 2H, NCH₂Ph), 3.59 (d, J=13.8 Hz, 2H, NCH₂Ph), 3.45 (m, 2H, C′H₂NCO), 3.15 (m, 1H, CHMe), 2.05 (m, 2H, CH₂CH₂N), 1.92 (m, 2H, CH₂CHN), 1.12 (d, J=6.8 Hz, 3H, Me).

¹³C NMR (100 MHz, CDCl₃) δ 155.8 (C═O), 155.4 (C═O), 140.3 (C_{Bn}), 130.4 (C_{Ar}), 128.6 (CH_{Bn}), 128.1 (CH_{Bn}), 127.7 (Cm), 126.8 (CH_{Bn}), 121.7 (CH_{Ar}), 121.6 (CH_{Ar}), 109.3 (CH_{Ar}), 108.9 (CH_{Ar}), 66.6 (CH₂O), 56.4 (CH₂CHN), 53.7 (NCH₂Ph), 51.9 (CHMe), 46.6 (CH₂NCO), 42.2 (CH₂CH₂N), 28.3 (CH₂CHN), 23.7 (CH₂CH₂N), 11.5 (Me).

LC/MS (ES⁺) m/z 499.2 (M+H)⁺

### 10) Carbamate Preparation (CDI Coupling)

General Procedure:

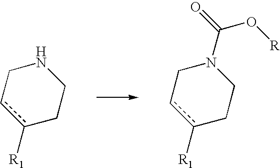

To a stirred solution of the appropriate alcohol (1 equiv.) in THF (2 mL/mmol) at 0° C. was added CDI (1.1 equiv.) under N₂ atmosphere. The reaction mixture was stirred for 2 h at room temperature. The appropriate amine (1.5 equiv.) in THF (10 mL/mmol) was added and the reaction mixture was stirred for 72 h at room temperature. The solvent was removed on vacuo and the residue was purified by flash chromatography on silica gel.

### Preparation of (S)-2-(dibenzylamino)propyl 4-(1,2-dihydro-2-oxobenzo[d]imidazol-3-yl)piperidine-1-carboxylate (Compound no 25)

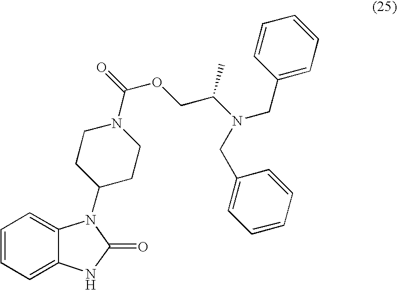

From 4-(2-keto-1-benzimidazolinyl)-piperidine and (S)-2-(dibenzylamino)propan-1-ol

White solid (100%).

Flash chromatography on preparative TLC (silica gel, cyclohexane/ethyl acetate 7/3)

¹H NMR (400 MHz, CDCl₃) δ 10.21 (s, 1H, NH), 7.39 (d, J=7.3 Hz, 4H, Ph), 7.28 (dd, J=12.7, 5.1 Hz, 4H, Ph), 7.21 (t, J=7.3, 2H, Ph), 7.15 (m, 2H, Ph), 7.05 (m, 2H, Ph), 4.53 (tt, J=12.5, 3.8 Hz, 1H, CHCH₂CH₂N), 4.40 (m, 2H, CHCH₂CH₂N), 4.28 (dd, J=11.0, 7.4 Hz, 1H, CH₂O), 4.08 (dd, J=11.0, 5.6 Hz, 1H, CH₂O), 3.78 (d, J=13.9 Hz, 2H, NCH₂Ph), 3.58 (d, J=13.9 Hz, 2H, NCH₂Ph), 3.16 (sext., J=6.3 Hz, 1H, CHCH₃), 2.99 (m, 2H, CHCH₂CH₂N), 2.30 (m, 2H, CHCH₂CH₂N), 1.90 (m, 21-1, CHCH₂CH₂N), 1.12 (d, J=6.8 Hz, 3H, CHCH₃)

¹³C NMR (100 MHz, CDCl₃) δ 155.2, 155.0 (C═O), 140.2, 128.8, 128.5, 128.1, 128.0, 126.7, 121.3, 121.1, 109.8, 109.2 (C_{Ph}), 66.8 (CH₂O), 53.7 (NCH₂Ph), 51.9 (CHCH₃), 50.7 (CHCH₂CH₂N), 43.6 (CHCH₂CH₂N), 29.2 (CHCH₂CH₂N), 11.0 (CHCH₃)

LC/MS (ES⁺) m/z 498.9 (M+H)⁺

### Preparation of (S)-2-(dibenzylamino)-3-phenylpropyl 4-(1,2-dihydro-2-oxobenzo[d]imidazol-3-yl)piperidine-1-carboxylate (Compound no 26)

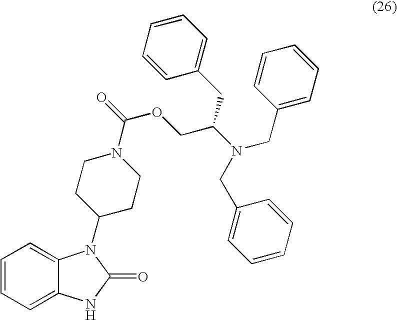

From 4-(2-keto-1-benzimidazolinyl)-piperidine and (S)-2-(dibenzylamino)-3-phenylpropan-1-ol

White solid (80%).

Flash chromatography on silica gel (cyclohexane/ethyl acetate 7/3)

¹H NMR (400 MHz, CDCl₃) δ 10.49 (s, 1H, NH), 7.30 (m, 12H, Ph), 7.10 (m, 7H, Ph), 4.46 (m, 1H, CHCH₂CH₂N), 4.45 (m, 2H, CHCH₂CH₂N), 4.38 (m, 1H, CH₂O), 4.22 (m, 1H, CH₂O), 3.83 (d, J=13.8 Hz, 2H, NCH₂Ph), 3.75 (d, J=13.8 Hz, 2H, NCH₂Ph), 3.30 (m, 1H, CHCH₂Ph), 3.10 (dd, J=13.6, 5.8 Hz, 1H, CHCH₂Ph), 2.94 (m, 2H, CHCH₂CH₂N), 2.71 (dd, J=13.6, 8.5 Hz, 1H, CHCH₂Ph), 2.38 (m, 2H, CHCH₂CH₂N), 1.88 (m, 2H, CHCH₂CH₂N)

¹³C NMR (100 MHz, CDCl₃) δ 155.1, 155.1 (C═O), 139.8, 139.5, 135.0, 129.2, 128.9, 128.8, 128.6, 128.3, 128.3, 128.1, 126.9, 126.8, 126.0, 121.4, 121.1, 121.0, 109.9, 109.3 (CPh), 64.7 (CH₂O), 58.5 (CHCH₂Ph), 54.0 (NCH₂Ph), 50.6 (CHCH₂CH₂N), 43.6 (CHCH₂CH₂N), 34.1 (CHCH₂Ph), 29.2 (CHCH₂CH₂N)

LC/MS (ES⁺) m/z 575.1 (M+H)⁺

### Preparation of 2-(N-benzyl-N-methylamino)ethyl 4-(1,2-dihydro-2-oxobenzo[d]imidazol-3-yl)piperidine-1-carboxylate (Compound no 27)

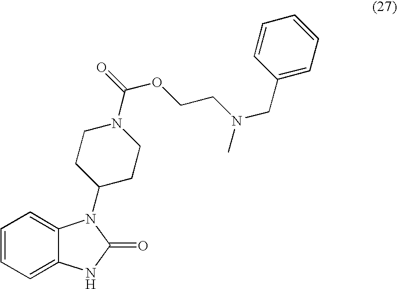

From 4-(2-keto-1-benzimidazolinyl)-piperidine and (S)-2-(N,N-benzylmethylamino)propan-1-ol

Pale Yellow oil (52%).

Flash chromatography on preparative TLC (silica gel, dichloromethane/methyl alcohol 85/15)

¹H NMR (400 MHz, CDCl₃) δ 10.48 (s, 1H, NH), 7.32 (m, 4H, Ph), 7.24 (m, 1H, Ph), 7.12 (dd, J=7.2, 1.4 Hz, 2H, Ph), 7.04 (m, 2H, Ph), 4.50 (tt, J=12.4, 3.8 Hz, 1H, CHCH₂CH₂N), 4.37 (m, 2H, CHCH₂CH₂N), 4.28 (t, J=5.8 Hz, 2H, OCH₂CH₂N), 3.59 (s, 2H, NCH₂Ph), 2.92 (m, 2H, CHCH₂CH₂N), 2.72 (t, J=5.8 Hz, 2H, OCH₂CH₂N), 2.35 (m, 5H, CHCH₂CH₂N, NCH₃), 1.85 (m, 2H, CHCH₂CH₂N)

¹³C NMR (100 MHz, CDCl₃) δ 155.2, 155.1 (C═O), 138.7, 128.9, 128.8, 128.2, 128.1, 127.0, 121.3, 121.0, 109.9, 109.2 (C_{Ph}), 63.4 (OCH₂CH₂N), 62.4 (NCH₂Ph), 55.6 (OCH₂CH₂N), 50.6 (CHCH₂CH₂N), 43.6 (CHCH₂CH₂N), 42.7 (NCH₃), 29.1 (CHCH₂CH₂N)

LC/MS (ES⁺) m/z 409.4 (M+H)⁺

### Preparation of (S)-2-(dibenzylamino)propyl 5,6-dihydro-4-(1,2-dihydro-2-oxobenzo[d]imidazol-3-yl)pyridine-1(2H)-carboxylate (Compound no 28)

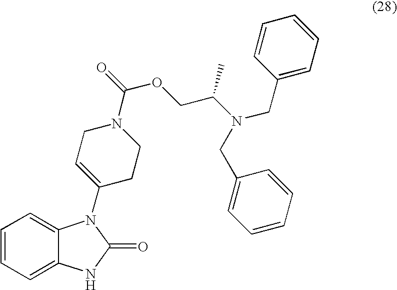

From 1-(1,2,3,6-tetrahydro-pyridin-4-yl)-1,3-dihydro-benzoimidazol-2-one and (S)-2-(dibenzylamino)propan-1-ol

Colorless oil (66%).

Flash chromatography on preparative TLC (silica gel, cyclohexane/ethyl acetate 1/1)

¹H NMR (400 MHz, CDCl₃) δ 9.97 (s, 1H, NH), 7.38 (d, J=7.3 Hz, 4H, Ph), 7.3 (m, 4H, Ph), 7.20 (m, 2H, Ph), 7.05 (m, 4H, Ph), 5.97 (s, 1H, NCH₂CH═C), 4.26 (m, 3H, CH₂O, NCH₂CH═C), 4.10 (dd, J=11.2, 5.6 Hz, 1H, CH₂O), 3.81 (m, 2H, NCH₂CH₂), 3.71 (d, J=13.9 Hz, 2H, NCH₂Ph), 3.57 (d, J=13.9 Hz, 2H, NCH₂Ph), 3.16 (sext., J=6.6 Hz, 1H, CHCH₃), 2.63 (m, 2H, NCH₂CH₂), 1.12 (d, J=6.6 Hz, 1H, CHCH₃)

¹³C NMR (100 MHz, CDCl₃) δ 171.2, 154.2 (C═O), 140.2, 129.9, 128.5, 128.1, 126.8, 122.0, 121.5, 109.8, 108.8 (C_{Ph}), 66.9 (CH₂O), 53.7 (NCH₂Ph), 50.6 (CHCH₃), 42.7 (NCH₂CH═C), 40.6 (NCH₂CH₂C_{q}) 26.9 (NCH₂CH₂C_{q}), 11.2 (CHCH₃),

LC/MS (ES⁺) m/z 496.9 (M+H)⁺

### Preparation of 3-(dibenzylamino)propyl 4-(1,2-dihydro-2-oxobenzo[d]imidazol-3-yl)piperidine-1-carboxylate (Compound no 29)

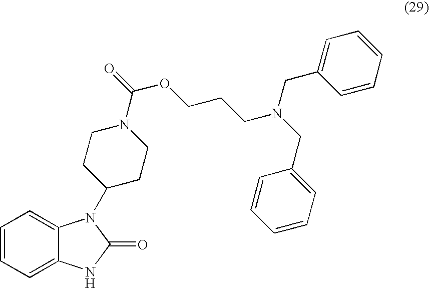

From 4-(2-keto-1-benzimidazolinyl)-piperidine and 3-dibenzylaminopropan-1-ol

White solid (54%).

Flash chromatography on preparative TLC (silica gel, cyclohexane/ethyl acetate 1/1)

¹H NMR (400 MHz, CDCl₃) δ 10.26 (s, 1H, NH), 7.38 (d, J=7.3 Hz, 4H, Ph), 7.30 (t, J=7.4 Hz, 4H, Ph), 7.23 (m, 2H, Ph), 7.10 (m, 4H, Ph), 4.47 (tt, J=12.4, 3.8 Hz, 1H, CHCH₂CH₂N), 4.39 (m, 1H, CHCH₂CH₂N), 4.20 (m, 2H, OCH₂CH₂CH₂N), 3.90 (m, 1H, CHCH₂CH₂N), 3.58 (s, 4H, NCH₂Ph), 2.79 (m, 2H, CHCH₂CH₂N), 2.54 (t, J=6.1 Hz, 2H, OCH₂CH₂CH₂N), 2.23 (m, 2H, CHCH₂CH₂N), 1.82 (m, 4H, CHCH₂CH₂N, OCH₂CH₂CH₂N)

¹³C NMR (100 MHz, CDCl₃) δ 155, 155.1 (C═O), 139.6, 128.8, 128.7, 128.1, 128.1, 126.8, 121.4, 121.1, 109.9, 109.3 (C_{Ph}), 63.5 (OCH₂CH₂CH₂N), 58.4 (NCH₂Ph), 50.6 (CHCH₂CH₂N), 49.5 (OCH₂CH₂CH₂N), 43.4 (CHCH₂CH₂N), 29.1 (CHCH₂CH₂N), 26.6 (OCH₂CH₂CH₂N)

LC/MS (ES⁺) m/z 499.1 (M+H)⁺

### Preparation of 2-(dibenzylamino)ethyl 4-(1,2-dihydro-2-oxobenzo[d]imidazol-3-yl)piperidine-1-carboxylate (Compound no 30)

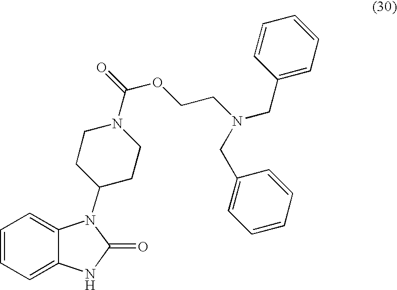

From 4-(2-keto-1-benzimidazolinyl)-piperidine and 2-dibenzylaminoethan-1-ol

White solid (60%).

Flash chromatography on preparative TLC (silica gel, cyclohexane/ethyl acetate 1/1)

¹H NMR (400 MHz, CDCl₃) δ 10.46 (s, 1H, NH), 7.40 (d, J=7.2 Hz, 4H, Ph), 7.32 (t, J=7.6 Hz, 4H, Ph), 7.24 (m, 2H, Ph), 7.15 (m, 2H, Ph), 7.06 (m, 2H, Ph), 4.54 (m, 1H, CHCH₂CH₂N), 4.44 (m, 1H, CHCH₂CH₂N), 4.31 (m, 1H, CHCH₂CH₂N), 4.26 (t, J=5.6 Hz, 2H, OCH₂CH₂N), 3.68 (s, 4H, CH₂Ph), 2.92 (t, J=11.2 Hz, 2H, CHCH₂CH₂N), 2.79 (t, J=5.6 Hz, 2H, OCH₂CH₂N), 2.35 (m, 2H, CHCH₂CH₂N), 1.86 (d, J=11.2 Hz, 2H, CHCH₂CH₂N)

¹³C NMR (100 MHz, CDCl₃) δ 155.2, 155.1 (C═O), 139.4, 128.8, 128.8, 128.7, 128.2, 128.1, 127.9, 126.9, 121.3, 121.1, 109.9, 109.3 (C_{Ph}), 63.5 (OCH₂CH₂N), 58.7 (NCH₂Ph), 52.1 (OCH₂CH₂N), 50.7 (CHCH₂CH₂N), 43.6 (CHCH₂CH₂N), 29.2 (CHCH₂CH₂N)

LC/MS (ES⁺) m/z 485.1 (M+H)⁺

### Preparation of 2-(benzyloxy)ethyl 4-(1,2-dihydro-2-oxobenzo[d]imidazol-3-yl)piperidine-1-carboxylate (Compound no 31)

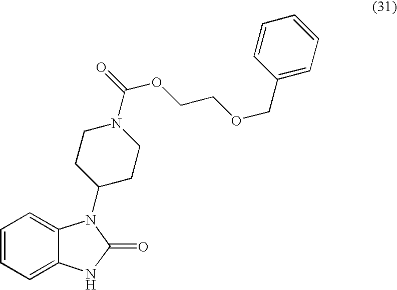

From 4-(2-keto-1-benzimidazolinyl)-piperidine and 9-benzyloxyethan-1-ol.

Colorless oil (36%).

Flash chromatography on preparative TLC (silica gel, dichloromethane/methyl alcohol 95/5)

¹H NMR (400 MHz, CDCl₃) δ 10.32 (s, 1H, NH), 7.35 (m, 4H, Ph), 7.30 (m, 1H, Ph), 7.13 (m, 2H, Ph), 7.05 (m, 2H, Ph), 4.60 (s, 2H, OCH₂Ph), 4.51 (tt, J=12.3, 4.0 Hz, 1H, CHCH₂CH₂N), 4.41 (m, 2H, CHCH₂CH₂N), 4.33 (t, J=4.6 Hz, 2H, OCH₂CH₂OBn), 3.74 (t, J=4.6 Hz, 2H, OCH₂CH₂OBn), 2.96 (m, 2H, CHCH₂CH₂N), 2.37 (td, J=12.2, 8.9 Hz, 2H, CHCH₂CH₂N), 1.85 (m, 2H, CHCH₂CH₂N)

¹³C NMR (100 MHz, CDCl₃) δ 155.2, 155.1 (C═O), 137.9, 128.8, 128.4, 128.1, 127.7, 127.6, 121.3, 121.1, 109.9, 109.3 (C_{Ph}), 73.0 (OCH₂Ph), 68.3 (OCH₂CH₂OBn), 64.7 (OCH₂CH₂OBn), 50.6 (CHCH₂CH₂N), 43.6 (CHCH₂CH₂N), 29.1 (CHCH₂CH₂N)

LC/MS (ES⁺) m/z 396.0 (M+H)⁺

### Preparation of 1-benzylpiperidin-3-yl 4-(1,2-dihydro-2-oxobenzo[d]imidazol-3-yl)piperidine-1-carboxylate (Compound no 33)

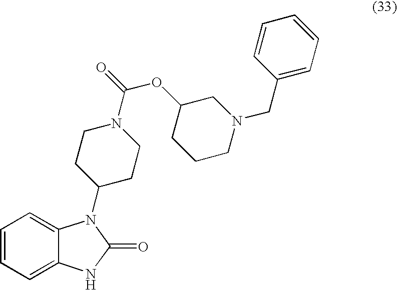

From 4-(2-keto-1-benzimidazolinyl)-piperidine and N-benzyl-3-hydroxypiperidine

White solid (100%).

Flash chromatography on preparative TLC (silica gel, cyclohexane/ethyl acetate 2/8)

¹H NMR (400 MHz, CDCl₃) δ 10.65 (s, 1H, NH), 7.31 (m, 4H, Ph), 7.23 (m, 1H, Ph), 7.14 (m, 2H, Ph), 7.05 (m, 2H, Ph), 4.85 (m, 1H, OCH), 4.51 (tt, J=12.2, 3.6 Hz, 1H, CHCH₂CH₂N), 4.38 (m, 2H, CHCH₂CH₂N), 3.55 (s, 2H, CH₂Ph), 2.91 (m, 3H, CHCH₂CH₂N, OCHCH₂N), 2.60 (m, 1H, OCHCH₂CH₂CH₂N), 2.35 (m, 2H, CHCH₂CH₂N), 2.20 (m, 2H, OCHCH₂CH₂CH₂N, OCHCH₂N), 1.96 (m, 1H, OCHCH₂CH₂CH₂N), 1.85 (d, J=11.8 Hz, 2H, CHCH₂CH₂N), 1.78 (m, 1H, OCHCH₂CH₂CH₂N), 1.64 (m, 1H, OCHCH₂CH₂CH₂N), 1.48 (m, 1H, OCHCH₂CH₂CH₂N)

¹³C NMR (100 MHz, CDCl₃) δ 155.2, 154.7 (C═O), 138.2, 128.8, 128.2, 128.1, 126.9, 121.3, 121.0, 109.9, 109.2 (C_{Ph}), 70.8 (OCH), 62.8 (NCH₂Ph), 57.5, 53.1 (OCHCH₂N, OCHCH₂CH₂CH₂N), 50.6 (CHCH₂CH₂N), 43.5 (CHCH₂CH₂N), 30.0 (OCHCH₂CH₂CH₂N), 29.1 (CHCH₂CH₂N), 22.9 (OCHCH₂CH₂CH₂N)

LC/MS (ES⁺) m/z 435.2 (M+H)⁺

### Preparation of 2-(diisopropylamino)ethyl 4-(1,2-dihydro-2-oxobenzo[d]imidazol-3-yl)piperidine-1-carboxylate (Compound no 34)

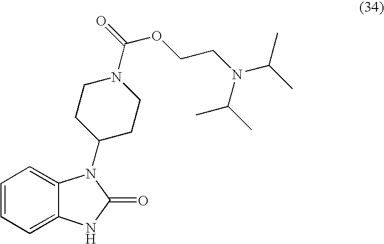

From 4-(2-keto-1-benzimidazolinyl)-piperidine and 2-(diisopropylamino)propan-1-ol

Colorless oil (58%).

Flash chromatography on preparative TLC (silica gel, cyclohexane/ethyl acetate 7/3)

1H NMR (400 MHz, CDCl₃) δ 9.56 (s, 1H, NH), 7.10 (m, 4H, Ph), 4.51 (tt, J=12.4, 3.8 Hz, 1H, CHCH₂CH₂N), 4.39 (m, 2H, CHCH₂CH₂N), 4.06 (t, J=6.8 Hz, 2H, OCH₂CH₂N), 3.00 (m, 2H, CH(CH₃)₂), 2.93 (m, 2H, CHCH₂CH₂N), 2.69 (t, J=6.8 Hz, 2H, OCH₂CH₂N), 2.34 (m, 2H, CHCH₂CH₂N), 1.86 (d, J=11.9 Hz, 2H, CHCH₂CH₂N), 1.02 (t, J=9.0 Hz, 12H, CH(CH₃)₂)

¹³C NMR (100 MHz, CDCl₃) δ 155.4, 154.8 (C═O), 128.9, 127.9, 121.3, 121.1, 109.7, 109.3 (C_{Ph}), 66.4 (OCH₂CH₂N), 50.8, 49.3 (CH(CH₃)₂, CHCH₂CH₂N), 44.1 (OCH₂CH₂N), 43.5 (CHCH₂CH₂N), 29.1 (CHCH₂CH₂N), 20.8 (CH(CH₃)₂)

LC/MS (ES⁺) m/z 389.3 (M+H)⁺

### Preparation of 2-(dimethylamino)ethyl 4-(1,2-dihydro-2-oxobenzo[d]imidazol-3-yl)piperidine-1-carboxylate (Compound no 35)

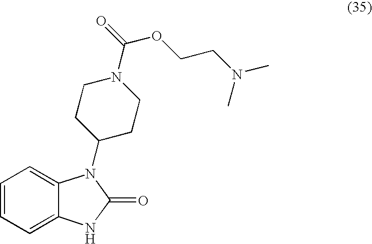

From 4-(2-keto-1-benzimidazolinyl)-piperidine and 2-(dimethylamino)propan-1-ol

Colorless oil (63%).

Flash chromatography on preparative TLC (silica gel, cyclohexane/ethyl acetate 7/3)

¹H NMR (400 MHz, CDCl₃) δ 9.85 (s, 1H, NH), 7.09 (m, 4H, Ph), 4.48 (tt, J=12.5, 4.0 Hz, 1H, CHCH₂CH₂N), 4.35 (m, 2H, CHCH₂CH₂N), 4.26 (m, 2H, OCH₂CH₂N), 2.91 (m, 2H, CHCH₂CH₂N), 2.65 (t, J=5.8 Hz, 2H, OCH₂CH₂N), 2.31 (m, 8H, CHCH₂CH₂N, NCH₃)₂), 1.83 (d, J=10.9 Hz, 2H, CHCH₂CH₂N)

¹³C NMR (100 MHz, CDCl₃) δ 155.2, 154.8 (C═O), 128.9, 128.0, 121.3, 121.1, 109.7, 109.3 (C_{Ph}), 63.4 (OCH₂CH₂N), 58.2 (OCH₂CH₂N), 50.7 (CHCH₂CH₂N), 45.7 (N(CH₃)₂), 43.6 (CHCH₂CH₂N), 29.1 (CHCH₂CH₂N)

LC/MS (ES⁺) m/z 333.2 (M+H)⁺

### Preparation of 3,3-diphenylpropyl 4-(1,2-dihydro-2-oxobenzo[d]imidazol-3-yl)piperidine-1-carboxylate (Compound no 36)

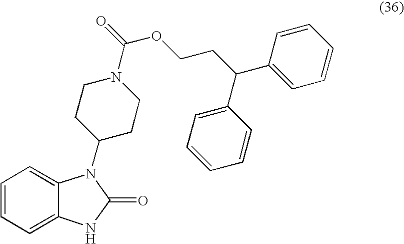

From 4-(2-keto-1-benzimidazolinyl)-piperidine and 3,3-diphenyl-propan-1-ol.

Colorless oil (86%).

Flash chromatography on preparative TLC (silica gel, cyclohexane/ethyl acetate 8/2)

¹H NMR (400 MHz, CDCl₃) δ 9.83 (s, 1H, NH), 7.29 (m, 8H, Ph), 7.19 (m, 2H, Ph), 7.13 (m, 2H, Ph), 7.06 (m, 2H, Ph), 4.49 (tt, J=12.4, 3.7 Hz, 1H, CHCH₂CH₂N), 4.36 (m, 1H, CHCH₂CH₂N), 4.13 (m, 4H, CHCH₂CH₂N, OCH₂CH₂CHPh₂), 2.89 (t, J=12.6 Hz, 2H, CHCH₂CH₂N), 2.46 (q, J=6.8 Hz, 2H, OCH₂CH₂CHPh₂), 2.32 (m, 2H, CHCH₂CH₂N), 1.83 (d, J=11.7 Hz, 2H, CHCH₂CH₂N)

¹³C NMR (100 MHz, CDCl₃) δ 155.2, 154.9 (C═O), 144.2, 128.8, 128.5, 127.9, 127.7, 126.3, 121.4, 121.1, 109.8, 109.3 (C_{Ph}), 64.2 (OCH₂CH₂CHPh₂), 50.8, (CHCH₂CH₂N), 48.0 (OCH₂CH₂CHPh₂), 43.5 (CHCH₂CH₂N), 34.7 (OCH₂CH₂CHPh₂), 29.2 (CHCH₂CH₂N)

LC/MS (ES⁺) m/z 456.1 (M+H)⁺

### Preparation of (S)-2-(dibenzylamino)propyl 4-(6-chloro-1,2-dihydro-2-oxobenzo[d]imidazol-3-yl)piperidine-1-carboxylate (Compound no 38)

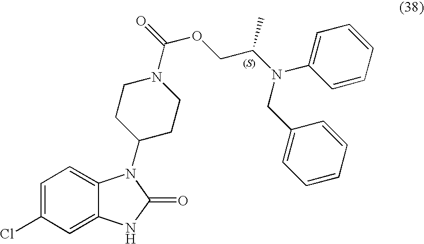

From 5-chloro-1-(piperidin-4-yl)-1H-benzo[d]imidazol-2(3H)-one and (S)-2-(dibenzylamino)propan-1-ol

Colorless oil (79%).

Flash chromatography on silica gel (cyclohexane/ethyl acetate 6/4)

¹H NMR (400 MHz, CDCl₃) δ 9.05 (bs, 1H, NH), 7.38 (d, J=7.6 Hz, 4H, Bn), 7.29 (t, J=7.2 Hz, 4H, Bn), 7.21 (t, J=7.2 Hz, 2H, Bn), 7.12 (s, 1H, Ar), 7.00 (m, 2H, Ar), 4.49 (m, 3H, CHCH₂CH₂Nₚᵢₚ, CHCH₂CH₂Nₚᵢₚ), 4.28 (dd, J=5.6, 10.8 Hz, 1H, CH₂O), 4.07 (dd, J=5.6, 10.8 Hz, 1H, CH₂O), 3.77 (d, J=14.0 Hz, 2H, NCH₂Ph), 3.57 (d, J=14.0 Hz, 2H, NCH₂Ph), 3.16 (m, 1H, CHMe), 2.95 (m, 2H, CHCH₂CH₂Nₚᵢₚ), 2.33 (m, 2H, CHCH₂CH₂Nₚᵢₚ), 1.87 (m, 2H, CHCH₂CH₂Nₚᵢₚ), 1.11 (d, J=6.4 Hz, 3H, Me).

¹³C NMR (100 MHz, CDCl₃) δ 155.1 (2×C=0), 140.2 (C_{Bn}), 129.2 (C_{Ar}), 128.5 (CH_{Bn}), 128.1 (CH_{Bn}), 127.4 (C_{Ar}), 127.0 (C_{Ar}), 126.8 (CH_{Bn}), 121.2 (CH_{Ar}), 110.3 (CH_{Ar}), 109.9 (CH_{Ar}), 67.4 (CH₂O), 53.7 (NCH₂Ph), 51.9 (CHMe), 50.9 (CHCH₂CH₂Nₚᵢₚ), 43.7 (CHCH₂CH₂Nₚᵢₚ), 29.5 (CHCH₂CH₂Nₚᵢₚ), 11.2 (Me).

LC/MS (ES⁺) m/z 533.2 (M+H)⁺

### Preparation of 2-(diethylamino)ethyl 4-(1,2-dihydro-2-oxobenzo[d]imidazol-3-yl)piperidine-1-carboxylate (Compound no 39)

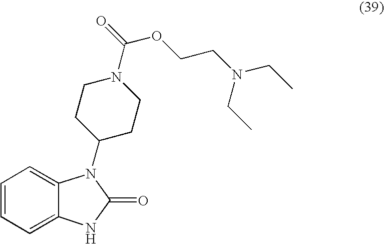

From 1-(piperidin-4-yl)-1H-benzo[d]imidazol-2(3H)-one and 2-(diethylamino)ethanol

White solid (69%).

Flash chromatography on silica gel (methylene chloride/methanol 85/15)

¹H NMR (400 MHz, CDCl₃) δ 10.0 (bs, 1H, NH), 7.12 (m, 2H, Ar), 7.06 (m, 2H, Ar), 4.51 (m, 1H, CHCH₂CH₂Nₚᵢₚ), 4.39 (m, 2H, CHCH₂CH₂Nₚᵢₚ), 4.22 (t, J=6.4 Hz, 2H, CH₂O), 2.93 (m, 2H, CHCH₂CH₂Nₚᵢₚ), 2.78 (t, J=6.4 Hz, 2H, CH₂CH₂O), 2.63 (q, J=7.2 Hz, 4H, CH₃CH₂N), 2.35 (m, 2H, CHCH₂CH₂Nₚᵢₚ), 1.85 (m, 2H, CHCH₂CH₂Nₚᵢₚ), 1.07 (t, J=7.2 Hz, 6H, CH₃CH₂N).

¹³C NMR (100 MHz, CDCl₃) δ 155.3 (C═O), 154.9 (C═O), 128.8 (C_{Ar}), 128.0 (C_{Ar}), 121.3 (CH_{Ar}), 121.0 (CH_{Ar}), 109.8 (CH_{Ar}), 109.3 (CH_{Ar}), 63.9 (CH₂O), 51.3 (CH₂CH₂O), 50.6 (CHCH₂CH₂Nₚᵢₚ), 47.7 (CH₃CH₂N), 43.6 (CHCH₂CH₂Nₚᵢₚ), 29.1 (CHCH₂CH₂Nₚᵢₚ), 11.9 (CH₃CH₂N).

LC/MS (ES⁺) m/z 361.2 (M+H)⁺

### Preparation of 2-(1-benzylpiperidin-4-yl)ethyl 4-(1,2-dihydro-2-oxobenzo[d]imidazol-3-yl)piperidine-1-carboxylate (Compound no 40)

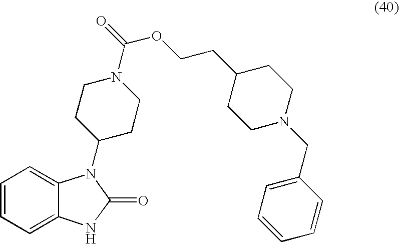

From 4-(2-keto-1-benzimidazolinyl)-piperidine and N-benzyl-4-(2-hydroxyethyl)piperidine

White solid (65%).

Flash chromatography on silica gel (methylene chloride/methanol 9/1)

¹H NMR (400 MHz, CDCl₃) δ 9.88 (bs, 1H, NH), 7.32 (m, 4H, Bn), 7.25 (m, 1H, Bn), 7.13 (dd, J=7.2, 4.0 Hz, 2H, Ar), 7.07 (dd, J=7.2, 4.0 Hz, 2H, Ar), 4.51 (m, 1H, CHCH₂CH₂Nₚᵢₚ), 4.35 (m, 2H, CHCH₂CH₂Nₚᵢₚ), 4.17 (t, J=6.8 Hz, 2H, CH₂CH₂O), 3.51 (s, 2H, NCH₂Ph), 2.90 (m, 4H, CHCH₂CH₂Nₚᵢₚ, CHCH₂CH₂N_{Bn}), 2.35 (m, 2H, CHCH₂CH₂Nₚᵢₚ), 1.98 (m, 2H, CHCH₂CH₂N_{Bn}), 1.85 (m, 2H, CHCH₂CH₂Nₚᵢₚ), 1.71 (m, 2H, CHCH₂CH₂N_{Bn}), 1.62 (dd, J=6.8, 6.4 Hz, 2H, CH₂CH₂O), 1.36 (m, 3H, CHCH₂CH₂N_{Bn}, CHCH₂CH₂N_{Bn}).

¹³C NMR (100 MHz, CDCl₃) δ 155.4 (C═O), 154.9 (C═O), 129.3 (CH_{Ar}), 128.9 (Cm), 128.1 (CH_{Ar}), 127.9 (2C_{Ar}), 126.9 (CH_{Ar}), 121.3 (CH_{Ar}), 121.1 (CH_{Ar}), 109.8 (CH_{Ar}), 109.3 (CH_{Ar}), 63.8 (CH₂CH₂O), 63.5 (NCH₂Ph), 53.7 (CHCH₂CH₂N_{Bn}), 50.7 (CHCH₂CH₂Nₚᵢₚ), 43.6 (CHCH₂CH₂Nₚᵢₚ), 35.6 (CH₂CH₂O), 32.9 (CHCH₂CH₂N_{Bn}), 32.2 (CHCH₂CH₂N_{Bn}), 29.2 (CHCH₂CH₂Nₚᵢₚ).

LC/MS (ES⁺) m/z 463.3 (M+H)⁺

### Preparation of 2-(ethyl(phenyl)amino)ethyl 4-(2-oxo-2,3-dihydro-1H-benzo[d]imidazol-1-yl)piperidine-1-carboxylate (Compound no 41)

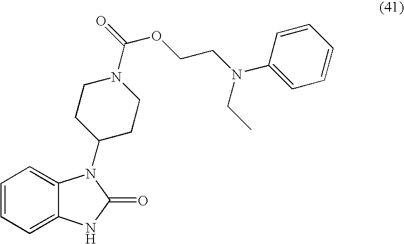

From 1-(piperidin-4-yl)-1H-benzo[d]imidazol-2(3H)-one and 2-(N-ethyl-N-phenylamino)ethanol

White solid (94%).

Flash chromatography on silica gel (cyclohexane/ethyl acetate 3/7)

¹H NMR (400 MHz, CDCl₃) δ 8.75 (bs, 1H, NH), 7.22 (m, 2H, Ar), 7.06 (m, 4H, Ar), 6.76 (d, J=8.0 Hz, 2H, Ar), 6.68 (t, J=6.8 Hz, 1H, Ar), 4.48 (m, 1H, CHCH₂CH₂Nₚᵢₚ), 4.30 (m, 4H, CHCH₂CH₂Nₚᵢₚ, CH₂CH₂O), 3.61 (t, J=6.4 Hz, 2H, CH₂CH₂O), 3.44 (q, J=7.2 Hz, 2H, CH₃CH₂N), 2.93 (m, 2H, CHCH₂CH₂Nₚᵢₚ), 2.31 (m, 2H, CHCH₂CH₂Nₚᵢₚ), 1.85 (m, 2H, CHCH₂CH₂Nₚᵢₚ), 1.20 (t, J=7.2 Hz, 3H, CH₃CH₂N).

¹³C NMR (100 MHz, CDCl₃) δ 155.1 (C═O), 154.9 (C═O), 147.7 (C_{Ar}), 129.3 (CH_{Ar}), 128.8 (C_{Ar}), 127.9 (C_{Ar}), 121.3 (CH_{Ar}), 121.2 (CH_{Ar}), 116.1 (CH_{Ar}), 111.9 (CH_{Ar}), 109.8 (CH_{Ar}), 109.3 (CH_{Ar}), 62.8 (CH₂CH₂O), 50.6 (CHCH₂CH₂Nₚᵢₚ), 49.1 (CH₂CH₂O), 45.0 (CH₃CH₂N), 43.6 (CHCH₂CH₂Nₚᵢₚ), 29.1 (CHCH₂CH₂Nₚᵢₚ), 12.3 (CH₃CH₂N).

LC/MS (ES⁺) m/z 409.2 (M+H)⁺

### Preparation of ((S)-1-benzylpyrrolidin-2-yl)methyl 4-(1,2-dihydro-2-oxobenzo[d]imidazol-3-yl)piperidine-1-carboxylate (Compound no 42)

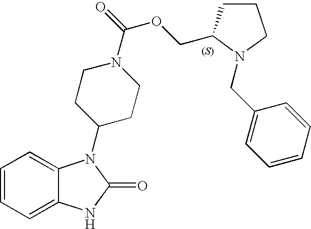

From 1-(piperidin-4-yl)-1H-benzo[d]imidazol-2(3H)-one and ((S)-1-benzylpyrrolidin-2-yl)methanol

Colorless oil (88%).

Flash chromatography on silica gel (methylene chloride/methanol 95/5)

¹H NMR (400 MHz, CDCl₃) δ 10.33 (bs, 1H, NH), 7.32 (m, 4H, Bn), 7.25 (m, 1H, Bn), 7.12 (m, 1H, Ar), 7.07 (m, 3H, Ar), 4.52 (m, 1H, CHCH₂CH₂Nₚᵢₚ), 4.48 (m, 2H, CHCH₂CH₂Nₚᵢₚ), 4.17 (m, 3H, CH₂O, NCH₂Ph), 3.42 (d, J=12.8 Hz, 1H, NCH₂Ph), 2.91 (m, 3H, CHCH₂CH₂Nₚᵢₚ, CH₂CHN_{Bn}), 2.35 (m, 2H, CHCH₂CH₂Nₚᵢₚ), 2.24 (m, 1H, CH₂CH₂N_{Bn}), 1.95 (m, 2H, CH₂CH₂N_{Bn}, CH₂CHN_{Bn}), 1.85 (m, 2H, CHCH₂CH₂Nₚᵢₚ), 1.72 (m, 3H, CH₂CH₂N_{Bn}, CH₂CHN_{Bn}).

¹³C NMR (100 MHz, CDCl₃) δ 155.4 (C═O), 155.1 (C═O), 139.6 (C_{Ar}), 128.8 (CH_{Ar}), 128.2 (CH_{Ar}), 128.1 (2C_{Ar}), 126.9 (CH_{Ar}), 121.3 (CH_{Ar}), 121.1 (CH_{Ar}), 109.9 (CH_{Ar}), 109.3 (CH_{Ar}), 62.4 (CH₂CHN_{Bn}), 59.4 (CH₂O), 54.5 (NCH₂Ph), 50.6 (CHCH₂CH₂Nₚᵢₚ), 43.7 (CHCH₂CH₂Nₚᵢₚ), 29.2 (CHCH₂CH₂Nₚᵢₚ), 28.4 (CH₂CHN_{Bn}), 23.0 (CH₂CH₂N_{Bn}).

LC/MS (ES⁺) m/z 435.1 (M+H)⁺

### Preparation of 2,2-diphenylethyl 4-(1,2-dihydro-2-oxobenzo imidazol-3-yl)piperidine-1-carboxylate (Compound no 43)

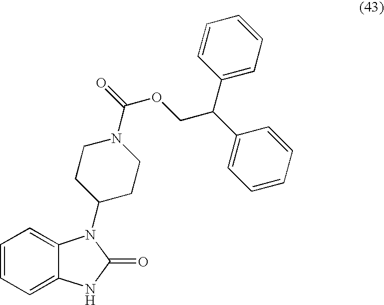

From 1-(piperidin-4-yl)-1H-benzo[d]imidazol-2(3H)-one and 2,2-diphenylethanol

White solid (75%).

Flash chromatography on silica gel (cyclohexane/ethyl acetate 5/5)

¹H NMR (400 MHz, CDCl₃) δ 10.3 (bs, 1H, NH), 7.31 (m, 8H, Ar), 7.23 (m, 2H, Ar), 7.12 (m, 1H, Ar), 7.06 (m, 2H, Ar), 7.01 (m, 1H, Ar), 4.73 (m, 2H, CH₂O), 4.45 (m, 3H, CHCH₂CH₂Nₚᵢₚ, CH(Ph)₂), 4.06 (m, 1H, CHCH₂CH₂Nₚᵢₚ), 2.83 (m, 2H, CHCH₂CH₂Nₚᵢₚ), 2.28 (m, 1H, CHCH₂CH₂Nₚᵢₚ), 2.06 (m, 1H, CHCH₂CH₂Nₚᵢₚ), 1.80-1.72 (m, 2H, CHCH₂CH₂Nₚᵢₚ).

¹³C NMR (100 MHz, CDCl₃) δ 155.1 (C═O), 154.9 (C═O), 141.2 (C_{Ar}), 128.7 (C_{Ar}), 128.5 (CH_{Ar}), 128.2 (CH_{Ar}), 128.1 (C_{Ar}), 126.7 (CH_{Ar}), 121.4 (CH_{Ar}), 121.1 (CH_{Ar}), 109.9 (CH_{Ar}), 109.4 (CH_{Ar}), 67.8 (CH₂O), 50.5 (CHCH₂CH₂Nₚᵢₚ), 50.1 (CH(Ph)₂), 43.6 (CHCH₂CH₂Nₚᵢₚ), 29.0 (CHCH₂CH₂Nₚᵢₚ).

LC/MS (ES⁺) m/z 442.1 (M+H)⁺

### Preparation of (S)-2-(dibenzylamino)propyl 4-(1H-indol-3-yl)piperidine-1-carboxylate (Compound no 44)

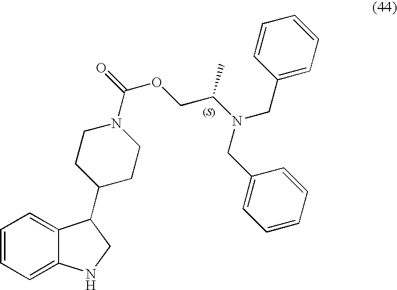

From (S)-2-(dibenzylamino)propan-1-ol and 3-(piperidin-4-yl)-1H-indole

Colorless oil (55%).

Flash chromatography on silica gel (cyclohexane/ethyl acetate 75/25)

¹H NMR (400 MHz, CDCl₃) δ 8.10 (bs, 1H, NH), 7.67 (d, J=7.6 Hz, 1H, Ar), 7.41 (d, J=7.6 Hz, 4H, Bn), 7.29 (t, J=7.6 Hz, 4H, Bn), 7.21 (m, 3H, Ar), 7.13 (t, J=7.2 Hz, 2H, Ar), 6.96 (s, 1H, Ar), 4.29 (m, 3H, CHCH₂CH₂Nₚᵢₚ, CH₂O), 4.09 (m, 1H, CH₂O), 3.77 (d, J=14.0 Hz, 2H, NCH₂Ph), 3.59 (d, J=14.0 Hz, 2H, NCH₂Ph), 3.17 (m, 1H, CHMe), 3.02 (m, 3H, CHCH₂CH₂Nₚᵢₚ, CHCH₂CH₂Nₚᵢₚ), 2.10 (m, 2H, CHCH₂CH₂Nₚᵢₚ), 1.73 (m, 2H, CHCH₂CH₂Nₚᵢₚ), 1.12 (d, J=6.8 Hz, 3H, Me).

¹³C NMR (100 MHz, CDCl₃) δ 155.5 (C═O), 140.3 (C_{Bn}), 136.4, 128.5 (CH_{Bn}), 128.1 (CH_{Bn}), 126.7 (CH_{Bn}), 126.4, 122.0, 120.7, 119.7, 119.2, 118.9, 111.3, 66.5 (CH₂O), 53.7 (NCH₂Ph), 51.9 (CHMe), 44.6 (CHCH₂CH₂Nₚᵢₚ), 33.6 (CHCH₂CH₂Nₚᵢₚ), 26.9 (CHCH₂CH₂Nₚᵢₚ), 11.2 (Me).

LC/MS (ES⁺) m/z 482.3 (M+H)⁺

### Preparation of (S)-2-(dibenzylamino)propyl 4-(N-phenylpropionamido)piperidine-1-carboxylate (Compound no 45)

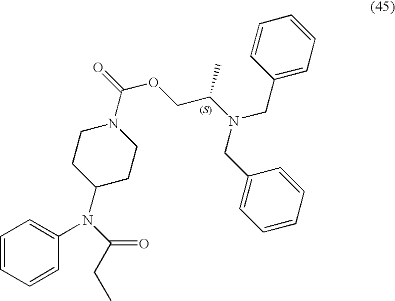

From N-phenyl-N-(piperidin-4-yl)propionamide hydrochloride salt and (S)-2-(dibenzylamino)propan-1-ol

Colorless oil (90%).

NEt₃ (2.7 equiv.) was added in the second step.

Flash chromatography on silica gel (cyclohexane/ethyl acetate 7/3)

¹H NMR (400 MHz, CDCl₃) δ 7.40 (m, 3H, Ar), 7.31 (m, 4H, Ar), to 7.21 (m, 6H, Ar), 7.07 (m, 2H, Ar), 4.84 (m, 1H, CHCH₂CH₂Nₚᵢₚ), 4.18 (m, 3H, CHCH₂CH₂Nₚᵢₚ, CH₂O), 3.93 (dd, J=5.6, 10.8 Hz, 1H, CH₂O), 3.68 (d, J=14.0 Hz, 2H, NCH₂Ph), 3.50 (d, J=14.0 Hz, 2H, NCH₂Ph), 3.04 (m, 1H, CHMe), 2.88 (m, 2H, CHCH₂CH₂Nₚᵢₚ), 1.93 (q, J=7.2 Hz, 2H, N(C═O)CH₂CH₃), 1.81 (m, 2H, CHCH₂CH₂Nₚᵢₚ), 1.26 (m, 2H, CHCH₂CH₂Nₚᵢₚ), 1.02 (m, 6H, N(C═O)CH₂CH₃, Me).

¹³C NMR (100 MHz, CDCl₃) δ 173.5 (C═O), 155.0 (C═O), 140.2 (CH_{Bn}), 138.6 (C_{Ar}), 130.2 (CH_{Ar}), 129.4 (CH_{Ar}), 128.5 (CH_{AD}), 128.4 (CH_{Bn}), 128.0 (CH_{Bn}), 126.7 (CH_{Bn}), 66.5 (CH₂O), 53.2 (NCH₂Ph), 52.1 (CHMe), 51.8 (CHCH₂CH₂Nₚᵢₚ), 43.4 (CHCH₂CH₂Nₚᵢₚ), 28.4 (CHCH₂CH₂Nₚᵢₚ), 26.9 (N(C═O)CH₂CH₃), 11.2 (Me), 9.6 (N(C═O)CH₂CH₃).

LC/MS (ES⁺) m/z 514.9 (M+H)⁺

### Preparation of 4-(dibenzylamino)phenyl 4-(1,2-dihydro-2-oxobenzo[d]imidazol-3-yl)piperidine-1-carboxylate (Compound no 178)

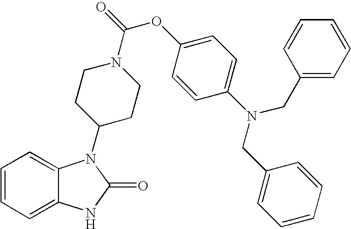

From 4-(2-keto-1-benzimidazolinyl)-piperidine and 4-(dibenzylamino)phenol

Pale yellow solid (30%).

Flash chromatography on silica gel (cyclohexane/ethyl acetate 5/5)

¹H NMR (400 MHz, CDCl₃) δ 9.86 (s, 1H, NH), 7.33 (d, J=7.2 Hz, 4H, Ph), 7.30 (m, 6H, Ph), 7.16 (m, 2H, Ph), 7.08 (m, 2H, Ph), 6.95 (d, J=9.2 Hz, 2H, Ph), 6.71 (d, J=9.2 Hz, 2H, Ph), 4.65 (s, 4H, NCH₂Ph), 4.50 (m, 3H, CHCH₂CH₂N, CHCH₂CH₂N), 3.12-3.00 (m, 2H, CHCH₂CH₂N), 2.46 (m, 2H, CHCH₂CH₂N), 1.91 (m, 2H, CHCH₂CH₂N)

¹³C NMR (100 MHz, CDCl₃) δ 154.9 (C═O), 154.4 (C═O), 146.9, 142.2, 138.4, 128.9, 128.6, 127.9, 126.9, 126.6, 122.2, 121.4, 121.2, 112.9, 109.8, 109.3, 54.5 (NCH₂Ph), 50.6 (CHCH₂CH₂N), 43.5 (CHCH₂CH₂N), 29.6 (CHCH₂CH₂N)

LC/MS (ES⁺) m/z 533.1 (M+H)⁺

### Preparation of 4-(dibenzylamino)benzyl 4-(1,2-dihydro-2-oxobenzo[d]imidazol-3-yl)piperidine-1-carboxylate (Compound no 191)

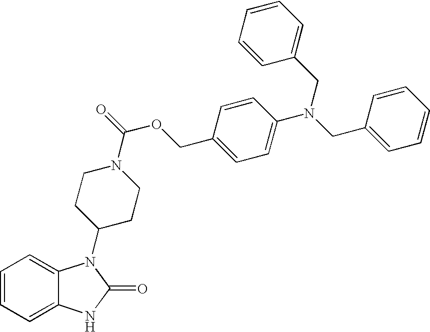

From 4-(2-keto-1-benzimidazolinyl)-piperidine and (4-(dibenzylamino)phenyl)methanol

Yellow solid (12%).

Flash chromatography on silica gel (cyclohexane/ethyl acetate 6/4)

¹H NMR (400 MHz, CDCl₃) δ 10.0 (s, 1H, NH), 7.31 (d, J=6.8 Hz, 4H, Ph), 7.25 (m, 6H, Ph), 7.16 (m, 2H, Ph), 7.07 (m, 4H, Ph), 6.73 (d, J=8.0 Hz, 2H, Ph), 5.07 (s, 2H, CH₂O), 4.68 (s, 4H, NCH₂Ph), 4.49 (m, 1H, CHCH₂CH₂N), 4.11-4.05 (m, 2H, CHCH₂CH₂N), 2.86 (m, 2H, CHCH₂CH₂N), 2.28 (m, 2H, CHCH₂CH₂N), 1.74 (m, 2H, CHCH₂CH₂N)

¹³C NMR (100 MHz, CDCl₃) δ 155.1 (C═O), 154.9 (C═O), 149.3, 138.3, 129.3, 128.8, 128.6, 126.9, 126.5, 121.4, 121.2, 115.9, 112.0, 111.3, 109.8, 109.4, 67.7 (CH₂O), 54.2 (NCH₂Ph), 50.5 (CHCH₂CH₂N), 43.6 (CHCH₂CH₂N), 29.1 (CHCH₂CH₂N)

LC/MS (ES⁺) m/z 547.2 (M+H)⁺

### Preparation of Benzyl 4-(1,2-dihydro-2-oxobenzo[d]imidazol-3-yl)piperidine-1-carboxylate (Compound no 181)

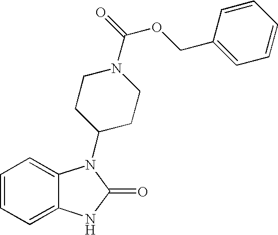

From 4-(2-keto-1-benzimidazolinyl)-piperidine and phenylmethanol

White solid (20%).

Flash chromatography on silica gel (cyclohexane/ethyl acetate 1/1)

¹H NMR (400 MHz, CDCl₃) δ 9.33 (bs, 1H, NH), 7.37 (m, 5H, Bn), 7.07 (m, 4H, Ar), 5.19 (s, CH₂O), 4.52-4.41 (m, 3H, CHCH₂CH₂N, CHCH₂CH₂N), 2.96 (m, 2H, CHCH₂CH₂N), 2.36 (m, 2H, CHCH₂CH₂N), 1.86 (m, 2H, CHCH₂CH₂N). ¹³C NMR (100 MHz, CDCl₃) δ 152.6 (C═O), 152.4 (C═O), 133.9, 126.0, 125.8, 125.5, 125.3, 125.2, 118.6, 118.3, 107.3, 106.5, 64.6 (CH₂O), 50.7 (CHCH₂CH₂N), 40.9 (CHCH₂CH₂N), 26.4 (CHCH₂CH₂N).

LC/MS (ES⁴) m/z 352.1 (M+H)⁺

### Preparation of (R)-2-(dibenzylamino)-3-phenylpropyl 4-(1,2-dihydro-2-oxobenzo[d]imidazol-3-yl)piperidine-1-carboxylate (Compound no 118)

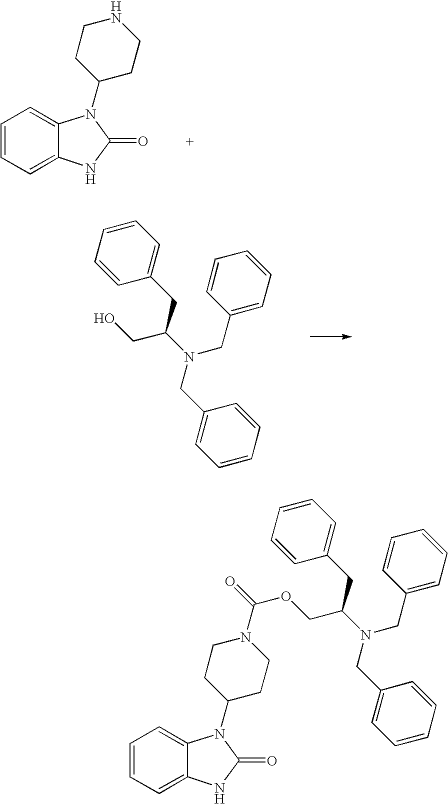

From 4-(2-keto-1-benzimidazolinyl)-piperidine and (R)-2-(dibenzylamino)-3-phenylpropan-1-ol

Pale brown oil (70%).

Flash chromatography on silica gel (cyclohexane/ethyl acetate 8/2)

¹H NMR (400 MHz, CDCl₃) δ 10.28 (s, 1H, NH), 7.30 (m, 12H, Ph), 7.10 (m, 7H, Ph), 4.53 (m, 1H, CHCH₂CH₂N), 4.48 (m, 2H, CHCH₂CH₂N), 4.38 (dd, J=11.2, 6.8 Hz, 1H, CH₂O), 4.18 (dd, J=11.2, 4.8 Hz, 1H, CH₂O), 3.83 (d, J=13.8 Hz, 2H, NCH₂Ph), 3.75 (d, J=13.8 Hz, 2H, NCH₂Ph), 3.29 (m, 1H, CHCH₂Ph), 3.10 (dd, J=13.6, 6.0 Hz, 1H, CHCH₂Ph), 2.92 (m, 2H, CHCH₂CH₂N), 2.71 (dd, J=13.6, 8.4 Hz, 1H, CHCH₂Ph), 2.38 (m, 2H, CHCH₂CH₂N), 1.88 (m, 2H, CHCH₂CH₂N)

¹³C NMR (100 MHz, CDCl₃) δ 155.3 (C═O), 155.1 (C═O), 139.9, 139.5, 129.3, 128.8, 128.6, 128.4, 128.3, 128.2, 126.9, 126.1, 121.4, 121.1, 110.0, 109.3, 64.8 (CH₂O), 58.5 (CHCH₂Ph), 54.0 (NCH₂Ph), 50.6 (CHCH₂CH₂N), 43.6 (CHCH₂CH₂N), 34.1 (CHCH₂Ph), 29.2 (CHCH₂CH₂N)

LC/MS (ES⁺) tn/z 575.2 (M+H)⁺

### 11) Dithiocarbonate Formation

General Procedure

To a solution of appropriate alcohol (2.5 mmol, 1 equiv.) in 5 ml of anhydrous THF was added 120 mg (3.0 mmol, 1.2 equiv.) of sodium hydride at 0° C. and allowed to warm to room temperature for 30′. After addition of 300 μL (5 mmol, 2 equiv.) of carbon disulfure at 0° C., the reaction was continued for 30′. After addition of 280 μL (4.5 mmol, 1.8 equiv.) of methyl iodide at 0° C., the reaction was continued for 30′. The reaction was stopped by addition of ice and the mixture was extracted by dichloromethane. The dichloromethane layer was dried over sodium sulphate, evaporated and used without further purification.

### Preparation of Dithiocarbonic acid O-(2-dibenzylamino-ethyl)ester S-methyl ester (Intermediate)

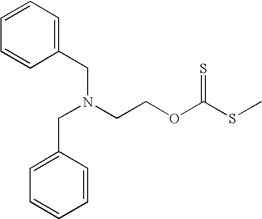

From dibenzylamino-ethan-1-ol

Pale Yellow oil (94%)

¹H NMR (400 MHz, CDCl₃) δ 7.30 (m, 10H, Ph), 4.72 (t, J=5.8 Hz, 2H, OCH₂CH₂N), 3.69 (s, 4H, NCH₂Ph), 2.92 (t, J=5.8 Hz, 2H, OCH₂CH₂N), 2.56 (s, 3H, SCH₃)

LC/MS (ES⁺) m/z 332.1 (M+H)⁺

### 12) Thiocarbamate Formation

General Procedure

To a solution of appropriate dithiocarbonate (1.0 mmol, 1 equiv.) in 1 ml of methyl alcohol was added 260 mg (1.2 mmol, 1.2 equiv.) of 4-(2-keto-1-benzimidazolinyl)-piperidine. The mixture was stirred at 50° C. overnight. The solvent was removed on vacuo and the residue was purified by flash chromatography on silica gel.

### Preparation of O-2-(dibenzylamino)ethyl 4-(1,2-dihydro-2-oxobenzo[d]imidazol-3-yl)piperidine-1-carbothioate (Compound no 46)

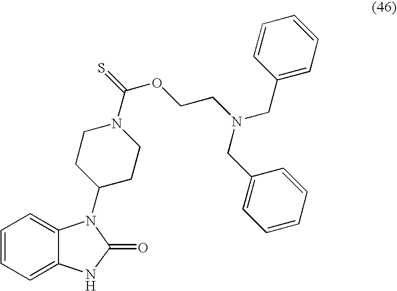

From 4-(2-keto-1-benzimidazolinyl)-piperidine and 2-dibenzylamino-ethan-1-ol.

White solid (59%).

Flash chromatography on silica gel (cyclohexane/ethyl acetate 7/3)

¹H NMR (400 MHz, CDCl₃) δ 10.44 (s, 1H, NH), 7.39 (d, J=7.4 Hz, 4H, Ph), 7.31 (t, J=7.4, 4H, Ph), 7.24 (m, 2H, Ph), 7.17 (d, J=6.8 Hz, 1H, Ph), 7.08 (m, 3H, Ph), 5.40 (d, J=13.5 Hz, 1H, CHCH₂CH₂N), 4.71 (d, J=13.5 Hz, 1H, CHCH₂CH₂N), 4.68-4.57 (m, 3H, CHCH₂CH₂N, OCH₂CH₂N), 3.68 (s, 4H, NCH₂Ph), 3.18 (t, J=12.5 Hz, 1H, CHCH₂CH₂N), 2.96 (t, J=12.5 Hz, 1H, CHCH₂CH₂N), 2.88 (t, J=5.6 Hz, 2H, OCH₂CH₂N), 2.49 (dq, J=12.6, 3.9 Hz, 1H, CHCH₂CH₂N), 2.31 (dq, J=12.6, 3.9 Hz, 1H, CHCH₂CH₂N), 1.96 (d, J=12.6 Hz, 1H, CHCH₂CH₂N), 1.89 (d, J=12.6 Hz, 1H, CHCH₂CH₂N).

¹³C NMR (100 MHz, CDCl₃) δ 187.4 (C═S), 155.2 (C═O), 139.3, 128.7, 128.6, 128.2, 128.0, 127.0, 121.5, 121.2, 110.0, 109.3 (C_{Ph}), 69.6 (OCH₂CH₂N), 58.7 (NCH₂Ph), 51.7 (OCH₂CH₂N), 50.4 (CHCH₂CH₂N), 49.5, 44.5 (CHCH₂CH₂N), 29.1, 28.7 (CHCH₂CH₂N)

LC/MS (ES⁺) m/z 500.8 (M+H)⁺

### Preparation of O-2-(benzyloxy)ethyl 4-(1,2-dihydro-2-oxobenzo[d]imidazol-3-yl)piperidine-1-carbothioate (Compound no 47)

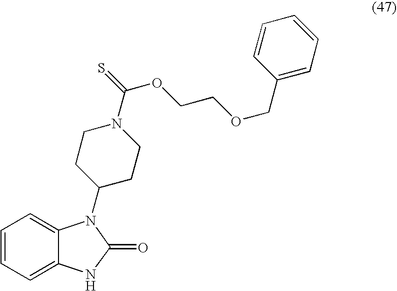

From 4-(2-keto-1-benzimidazolinyl)-piperidine and 2-benzyloxy-ethan-1-ol.

White solid (91%).

Flash chromatography on silica gel (cyclohexane/ethyl acetate 1/1)

¹H NMR (400 MHz, CDCl₃) δ 10.42 (s, 1H, NH), 7.39-7.24 (m, 5H, Ph), 7.20-7.02 (m, 4H, Ph), 5.39 (d, J=13.3 Hz, 1H, CHCH₂CH₂N), 4.84 (d, J=13.3 Hz, 1H, CHCH₂CHN), 4.73 (m, 2H, OCH₂CH₂OBn), 4.61 (m, 3H, OCH₂Ph, CHCH₂CH₂N), 3.80 (dd, J=11.1, 6.6 Hz, 2H, OCH₂CH₂OBn), 3.19 (t, J=12.5 Hz, 1H, CHCH₂CH₂N), 3.02 (t, J=12.5 Hz, 1H, CHCH₂CH₂N), 2.50 (dq, J=12.5, 4.0 Hz, 1H, CHCH₂CH₂N), 2.36 (dq, J=12.5, 4.0 Hz, 1H, CHCH₂CH₂N), 1.86 (m, 2H, CHCH₂CH₂N)

¹³C NMR (100 MHz, CDCl₃) δ 187.3 (C═S), 155.1 (C═O), 137.8, 128.6, 128.4, 128.0, 127.8, 127.7, 121.5, 121.2, 110.0, 109. 3 (C_{Ph}), 73.0 (OCH₂Ph), 70.6 (OCH₂CH₂OBn), 67.9 (OCH₂CH₂OBn), 50.4 (CHCH₂CH₂N), 49.6, 44.6 (CHCH₂CH₂N), 29.1, 28.6 (CHCH₂CH₂N)

LC/MS (ES⁺) m/z 412.1 (M+H)⁺

### Preparation of O—(S)-2-(dibenzylamino)propyl 4-(1,2-dihydro-2-oxobenzo[d]imidazol-3-yl)piperidine-1-carbothioate (Compound no 48)

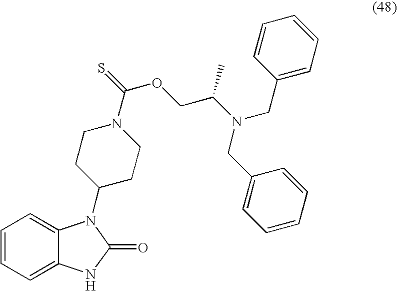

From 4-(2-keto-1-benzimidazolinyl)-piperidine and (S)-2-(dibenzylamino)propan-1-ol

Colorless oil (80%).

Flash chromatography on silica gel (cyclohexane/ethyl acetate 1/1)

¹H NMR (400 MHz, CDCl₃) δ 9.19 (m, 1H, NH), 7.37 (d, J=7.3 Hz, 4H, Ph), 7.32-7.24 (m, 4H, Ph), 7.20 (m, 2H, Ph), 7.16-7.02 (m, 4H, Ph), 5.39 (d, J=13.3 Hz, 1H, CHCH₂CH₂N), 4.76 (d, J=13.3 Hz, 1H, CHCH₂CH₂N), 4.62 (m, 2H, CHCH₂CH₂N, OCH₂), 4.40 (m, 1H, OCH₂), 3.78 (d, J=13.8 Hz, 2H, NCH₂Ph), 3.56 (dd, J=13.8, 3.9 Hz, 2H, NCH₂Ph), 3.27 (m, 1H, CHCH₃), 3.17 (dd, J=25.5, 14.5 Hz, 1H, CHCH₂CH₂N), 3.97 (ddd, J=25.5, 13.2, 12.3 Hz, 1H, CHCH₂CH₂N), 2.57-2.32 (m, 1.5H, CHCH₂CH₂N), 2.32-2.19 (m, 0.5H, CHCH₂CH₂N), 1.96 (d, J=10.9 Hz, 1.5H, CHCH₂CH₂N), 1.92-1.82 (d, J=12.5 Hz, 0.5H), 1.15 (dd, J=6.4, 1.7 Hz, 3H, CHCH₃)

LC/MS (ES⁺) m/z 514.8 (M+H)⁺

### 13) “Alkyl Link” Procedure

To a suspension of 217 mg (1.0 mmol, 1.0 equiv.) of 4-(2-keto-1-benzimidazolinyl)-piperidine, 17 mg (0.1 mmol., 0.1 equiv.) of potassium iodide, 276 mg (2.0 mmol., 2 equiv.) of potassium carbonate in 4 ml of anhydrous DMF was added the appropriate bromide derivative (1.0 mmol, 1 equiv.) in 1 ml of anhydrous DMF. The mixture was stirred at 70° C. overnight. The solvent was removed in vacuo at room temperature and the slurry was partitioned with water and ethyl acetate. The organic layer was dried over sodium sulfate and the solvent was evaporated in vacuum. The residue was purified by flash chromatography on silica gel.

### Preparation of 1-(1-(3-(dibenzylamino)propyl)piperidin-4-yl)-1H-benzo[d]imidazol-2(3H)-one (Compound no 49)

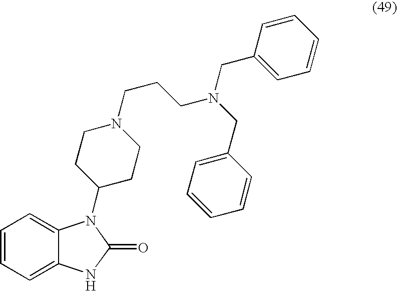

From 4-(2-keto-1-benzimidazolinyl)-piperidine and 3-dibenzylamino-1-bromopropane

Colorless oil (41%).

Preparative LCMS

¹H NMR (400 MHz, CDCl₃) δ 9.56 (s, 1H, NH), 7.31 (m, 11H, Ph), 7.08 (m, 3H, Ph), 4.58 (tt, J=12.2, 3.8 Hz, 1H, CHCH₂CH₂N), 3.67 (s, 4H, NCH₂Ph), 3.56 (d, J=11.7 Hz, 2H, CHCH₂CH₂N), 2.85 (m, 4H, CHCH₂CH₂N, NCH₂CH₂CH₂NBn₂), 2.62 (m, 4H, CHCH₂CH₂N, NCH₂CH₂CH₂NBn₂). 1.93 (m, 4H, CHCH₂CH₂N, NCH₂CH₂CH₂NBn₂)

¹³C NMR (100 MHz, CDCl₃) δ 154.7 (C═O), 137.7, 137.6, 129.3, 128.5, 128.0, 127.6, 122.0, 121.9, 110.0, 109.9 (C_{Ph}), 58.7 (NCH₂Ph), 54.8 (CHCH₂CH₂N), 51.7, 50.6 (NCH₂CH₂CH₂NBn₂), 47.7 (CHCH₂CH₂N), 26.0 (CHCH₂CH₂N), 21.7 (NCH₂CH₂CH₂NBn₂)

LC/MS (ES⁺) m/z 455.2 (M+H)⁺

### Preparation of 1-(14(S)-2-(dibenzylamino)propyl)piperidin-4-0)-1H-benzo[d]imidazol-2(31-1)-one (Compound no 50)

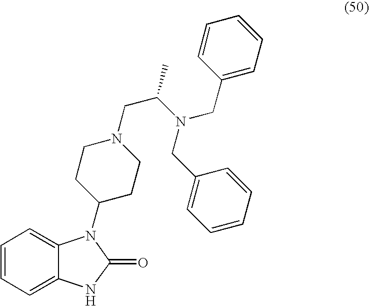

From 4-(2-keto-1-benzimidazolinyl)-piperidine and (S)-2-(dibenzylamino)-1-bromopropane

Yellow oil (6%).

preparative LCMS

LC/MS (ES⁺) m/z 455.2 (M+H)⁺

### Preparation of 1-(1-((S)-2-(dibenzylamino)-3-phenylpropyl)piperidin-4-yl)-1H-benzo[d]imidazol-2(3H)-one (Compound no 51)

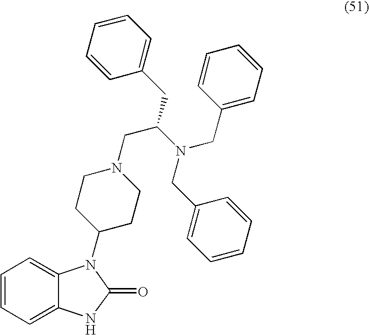

From 4-(2-keto-1-benzimidazolinyl)-piperidine and (S)-2-(dibenzylamino)-3-phenyl-1-bromopropane.

Colorless oil (4%).

preparative LCMS

LC/MS (ES⁺) m/z 631.1 (M+H)⁺

### Preparation of 1-(1-(2-(dibenzylamino)ethyl)piperidin-4-yl)-1H-benzo[d]imidazol-2(3H)-one (Compound no 52)

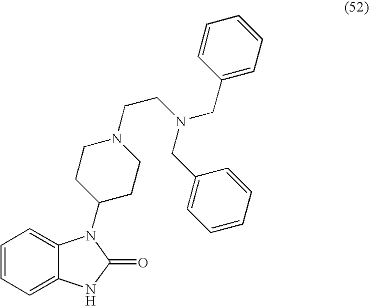

From 4-(2-keto-1-benzimidazolinyl)-piperidine and 2-(dibenzylamino)-1-bromoethane

Colorless oil (20%).

Flash chromatography on TLC preparative silica gel (cyclohexane/ethyl acetate 2/8)

¹H NMR (400 MHz, CDCl₃) δ 10.32 (s, 1H, NH), 7.41 (d, J=7.2 Hz, 4H, Ph), 7.33 (m, 4H, Ph), 7.25 (m, 3H, Ph), 7.12 (m, 1H, Ph), 7.08-7.00 (m, 2H, Ph), 4.35 (tt, J=12.4, 4.1 Hz, 1H, CHCH₂CH₂N), 3.64 (s, 4H, NCH₂Ph), 3.00 (d, J=11.1 Hz, 1H, CHCH₂CH₂N), 2.62 (m, 4H, NCH₂CH₂NBn₂), 2.43 (m, 2H, CHCH₂CH₂N), 2.13 (t, J=11.4, 11.4 Hz, 2H, CHCH₂CH₂N), 1.78 (d, J=11.8 Hz, 2H, CHCH₂CH₂N)

¹³C NMR (100 MHz, CDCl₃) δ 154.7 (C═O), 137.7, 137.6, 129.3, 128.5, 128.0, 127.6, 122.0, 121.9, 110.0, 109.9 (C_{Ph}), 58.7 (NCH₂Ph), 56.3 (CHCH₂CH₂N), 51.0, 53.5 (NCH₂CH₂NBn₂), 50.7 (CHCH₂CH₂N), 29.3 (CHCH₂CH₂N)

LC/MS (ES⁺) m/z 441.1 (M+H)⁺

### 14) N-sulfonyl- and N-acyl-Benzimidazolone Preparation

General Procedure

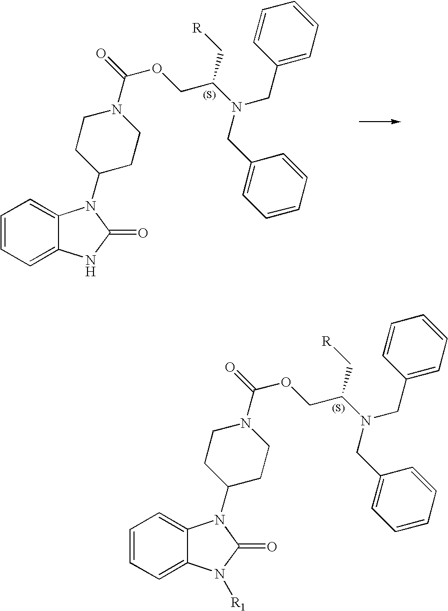

To a stirred solution of the appropriate benzimidazolone derivative (1 equiv.) in THF (20 mL/mmol) at 0° C. was added NaH (60% in mineral oil) (1.2 equiv.) under N₂ atmosphere. The reaction mixture was stirred for 1 h at room temperature. The appropriate sulfonyl chloride or acyl chloride or tert-butyloxycarbonyl anhydride (1.2 equiv.) was added and the reaction mixture was stirred overnight at room temperature. The solvent was removed on vacuo and the residue was purified by flash chromatography on silica gel.

### Preparation of (S)-2-(dibenzylamino)-3-phenylpropyl 4-(1,2-dihydro-3-phenylsulfonyl-2-oxobenzo[d]imidazol-1-yl)piperidine-1-carboxylate (Compound no 53)

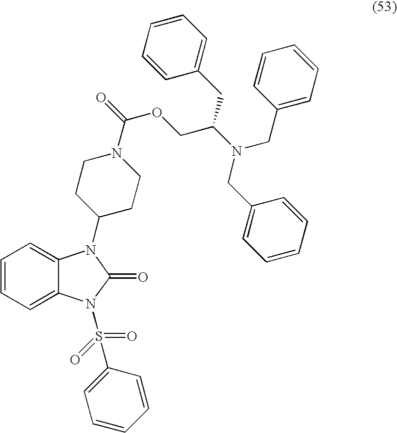

From (S)-2-(dibenzylamino)-3-phenylpropyl 4-(1,2-dihydro-2-oxobenzo[d]imidazol-3-yl)piperidine-1-carboxylate and phenylsulfonyl chloride.

White solid (80%).

Flash chromatography on preparative TLC (silica gel, cyclohexane/ethyl acetate 8/2).

¹H NMR (400 MHz, CDCl₃) δ 8.16 (d, J=7.6 Hz, 2H, Ph), 8.04-7.96 (dd, J=6.9, 2.0, 1H, Ph), 7.67 (t, J=7.6 Hz, 1H, Ph), 7.55 (t, J=7.6 Hz, 2H, Ph), 7.36-7.12 (m, 15H, Ph), 7.08 (m, 3H, Ph), 4.34 (m, 3H, CH₂O, CHCH₂CH₂N), 4.13 (dd_{b}, J=11.5, 4.5 Hz, 2H, CH₂O, CHCH₂CH₂N), 3.76 (2d, J=13.8 Hz, 4H, NCH₂Ph), 3.24 (m, 1H, CHCH₂Ph), 3.08 (dd, J=13.7, 5.8 Hz, 1H, CHCH₂Ph), 2.82 (m, 2H, CHCH)CH₂N), 2.68 (dd, J=13.7, 8.5 Hz, 1H, CHCH₂Ph), 2.27 (m, 2H, CHCH₂CH₂N), 1.74 (d, J=12.2 Hz, 2H, CHCH₂CH₂N)

¹³C NMR (100 MHz, CDCl₃) δ 155.0, 150.0 (C═O), 139.8, 139.5, 137.9, 134.5, 129.2, 129.1, 128.6, 128.3, 128.1, 128.0, 126.8, 126.1, 126.0, 124.0, 122.4, 113.2, 109.4 (C_{Ph}), 64.8 (CH₂O), 58.4 (CHCH₂Ph), 53.9 (NCH₂Ph), 51.4 (CHCH₂CH₂N), 43.4 (CHCH₂CH₂N), 34.1 (CHCH₂Ph), 28.6 (CHCH₂CH₂N)

LC/MS (ES⁺) m/z 715.1 (M+H)⁺

### Preparation of (S)-2-(dibenzylamino)propyl 4-(2-oxo-3-phenylsulfonyl-2,3-dihydro-1H-benzo[d]imidazol-1-yl)piperidine-1-carboxylate (Compound no 54)

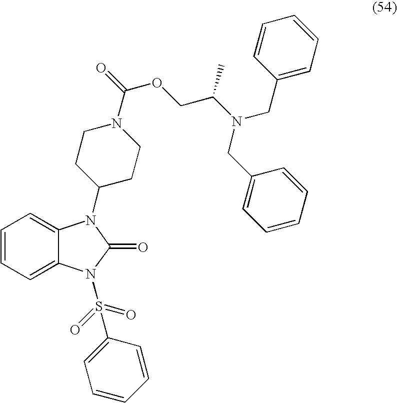

From (S)-2-(dibenzylamino)propyl 4-(1,2-dihydro-2-oxobenzo[d]imidazol-3-yl)piperidine-1-carboxylate and phenylsulfonyl chloride.

White solid (55%).

Flash chromatography on preparative TLC (silica gel, cyclohexane/ethyl acetate 7/3).

¹H NMR (400 MHz, CDCl₃) δ 8.16 (d, J=7.6 Hz, 2H, Ph), 8.00 (m, 1H, Ph), 7.67 (t, J=7.5 Hz, 1H, Ph), 7.55 (t, J=7.8 Hz, 21-1, Ph), 7.37 (d, J=7.3 Hz, 4H), 7.27 (m, 4H, Ph). 7.18 (m, 4H, Ph), 7.08 (m, 1H, Ph), 4.45-4.19 (m, 4H, CHCH₂CH₂N, OCH₂), 4.04 (dd, J=11.1, 5.7 Hz, 1H, OCH₂), 3.75 (d, J=13.9 Hz, 2H, NCH₂Ph), 3.56 (d, J=13.9 Hz, 2H, NCH₂Ph) 3.12 (sext., J=6.7 Hz, 1H, CHCH₃), 2.84 (m, 2H, CHCH₂CH₂N), 2.27 (m, 2H, CHCH₂CH₂N), 1.71 (m, 2H, CHCH₂CH₂N), 1.11 (d, J=6.8 Hz, 3H, CHCH₃)

¹³C NMR (100 MHz, CDCl₃) δ 155.1, 150.0 (C═O), 140.2, 137.9, 134.5, 129.1, 128.5, 128.3, 128.1, 128.1, 126.8, 126.1, 124.1, 122.4, 113.2, 109.4 (C_{Ph}), 66.9 (CH₂O), 53.7 (NCH₂Ph), 51.9 (CHCH₃), 51.5 (CHCH₂CH₂N), 43.4 (CHCH₂CH₂N), 28.6 (CHCH₂CH₂N), 11.2 (CHCH₃)

LC/MS (ES⁺) m/z 638.9 (M+H)⁺

### Preparation of (S)-2-(dibenzylamino)propyl 4-(2-oxo-3-(4-propylphenylsulfonyl)-2,3-dihydro-1H-benzo[d]imidazol-1-yl)piperidine-1-carboxylate (Compound no 55)

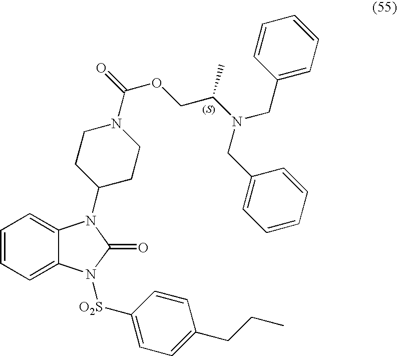

From (S)-2-(dibenzylamino)propyl 4-(1,2-dihydro-2-oxobenzo[d]imidazol-3-yl)piperidine-1-carboxylate and 4-propylphenylsulfonyl chloride.

Colorless oil (70%).

Flash chromatography on silica gel (cyclohexane/ethyl acetate 8/2)

¹H NMR (400 MHz, CDCl₃) δ 8.04 (d, J=8.4 Hz, 2H, Ar), 8.00 (d, J=6.4 Hz, 1H, Ar), 7.36 (m, 6H, Ar), 7.27 (m, 4H. Ar), 7.20 (d, J=6.8 Hz, 2H, Ar), 7.16 (m, 2H, Ar), 7.07 (m, 1H, Ar), 4.36-4.24 (m, 4H, CHCH₂CH₂Nₚᵢₚ, CHCH₂CH₂Nₚᵢₚ, CH₂O), 4.05 (m, 1H, CH₂O), 3.75 (d, J=14.0 Hz, 2H, NCH₂Ph), 3.55 (d, J=14.0 Hz, 21-1, NCH₂Ph), 3.12 (m, 1H, CHMe), 2.85 (m, 2H, CHCH₂CH₂Nₚᵢₚ), 2.65 (t, J=7.6 Hz, 2H, CH₂CH₂CH₃). 2.26 (m, 2H, CHCH₂CH₂Nₚᵢₚ), 1.77 (m, 2H, CHCH₂CH₂Nₚᵢₚ), 1.65 (dd, J=7.2, 7.6 Hz, 2H, CH₂CH₂CH₃), 1.09 (d, J=6.8 Hz, 3H, Me), 0.94 (t. J=7.6 Hz, 3H, CH₂CH₂CH₃).

¹³C NMR (100 MHz, CDCl₃) δ 155.1 (2)(C═O), 150.3, 150.0, 140.2, 135.9, 129.2, 128.5, 128.2, 128.1, 126.8, 126.2, 123.9, 122.4, 113.2, 109.4, 66.9 (CH₂O), 53.7 (NCH₂Ph), 51.9 (CHMe), 51.4 (CHCH₂CH₂Nₚᵢₚ), 43.4 (CHCH₂CH₂Nₚᵢₚ), 38.0 (CH₂CH₂CH₃), 28.6 (CHCH₂CH₂Nₚᵢₚ), 24.0 (CH₂CH₂CH₃), 13.7 (CH₂CH₂CH₃), 11.2 (Me).

LC/MS (ES⁺) m/z 681.3 (M+H)⁺

### Preparation of (S)-2-(dibenzylamino)propyl 4-(3-(4-fluorophenylsulfonyl)-2-oxo-2,3-dihydro-1H-benzo[d]imidazol-1-yl)piperidine-1-carboxylate (Compound no 56)

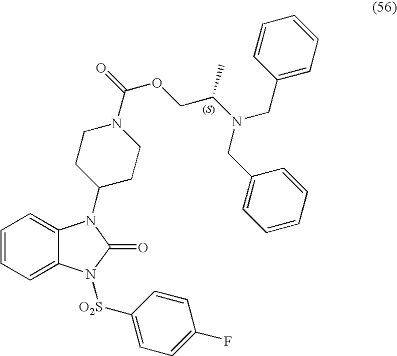

From (S)-2-(dibenzylamino)propyl 4-(1,2-dihydro-2-oxobenzo[d]imidazol-3-yl)piperidine-1-carboxylate and 4-fluorophenylsulfonyl chloride.

White solid (77%).

Flash chromatography on silica gel (cyclohexane/ethyl acetate 8/2)

¹H NMR (400 MHz, CDCl₃) δ 8.20 (dd, J=8.8, 4.8 Hz, 2H, Ar), 7.98 (m, 1H, Ar), 7.36 (m, 4H, Ar), 7.27 (m, 6H, Ar), 7.18 (m, 4H, Ar), 7.07 (m, 1H, Ar), 4.38-4.26 (m, 41-1, CHCH₂CH₂Nₚᵢₚ, CHCH₂CH₂Nₚᵢₚ, CH₂O), 4.05 (dd, J=10.8, 5.6 Hz, 1H, CH₂O), 3.74 (d, J=13.6 Hz, 2H, NCH₂Ph), 3.55 (d, J=13.6 Hz, 2H, NCH₂Ph), 3.12 (m, 1H, CHMe), 2.85 (m, 2H, CHCH₂CH₂Nₚᵢₚ), 2.27 (m, 2H, CHCH₂CH₂Nₚᵢₚ), 1.77 (m, 2H, CHCH₂CH₂Nₚᵢₚ), 1.09 (d, J=6.8 Hz, 3H, Me).

¹³C NMR (100 MHz, CDCl₃) δ 166.2 (J_{C-F}=256.2 Hz, C_{Ar}), 155.1 (C═O), 149.9 (C═O), 140.2, 133.8 (J_{C-F}=3.1 Hz, Cm), 131.2 (J_{C-F}=9.7 Hz, CH_{Ar}), 128.5, 128.3, 128.1, 126.7, 126.0, 124.2, 122.5, 116.5 (J_{C-F}=22.8 Hz, CH_{Ar}), 113.2, 109.5, 66.9 (CH₂O), 53.7 (NCH₂Ph), 51.9 (CHMe), 51.6 (CHCH₂CH₂Nₚᵢₚ), 43.4 (CHCH₂CH₂Nₚᵢₚ), 28.6 (CHCH₂CH₂Nₚᵢₚ), 11.2 (Me).

LC/MS (ES⁺) m/z 657.2 (M+H)⁺

### Preparation of (S)-2-(dibenzylamino)propyl 4-(3-(4-methoxyphenylsulfonyl)-2-oxo-2,3-dihydro-1H-benzo[d]imidazol-1-yl)piperidine-1-carboxylate (Compound no 57)

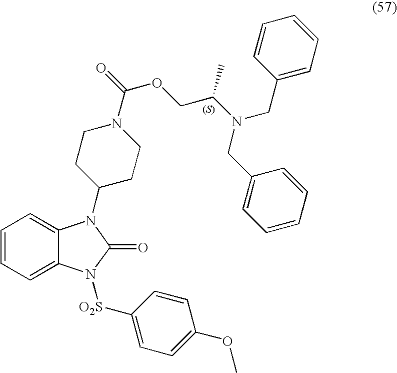

From (S)-2-(dibenzylamino)propyl 4-(1,2-dihydro-2-oxobenzo[d]imidazol-3-yl)piperidine-1-carboxylate and 4-methoxyphenylsulfonyl chloride.

Colorless oil (79%).

Flash chromatography on silica gel (cyclohexane/ethyl acetate 8/2)

¹H NMR (400 MHz, CDCl₃) δ 8.08 (d, J=9.2 Hz, 2H, Ar), 8.00 (m, 1H, Ar), 7.36 (m, 4H, Ar), 7.27 (m, 4H, Ar), 7.21 (d, J=7.2 Hz, 2H, Ar), 7.16 (m, 2H. Ar), 7.07 (m, 1H, Ar), 6.98 (d, J=9.2 Hz, 2H, Ar), 4.40-4.24 (m, 4H, CHCH₂CH₂Nₚᵢₚ, CHCH₂CH₂Nₚᵢₚ, CH₂O), 4.04 (dd, J=10.8, 5.6 Hz, 1H, CH₂O), 3.86 (s, 3H, OMe), 3.74 (d, J=14.0 Hz, 2H, NCH₂Ph), 3.55 (d, J=14.0 Hz, 2H, NCH₂Ph), 3.12 (m, 1H, CHMe), 2.85 (m, 2H, CHCH₂CH₂Nₚᵢₚ), 2.26 (m, 2H, CHCH₂CH₂Nₚᵢₚ), 1.77 (m, 2H, CHCH₂CH₂Nₚᵢₚ), 1.09 (d, J=6.8 Hz, 3H, Me).

¹³C NMR (100 MHz, CDCl₃) δ 164.4 (C_{Ar}), 155.1 (C═O), 150.3 (C═O), 140.2, 130.5, 129.2, 128.5, 128.2, 128.1, 126.8, 126.2, 123.9, 122.4, 114.3, 113.2, 109.4, 66.9 (CH₂O), 55.7 (OMe), 53.7 (NCH₂Ph), 51.9 (CHMe), 51.4 (CHCH₂CH₂Nₚᵢₚ), 43.4 (CHCH₂CH₂Nₚᵢₚ), 28.6 (CHCH₂CH₂Nₚᵢₚ), 11.2 (Me).

LC/MS (ES⁺) m/z 669.3 (M+H)⁺

### Preparation of (S)-2-(dibenzylamino)propyl 4-(2-oxo-3-(4-(trifluoromethyl)phenylsulfonyl)-2,3-dihydro-1H-benzo[d]imidazol-1-yl)piperidine-1-carboxylate (Compound no 58)

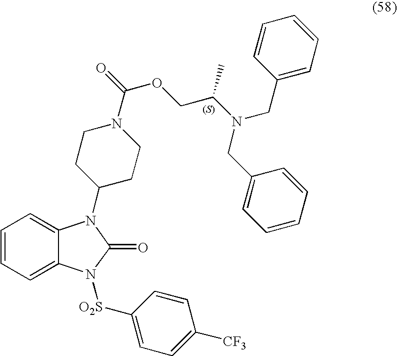

From (S)-2-(dibenzylamino)propyl 4-(1,2-dihydro-2-oxobenzo[d]imidazol-3-yl)piperidine-1-carboxylate and 4-trifluoromethylphenylsulfonyl chloride.

White solid (87%).

Flash chromatography on silica gel (cyclohexane/ethyl acetate 8/2)

¹H NMR (400 MHz, CDCl₃) δ 8.29 (d, J=8.4 Hz, 2H, Ar), 7.99 (dd, J=5.6, 3.2 Hz, 1H, Ar), 7.81 (d, J=8.4 Hz, 2H, Ar), 7.36 (m, 4H, Ar), 7.26 (m, Ar), 7.19 (m, 4H, Ar), 7.09 (m, 1H, Ar), 4.38-4.22 (m, 4H, CHCH₂CH₂Nₚᵢₚ, CHCH₂CH₂Nₚᵢₚ, CH₂O), 4.04 (dd, J=10.8, 5.6 Hz, 1H, CH₂O), 3.74 (d, J=13.6 Hz, 2H, NCH₂Ph), 3.55 (d, J=13.6 Hz, 2H, NCH₂Ph), 3.12 (m, 1H, CHMe), 2.85 (m, 2H, CHCH₂CH₂Nₚᵢₚ), 2.27 (m, 2H, CHCH₂CH₂Nₚᵢₚ), 1.77 (m, 2H, CHCH₂CH₂Nₚᵢₚ), 1.09 (d, J=6.8 Hz, 3H, Me).

¹³C NMR (100 MHz, CDCl₃) δ 155.1 (C═O), 149.8 (C═O), 141.1, 140.2, 136.0 (J_{C-F}=33.0 Hz, C_{Ar}), 128.8, 128.5, 128.3, 128.1, 126.8, 126.3 (J_{C-F}=3.7 Hz, CH_{Ar}), 125.8, 124.4, 122.7, 113.2, 109.7, 66.9 (CH₂O), 53.7 (NCH₂Ph), 51.9 (CHMe), 51.7 (CHCH₂CH₂Nₚᵢₚ), 43.4 (CHCH₂CH₂Nₚᵢₚ), 28.6 (CHCH₂CH₂Nₚᵢₚ), 11.2 (Me).

LC/MS (ES⁺) m/z 707.3 (M+H)⁺

### Preparation of (S)-2-(dibenzylamino)propyl 4-(2-oxo-3-(thiophen-2-ylsulfonyl)-2,3-dihydro-1H-benzo[d]imidazol-1-yl)piperidine-1-carboxylate (Compound no 59)

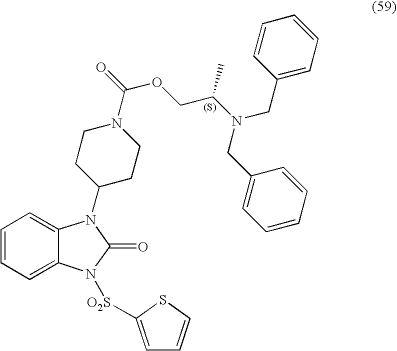

From (S)-2-(dibenzylamino)propyl 4-(1,2-dihydro-2-oxobenzo[d]imidazol-3-yl)piperidine-1-carboxylate and thiophene-2-sulfonyl chloride.

White solid (87%).

Flash chromatography on silica gel (cyclohexane/ethyl acetate 8/2)

¹H NMR (400 MHz, CDCl₃) δ 8.00 (dd, J=4.0, 1.2 Hz, 1H, Ar_{Thio}), 7.94 (m, 1H, Ar), 7.70 (dd, J=4.8, 1.2 Hz, 1H, Ar_{Thio}), 7.36 (d, J=7.2 Hz, 4H, Bn), 7.27 (t, J=7.2 Hz, 4H, Bn), 7.22-7.14 (m, 4H, Ar), 7.13 (dd, J=4.8, 4.0 Hz, 1H, Ar_{Thio}), 7.09 (m, 1H, Ar), 4.40-4.24 (m, 4H, CHCH₂CH₂Nₚᵢₚ, CHCH₂CH₂Nₚᵢₚ, CH₂O), 4.05 (dd, J=10.8, 5.6 Hz, 1H, CH₂O), 3.75 (d, J=14.0 Hz, 2H, NCH₂Ph), 3.56 (d, J=14.0 Hz, 21-1, NCH₂Ph), 3.13 (m, 1H, CHMe), 2.87 (m, 2H, CHCH₂CH₂Nₚᵢₚ), 2.29 (m, 2H, CHCH₂CH₂Nₚᵢₚ), 1.79 (m, 2H, CHCH₂CH₂Nₚᵢₚ), 1.10 (d, J=6.8 Hz, 3H, Me).

¹³C NMR (100 MHz, CDCl₃) δ 155.1 (C═O), 149.8 (C═O), 140.2, 137.5, 135.1, 134.7, 128.5, 128.3, 128.1, 127.6, 126.8, 125.7, 124.2, 122.5, 113.2, 109.4, 66.9 (CH₂O), 53.7 (NCH₂Ph), 51.9 (CHMe), 51.6 (CHCH₂CH₂Nₚᵢₚ), 43.4 (CHCH₂CH₂Nₚᵢₚ), 28.6 (CHCH₂CH₂Nₚᵢₚ), 11.2 (Me).

LC/MS (ES⁺) m/z 645.3 (M+H)⁺

### Preparation of (S)-2-(dibenzylamino)propyl 4-(2-oxo-3-(quinolin-8-ylsulfonyl)-2,3-dihydro-1H-benzo[d]imidazol-1-yl)piperidine-1-carboxylate (Compound no 60)

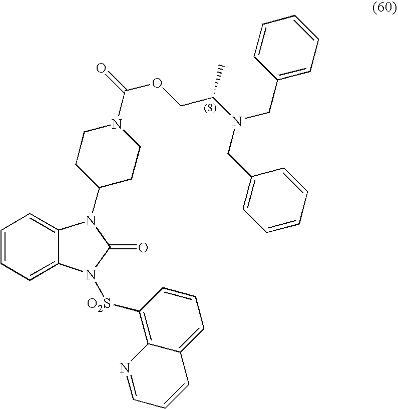

From (S)-2-(dibenzylamino)propyl 4-(1,2-dihydro-2-oxobenzo[d]imidazol-3-yl)piperidine-1-carboxylate and quinoline-8-sulfonyl chloride.

White solid (47%).

Flash chromatography on silica gel (methylene chloride/acetone 95/5)

¹H NMR (400 MHz, CDCl₃) δ 8.78 (dd, J=7.2, 0.8 Hz, 1H, Quino.), 8.56 (dd, J=4.0, 1.6 Hz, 1H, Quino.), 8.20 (m, 2H, Ar), 8.12 (dd, J=8.0, 0.8 Hz, 1H, Ar), 7.74 (t, J=8.0 Hz, 1H, Ar), 7.39 (m, 1H, Quino.), 7.35 (d, J=7.2 Hz, 4H, Bn), 7.27 (t, J=7.2 Hz, 4H, Bn), 7.22-7.18 (m, 4H, Ar), 7.05 (m, 1H, Ar), 4.40-4.15 (m, 4H, CHCH₂CH₂Nₚᵢₚ, CHCH₂CH₂Nₚᵢₚ, CH₂O), 4.02 (dd, J=10.8, 5.6 Hz, 1H, CH₂O), 3.72 (d, J=13.6 Hz, 2H, NCH₂Ph), 3.54 (d, J=13.6 Hz, 2H, NCH₂Ph), 3.09 (m, 1H, CHMe), 2.77 (m, 2H, CHCH₂CH₂Nₚᵢₚ), 2.18 (m, 2H, CHCH₂CH₂Nₚᵢₚ), 1.62 (bd, 2H, CHCH₂CH₂Nₚᵢₚ), 1.08 (d, J=6.4 Hz, 3H, Me).

¹³C NMR (100 MHz, CDCl₃) δ 155.1 (C═O), 151.3 (C═O), 150.3 (Quino.), 143.8, 140.2, 136.3, 135.0, 134.8, 134.1, 128.5, 128.4, 128.1, 128.0, 127.8, 126.8, 125.5, 123.4, 122.1, 114.9, 109.0, 66.9 (CH₂O), 53.7 (NCH₂Ph), 51.9 (CHMe), 51.2 (CHCH₂CH₂Nₚᵢₚ), 43.4 (CHCH₂CH₂Nₚᵢₚ), 28.5 (CHCH₂CH₂Nₚᵢₚ), 11.2 (Me).

LC/MS (ES⁺) m/z 690.3 (M+H)⁺

### Preparation of (S)-2-(dibenzylamino)propyl 4-(3-(naphthalen-1-ylsulfonyl)-2-oxo-2,3-dihydro-1H-benzo[d]imidazol-1-yl)piperidine-1-carboxy late (Compound no 61)

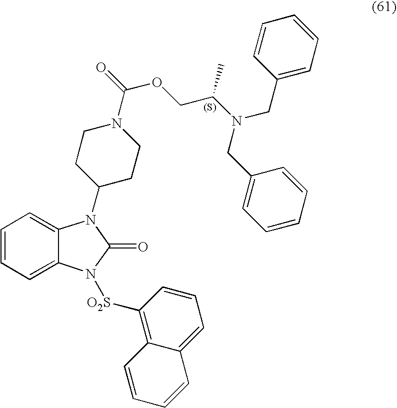

From (S)-2-(dibenzylamino)propyl 4-(1,2-dihydro-2-oxobenzo[d]imidazol-3-yl)piperidine-1-carboxylate and naphthalene-1-sulfonyl chloride.

White solid (56%).

Flash chromatography on silica gel (cyclohexane/ethyl acetate 8/2)

¹H NMR (400 MHz, CDCl₃) δ 8.66 (d, 1=7.2 Hz, 1H, Napht.), 8.56 (m, 1H, Napht.), 8.15 (dd, J=11.6, 8.0 Hz, 2H, Napht.), 7.94 (m, 1H, Ar), 7.66 (t, J=8.0 Hz, 1H, Napht.), 7.39 (m, 2H, Napht.), 7.35 (d, J=7.2 Hz, 4H, Bn), 7.26 (t, J=6.8 Hz, 4H, Bn), 7.20-7.17 (m, 4H, Ar), 7.09 (m, 1H, Ar), 4.28-4.19 (m, 4H, CHCH₂CH₂Nₚᵢₚ, CHCH₂CH₂Nₚᵢₚ, CH₂O), 4.01 (dd, J=10.8, 5.6 Hz, 1H, CH₂O), 3.73 (d, J=13.6 Hz, 2H, NCH₂Ph), 3.54 (d, J=13.6 Hz, 2H, NCH₂Ph), 3.10 (m, 1H, CHMe), 2.78 (m, 2H, CHCH₂CH₂Nₚᵢₚ), 2.18 (m, 2H, CHCH₂CH₂Nₚᵢₚ), 1.67 (bd, 2H, CHCH₂CH₂Nₚᵢₚ), 1.08 (d, J=6.8 Hz, 3H, Me).

¹³C NMR (100 MHz, CDCl₃) δ 155.0 (C═O), 149.8 (C═O), 140.2, 136.2, 134.0, 132.9, 132.6, 129.2, 128.9, 128.5, 128.2, 128.1, 126.9, 126.8, 126.5, 124.1, 124.0, 123.4, 122.5, 113.4, 109.6, 66.9 (CH₂O), 53.7 (NCH₂Ph), 51.9 (CHMe), 51.4 (CHCH₂CH₂Nₚᵢₚ), 43.3 (CHCH₂CH₂Nₚᵢₚ), 28.5 (CHCH₂CH₂Nₚᵢₚ), 11.2 (Me).

LC/MS (ES⁺) m/z 689.3 (M+H)⁺

### Preparation of (S)-2-(dibenzylamino)propyl 4-(2-oxo-3-methylsulfonyl-2,3-dihydro-1H-benzo[d]imidazol-1-yl)piperidine-1-carboxylate (Compound no 62)

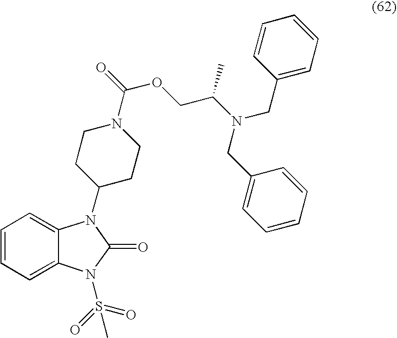

From (S)-2-(dibenzylamino)propyl 4-(1,2-dihydro-2-oxobenzo[d]imidazol-3-yl)piperidine-1-carboxylate and methylsulfonyl chloride.

White solid (45%).

Flash chromatography on preparative TLC (silica gel, cyclohexane/ethyl acetate 1/1)

¹H NMR (400 MHz, CDCl₃) δ 7.84 (d, J=7.5 Hz, 1H, Ph), 7.38 (d, 1=7.4 Hz, 4H, Ph), 7.28 (m, 4H, Ph), 7.16 (m, 5H, Ph), 4.45 (tt, J=12.3, 3.7 Hz, 1H, CHCH₇CH₂N), 4.39 (m, 2H, CHCH₂CH₂N), 4.27 (dd, J=11.1, 7.6 Hz, 1H, OCH₂), 4.07 (dd, J=11.1, 5.7 Hz, 1H, OCH₂), 3.76 (d, J=13.9 Hz, 2H, NCH₂Ph), 3.57 (d, J=13.9 Hz, 2H, NCH₂Ph), 3.52 (s, 3H, SO₂CH₃), 3.15 (sext., J=6.7 Hz, 1H, CHCH₃), 2.93 (m, 2H, CHCH₂CH₂N), 2.36 (m, 2H, CHCH₂CH₂N), 1.88 (d, J=10.1 Hz, 2H, CHCH₂CH₂N), 1.11 (d, J=6.8 Hz, 3H, CHCH₃)

¹³C NMR (100 MHz, CDCl₃) δ 155.1, 150.6 (C═O), 140.2, 128.5, 128.1, 126.8, 126.0, 124.1, 122.5, 113.2, 109.5 (C_{Ph}), 66.9 (CH₂O), 53.7 (NCH₂Ph), 51.9 (CHCH₃), 51.6 (CHCH₂CH₂N), 43.4 (CHCH₂CH₂N), 41.7 (SO₂CH₃), 28.9 (CHCH₂CH₂N), 11.2 (CHCH₃)

LC/MS (ES⁺) m/z 576.9 (M+H)⁺

### Preparation of (S)-2-(dibenzylamino)propyl 2-(2-oxo-3-(4-acetamidylphenylsulfonyl)-2,3-dihydro-1H-benzo[d]imidazol-1-yl)piperidine-1-carboxylate (Compound no 63)

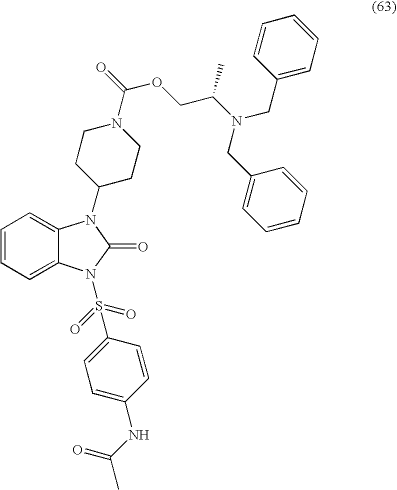

From (S)-2-(dibenzylamino)propyl 4-(1,2-dihydro-2-oxobenzo[d]imidazol-3-yl)piperidine-1-carboxylate and 4-acetylaminophenylsulfonyl chloride.

Yellow oil (56%).

Flash chromatography on preparative TLC (silica gel, cyclohexane/ethyl acetate 1/1)

¹H NMR (400 MHz, CDCl₃) δ 8.58 (s, 1H, NH), 8.02 (d, J=8.8 Hz, 2H, Ph), 7.97 (dd, J=6.2, 3.0 Hz, 1H, Ph), 7.72 (d, J=8.8 Hz, 2H, Ph), 7.36 (d, J=7.3 Hz, 4H, Ph), 7.26 (t, J=7.4 Hz, 4H, Ph), 7.17 (m, 4H, Ph), 7.07 (m, 1H, Ph), 4.32 (tt_{b}, J=12.3, 4.1 Hz, 2H, CHCH₂CH₂N), 4.24 (m, 2H, CHCH₂CH₂N, OCH₂), 4.02 (dd, J=11.1, 5.7 Hz, 1H, OCH₂), 3.74 (d, J=13.9 Hz, 2H, NCH₂Ph). 3.54 (d, J=13.9 Hz, 2H, NCH₂Ph), 3.12 (next., J=6.7 Hz, 1H, CHCH₃). 2.89 (m, 2H, CHCH₂CH₂N), 2.29 (m, 2H, CHCH₂CH₂N), 2.18 (s, 3H, COCH₃), 1.75 (m, 2H, CHCH₂CH₂N), 1.08 (d, J=6.7 Hz, 3H, CHCH₃)

¹³C NMR (100 MHz, CDCl₃) δ 169.2 (NHC═O), 155.2, 150.1 (C═O), 144.1, 140.1, 131.7, 129.4, 128.5, 128.2, 128.1, 126.8, 126.0, 124.1, 122.6, 119.2, 113.2, 109.4 (C_{Ph}), 66.9 (CH₂O), 53.6 (NCH₂Ph), 51.8 (CHCH₃), 51.5 (CHCH₂CH₂N), 43.4 (CHCH₂CH₂N), 28.5 (CHCH₂CH₂N), 24.2 (NHCOCH₃), 11.0 (CHCH₃)

LC/MS (ES⁺) m/z 696.3 (M+H)⁺

### Preparation of (S)-2-(dibenzylamino)propyl 4-(1,2-dihydro-2-oxo-1-(benzoyl)benzo[d]imidazol-3-yl)piperidine-1-carboxylate (Compound no 64)

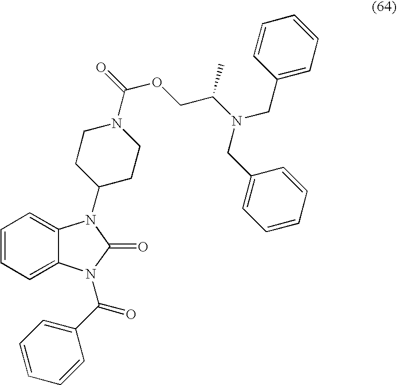

From (S)-2-(dibenzylamino)propyl 4-(1,2-dihydro-2-oxobenzo[d]imidazol-3-yl)piperidine-1-carboxylate and benzoyl chloride.

White solid (67%).

Flash chromatography on TLC preparative, silica gel (cyclohexane/ethyl acetate 7/3)

¹H NMR (400 MHz, CDCl₃) δ 8.00 (dd, J=6.4, 2.4 Hz, 1H, Ph), 7.79 (d, J=7.3 Hz, 2H, Ph), 7.61 (t, J=7.5 Hz, 1H, Ph), 7.49 (m, 21-1, Ph), 7.39 (d, J=7.3 Hz, 4H), 7.28 (m, 4H, Ph), 7.20 (m, 5H, Ph), 4.43 (tt, J=12.2, 3.6 Hz, 1H, CHCH₂CH₂N), 4.34 (m, 2H, CHCH₂CH₂N), 4.26 (m, 1H, OCH₂), 4.06 (dd, J=11.1, 5.7 Hz, 1H, OCH₂), 3.77 (d, J=13.9 Hz, 2H, NCH₂Ph), 3.57 (d, J=13.9 Hz, 2H, NCH₂Ph), 3.14 (seat., J=6.7 Hz, 1H, CHCH₃), 2.89 (m, 2H, CHCH₂CH₂N), 2.37 (m, 2H, CHCH₂CH₂N), 1.87 (d, J=10.7 Hz, 2H, CHCH₂CH₂N), 1.12 (d, J=6.8 Hz, 3H, CHCH₃)

¹³C NMR (100 MHz, CDCl₃) δ 168.9, 155.1, 151.4 (C═O), 140.2, 133.8, 132.6, 130.0, 129.2, 129.0, 128.5, 128.4, 128.1, 128.0, 126.9, 126.8, 124.3, 122.4, 114.9, 109.1 (C_{Ph}), 66.9 (CH₂O), 53.7 (NCH₂Ph), 51.9 (CHCH₃), 51.5 (CHCH₂CH₂N), 43.5 (CHCH₂CH₂N), 28.7 (CHCH₂CH₂N), 11.2 (CHCH₃)

LC/MS (ES⁺) m/z 602.9 (M+H)⁺

### Preparation of (S)-2-(dibenzylamino)propyl 4-(1-acetyl-1,2-dihydro-2-oxobenzo[d]imidazol-3-yl)piperidine-1-carboxylate (Compound no 65)

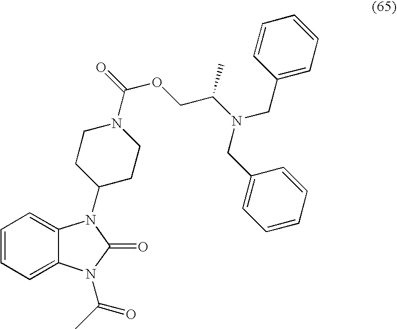

From (S)-2-(dibenzylamino)propyl 4-(1,2-dihydro-2-oxobenzo[d]imidazol-3-yl)piperidine-1-carboxylate and acetyl chloride.

White solid (68%).

Flash chromatography on preparative TLC (silica gel, cyclohexane/ethyl acetate 7/3)

¹H NMR (400 MHz, CDCl₃) δ 8.26 (d, J=7.6 Hz, 1H, Ph), 7.39 (d, J=7.4 Hz, 4H, Ph), 7.29 (m, 4H, Ph), 7.16 (m, 5H, Ph), 4.47 (tt, J=12.2, 3.6 Hz, 1H, CHCH₂CH₂N), 4.42 (m, 2H, CHCH₂CH₂N), 4.27 (dd, J=11.0, 7.5 Hz, 1H, OCH₂), 4.08 (dd, J=11.0, 5.7 Hz, 1H, OCH₂), 3.77 (d, J=13.9 Hz, 2H, NCH₂Ph), 3.57 (d, J=13.9 Hz, 2H, NCH₂Ph), 3.15 (next., J=6.6 Hz, 1H, CHCH₃), 2.94 (m, 2H, CHCH₂CH₂N), 3.52 (s, 3H, COCH₃), 2.39 (m, 2H, CHCH₂CH₂N), 1.87 (d, J=10.6 Hz, 2H, CHCH₂CH₂N), 1.12 (d, J=6.8 Hz, 3H, CHCH₃)

¹³C NMR (100 MHz, CDCl₃) δ 170.5 (C═O), 155.1 (C═O), 151.8 (C═O), 140.2, 128.5, 128.1, 126.8, 126.3, 124.4, 122.5, 116.0, 108.7 (C_{Ph}), 66.9 (CH₂O), 53.7 (NCH₂Ph), 51.9 (CHCH₃), 51.3 (CHCH₂CH₂N), 43.5 (CHCH₂CH₂N), 28.6 (CHCH₂CH₂N), 25.8 (COCH₃), 11.2 (CHCH₃)

LC/MS (ES⁺) m/z 540.9 (M+H)⁺

### Preparation of (S)-2-(dibenzylamino)propyl 4-(1,2-dihydro-2-oxo-1-(pivaloyl)benzo[d]imidazol-3-yl)piperidine-1-carboxylate (Compound no 66)

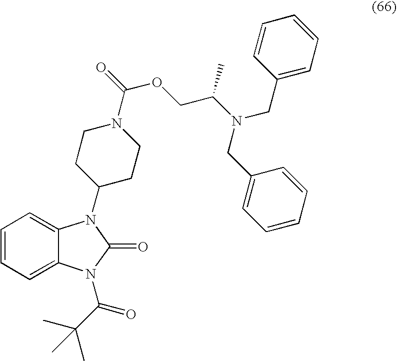

From (S)-2-(dibenzylamino)propyl 4-(1,2-dihydro-2-oxobenzo[d]imidazol-3-yl)piperidine-1-carboxylate and pivaloyl chloride.

White solid (6%) instable on silica gel.

HPLC preparative

LC/MS (ES⁺) m/z 483.3 (M+H)⁺

### Preparation of tert-butyl 3-(1-(((S)-2-(dibenzylamino) propoxy)carbonyl)piperidin-4-yl)-2,3-dihydro-2-oxobenzo[d]imidazole-1-carboxylate (Compound no 67)

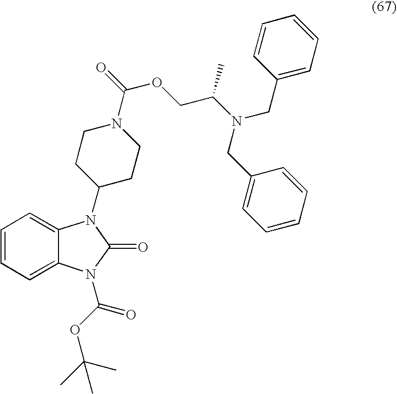

From (S)-2-(dibenzylamino)propyl 4-(1,2-dihydro-2-oxobenzo[d]imidazol-3-yl)piperidine-1-carboxylate and di-tent-butyl-dicarbonate.

White solid (63%).

Flash chromatography on silica gel (cyclohexane/ethyl acetate 7/3)

¹H NMR (400 MHz, CDCl₃) δ 7.89 (dd, J=7.2, 1.6 Hz, 1H, Ph), 7.38 (d, J=7.3 Hz, 4H, Ph), 7.28 (dd, J=12.1, 4.4 Hz, 4H, Ph), 7.21 (m, 2H, Ph), 7.10 (m, 3H, Ph), 4.52 (tt, J=12.6, 3.8 Hz, 1H, CHCH₂CH₂N), 4.38 (m, 2H, CHCH₂CH₂N), 4.26 (dd, J=11.0, 7.7 Hz, 1H, OCH₂), 4.07 (dd, J=11.0, 5.8 Hz, 1H, OCH₂), 3.76 (d, J=13.9 Hz, 21-1, NCH₂Ph), 3.57 (d, J=13.9 Hz, 2H, NCH₂Ph), 3.14 (seat., J=6.7 Hz, 1H, CHCH₃), 2.92 (m, 2H, CHCH₂CH₂N), 2.37 (m, 2H, CHCH₂CH₂N), 1.83 (m, 2H, CHCH₂CH₂N), 1.69 (s, 9H, tBu), 1.11 (d, J=6.8 Hz, 3H, CHCH₃)

¹³C NMR (100 MHz, CDCl₃) δ 155.1, 150.5 (C═O), 148.8 (C═O), 140.2, 128.5, 128.1, 128.1, 126.8, 126.3, 123.7, 121.9, 114.6, 108.8 (C_{Ph}), 84.7 (C_{q}tBu), 66.8 (CH₂O), 53.7 (NCH₂Ph), 51.9 (CHCH₃), 51.1 (CHCH₂CH₂N), 43.5 (CHCH₂CH₂N), 28.7 (tBu), 26.9 (CHCH₂CH₂N), 11.2 (CHCH₃)

LC/MS (ES⁺) m/z 599.2 (M+H)⁺

### 15) N-alkyl Benzimidazolone Preparation

To a stirred solution of (0.48 mmol, 1 equiv.) of benzimidazolone derivative in 1 ml of anhydrous DMF was added at 0° C. 21 mg (0.53 mmol, 1.1 equiv.) of sodium hydride. The reaction was pursued for 1 hour. An appropriate bromide (in these cases, 0.1 equiv. of potassium iodide was added) or iodide in 1 ml of DMF was added at 0° C. The reaction was allowed to warm to room temperature overnight. The mixture was evaporated on vacuum at room temperature and purified on silica gel.

### Preparation of (S)-2-(dibenzylamino)-3-phenylpropyl 4-(1,2-dihydro-1-methyl-2-oxobenzo[d]imidazol-3-yl)piperidine-1-carboxylate (Compound no 68)

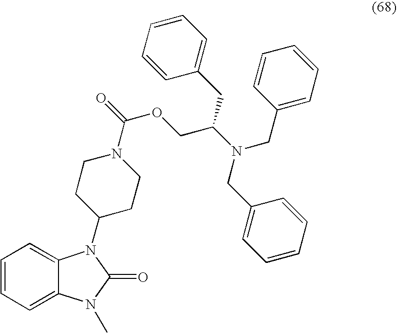

From (S)-2-(dibenzylamino)-3-phenylpropyl 4-(1,2-dihydro-2-oxobenzo[d]imidazol-3-yl)piperidine-1-carboxylate and methyliodide.

White solid (85%).

Flash chromatography on preparative TLC (silica gel, cyclohexane/ethyl acetate 1/1)

¹H NMR (400 MHz, CDCl₃) δ 7.34-7.17 (m, 13H, Ph), 7.15-6.97 (m, 6H, Ph), 4.54 (tt, J=12.4, 3.8 Hz, 1H, CHCH₂CH₂N), 4.42 (m, 1H, CHCH₂CH₂N), 4.36 (dd, J=11.2, 7.0 Hz, 1H, CH₂O), 4.26-4.09 (dd_{b}, J=11.2, 4.8 Hz, 2H, CH₂O, CHCH₂CH₂N), 3.78 (2d, J=13.8 Hz, 4H, NCH₂Ph), 3.43 (s, 3H, NCH₃), 3.28 (m, 1H, CHCH₂Ph), 3.09 (dd, J=13.7, 5.9 Hz, 1H, CHCH₂Ph), 2.90 (m, 2H, CHCH₂CH₂N), 2.70 (dd, J=13.7, 8.5 Hz, 1H, CHCH₂Ph), 2.33 (m, 2H, CHCH₂CH₂N), 1.85 (d, J=12.6 Hz, 2H, CHCH₂CH₂N)

¹³C NMR (100 MHz, CDCl₃) δ 155.1, 153.8 (C═O), 139.8, 139.5, 130.1, 129.2, 128.6, 128.3, 128.1, 127.9, 126.8, 126.0, 121.1, 121.0, 109.0, 107.6 (C_{Ph}), 64.7 (CH₂O), 58.4 (CHCH₂Ph), 53.9 (NCH₂Ph), 50.9 (CHCH₂CH₂N), 43.6 (CHCH₂CH₂N), 34.1 (CHCH₂Ph), 29.2 (CHCH₂CH₂N), 27.1 (NCH₃)

LC/MS (ES⁺) m/z 589.3 (M+H)⁺

### Preparation of (S)-2-(dibenzylamino)propyl 4-(1,2-dihydro-1-methyl-2-oxobenzo[d]imidazol-3-yl)piperidine-1-carboxylate (Compound no 69)

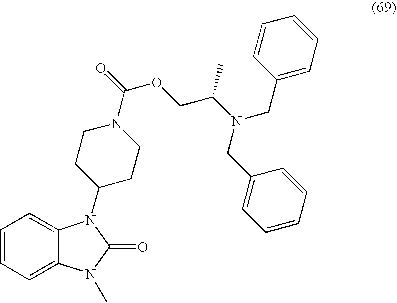

From (S)-2-(dibenzylamino)propyl 4-(1,2-dihydro-2-oxobenzo[d]imidazol-3-yl)piperidine-1-carboxylate and methyliodide.

White solid (95%).

Flash chromatography on silica gel (cyclohexane/ethyl acetate 1/1)

¹H NMR (400 MHz, CDCl₃) δ 7.38 (d, J=7.3 Hz, 4H, Ph), 7.28 (m, 4H, Ph), 7.21 (m, 2H, Ph), 7.06 (m, 4H, Ph), 4.53 (tt, J=12.4, 3.7 Hz, 1H, CHCH₂CH₂N), 4.49-4.30 (m, 2H, CHCH₂CH₂N), 4.27 (dd, J=11.0, 7.5 Hz, 1H, CH₂O), 4.07 (dd, J=11.0, 5.7 Hz, 1H, CH₂O), 3.76 (d, J=13.9 Hz, 2H, NCH₂Ph), 3.57 (d, J=13.9 Hz, 2H, NCH₂Ph), 3.43 (s, 3H, NCH₃), 3.14 (sext., J=6.7 Hz, 1H, CHCH₃), 2.94 (m, 2H, CHCH₂CH₂N), 2.31 (m, 2H, CHCH₂CH₂N), 1.86 (d, J=6.8 Hz, 2H, CHCH₂CH₂N), 1.12 (d, J=6.8 Hz, 3H, CHCH₃)

LC/MS (ES⁺) m/z 512.9 (M+H)⁺

### Preparation of (S)-2-(dibenzylamino)-3-phenylpropyl 4-(1-benzyl-1,2-dihydro-2-oxobenzo[d]imidazol-3-yl)piperidine-1-carboxylate (Compound no 70)

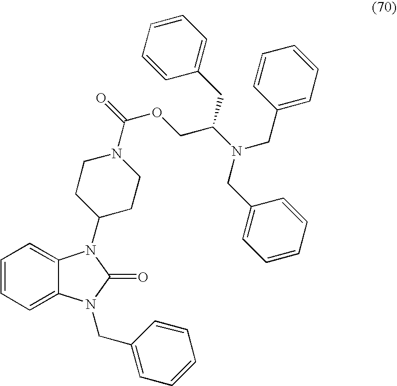

From (S)-2-(dibenzylamino)-3-phenylpropyl 4-(1,2-dihydro-2-oxobenzo[d]imidazol-3-yl)piperidine-1-carboxylate and benzylbromide.

White solid (92%).

Flash chromatography on silica gel (cyclohexane/ethyl acetate 8/2)

¹H NMR (400 MHz, CDCl₃) δ 7.37-7.17 (m, 18H, Ph), 7.09 (d, J=7.0 Hz, 3H, Ph), 7.00 (m, 2H, Ph), 6.91 (m, 1H, Ph), 5.08 (s, 2H, NCH₂Ph), 4.58 (tt, J=12.5, 3.8 Hz, 1H, CHCH₂CH₂N), 4.44 (m, 1H, CHCH₂CH₂N), 4.35 (dd, J=11.2, 7.1 Hz, 1H, OCH₂). 4.20 (m, 2H, CH₂O, CHCH₂CH₂N), 3.81 (d, J=13.8 Hz, 2H, NCH₂Ph), 3.74 (d, J=13.8 Hz, 2H, NCH₂Ph), 3.26 (m, 1H, CHCH₂Ph), 3.09 (dd, J=13.7, 5.9 Hz, 1H, CHCH₂Ph), 2.90 (m, 2H, CHCF₂CH₂N), 2.70 (dd, J=13.7, 8.5 Hz, 1H, CHCH₂Ph), 2.35 (m, 2H, CHCH₂CH₂N), 1.89 (d, J=11.1 Hz, 2H, CHCH₂CH₂N)

¹³C NMR (100 MHz, CDCl₃) δ 155.1, 153.8 (C═O), 139.8, 139.5, 136.1, 129.3, 129.2, 128.7, 128.6, 128.3, 128.1, 128.0, 127.7, 127.4, 126.8, 126.0, 121.1, 121.0, 109.2, 108.5 (C_{Ph}), 64.7 (CH₂O), 58.4 (CHCH₂Ph), 53.9 (NCH₂Ph), 51.0 (CHCH₂CH₂N), 44.9 (O═CNCH₂Ph), 43.6 (CHCH₂CH₂N), 34.1 (CHCH₂Ph), 29.2 (CHCH₂CH₂N)

LC/MS (ES⁺) m/z 665.4 (M+H)⁺

### Preparation of (S)-2-(dibenzylamino)propyl 4-(1-benzyl-1,2-dihydro-2-oxobenzo[d]imidazol-3-yl)piperidine-1-carboxylate (Compound no 71)

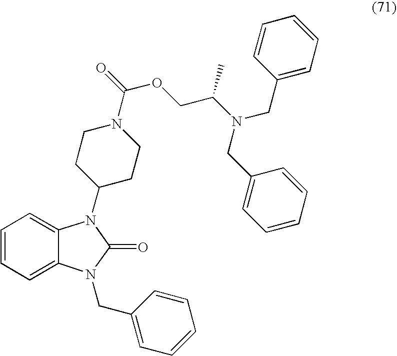

From (S)-2-(dibenzylamino)propyl 4-(1,2-dihydro-2-oxobenzo[d]imidazol-3-yl)piperidine-1-carboxylate and benzylbromide.

Yellow oil (73%).

Flash chromatography on preparative TLC (silica gel, cyclohexane/ethyl acetate 7/3)

¹H NMR (400 MHz, CDCl₃) δ 7.40 (d, J=7.3 Hz, 4H, Ph), 7.31 (m, 9H, Ph), 7.22 (t, J=7.2 Hz, 2H, Ph), 7.13 (m, 1H, Ph), 7.02 (m, 2H, Ph), 6.93 (m, 1H, Ph), 5.09 (s, 2H, NCH₂Ph), 4.60 (tt, J=12.4, 3.8 Hz, 1H, CHCH₂CH₂N), 4.39 (m, 2H, CHCH₂CH₂N), 4.28 (dd, J=11.0, 7.6 Hz, 1H, OCH₂), 4.08 (dd, J=11.0, 5.7 Hz, 1H, OCH₂), 3.78 (d, J=13.9 Hz, 2H, NCH₂Ph), 3.58 (d, J=13.9 Hz, 2H, NCH₂Ph), 3.16 (sext., J=6.7 Hz, 1H, CHCH₃), 2.95 (m. 2H, CHCH₂CH₂N). 2.38 (m, 2H, CHCH₂CH₂N), 1.91 (d, J=10.2 Hz, 2H, CHCH₂CH₂N), 1.13 (d, J=6.8 Hz, 3H, CHCH₃)

¹³C NMR (100 MHz, CDCl₃) δ 155.2, 153.8 (C═O), 140.2, 136.2, 129.3, 128.7, 128.5, 128.1, 128.0, 127.7, 127.5, 126.8, 121.1, 121.0, 109.2, 108.5 (C_{Ph}), 64.8 (CH₂O), 53.4 (NCH₂Ph), 51.9 (CHCH₃), 51.1 (CHCH₂CH₂N), 44.9 (O═CNCH₂Ph), 43.6 (CHCH₂CH₂N), 29.2 (CHCH₂CH₂N), 11.3 (CHCH₃)

LC/MS (ES⁺) m/z 588.9 (M+H)⁺

### Preparation of (S)-2-(dibenzylamino)propyl 4-(1-(cyclohexylmethyl)-1,2-dihydro-2-oxobenzo[d]imidazol-3-yl)piperidine-1-carboxylate (Compound no 72)

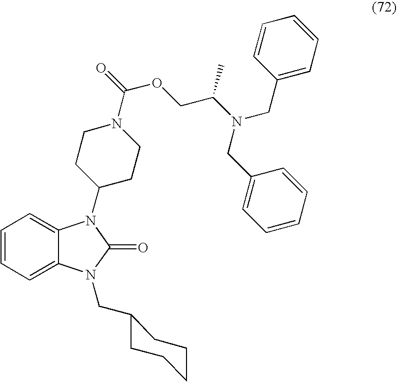

From (S)-2-(dibenzylamino)propyl 4-(1,2-dihydro-2-oxobenzo[d]imidazol-3-yl)piperidine-1-carboxylate and cyclohexylmethylbromide.

Yellow oil (29%).

Flash chromatography on preparative TLC (silica gel, cyclohexane/ethyl acetate 1/1)

¹H NMR (400 MHz, CDCl₃) δ 7.38 (d, J=7.3 Hz, 4H, Ph), 7.28 (t, J=7.2, 4H, Ph), 7.21 (t, J=7.2 Hz, 2H, Ph), 7.16-6.98 (m, 4H, Ph), 4.60 (tt, J=12.4, 3.7 Hz, 1H, CHCH₂CH₂N, 1H), 4.39 (m, 2H, CHCH₂CH₂N), 4.26 (dd, J=11.0, 7.7 Hz, 1H, OCH₂), 4.08 (dd, J=11.0, 5.7 Hz, 1H, OCH₂), 3.76 (d, J=13.9 Hz, 2H, NCH₂Ph), 3.70 (d, J=7.3 Hz, 2H, NCH₂C₆H₁₁), 3.57 (d, J=13.9 Hz, 2H, NCH₂Ph), 3.14 (seat., J=6.7 Hz, 1H, CHCH₃), 2.94 (m, 2H, CHCH₂CH₂N), 2.26 (m, 2H, CHCH₂CH₂N), 1.87 (m, 3H, CHCH₂CH₂N, C₆H₁₁), 1.67 (m, 3H, C₆H₁₁), 1.23 (m, 4H, C₆H₁₁), 1.09 (m, 6H, C₆H₁₁, CHCH₃)

¹³C NMR (100 MHz, CDCl₃) δ 155.2, 153.9 (C═O), 140.2, 130.0, 128.5, 128.1, 127.9, 126.8, 120.8, 109.0, 108.1 (C_{Ph}), 66.8 (CH₂O), 53.4 (NCH₂Ph), 51.9 (CHCH₃), 50.8 (CHCH₂CH₂N), 47.4 (NCH₂C₆H₁₁), 43.6 (CHCH₂CH₂N), 37.1 (NCH₂CH(CH₂)₅), 30.8 (NCH₂CH(CH₂)₅), 29.2 (CHCH₂CH₂N), 26.2, 25.7 (NCH₂CH(CH₂)₅), 11.2 (CHCH₃)

LC/MS (ES⁺) m/z 595.0 (M+H)⁺

### Preparation of (S)-2-(dibenzylamino)propyl 4-(3-(2-tert-butyloxycarbonylaminoethyl)-2-oxo-2,3-dihydro-1H-benzo[d]imidazol-1-yl)piperidine-1-carboxylate (Compound no 73)

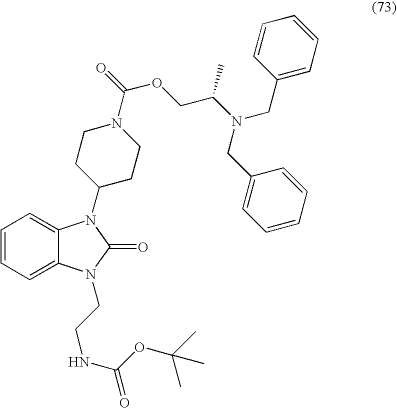

From (S)-2-(dibenzylamino)propyl 4-(1,2-dihydro-2-oxobenzo[d]imidazol-3-yl)piperidine-1-carboxylate and 2-tent-butyloxycarbonylamino-1-bromoethane.

Yellow oil (32%).

Flash chromatography on preparative TLC (silica gel, dichloromethane/methyl alcohol 95/5)

¹H NMR (400 MHz, CDCl₃) δ 7.38 (d, J=7.3 Hz, 4H, Ph), 7.34-7.24 (2d, J=7.2, 7.9 Hz, 4H, Ph), 7.20 (t, J=7.2 Hz, 2H, Ph), 7.07 (m, 4H, Ph), 4.92 (s, 1H, NH), 4.52 (tt, J=12.3, 3.7 Hz, 1H, CHCH₂CH₂N), 4.37 (m, 2H, CHCH₂CH₂N), 4.27 (dd, J=11.0, 7.4 Hz, 1H, CH₂O), 4.03 (m, 3H, CH₂O, NCH₂CH₂NH), 3.77 (d, J=13.9 Hz, 2H, NCH₂Ph), 3.57 (d, J=13.9 Hz, 2H, NCH₂Ph), 3.47 (dd, J=11.4, 5.6 Hz, 2H NCH₂CH₂NH), 3.15 (sext., J=6.8 Hz, 1H, CHCH₃), 2.94 (t_{b}, J=12.0 Hz, 2H, CHCH₂CH₂N), 2.34 (m, 2H, CHCH₂CH₂N), 1.87 (d, J=9.2 Hz, 2H, CHCH₂CH₂N), 1.42 (s, 9H, OtBu), 1.11 (d, J=6.8 Hz, 3H, CHCH₃)

¹³C NMR (100 MHz, CDCl₃) δ 155.9, 155.2, 153.8 (C═O), 140.2, 129.7, 128.5, 128.1, 128.0, 127.9, 127.8, 126.8, 121.2, 121.1, 109.1, 108.0 (C_{Ph}), 79.5 (OC_{q}(CH₃)₃), 66.8 (CH₂O), 53.7 (NCH₂Ph), 53.3 (NCH₂CH₂NH), 51.9 (CHCH₃), 50.9 (CHCH₂CH₂N), 43.6 (CHCH₂CH₂N), 40.8, 39.6 (NCH₂CH₂NH), 29.2 (CHCH₂CH₂N), 28.3 (OC_{q}(CH₃)₃), 11.3 (CHCH₃)

LC/MS (ES⁺) m/z 642.3 (M+H)⁺

### Preparation of (S)-2-(dibenzylamino)-3-phenylpropyl 4-(3-(2-tert-butyloxycarbonylaminoethyl)-2-oxo-2,3-dihydro-1H-benzo[d]imidazol-1-yl)piperidine-1-carboxylate (Compound no 74)

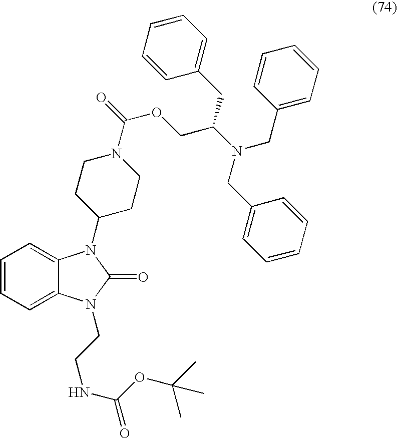

From (S)-2-(dibenzylamino)-3-phenylpropyl 4-(1,2-dihydro-2-oxobenzo[d]imidazol-3-yl)piperidine-1-carboxylate and 2-tert-butyloxycarbonylamino-1-bromoethane.

White solid (56%, 78%/recovered starting material).

Flash chromatography on silica gel (dichloromethane/methyl alcohol 98/2)

¹H NMR (400 MHz, CDCl₃) δ 7.33-7.18 (m, 13H, Ph), 7.16-6.99 (m, 6H, Ph), 4.96 (s, 1H, NH), 4.53 (tt, J=12.4, 3.7 Hz, 1H, CHCH₂CH₂N), 4.42 (m, 1H, CHCH₂CH₂N), 4.36 (dd, J=11.2, 7.1 Hz, 1H, OCH₂), 4.16 (dd_{b}, J=11.2, 4.7 Hz, 2H, OCH₂, CHCH₂CH₂N), 4.03 (t, J=5.2 Hz, 2H, NCH₂CH₂NH), 3.82 (d, J=13.8 Hz, 2H, NCH₂Ph), 3.74 (d, J=13.8 Hz, 2H, NCH₂Ph), 3.48 (dd, J=10.5, 5.2 Hz, 2H, NCH₂CH₂NH), 3.27 (m, 1H, CHCH₂Ph), 3.09 (dd, J=13.7, 5.8 Hz, 1H, CHCH₂Ph), 2.90 (m, 2H, CHCH₂CH₂N), 2.70 (dd, J=13.7, 8.5 Hz, 1H, CHCH₂Ph), 2.33 (m, 2H, CHCH₂CH₂N), 1.86 (d, J=11.8 Hz, 2H, CHCH₂CH₂N), 1.42 (s, 9H, OtBu)

¹³C NMR (100 MHz, CDCl₃) δ 155.9, 155.1, 153.7 (C═O), 139.8, 139.5, 129.8, 129.2, 128.6, 128.4, 128.3, 128.1, 128.0, 127.8, 126.9, 126.0, 121.2, 121.1, 109.2, 108.0 (C_{Ph}), 79.5 (OC_{q}(CH₃)₃), 64.7 (CH₂O), 58.4 (CHCH₂Ph), 53.9 (NCH₂Ph), 53.4 (NCH₂CH₂NH), 50.9 (CHCH₂CH₂N), 43.6 (CHCH₂CH₂N), 40.8, 39.6 (NCH₂CH₂NH). 34.1 (CHCH₂Ph), 29.3 (CHCH₂CH₂N), 28.3 (OC_{q}(CH₃)₃)

LC/MS (ES⁺) m/z 718.4 (M+H)⁺

### 16) N-aryl benzimidazolone preparation

### Preparation of (S)-2-(dibenzylamino)propyl 4-(1,2-dihydro-2-oxo-1-phenylbenzo[d]imidazol-3-yl)piperidine-1-carboxylate (Compound no 75)

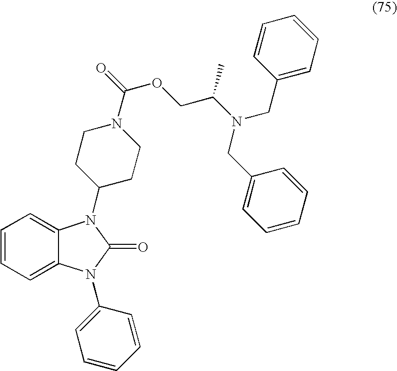

To 20 mg of 4 A° MS, 24 mg (0.2 mmol, 2 equiv.) of phenylboronic acid, 28 μL (0.2 mmol, 2 equiv.) of triethylamine in 500 μL of dry dichloromethane were successively added 50 mg (0.1 mmol, 1 equiv.) of (S)-2-(dibenzylamino)propyl 4-(1,2-dihydro-2-oxo-benzo[d]imidazol-3-yl)piperidine-1-carboxylate, 2 mg (0.01 mmol, 0.1 equiv.) of copper (II) sulfate and 17 mg (0.11 mmol. 1.1 equiv.) of 2,2,2′,2′-tetramethylpiperidine-N-oxide. The resulting mixture was stirred for 48 hours at room temperature on air. After filtration on celite, the solvant was evaporated on vacuum and the residue was purified on silica gel.

White solid (60%).

Flash chromatography on preparative TLC (silica gel, cyclohexane/ethyl acetate 7/3)

¹H NMR (400 MHz, CDCl₃) δ 7.53 (m, 4H, Ph), 7.41 (m, 5H, Ph), 7.29 (t, J=7.4 Hz, 4H, Ph), 7.21 (t, J=7.2 Hz, 3H, Ph), 7.07 (m, 3H, Ph), 4.62 (tt, J=12.4, 4.0 Hz, 1H, CHCH₂CH₂N, 1H), 4.43 (m, 2H, CHCH₂CH₂N), 4.28 (dd, J=10.9, 7.5 Hz, 1H, OCH₂), 4.08 (dd, J=10.9, 5.7 Hz, 1H, OCH₂), 3.77 (d, J=13.9 Hz, 2H, NCH₂Ph), 3.58 (d, J=13.9 Hz, 2H, NCH₂Ph), 3.15 (seat., J=6.6 Hz, 1H, CHCH₃), 2.97 (m, 2H, CHCH₂CH₂N), 2.43 (dq, J=12.5, 4.1 Hz, 2H, CHCH₂CH₂N), 1.95 (d, J=10.2 Hz, 2H, CHCH₂CH₂N), 1.12 (d, J=6.7 Hz, 3H, CHCH₃)

¹³C NMR (100 MHz, CDCl₃) δ 155.2, 152.7 (C═O), 140.2, 134.5, 129.6, 129.5, 128.5, 128.1, 127.7, 126.8, 126.1, 121.7, 121.2, 114.9, 109.2, 109.0 (C_{Ph}), 66.8 (CH₂O), 53.7 (NCH₂Ph), 51.9 (CHCH₃), 51.0 (CHCH₂CH₂N), 43.6 (CHCH₂CH₂N), 29.1 (CHCH₂CH₂N), 11.2 (CHCH₃)

LC/MS (ES⁺) m z 575.0 (M+H)⁺

### 17) Preparation of biotinylated derivative

### Preparation of (S)-2-(dibenzylamino)-3-phenylpropyl 4-(3-(2 aminoethyl)-2-oxo-2,3-dihydro-1H-benzo[d]imidazol-1-yl)piperidine-1-carboxylate D-biotin amid (Compound no 76)

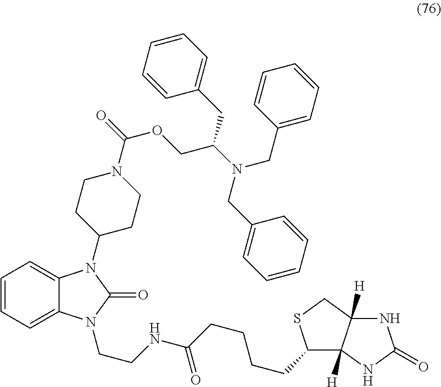

To a stirred solution of (S)-2-(dibenzylamino)-3-phenylpropyl 4-(3-(2-tert-butyloxycarbonylaminoethyl)-2-oxo-2,3-dihydro-1H-benzo[d]imidazol-1-yl)piperidine-1-carboxylate at 0° C. was treated with 5 ml of a solution of trifluoroacetic acid/dichloromethane (1/4) and stirred 1 h. The acid and solvent were evaporated in vacuum and amine hydrochloride was precipitated in dry diethyl ether and filtered.

To a stirred solution of 48 mg (0.195 mmol, 1.5 equiv.) of D-biotin, 64 mg (0.17 mmol, 1.3 equiv.) of HBTU, 36 μL (0.21 mmol, 1.6 equiv.) of DIEA in 2 ml of dry DMF was added the precedent crude amine salt in 2 ml of dry DMF. After 1 hour, the solvent was evaporated in vacuum at room temperature and the residue purified on silica gel.

White solid (95%).

Flash chromatography on preparative TLC (silica gel, dichloromethane/methyl alcohol 98/2)

¹H NMR (400 MHz, CDCl₃) δ 7.36-7.16 (m, 13H, Ph), 7.08 (m, 714, Ph, NH), 6.78 (s, 1H, NH), 5.84 (s, 1H, NH), 4.45 (m, 4H, CHCH₂CH₂N, CHCH₂CH₂N, OCH₂, NHCHCHNH), 4.25 (m, 1H, NHCHCHNH), 4.16 (m, 2H, OCH₂, CHCH₂CH₂N), 4.02 (m, 2H, NCH₂CH₂NH), 3.76 (2d, J=13.7 Hz, 4H, NCH₂Ph), 3.58 (m, 2H, NCH₂CH₂NH), 3.26 (m, 11-1, CHCH₂Ph), 3.08 (m, 2H, CHCH₂Ph, NHCHCH₂S), 2.89 (m, 3H, CHCH₂CH₂N, NHCHCH₂S), 2.67 (m, 2H, CHCH₂Ph, NHCHCHS), 2.32 (m, 2H, CHCH₂CH₂N), 2.15 (m, 2H₂CH₂CONH), 1.84 (d, J=11.4 Hz, 2H, CHCH₂CH₂N), 1.66 (m, 4H, SCH(CH₂)₃CH₂CO), 1.38 (m, 2H, SCH(CH₂)₃CH₂CO)

¹³C NMR (100 MHz, CDCl₃) δ 173.8 (NHC═O), 164.1 (NHC═ONH), 155.1, 154.0 (C═O), 139.8, 139.5, 129.4, 129.2, 128.6, 128.3, 128.2, 128.1, 127.8, 126.8, 126.7, 126.0, 121.4, 121.3, 109.2, 108.1 (C_{Ph}), 64.7 (CH₂O), 61.6, 60.1 (NHCHCHCHS), 58.4 (CHCH₂Ph), 55.7 (NHCHCHCHS), 53.9 (NCH₂Ph), 53.4 (NCH₂CH₂NH), 51.0 (CHCH₂CH₂N), 43.6 (CHCH₂CH₂N), 40.4 (NCH₂CH₂NH), 38.4 (NHCHCH₂S), 35.8 (CH₂CONH), 34.1 (CHCH₂Ph), 29.3 (CHCH₂CH₂N), 28.1, 28.0, 25.4 (SCH(CH₂)₃CH₂CO)

LC/MS (ES⁺) m/z 844.5 (M+H)⁺

### 18) Alkylation/Hydrolysis/Cyclisation

### Preparation of (S)-2-(dibenzylamino)propyl 4-(3-(4-ethoxy-4-oxobutyl)-2-oxo-2,3-dihydro-1H-benzo[d]imidazol-1-yl)piperidine-1-carboxylate (Compound no 77)

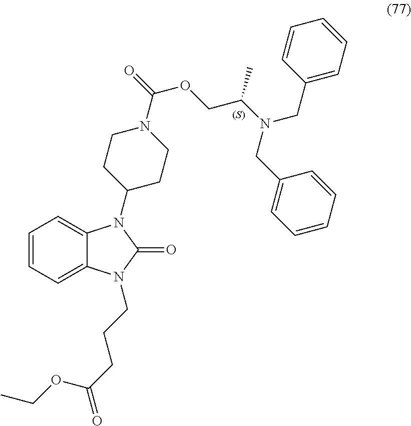

To a stirred solution of (S)-2-(dibenzylamino)propyl 4-(1,2-dihydro-2-oxobenzo[d]imidazol-3-yl)piperidine-1-carboxylate (350 mg, 0.70 mmol) in anhydrous DMF (3 mL) at 0° C. was added NaH in mineral oil (60%, 31 mg, 0.77 mmol) under N₂ atmosphere. The reaction mixture was stirred for 1 h at room temperature and cooled at 0° C. The ethyl 4-bromobutanoate (115 μL, 0.80 mmol) and potassium iodide (10 mg, 0.07 mmol) were added and the reaction mixture was stirred overnight at room temperature. Cool ice (5 mL) was added and the residue was filtered and dried on vacuo. The crude material was purified by flash chromatography (silica gel, cyclohexane/ethyl acetate 5/5) to afford a white solid (290 mg, 68%).

¹H NMR (400 MHz, CDCl₃) δ 7.38 (d, J=7.2 Hz, 4H, Bn), 7.28 (m, 4H, Bn), 7.20 (d, J=6.8 Hz, 2H, Bn), 7.08 (m, 4H, Ar), 4.53 (m, 1H, CHCH₂CH₂Nₚᵢₚ), 4.30 (m, 2H, CHCH₂CH₂Nₚᵢₚ), 4.26 (dd, J=10.8, 7.2 Hz, 1H, CH₂O), 4.12 (m, 3H, CH₂O, CH₃CH₂O), 3.94 (t, J=7.2 Hz, 2H, NCH₂CH₂CH₂), 3.76 (d, J=14.0 Hz, 2H, NCH₂Ph), 3.57 (d, J=14.0 Hz, 2H, NCH₂Ph), 3.14 (m, 1H, CHMe), 2.93 (m, 2H, CHCH₂CH₂Nₚᵢₚ), 2.40 (m, 4H, NCH₂CH₂CH₂, CHCH₂CH₂Nₚᵢₚ), 2.05 (t, J=7.2 Hz, 2H, NCH₂CH₂CH₂), 1.86 (m, 2H, CHCH₂CH₂Nₚᵢₚ), 1.25 (t, J=7.2 Hz, 3H, CH₃CH₂O), 1.11 (d, J=6.8 Hz, 3H, Me).

¹³C NMR (100 MHz, CDCl₃) δ 172.7 (C═O_{Ester}), 155.2 (C═O), 153.5 (C═O), 140.2 (C_{Bn}), 129.4 (C_{Ar}), 128.6 (CH_{Bn}), 128.2 (CH_{Bn}), 127.9 (C_{Ar}), 126.8 (CH_{Bn}), 121.0 (CH_{AD}), 120.9 (CH_{Ar}), 109.1 (CH_{Ar}), 107.9 (CHO, 66.9 (CH₂O), 60.4 (CH₃CH₂O), 53.7 (NCH₂Ph), 51.9 (CHMe), 50.9 (CHCH₂CH₂Nₚᵢₚ), 43.6 (CHCH₂CH₂Nₚᵢₚ), 40.3 (NCH₂CH₂CH₂), 31.2 (NCH₂CH₂CH₂), 29.3 (CHCH₂CH₂Nₚᵢₚ), 23.5 (NCH₂CH₂CH₂), 14.2 (CH₃CH₂O), 11.3 (Me).

LC/MS (ES⁺) m/z. 613.4 (M+H)⁺

### Preparation of (S)-4-(3-(14(2-(dibenzylamino)propoxy)carbonyl)piperidin-1-O-2-oxo-2,3-dihydro-1H-benzo[d]imidazol-1-yl)butanoic acid (Compound no 78)

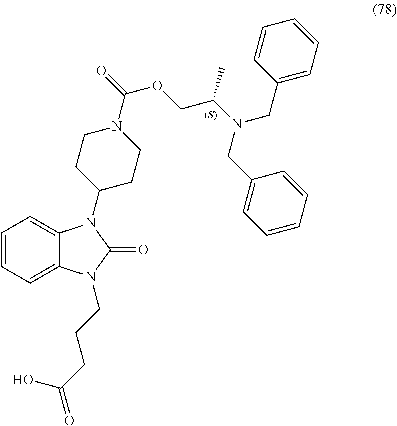

To a stirred solution of the (S)-2-(dibenzylamino)propyl 4-(3-(4-ethoxy-4-oxobutyl)-2-oxo-2,3-dihydro-1H-benzo[d]imidazol-1-yl)piperidine-1-carboxylate (280 mg, 0.46 mmol) in a solution MeOH/H₂O (1:1, 7 mL) and in THF (1 mL) was added NaOH (96 mg, 2.40 mmol). The reaction mixture was stirred for 5 h at room temperature. The solvent was removed on vacuo and HCl (6N) was added to pH 2. Saturated NaHCO₃ was added to pH 7 and the aqueous phase was extracted with ethyl acetate (3×10 mL). The combined organic layers were dried (Na₂SO₄), filtered and concentrated on vacuo to afford a white solid (180 mg, 67%).

¹H NMR (400 MHz, CDCl₃) δ 7.37 (d, J=7.2 Hz, 41-1, Bn), 7.28 (m. 4H, Bn), 7.21 (d, J=7.2 Hz, 2H, Bn), 7.11-7.06 (m, 4H, Ar), 4.54 (m, 1H, CHCH₂CH₂Nₚᵢₚ), 4.40 (m, 2H, CHCH₂CH₂Nₚᵢₚ), 4.27 (dd, J=10.8, 7.2 Hz, 1H, CH₂O), 4.07 (dd, J=10.8, 6.0 Hz, 1H, CH₂O), 3.99 (bt, 2H, NCH₂CH₂CH₂), 3.75 (d, J=14.0 Hz, 2H, NCH₂Ph), 3.58 (d, J=14.0 Hz, 2H, NCH₂Ph), 3.14 (m, 1H, CHMe), 2.94 (m, 2H, CHCH₂CH₂Nₚᵢₚ), 2.46 (bt, 2H, NCH₂CH₂CH₂), 2.25 (m, 2H, CHCH₂CH₂Nₚᵢₚ), 2.09 (bt, J=6.0 Hz, 2H, NCH₂CH₂CH₂), 1.87 (m, 2H, CHCH₂CH₂NOₚᵢₚ), 1.11 (d, J=6.8 Hz, 3H, Me).

¹³C NMR (100 MHz, CDCl₃) δ 176.8 (C═O_{Acide}), 155.3 (C═O), 153.7 (C═O), 140.2 (CH_{Bn}), 129.2 (C_{Ar}), 128.6 (CH_{Bn}), 128.2 (CH_{Bn}), 127.9 (C_{Ar}), 126.8 (CH_{Bn}), 121.3 (2×CH_{Ar}), 109.4 (CH_{A}), 108.0 (CH_{Ar}), 66.9 (CH₂O), 53.7 (NCH₂Ph), 51.9 (CHMe), 51.0 (CHCH₂CH₂NOₚᵢₚ), 43.6 (CHCH₂CH₂Nₚᵢₚ), 40.1 (NCH₂CH₂CH₂), 31.0 (NCH₂CH₂CH₂), 29.2 (CHCH₂CH₂Nₚᵢₚ), 23.5 (NCH₂CH₂CH₂), 11.2 (Me).

LC/MS (ES⁺) m/z 585.3 (M+H)⁺

### Preparation of (S)(1-(2-(dibenzylamino)propoxy)carbonyl)piperidin-4-yl)-5,6-dihydro-imidazo[4,5,1,j,k][1]benzazepine-2,7(1H,4H)-dione (Compound no 79)

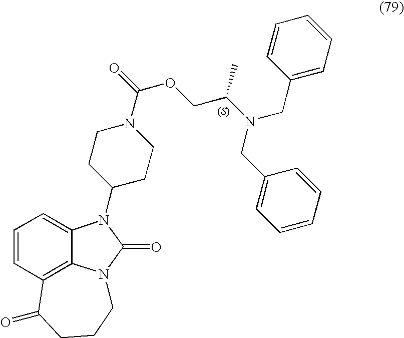

To SOCl₂ (0.2 mL) cooled at 0° C. was added (S)-4-(3-(1-((2-(dibenzylamino)propoxy)carbonyl)piperidin-4-yl)-2-oxo-2,3-dihydro-1H-benzo[d]imidazol-1-yl)butanoic acid (90 mg, 0.15 mmol). The reaction mixture was stirred for 1 h30 at room temperature and SOCl₂ was removed on vacuo. 1,2-dichloroethane (1.5 mL) was added, the mixture was cooled at 0° C. and AlCl₃ (104 mg, 0.78 mmol) was added. The reaction mixture was stirred for 3 h at room temperature. The solvent was removed on vacuo and the residue was purified by flash chromatography (silica gel, cyclohexane/ethyl acetate 3/7) to obtain a pale yellow solid (17 mg, 20%).

¹H NMR (400 MHz, CDCl₃) δ 7.80 (d, J=8.0 Hz, 1H, Ar), 7.38 (d, J=7.2 Hz, 4H, Bn). 7.28 (m, 5H, Ar), 7.21 (t, J=7.2 Hz, 2H, Bn), 7.09 (t, J=8.0 Hz, 1H, Ar), 4.52 (m, 1H, CHCH₂CH₂NOₚᵢₚ), 4.40 (m, 2H, CHCH₂CH₂Nₚᵢₚ). 4.25 (m, 1H, CH₂O). 4.13-4.06 (m, 3H, CH₂O, NCH₂CH₂CH₂), 3.75 (d, J=14.0 Hz, 2H, NCH₂Ph), 3.57 (d, J=14.0 Hz, 2H, NCH₂Ph), 3.14 (m, 1H, CHMe). 3.08 (m, 2H, NCH₂CH₂CH₂), 2.94 (m, 2H, CHCH₂CH₂Nₚᵢₚ), 2.38 (m, 2H, CHCH₂CH₂Nₚᵢₚ), 2.24 (m, 2H, NCH₂CH₂CH₂), 1.87 (m, 2H, CHCH₂CH₂Nₚᵢₚ), 1.11 (d. J=6.8 Hz, 3H, Me).

¹³C NMR (100 MHz, CDCl₃) δ 197.1 (C═O_{Cetone}), 155.2 (C═O), 153.0 (C═O), 140.2 (C_{Bn}), 129.2 (C_{Ar}), 129.1 (C_{Ar}), 128.6 (CH_{Bn}), 128.2 (CH_{Bn}), 126.8 (CH_{Bn}), 122.7 (CH_{Ar}), 120.5 (CH_{Ar}), 119.0 (C_{Ar}), 113.0 (CH_{Ar}), 66.9 (CH₂O), 53.7 (NCH₂Ph), 51.9 (CHMe), 51.4 (CHCH₂CH₂Nₚᵢₚ), 45.4 (NCH₂CH₂CH₂), 44.4 (NCH₂CH₂CH₂), 43.6 (CHCH₂CH₂Nₚᵢₚ), 29.0 (CHCH₂CH₂Nₚᵢₚ), 20.3 (NCH₂CH₂CH₂). 11.3 (Me).

LC/MS (ES⁺) in Z 567.3 (M+H)⁺

### 19) Aniline Derivatives Preparation (Reductive Amination)

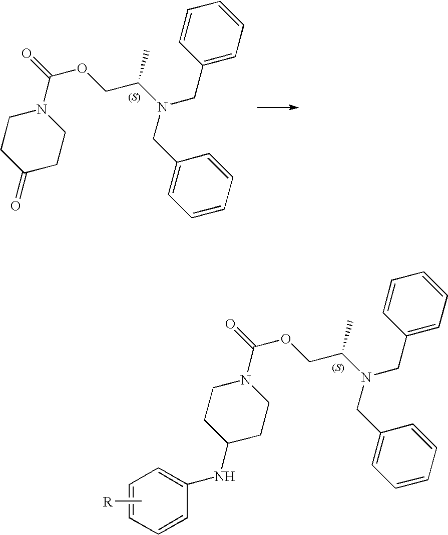

General Procedure:

To a stirred solution of (S)-2-(dibenzylamino)propyl 4-oxopiperidine-1-carboxylate (1 equiv.) in 1,2-dichloroethane (1 mL/mmol) were added appropriate aniline (1 equiv.), acetic acid (1.2 equiv.) followed by sodium triacetoxyborohydride (1.2 equiv.) under a nitrogen atmosphere. The reaction mixture was stirred for 16 h at room temperature. The solvent was removed on vacuo and the residue was partitioned between ethyl acetate and saturated aqueous Na₂CO₃ solution (5 mL/mmol). The organic phase was separated and washed by saturated aqueous Na₂CO₃ solution (2×5 mL/mmol). The organic layer was dried over anhydrous Na₂SO₄, filtered and concentrated under vacuum. The crude material was purified by flash chromatography on silica gel.

### Preparation of (S)-2-(dibenzylamino)propyl 4-(phenylamino)piperidine-1-carboxylate (Compound no 80)

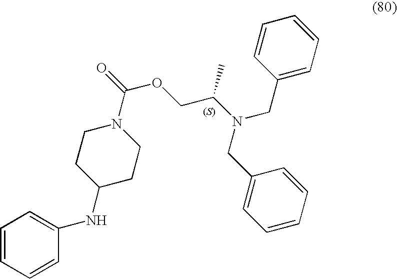

From aniline

Colorless oil (49%).

Flash chromatography on silica gel (pentane/acetone/triethylamine 95/4/1)

¹H NMR (400 MHz, CDCl₃) δ 7.37 (d. J=7.2 Hz, 4H, Bn), 7.26 (m, 4H, Bn), 7.20 (m, 4H, Ar), 6.72 (t, J=7.6 Hz, 1H, Ar), 6.62 (d, J=8.0 Hz, 2H, Ar), 4.23 (dd, J=10.8, 7.6 Hz, 1H, CH₂O), 4.11-4.03 (m, 3H, CHCH₂CH₂Nₚᵢₚ, CH₂O), 3.75 (d, J=14.0 Hz, 2H, NCH₂Ph), 3.55 (d, J=14.0 Hz, 2H, NCH₂Ph), 3.50 (m, 1H, CHCH₂CH₂Nₚᵢₚ), 3.12 (m, 1H, CHMe), 3.00 (bt, 2H, CHCH₂CH₂Nₚᵢₚ), 2.08 (bd, 2H, CHCH₂CH₂NOₚᵢₚ), 1.38 (m, 21-1, CHCH₂CH₂Nₚᵢₚ), 1.08 (d, J=6.8 Hz, 3H, Me).

¹³C NMR (100 MHz, CDCl₃) δ 155.3 (C═O), 146.7, 140.3, 129.4, 128.5, 128.1, 126.8, 117.5, 113.3, 66.7 (CH₂O), 53.7 (NCH₂Ph), 51.9 (CHMe), 49.9 (CHCH₂CH₂Nₚᵢₚ), 42.8 (CHCH₂CH₂Nₚᵢₚ), 32.4 (CHCH₂CH₂Nₚᵢₚ), 11.3 (Me).

LC/MS (ES⁺) m/z 458.3 (M+H)⁺

### Preparation of (S)-2-(dibenzylamino)propyl 4-(4-methoxyphenylamino)piperidine-1-carboxylate (Compound no 81)

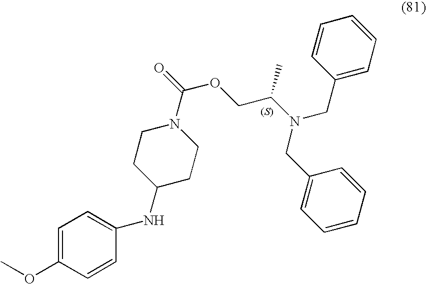

From 4-methoxybenzenamine

Colorless oil (49%).

Flash chromatography on silica gel (pentane/acetone/triethylamine 95/4/1)

¹H NMR (400 MHz, CDCl₃) δ 7.41 (d, J=7.2 Hz, 4H, Bn), 7.32 (t, J=7.2 Hz, 4H, Bn), 7.24 (t, J=7.2 Hz, 2H, Bn), 6.83 (d, J=9.0 Hz, 2H, Ar), 6.64 (d, J=9.0 Hz, 2H, Ar), 4.27 (dd, J=10.8, 7.2 Hz, 1H, CH₂O), 4.17-4.08 (m, 3H, CHCH₂CH₂Nₚᵢₚ, CH₂O), 3.78 (m, 5H, Ome, NCH₂Ph), 3.58 (d, J=14.0 Hz, 2H, NCH₂Ph), 3.40 (m, 1H, CHCH₂CH₂Nₚᵢₚ), 3.16 (m, 11-1, CHMe), 3.01 (bt, J=12.0 Hz, 2H, CHCH₂CH₂Nₚᵢₚ), 2.10 (bd, J=14.4 Hz, 2H, CHCH₂CH₇Nₚᵢₚ), 1.36 (m, 2H, CHCH₂CH₂Nₚᵢₚ), 1.11 (d, J=6.8 Hz, 3H, Me).

¹³C NMR (100 MHz, CDCl₃) δ 155.3 (C═O), 152.3, 140.8, 140.3, 128.5, 128.1, 126.8, 115.1, 115.0, 66.7 (CH₂O), 55.8 (Ome), 53.7 (NCH₂Ph), 51.9 (CHMe), 51.1 (CHCH₂CH₂Nₚᵢₚ), 42.8 (CHCH₂CH₂Nₚᵢₚ), 32.5 (CHCH₂CH₂Nₚᵢₚ), 11.3 (Me).

LC/MS (ES⁺) m/Z 488.3 (M+H)⁺

### Preparation of (S)-2-(dibenzylamino)propyl 4-(2,4-dimethoxyphenylamino)piperidine-1-carboxylate (Compound no 82)

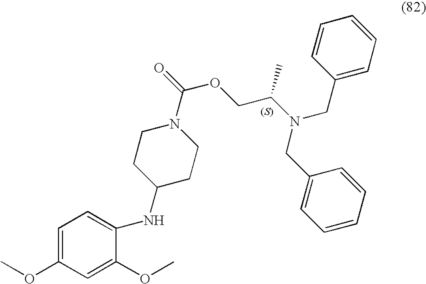

From 2,4-dimethoxybenzenamine

Colorless oil (49%).

Flash chromatography on silica gel (pentane/acetone/triethylamine 95/4/1)

¹H NMR (400 MHz, CDCl₃) δ 7.39 (d, J=7.2 Hz, 4H, Bn), 7.29 (m, 4H, Bn), 7.22 (m, 2H, Bn), 6.60 (d, J=7.8 Hz, 1H, Ar), 6.50 (s, 1H, Ar), 6.44 (d, J=7.8 Hz, 1H, Ar), 4.24 (m, 1H, CH₂O), 4.11-4.03 (m, 3H, CHCH₂CH₂Nₚᵢₚ, CH₂O), 3.84 (s, 3H, OMe), 3.78 (m, 5H, NCH₂Ph, OMe), 3.58 (d, J=14.0 Hz, 2H, NCH₂Ph), 3.41 (m, 1H, CHCH₂CH₂Nₚᵢₚ), 3.13 (m, 1H, CHMe), 3.00 (m, 2H, CHCH₂CH₂Nₚᵢₚ), 2.09 (m, 2H, CHCH₂CH₂Nₚᵢₚ), 1.38 (m, 2H, CHC′H₂CH₂Nₚᵢₚ), 1.10 (d, J=6.4 Hz, 3H, Me).

¹³C NMR (100 MHz, CDCl₃) δ 155.3 (C═O), 152.1, 148.3, 140.3, 130.6, 128.5, 128.1, 126.8, 111.4, 103.8, 99.3, 66.6 (CH₂O), 55.8 (OMe), 55.4 (OMe), 53.7 (NCH₂Ph), 51.8 (CHMe), 49.9 (CHCH₂CH₂Nₚᵢₚ), 42.8 (CHCH₂CH₂Nₚᵢₚ), 32.5 (CHCH₂CH₂Nₚᵢₚ), 11.3 (Me).

LC/MS (ES⁺) m/z 518.3 (M+H)⁺

### Preparation of (S)-2-(dibenzylamino)propyl 4-(3,4,5-trimethoxyphenylamino)piperidine-1-carboxylate (Compound no 83)

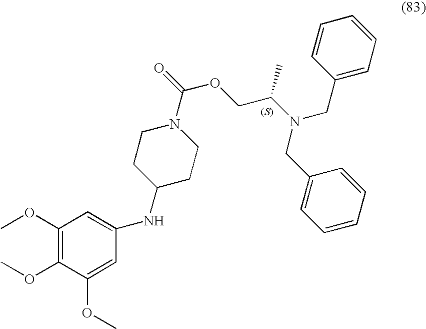

From 3,4,5-trimethoxybenzenamine

Brown oil (30%).

Flash chromatography on silica gel (pentane/acetone/triethylamine 95/4/1)

¹H NMR (400 MHz, CDCl₃) δ 7.38 (d, J=7.2 Hz, 4H, Bn), 7.29 (t, J=7.2 Hz, 4H, Bn), 7.22 (t, J=7.2 Hz, 2H, Bn), 5.87 (s, 2H, Ar), 4.22 (dd, J=10.8, 7.2 Hz, 1H, CH₂O), 4.14-4.03 (m, 3H, CHCH₂CH₂Nₚᵢₚ, CH₂O), 3.84 (s, 6H, OMe), 3.78 (m, 5H, NCH₂Ph, OMe), 3.57 (d, J=14.0 Hz, 2H, NCH₂Ph), 3.42 (m, 1H, CHCH₂CH₂Nₚᵢₚ), 3.13 (m, 1H, CHMe), 3.03 (m, 2H, CHCH₂CH₂Nₚᵢₚ), 2.05 (m, 2H, CHCH₂CH₂Nₚᵢₚ), 1.38 (m, 2H, CHCH₂CH₂Nₚᵢₚ), 1.09 (d, J=6.8 Hz, 3H, Me).

¹³C NMR (100 MHz, CDCl₃) δ 155.3 (C═O), 154.0, 143.4, 140.3, 130.3, 128.5, 128.1, 126.8, 91.2, 66.7 (CH₂O), 61.1 (OMe), 56.0 (OMe), 53.7 (NCH₂Ph), 51.9 (CHMe), 50.4 (CHCH₂CH₂Nₚᵢₚ). 42.7 (CHCH₂CH₂Nₚᵢₚ), 32.5 (CHCH₂CH₂Nₚᵢₚ), 11.2 (Me).

LC/MS (ES⁺) m/z 548.1 (M+H)⁺

### Preparation of (S)-2-(dibenzylamino)propyl 4-(3-fluorophenylamino)piperidine-1-carboxylate (Compound no 84)

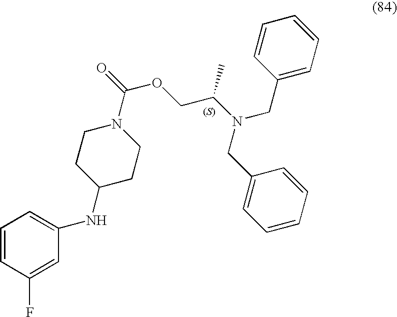

From 3-fluorobenzenamine

Brown oil (90%).

Flash chromatography on silica gel (pentane/acetone/triethylamine 95/4/1)

¹H NMR (400 MHz, CDCl₃) δ 7.38 (d. J=7.2 Hz, 4H, Bn), 7.29 (t, J=7.2 Hz, 4H, Bn), 7.22 (t, J=7.2 Hz, 2H, Bn), 7.10 (q, J=8.0 Hz, 1H, Ar), 6.38 (m, 2H. Ar), 6.62 (dd, J=11.6, 2.0 Hz, 1H, Ar), 4.23 (dd, J=11.2, 7.6 Hz, 1H, CH₂O), 4.14-4.03 (m, 3H, CHCH₂CH₂Nₚᵢₚ, CH₂O), 3.76 (d, J=14.0 Hz, 2H, NCH₂Ph), 3.57 (d, J=14.0 Hz, 2H, NCH₂Ph), 3.43 (m, 1H, CHCH₂CH₂NOₚᵢₚ), 3.13 (m, 1H, CHMe), 3.00 (bt, J=12.0 Hz, 2H, CHCH₂CH₂Nₚᵢₚ), 2.08 (m, 2H, CHCH₂CH₂Nₚᵢₚ), 1.35 (m. 2H, CHCH₂CH₂Nₚᵢₚ), 1.09 (d, J=6.8 Hz, 3H, Me).

¹³C NMR (100 MHz, CDCl₃) δ 164.1 (J_{C-F}=241.4 Hz), 155.3 (C═O), 148.4 (J_{C-F}=10.6 Hz), 140.2, 130.4 (J_{C-F}=10.2 Hz), 128.5, 128.1, 126.8, 109.0, 103.9 (J_{C-F}=21.4 Hz), 99.7 (J_{C-F}=25.2 Hz), 66.7 (CH₂O), 53.7 (NCH₂Ph), 51.9 (CHMe), 50.0 (CHCH₂CH₂Nₚᵢₚ), 42.7 (CHCH₂CH₂Nₚᵢₚ), 32.2 (CHCH₂CH₂Nₚᵢₚ), 11.2 (Me).

LC/MS (ES⁺) m/z 476.3 (M+H)⁺

### Preparation of (S)-2-(dibenzylamino)propyl 4-(4-iodophenylamino)piperidine-1-carboxylate (Compound no 85)

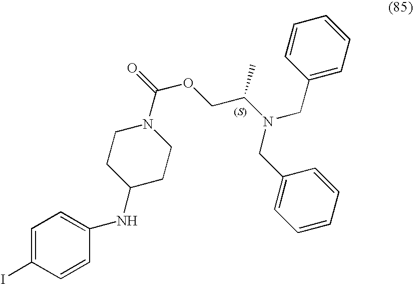

From 4-iodobenzenamine

Colorless oil (30%).

Flash chromatography on silica gel (pentane/acetone/triethylamine 95/4/1)

¹H NMR (400 MHz, CDCl₃) δ 7.42 (d, J=8.4 Hz, 2H, Ar), 7.36 (d, J=7.2 Hz, 4H, Bn), 7.28 (t, J=7.2 Hz Bn), 7.20 (t, J=7.2 Hz, 2H, Bn), 6.40 (d, J=8.4 Hz, 2H, Ar), 4.22 (dd, J=11.2, 7.6 Hz, 1H, CH₂O), 4.13-4.03 (m, 3H, CHCH₂CH₂Nₚᵢₚ, CH₂O), 3.74 (d, J=14.0 Hz, 2H, NCH₂Ph), 3.55 (d, J=14.0 Hz, 2H, NCH₂Ph), 3.41 (m, 1H, CHCH₂CH₂Nₚᵢₚ), 3.11 (m, 1H, CHMe), 2.99 (bt, J=11.2 Hz, 2H, CHCH₂CH₂Nₚᵢₚ), 2.05 (bd, J=11.2 Hz, 2H, CHCH₂CH₂Nₚᵢₚ), 1.38 (m, 2H, CHCH₂CH₂Nₚᵢₚ), 1.08 (d, J=6.8 Hz, 3H, Me).

LC/MS (ES⁺) m/z 584.2 (M+H)⁺

### Preparation of (S)-2-(dibenzylamino)propyl dichlorophenylamino)piperidine-1-carboxylate (Compound no 86)

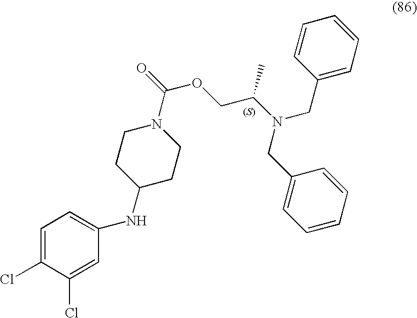

From 3,4-dichlorobenzenamine

Colorless oil (10%).

Flash chromatography on silica gel (pentane/acetone/triethylamine 95/4/1)

¹H NMR (400 MHz, CDCl₃) δ 7.37 (d, J=7.2 Hz, 4H, Bn), 7.29 (t, J=7.2 Hz, 4H, Bn), 7.20 (m, 3H, Ar), 6.67 (d, J=2.8 Hz, 1H, Ar), 6.43 (dd, J=8.8, 2.8 Hz, 1H, Ar), 4.23 (dd, J=10.8, 7.2 Hz, 1H, CH₂O), 4.13 (m, 2H, CHCH₂CH₂Nₚᵢₚ), 4.03 (dd, J=10.8, 5.6 Hz, 1N, CH₂O), 3.75 (d, J=13.8 Hz, 21-1, NCH₂Ph), 3.56 (d, J=13.8 Hz, 2H, NCH₂Ph), 3.39 (m, 1H, CHCH₂CH₂Nₚᵢₚ), 3.13 (m, 1H, CHMe), 3.00 (bt, J=11.6 Hz, 2H, CHCH₂CH₂Nₚᵢₚ), 2.05 (bd, J=12.4 Hz, 2H, CHCH₂CH₂Nₚᵢₚ), 1.35 (m, 2H, CHCH₂CH₂Nₚᵢₚ), 1.08 (d, J=6.8 Hz, 3H, Me).

¹³C NMR (100 MHz, CDCl₃) δ 155.3 (C═O), 146.1, 140.2, 132.9, 130.7, 128.5, 128.1, 126.8, 119.9, 114.0, 112.9, 66.7 (CH₂O), 53.7 (NCH₂Ph), 51.9 (CHMe), 50.0 (CHCH₂CH₂Nₚᵢₚ), 42.5 (CHCH₂CH₂Nₚᵢₚ), 32.0 (CHCH₂CH₂Nₚᵢₚ), 11.2 (Me).

LC/MS (ES⁺) m/z 526.2 (M+H)⁺

### Preparation of (S)-2-(dibenzylamino)propyl 4-(2-aminophenylamino)piperidine-1-carboxylate (Compound no 87)

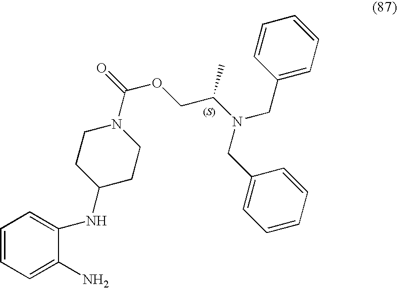

From benzene-1,2-diamine

Brown oil (65%).

Flash chromatography on silica gel (Cyclohexane/Ethyl acetate 1/1)

¹H NMR (400 MHz, CDCl₃) δ 7.37 (d, J=7.2 Hz, 4H, Bn), 7.26 (t, J=7.2 Hz, 4H, Bn), 7.20 (t, J=7.2 Hz, 2H, Bn), 6.72 (t, J=7.2 Hz, 1H, Ar), 6.62 (m, 3H, Ar), 4.23 (dd, J=10.8, 8.0 Hz, 1H, CH₂O), 4.12-4.03 (m, 3H, CHCH₂CH₂Nₚᵢₚ, CH₂O), 3.75 (d, J=13.8 Hz, 2H, NCH₂Ph), 3.56 (d, J=13.8 Hz, 2H, NCH₂Ph), 3.44 (m, 1H, CHCH₂CH₂Nₚᵢₚ), 3.13-3.03 (m, 3H, CHMe, CHCH₂CH₂Nₚᵢₚ), 2.06 (bd, 2H, CHCH₂CH₂Nₚᵢₚ), 1.40 (m, 2H, CHCH₂CH₂Nₚᵢₚ), 1.08 (d, J=6.8 Hz, 3H, Me).

¹³C NMR (100 MHz, CDCl₃) δ 155.4 (C═O), 140.3, 135.9, 134.8, 128.5, 128.1, 126.8, 120.6, 119.2, 117.1, 113.4, 66.6 (CH₂O), 53.7 (NCH₂Ph), 51.9 (CHMe), 50.0 (CHCH₂CH₂Nₚᵢₚ), 42.6 (CHCH₂CH₂Nₚᵢₚ), 32.4 (CHCH₂CH₂Nₚᵢₚ), 11.3 (Me).

LC/MS (ES⁺) m/z 473.3 (M+H)⁺

### Preparation of (S)-2-(dibenzylamino)propyl 4-(2-(phenylsulfonyl)phenylamino)piperidine-1-carboxylate (Compound no 164)

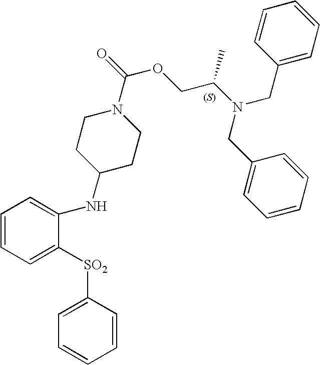

From 2-(phenylsulfonyl)benzenamine and (S)-2-(dibenzylamino)propyl 4-oxopiperidine-1-carboxylate

Colorless oil (6%).

Preparative HPLC.

¹H NMR (400 MHz, CDCl₃) δ 7.92 (dd, J=8.0, 1.2 Hz, 1H, Ar), 7.85 (d, J=7.2 Hz, 2H, Ar), 7.54 (t, J=7.6 Hz, 1H, Ar), 7.46 (t, J=7.2 Hz, 2H, Ar), 7.39 (d, J=7.6 Hz, 4H, Ar), 7.36-7.22 (m, 7H, Ar), 6.76 (t, J=7.2 Hz, 1H, Ar), 6.67 (d, J=8.4 Hz, 1H, Ar), 6.27 (d, J=6.8 Hz, 1H, NH), 4.25 (dd, J=11.2, 7.2 Hz, 1H, CH₂O), 4.07 (dd, J=11.2, 5.6 Hz, 1H, CH₂O), 3.90 (m, 2H, CHCH₂CH₂Nₚᵢₚ), 3.79 (d, J=13.8 Hz, 2H, NCH₂Ph), 3.62 (d, J=13.8 Hz, 2H, NCH₂Ph), 3.46 (m, 1H, CHCH₂CH₂Nₚᵢₚ), 3.12 (m, 3H, CHMe, CHCH₂CH₂Nₚᵢₚ), 1.95 (bd, 2H, CHCH₂CH₂Nₚᵢₚ), 1.44 (m, 2H, CHCH₂CH₂Nₚᵢₚ), 1.12 (d, J=6.8 Hz, 3H, Me).

LC/MS (ES⁺) m/z 598.3 (M+H)⁺

### Preparation of (S)-2-(dibenzylamino)-3-phenylpropyl 4-(phenylamino)piperidine-1-carboxylate (Compound no 152)

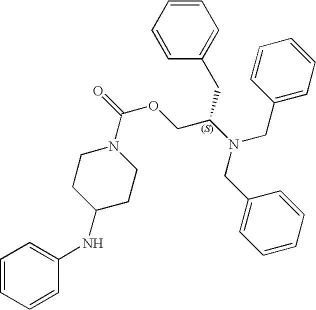

From aniline and (S)-2-(dibenzylamino)-3-phenylpropyl 4-oxopiperidine-1-carboxylate

Pale yellow oil (80%).

Flash chromatography on silica gel (pentane/acetone/triethylamine 95/4/1)

¹H NMR (400 MHz, CDCl₃) δ 7.29-7.17 (m, 15H, Ar), 7.07 (d, J=6.8 Hz, 2H, Ar), 6.72 (t, J=7.6 Hz, 1H, Ar), 6.62 (d, J=7.6 Hz, 2H, Ar), 4.31 (dd, J=11.2, 7.2 Hz, 1H, CH₂O), 4.13 (dd, J=11.2, 4.8 Hz, 1H, CH₂O), 4.05 (m, 2H, CHCH₂CH₂Nₚᵢₚ), 3.79 (d, J=13.8 Hz, 2H, NCH₂Ph), 3.72 (d, J=13.8 Hz, 2H, NCH₂Ph), 3.47 (m, 2H, CHCH₂CH₂Nₚᵢₚ), 3.24 (m, 1H, CHBn), 3.05 (dd, J=13.6, 6.0 Hz, 1H, CHCH₂Ph), 2.95 (m, 1H, CHCH₂CH₂Nₚᵢₚ), 2.68 (dd, J=13.6, 8.4 Hz, 1H, CHCH₂Ph), 2.08 (bd, 2H, CHCH₂CH₂Nₚᵢₚ), 1.35 (m, 2H, CHCH₂CH₂Nₚᵢₚ).

¹³C NMR (100 MHz, CDCl₃) δ 155.3 (C═O), 146.7, 139.9, 139.6, 129.4, 129.3, 128.6, 128.4, 128.2, 126.9, 126.1, 117.6, 113.3, 64.6 (CH₂O), 58.4 (CHCH₂Ph), 54.0 (NCH₂Ph), 49.9 (CHCH₂CH₂N), 42.8 (CHCH₂CH₂N), 34.1 (CHCH₂Ph), 32.4 (CHCH₂CH₂N).

LC/MS (ES⁺) m/z 534.2 (M+H)⁺

### 20) Aniline Derivatives Preparation

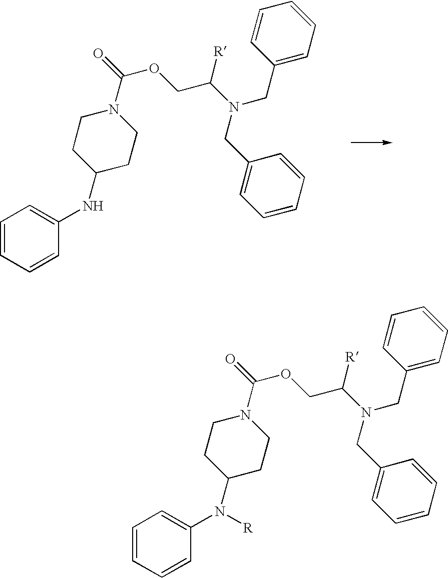

General Procedure:

To a stirred solution of the aniline derivative (1 equiv.) in CH₂Cl₂ (3 mL/mmol) was added NEt₃ (3 equiv.) and appropriate electrophile (2 equiv.) under N₂ atmosphere. The reaction mixture was stirred for 16 h at room temperature. The solvent was removed on vacuo and the residue was purified by flash chromatography on silica gel.

### Preparation of (S)-2-(dibenzylamino)propyl 4-(1,3-diphenylureido)piperidine-1-carboxylate (Compound no 88)

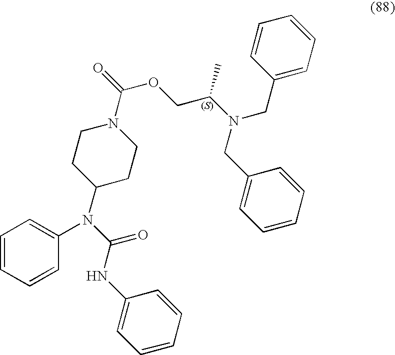

From phenylisocyanate

White solid (79%).

Flash chromatography on silica gel (cyclohexane/ethyl acetate 7/3)

¹H NMR (400 MHz, CDCl₃) δ 7.50 (m, 3H, Ar), 7.32 (d, J=7.2 Hz, 4H, Bn), 7.26-7.19 (m, 12H, Ar), 7.0 (m, 1H, Ar), 5.87 (s, 1H, NH), 4.74 (m, 1H, CHCH₂CH₂Nₚᵢₚ), 4.24-4.14 (m, 3H, CHCH₂CH₂Nₚᵢₚ, CH₂O), 3.95 (dd. J=11.2, 5.6 Hz, 1H, CH₂O), 3.69 (d, J=14.0 Hz, 2H, NCH₂Ph), 3.51 (d, J=14.0 Hz, 2H, NCH₂Ph), 3.05 (m, 1H, CHMe), 2.89 (m, 2H, CHCH₂CH₂Nₚᵢₚ), 1.91 (bd, 2H, CHCH₂CH₂Nₚᵢₚ), 1.27 (m, 2H, CHCH₂CH₂Nₚᵢₚ), 1.03 (d, J=6.8 Hz, 3H, Me).

¹³C NMR (100 MHz, CDCl₃) δ 155.1 (C═O), 154.0 (C═O), 140.2, 138.6, 137.0, 131.1, 130.2, 129.3, 128.8, 128.4, 128.1, 126.7, 123.0, 119.3, 66.5 (CH₂O), 53.6 (NCH₂Ph), 52.7 (CHMe), 51.8 (CHCH₂CH₂Nₚᵢₚ), 43.4 (CHCH₂CH₂Nₚᵢₚ), 31.1 (CHCH₂CH₂Nₚᵢₚ), 11.1 (Me).

LC/MS (ES⁺) M Z 577.3 (M+H)⁺

### Preparation of (S)-2-(dibenzylamino)propyl 4-(N-phenylbenzamido)piperidine-1-carboxylate (Compound no 89)

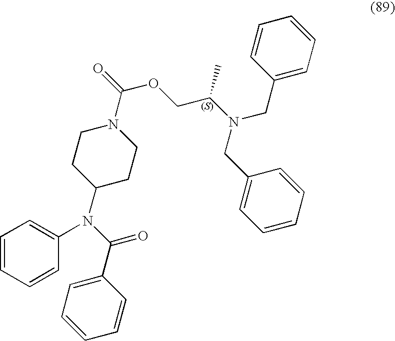

From benzoyl chloride

White solid (64%).

Flash chromatography on silica gel (cyclohexane/ethyl acetate 7/3)

¹H NMR (400 MHz, CDCl₃) δ 7.32 (d, J=7.2 Hz, 4H, Bn), 7.26-7.11 (m, 14H, Ar), 6.99 (d, J=6.4 Hz, 2H, Ar), 4.96 (m, 1H, CHCH₂CH₂Nₚᵢₚ), 4.29-4.11 (m, 3H, CHCH₂CH₂Nₚᵢₚ, CH₂O), 3.96 (dd, J=10.8, 5.6 Hz, 1H, CH₂O), 3.69 (d, J=14.0 Hz, 2H, NCH₂Ph), 3.52 (d, J=14.0 Hz, 2H, NCH₂Ph), 3.06 (m, 1H, CHMe), 2.94 (m, 2H, CHCH₂CH₂Nₚᵢₚ), 1.97 (bd, 2H, CHCH₂CH₂Nₚᵢₚ), 1.45 (m, 2H, CHCH₂CH₂Nₚᵢₚ), 1.03 (d, J=6.8 Hz, 3H, Me).

¹³C NMR (100 MHz, CDCl₃) δ 170.8 (C═O_{Amide}), 155.1 (C═O), 140.2, 139.2, 136.6, 130.6, 129.1, 128.8, 128.4, 128.1 (2C), 127.7, 127.6, 126.7, 66.5 (CH₂O), 53.6 (NCH₂Ph), 51.8 (CHMe, CHCH₂CH₂Nₚᵢₚ), 43.4 (CHCH₂CH₂Nₚᵢₚ), 30.6 (CHCH₂CH₂NOₚᵢₚ), 11.2 (Me).

LC/MS (ES⁺) m/z 562.3 (M+H)⁺

### Preparation of (S)-2-(dibenzylamino)propyl 4-(N-phenylphenylsulfonamido)piperidine-1-carboxylate (Compound no 90)

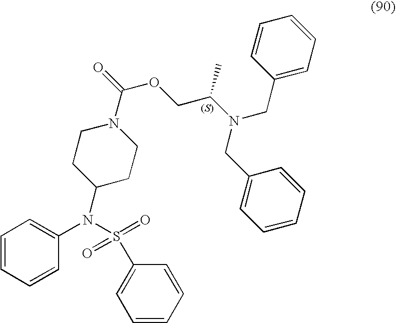

From benzenesulfonyl chloride

White solid (60%).

Flash chromatography on silica gel (cyclohexane/ethyl acetate 8/2)

¹H NMR (400 MHz, CDCl₃) δ 7.74 (d, J=7.2 Hz, 2H, Ar), 7.58 (t, J=7.2 Hz, 1H, Ar), 7.48 (t, J=8.0 Hz, 2H, Ar), 7.36 (m, 2H, Ar), 7.30 (m, 5H, Ar), 7.20 (m, 6H, Ar), 6.99 (d, J=7.2 Hz, 2H, Ar), 4.38 (m, 1H, CHCH₂CH₂Nₚᵢₚ), 4.17-4.09 (m, 3H, CHCH₂CH₂Nₚᵢₚ, CH₂O), 3.91 (dd, J=11.2, 5.6 Hz, 1H, CH₂O), 3.66 (d, J=14.0 Hz, 2H, NCH₂Ph), 3.49 (d, J=14.0 Hz, 2H, NCH₂Ph), 3.03 (m, 1H, CHMe), 2.83 (m, 2H, CHCH₂CH₂Nₚᵢₚ), 1.81 (bd, 2H, CHCH₂CH₂No), 1.29 (m, 2H, CHCH₂CH₂Nₚᵢₚ), 1.00 (d, J=6.8 Hz, 3H, Me).

¹³C NMR (100 MHz, CDCl₃) δ 154.9 (C═O), 141.1, 140.2, 134.9, 132.5, 132.4, 129.0, 128.9, 128.4, 128.0, 127.2 (2C), 126.7, 66.6 (CH₂O), 57.1 (CHCH₂CH₂Nₚᵢₚ), 53.6 (NCH₂Ph), 51.7 (CHMe), 43.3 (CHCH₂CH₂Nₚᵢₚ), 31.8 (CHCH₂CH₂NOₚᵢₚ), 11.1 (Me).

LC/MS (ES⁺) m/z 598.3 (M+H)⁺

### Preparation of (S)-2-(dibenzylamino)propyl 4-(N-phenyl-4-propylphenylsulfonamido)piperidine-1-carboxylate (Compound no 154)

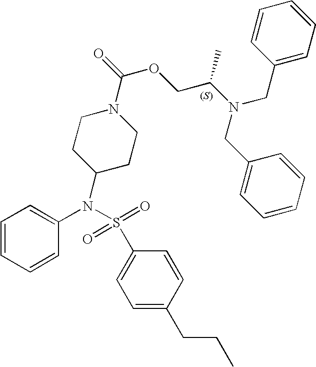

From 4-propylphenylsulfonyl chloride and (S)-2-(dibenzylamino)propyl 4-(phenylamino)piperidine-1-carboxylate.

White solid (35%).

Flash chromatography on silica gel (cyclohexane/ethyl acetate 7/3)

¹H NMR (400 MHz, CDCl₃) δ 7.64 (d, J=8.4 Hz, 2H, Ar), 7.36-7.28 (m, 9H, Ar), 7.19 (m, 6H, Ar), 6.99 (d, J=7.2 Hz, 2H, Ar), 4.38 (m, 1H, CHCH₂CH₂Nₚᵢₚ), 4.12-4.02 (m, 3H, CHCH₂CH₂Nₚᵢₚ, CH₂O), 3.91 (dd, J=11.2, 5.6 Hz, 1H, CH₂O), 3.66 (d, J=14.0 Hz, 2H, NCH₂Ph), 3.49 (d, J=14.0 Hz, 2H, NCH₂Ph), 3.02 (m, 1H, CHMe), 2.83 (m, 2H, CHCH₂CH₂Nₚᵢₚ), 2.66 (t, J=7.6 Hz, 2H, CH₂CH₂CH₃), 1.81 (bd, 2H, CHCH₂CH₂Nₚᵢₚ), 1.67 (m, 2H, CH₂CH₂CH₃), 1.29 (m, 2H, CHCH₂CH₂Nₚᵢₚ), 1.00 (d, J=6.8 Hz, 3H, Me), 0.94 (t, J=7.6 Hz, 3H, CH₂CH₂CH₃).

¹³C NMR (100 MHz, CDCl₃) δ 154.9 (C═O), 147.9, 140.2, 138.4, 135.0, 132.4, 129.0, 128.9, 128.4, 128.0, 127.3, 126.7, 66.5 (CH₂O), 57.0 (CHCH₂CH₂Nₚᵢₚ), 53.6 (NCH₂Ph), 51.7 (CHMe), 43.3 (CHCH₂CH₂Nₚᵢₚ), 37.8 (CH₂CH₂CH₃). 31.8 (CHCH₂CH₂Nₚᵢₚ), 24.1 (CH₂CH₂CH₃), 13.6 (CH₂CH₂CH₃), 11.1 (Me).

LC/MS (ES⁺) 171/Z 640.3 (M+H)⁺

### Preparation of (S)-2-(dibenzylamino)propyl 4-(N-phenyl-4-fluorophenylsulfonamido)piperidine-1-carboxylate (Compound no 155)

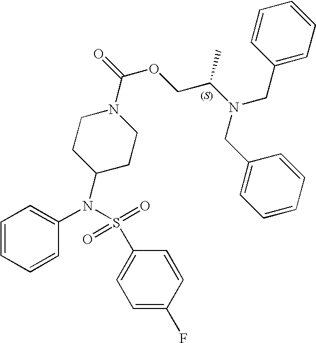

From 4-fluorophenylsulfonyl chloride and (S)-2-(dibenzylamino)propyl 4-(phenylamino)piperidine-1-carboxylate

White solid (43%).

Flash chromatography on silica gel (cyclohexane/ethyl acetate 7/3).

¹H NMR (400 MHz, CDCl₃) δ 7.75 (dd, J=7.2, 5.2 Hz, 2H, Ar), 7.38-7.29 (m, 7H, Ar), 7.22-7.13 (m, 8H, Ar), 6.99 (d, J=7.2 Hz, 2H, Ar), 4.38 (m, 1H, CHCH₇CH₂NOₚᵢₚ), 4.17-4.10 (m, 3H, CHCH₂CH₂Nₚᵢₚ, CH₂O), 3.91 (dd, J=11.2, 5.2 Hz, 1H, CH₂O), 3.66 (d, J=14.0 Hz, 2H, NCH₂Ph), 3.49 (d, J=14.0 Hz, 2H, NCH₂Ph), 3.02 (m, 1H, CHMe), 2.83 (m, 2H, CHCH₂CH₂Nₚᵢₚ), 1.81 (bd, 2H, CHCH₂CH₂Nₚᵢₚ), 1.29 (m, 2H, CHCH₂CH₂Nₚᵢₚ), 1.00 (d, J=6.8 Hz, 3H, Me).

¹³C NMR (100 MHz, CDCl₃) δ 164.2 (J_{C-F}=260.0 Hz, C_{Ar}). 154.9 (C═O), 140.2, 137.1, 134.7, 132.3, 129.9 (J_{C-F}=9.1 Hz, CH_{Ar}), 129.1, 128.4, 128.0, 126.7, 116.1 (J_{C-F}=22.5 Hz, CH_{Ar}), 114.9, 66.6 (CH₂O), 57.2 (CHCH₂CH₂Nₚᵢₚ), 53.6 (NCH₂Ph), 51.7 (CHMe), 43.3 (CHCH₂CH₂Nₚᵢₚ), 31.8 (CHCH₂CH₂Nₚᵢₚ). 11.1 (Me).

LC/MS (ES⁺) m/z 616.2 (M+H)⁺

### Preparation of (S)-2-(dibenzylamino)propyl 4-(N-phenyl-4-methoxyphenylsulfonamido)piperidine-1-carboxylate (Compound no 156)

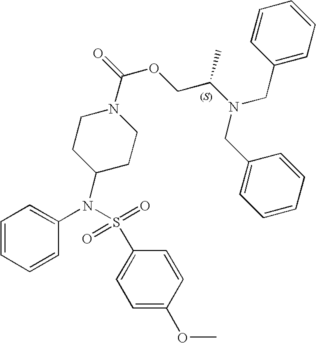

From 4-methoxyphenylsulfonyl chloride and (S)-2-(dibenzylamino)propyl 4-(phenylamino)piperidine-1-carboxylate

White solid (15%).

Flash chromatography on silica gel (cyclohexane/ethyl acetate 7/3)

¹H NMR (400 MHz, CDCl₃) δ 7.66 (d, J=9.2 Hz, 2H, Ar), 7.33-7.27 (m, 8H, Ar), 7.20 (m, 5H, Ar), 7.00 (d, J=7.2 Hz, 2H, Ar), 6.93 (d, J=8.8 Hz, 2H, Ar), 4.32 (m, 1H, CHCH₂CH₂Nₚᵢₚ), 4.13 (m, 3H, CHCH₂CH₂Nₚᵢₚ, CH₂O), 3.91 (m, 1H, CH₂O), 3.88 (s, 3H, OMe), 3.66 (d, J=14.0 Hz, 2H, NCH₂Ph), 3.49 (d, J=14.0 Hz, 2H, NCH₂Ph), 3.01 (m, 1H, CHMe), 2.82 (m, 2H, CHCH₂CH₂Nₚᵢₚ), 1.81 (bd, 2H, CHCH₂CH₂Nₚᵢₚ), 1.27 (m, 2H, CHCH₂CH₂Nₚᵢₚ), 1.00 (d, J=6.8 Hz, 3H, Me).

¹³C NMR (100 MHz, CDCl₃) δ 162.7, 154.9 (C═O), 140.2, 135.2, 132.8, 132.4, 129.4, 129.0, 128.9, 128.4, 128.0, 126.7, 114.0, 66.5 (CH₂O), 56.9 (OMe), 55.5 (CHCH₂CH₂Nₚᵢₚ), 53.6 (NCH₂Ph), 52.2 (CHMe), 43.4 (CHCH₂CH₂Nₚᵢₚ), 31.8 (CHCH₂CH₂Nₚᵢₚ), 11.0 (Me).

LC/MS (ES⁺) m/z 628.2 (M+H)⁺

### Preparation of (S)-2-(dibenzylamino)propyl 4-(N-phenyl-4-(trifluoromethyl)phenylsulfonamido)piperidine-1-carboxylate (Compound no 158)

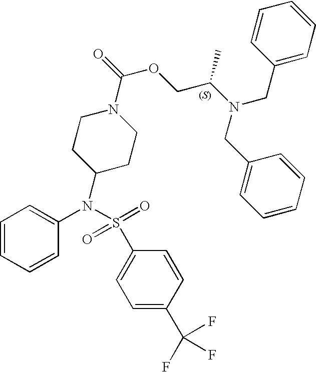

From 4-(trifluoromethyl)phenylsulfonyl chloride and (S)-2-(dibenzylamino)propyl 4-(phenylamino)piperidine-1-carboxylate

White solid (23%).

Flash chromatography on silica gel (cyclohexane/ethyl acetate 7/3)

¹H NMR (400 MHz, CDCl₃) δ 7.85 (d, J=8.4 Hz, 2H, Ar), 7.75 (d, J=8.0 Hz, 2H, Ar), 7.40-7.28 (m, 6H, Ar), 7.20 (m, 5H, Ar), 6.98 (d, J=7.6 Hz, 2H, Ar), 4.40 (m, 1H, CHCH₂CH₂Nₚᵢₚ), 4.17-4.09 (m, 3H, CHCH₂CH₂Nₚᵢₚ, CH₂O), 3.91 (dd, J=10.4, 5.2 Hz, 1H, CH₂O), 3.66 (d, J=14.0 Hz, 2H, NCH₂Ph), 3.49 (d, J=14.0 Hz, 2H, NCH₂Ph), 3.02 (m, 1H, CHMe), 2.84 (m, 2H, CHCH₂CH₂Nₚᵢₚ), 1.81 (bd, 2H, CHCH₂CH₂Nₚᵢₚ), 1.29 (m, 2H, CHCH₂CH₂Nₚᵢₚ), 1.00 (d, J=6.8 Hz, 3H, Me).

LC/MS (ES⁺) m/z 666.2 (M+H)⁺

### Preparation of (S)-2-(dibenzylamino)propyl 4-(N-phenylthiophen-2-ylsulfonamido)piperidine-1-carboxylate (Compound no 159)

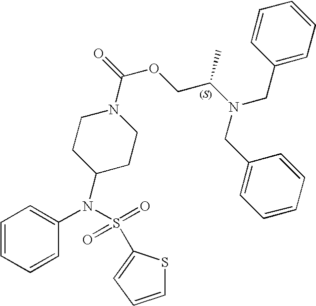

From thiophene-2-sulfonyl chloride and (S)-2-(dibenzylamino)propyl 4-(phenylamino)piperidine-1-carboxy late.

White solid (37%).

Flash chromatography on silica gel (cyclohexane/ethyl acetate 8/2)

¹H NMR (400 MHz, CDCl₃) δ 7.60 (dd, J=4.8, 1.2 Hz, 1H, Ar_{Thio}), 7.46 (dd, J=4.0, 1.2 Hz, 1H, Ar_{Thio}), 7.37 (m, 3H, Ar), 7.29 (m, 4H, Ar), 7.22-7.16 (m, 6H, Ar), 7.06 (m, 3H, Ar), 4.38 (m, 1H, CHCH₂CH₂Nₚᵢₚ), 4.19-4.10 (m, 3H, CHCH₂CH₂Nₚᵢₚ, CH₂O), 3.92 (dd, J=11.2, 5.6 Hz, 1H, CH₂O), 3.66 (d, J=14.0 Hz, 2H, NCH₂Ph), 3.49 (d, J=14.0 Hz, 2H, NCH₂Ph), 3.02 (m, 1H, CHMe), 2.83 (m, 2H, CHCH₂CH₂Nₚᵢₚ), 1.87 (bd, 2H, CHCH₂CH₂Nₚᵢₚ), 1.29 (m, 2H, CHCH₂CH₂Nₚᵢₚ), 1.00 (d, J=6.8 Hz, 3H, Me).

¹³C NMR (100 MHz, CDCl₃) δ 154.9 (C═O), 141.9, 140.2, 134.9, 132.6, 132.1, 131.8, 129.2, 129.1, 128.4, 128.1, 127.2, 126.7, 66.4 (CH₂O), 57.8 (CHCH₂CH₂Nₚᵢₚ), 53.7 (NCH₂Ph), 51.9 (CHMe), 43.3 (CHCH₂CH₂Nₚᵢₚ), 31.8 (CHCH₂CH₂Nₚᵢₚ), 11.0 (Me).

LC/MS (ES⁺) m/z 604.2 (M+H)⁺

### Preparation of (S)-2-(dibenzylamino)propyl 4-(N-phenylquinolin-8-ylsulfonamido)piperidine-1-carboxylate (Compound no 160)

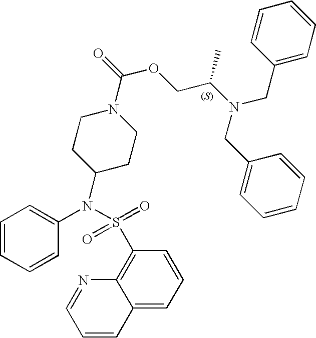

From quinoline-8-sulfonyl chloride and (S)-2-(dibenzylamino)propyl 4-(phenylamino)piperidine-1-carboxylate.

Colorless oil (38%).

Flash chromatography on silica gel (cyclohexane/ethyl acetate 8/2)

¹H NMR (400 MHz, CDCl₃) δ 9.12 (dd, J=4.0, 1.6 Hz, 1H, Quino.), 8.32 (dd, J=8.4, 1.6 Hz, 1H, Quino.), 8.22 (dd, J=7.2, 0.8 Hz, 1H, Quino.), 8.05 (dd, J=7.2, 1.6 Hz, 1H, Quino.), 7.61 (dd, J=8.0, 4.0 Hz, 1H, Quino.), 7.52 (t, J=8.0 Hz, 1H, Ar), 7.31 (d, J=7.2 Hz, 4H, Ar), 7.24 (m, 9H, Ar), 6.77 (d, J=7.6 Hz, 2H, Ar), 4.21-4.10 (m, 4H, CHCH₂CH₂Nₚᵢₚ, CHCH₂CH₂Nₚᵢₚ, CH₂O), 3.93 (dd, J=10.8, 5.6 Hz, 1H, CH₂O), 3.68 (d, J=14.0 Hz, 2H, NCH₂Ph), 3.51 (d, J=14.0 Hz, 2H, NCH₂Ph), 3.03 (m, 3H, CHMe, CHCH₂CH₂Nₚᵢₚ), 2.00 (m, 2H, CHCH₂CH₂Nₚᵢₚ), 1.27 (m, 2H, CHCH₂CH₂Nₚᵢₚ), 1.02 (d, J=6.8 Hz, 31-1, Me).

¹³C NMR (100 MHz, CDCl₃) δ 154.9 (C═O), 150.9, 153.8, 140.2, 138.0, 136.8, 135.1, 133.3, 133.2, 132.2, 129.0, 128.9, 128.7, 128.4, 128.0, 126.9, 125.8, 122.1, 66.2 (CH₂O), 56.9 (CHCH₂CH₂Nₚᵢₚ), 53.9 (NCH₂Ph), 51.9 (CHMe), 43.8 (CHCH₂CH₂Nₚᵢₚ), 32.1 (CHCH₂CH₂Nₚᵢₚ), 11.1 (Me).

LC/MS (ES⁺) m/z 649.2 (M+H)⁺

### Preparation of (S)-2-(dibenzylamino)propyl 4-(N-phenyl-naphthalen-1-ylsulfonamido)piperidine-1-carboxylate (Compound no 161)

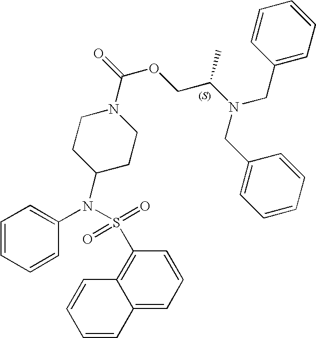

From naphthalene-1-sulfonyl chloride and (S)-2-(dibenzylamino)propyl 4-(phenylamino)piperidine-1-carboxylate

Colorless oil (37%).

Flash chromatography on silica gel (cyclohexane/ethyl acetate 8/2)

¹H NMR (400 MHz, CDCl₃) δ 8.63 (d, J=8.4 Hz, 1H, Napht.), 8.15 (dd, J=9.6, 7.6 Hz, 2H, Napht.), 7.94 (d, J=8.4 Hz, 1H, Ar), 7.63 (m, 2H, Napht.), 7.43 (t, J=8.0 Hz, 1H, Napht.), 7.31 (m, 6H, Ar), 7.21 (m, 7H, Ar), 6.91 (d, J=7.6 Hz, 2H, Ar), 4.49 (m, 1H, CHCH₂CH₂Nₚᵢₚ), 4.14 (m, 3H, CHCH₂CH₂Nₚᵢₚ, CH₂O), 3.95 (dd, J=10.8, 5.2 Hz, 1H, CH₂O), 3.77 (d, J=13.4 Hz, 2H, NCH₂Ph), 3.61 (d, J=13.4 Hz, 2H, NCH₂Ph), 3.11 (m, 1H, CHMe), 2.83 (m, 2H, CHCH₂CH₂Nₚᵢₚ), 1.85 (bd, 2H, CHCH₂CH₂Nₚᵢₚ), 1.27 (m, 2H, CHCH₂CH₂Nₚᵢₚ), 1.05 (d, J=6.8 Hz, 3H, Me).

¹³C NMR (100 MHz, CDCl₃) δ 154.9 (C═O), 135.2, 135.1, 135.0, 134.9, 132.0, 131.1, 129.1, 129.0, 128.9, 128.8, 128.7, 128.3, 128.2, 127.9, 127.1, 126.8, 124.9, 123.9, 66.2 (CH₂O), 56.3 (CHCH₂CH₂Nₚᵢₚ), 53.8 (NCH₂Ph), 51.9 (CHMe), 43.4 (CHCH₂CH—Nₚᵢₚ), 31.9 (CHCH₂CH₂Nₚᵢₚ), 11.1 (Me).

LC/MS (ES⁺) m/z 648.2 (M+H)⁺

### Preparation of (S)-2-(dibenzylamino)propyl 4-(N-phenyl-2-methylphenylsulfonamido)piperidine-1-carboxylate (Compound no 167)

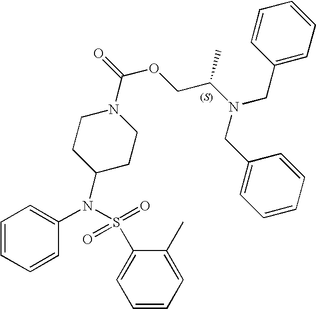

From 2-methylphenylsulfonyl chloride and (S)-2-(dibenzylamino)propyl 4-(phenylamino)piperidine-1-carboxylate.

White solid (9%).

Flash chromatography on silica gel (cyclohexane/ethyl acetate 9/1)

¹H NMR (400 MHz, CDCl₃) δ 7.74 (d, J=8.0 Hz, 1H, Ar), 7.40 (td, J=7.6, 0.8 Hz, 1H, Ar), 7.32-7.16 (m, 15H, Ar), 7.05 (d, J=7.2 Hz, 2H, Ar), 4.39 (m, 1H, CHCH₂CH₂Nₚᵢₚ), 4.25-4.09 (m. 3H, CHCH₂CH₂Nₚᵢₚ, CH₂O), 3.91 (dd, J=11.2, 5.6 Hz, 1H, CH₂O), 3.67 (d, J=14.0 Hz, 2H, NCH₂Ph), 3.50 (d, J=14.0 Hz, 2H, NCH₂Ph), 3.03 (m, 1H, CHMe), 2.84 (m, 2H, CHCH₂CH₂Nₚᵢₚ), 2.58 (s, 3H, Me), 1.92 (bd, 2H, CHCH₂CH₂Nₚᵢₚ), 1.32 (m, 2H, CHCH₂CH₂Nₚᵢₚ), 1.00 (d, J=6.8 Hz, 3H, Me).

LC/MS (ES⁺) m/z 612.3 (M+H)⁺

### Preparation of (S)-2-(dibenzylamino)propyl 4-(N-phenyl-3-methylphenylsulfonamido)piperidine-1-carboxylate (Compound no 168)

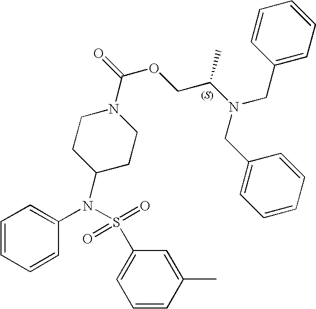

From 3-methylphenylsulfonyl chloride and (S)-2-(dibenzylamino)propyl 4-(phenylamino)piperidine-1-carboxylate.

White solid (50%).

Flash chromatography on silica gel (cyclohexane/ethyl acetate 9/1)

¹H NMR (400 MHz, CDCl₃) δ 7.54 (m, 2H, Ar), 7.38-7.27 (m, 9H, Ar), 7.22-7.17 (m, 6H, Ar), 7.00 (d, J=7.6 Hz, 2H, Ar), 4.37 (m, 1H, CHCH₂CH₂Nₚᵢₚ), 4.20-4.05 (m, 3H, CHCH₂CH₂Nₚᵢₚ, CH₂O), 3.91 (dd, J=11.2, 5.6 Hz, 1H, CH₂O), 3.66 (d, J=14.0 Hz, 2H, NCH₂Ph), 3.49 (d, J=14.0 Hz, 2H, NCH₂Ph), 3.02 (m, 1H, CHMe), 2.83 (m, 2H, CHCH₁CH₂Nₚᵢₚ), 2.40 (s, 3H, Me), 1.82 (bd, 2H, CHCH₂CH₂Nₚᵢₚ), 1.28 (m, 2H, CHCH₂CH₂Nₚᵢₚ), 1.00 (d, J=6.8 Hz, 3H, Me).).

¹³C NMR (100 MHz, CDCl₃) δ 154.9 (C═O), 140.9, 140.2, 139.0, 134.9, 133.3, 132.4, 129.0, 128.8, 128.4, 128.0, 127.7, 126.7, 124.4, 66.6 (CH₂O), 57.1 (CHCH₂CH₂Nₚᵢₚ), 53.6 (NCH₂Ph), 51.7 (CHMe), 43.3 (CHCH₂CH₂Nₚᵢₚ), 31.8 (CHCH₂CH₂Nₚᵢₚ), 21.3 (Me), 11.1 (Me).

LC/MS (ES⁺) m/z 612.4 (M+H)⁺

### Preparation of (S)-2-(dibenzylamino)propyl 4-(N-phenyl-3-(trifluoromethyl)phenylsulfonamido)piperidine-1-carboxylate (Compound no 169)

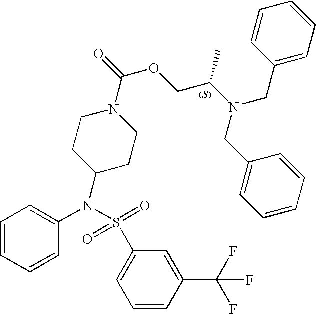

From 3-(trifluoromethyl)phenylsulfonyl chloride and (S)-2-(dibenzylamino)propyl 4-(phenylamino)piperidine-1-carboxylate.

White solid (49%).

Flash chromatography on silica gel (cyclohexane/ethyl acetate 9/1)

¹H NMR (400 MHz, CDCl₃) δ 7.96 (s, 1H, Ar), 7.91 (d, J=8.0 Hz, 1H, Ar), 7.83 (d, J=8.0 Hz, 1H, Ar), 7.63 (t, J=8.0 Hz, 1H, Ar), 7.36-7.26 (m, 7H, Ar), 7.20 (m, 6H, Ar), 6.96 (d, J=7.6 Hz, 21-1, Ar), 4.40 (m, 1H, CHCH₂CH₂Nₚᵢₚ), 4.17-4.09 (m, 3H, CHCH₂CH₂Nₚᵢₚ, CH₂O), 3.81 (m, 1H, CH₂O), 3.66 (d, J=14.0 Hz, 2H, NCH₂Ph), 3.49 (d, J=14.0 Hz, 2H, NCH₂Ph), 3.02 (m, 1H, CHMe), 2.84 (m, 2H, CHCH₂CH₂Nₚᵢₚ), 1.80 (bd, 2H, CHCH₂CH₂Nₚᵢₚ), 1.28 (m, 2H, CHCH₂CH₂Nₚᵢₚ), 1.00 (d, J=6.8 Hz, 3H, Me).

¹³C NMR (100 MHz, CDCl₃) δ 154.9 (C═O), 142.2, 140.2, 134.3, 132.2, 130.3, 129.7, 129.4, 129.2, 129.1, 128.4, 128.0, 126.7, 124.5, 124.4, 66.6 (CH₂O), 57.4 (CHCH₂CH₂Nₚᵢₚ), 53.6 (NCH₂Ph), 51.7 (CHMe), 43.2 (CHCH₂CH₂Nₚᵢₚ), 31.8 (CHCH₂CH₂Nₚᵢₚ), 11.0 (Me).

LC/MS (ES⁺) m/z 666.3 (M+H)⁺

### Preparation of (S)-2-(dibenzylamino)propyl 4-(N-phenyl-biphenyl-4-sulfonamido)piperidine-1-carboxylate (Compound no 170)

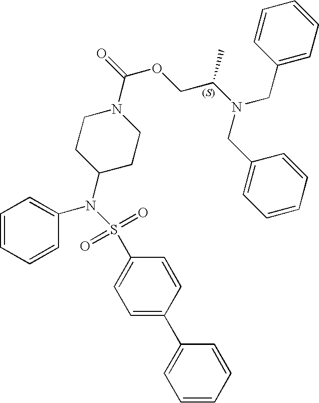

From biphenyl-4-sulfonyl chloride and (S)-2-(dibenzylamino)propyl 4-(phenylamino)piperidine-1-carboxylate.

White solid (62%),

Flash chromatography on silica gel (cyclohexane/ethyl acetate 9/1)

¹H NMR (400 MHz, CDCl₃) δ 7.80 (d, J=8.4 Hz, 2H, Ar), 7.70 (d, J=8.4 Hz, 2H, Ar), 7.64 (d, J=8.4 Hz, 2H, Ar), 7.49 (t, J=7.2 Hz, 2H, Ar), 7.43 (d, J=7.2 Hz, 1H, Ar), 7.39-7.29 (m, 7H, Ar), 7.21 (m, 6H, Ar), 7.05 (d, J=7.2 Hz, 2H, Ar), 4.41 (m, 1H, CHCH₂CH₂Nₚᵢₚ), 4.14 (m, 3H, CHCH₂CH₂Nₚᵢₚ, CH₂O), 3.92 (dd, J=11.2; 5.6 Hz, 1H, CH₂O), 3.67 (d, J=13.6 Hz, 2H, NCH₂Ph), 3.49 (d, J=13.6 Hz, 2H, NCH₂Ph), 3.04 (m, 1H, CHMe), 2.83 (m, 2H, CHCH₂CH₂Nₚᵢₚ), 1.85 (bd, 2H, CHCH₂CH₂Nₚᵢₚ), 1.29 (m, 2H, CHCH₂CH₂Nₚᵢₚ), 1.00 (d, J=6.8 Hz, 3H, Me).

¹³C NMR (100 MHz, CDCl₃) δ 154.9 (C═O), 145.3, 140.2, 139.7, 139.1, 134.9, 132.4, 129.1, 129.0, 128.5, 128.4, 128.0, 127.8, 127.5, 127.3, 126.7, 66.6 (CH₂O), 57.2 (CHCH₂CH₂Nₚᵢₚ), 53.6 (NCH₂Ph), 51.8 (CHMe), 43.3 (CHCH₂CH₂Nₚᵢₚ), 31.8 (CHCH₂CH₂Nₚᵢₚ), 11.1 (Me).

LC/MS (ES⁺) m/z 674.3 (M+H)⁺

### Preparation of (S)-2-(dibenzylamino)propyl 4-(N-phenyl-naphthalen-2-ylsulfonamido)piperidine-1-carboxylate (Compound no 171)

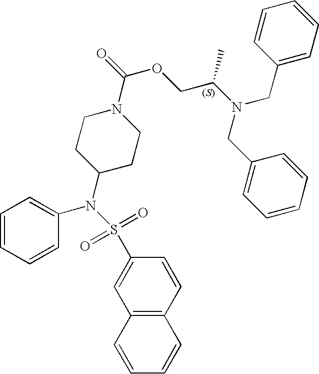

From naphthalene-2-sulfonyl chloride and (S)-2-(dibenzylamino)propyl 4-(phenylamino)piperidine-1-carboxylate.

White solid (36%).

Flash chromatography on silica gel (cyclohexane/ethyl acetate 9/1)

¹H NMR (400 MHz, CDCl₃) δ 8.26 (s, 1H, Napht.), 7.96 (d, J=8.8 Hz, 1H, Napht.), 7.92 (t, J=8.4 Hz, 2H, Ar), 7.76 (dd, J=8.8, 1.6 Hz, 1H, Napht.), 7.63 (m, 2H, Napht.), 7.36-7.26 (m, 7H, Ar), 7.20 (m, 61-1, Ar), 7.01 (d, J=7.2 Hz, 2H, Ar), 4.44 (m, 1H, CHCH₂CH₂NOₚᵢₚ), 4.15-4.09 (m, 3H, CHCH₂CH₂NOₚᵢₚ, CH₂O), 3.90 (dd, J=10.8, 5.6 Hz, 1H, CH₂O), 3.65 (d, J=13.6 Hz, 2H, NCH₂Ph), 3.48 (d, J=13.6 Hz, 2H, NCH₂Ph), 3.01 (m, 1H, CHMe), 2.84 (m, 2H, CHCH₂CH₂Nₚᵢₚ), 1.84 (bd, 2H, CHCH₂CH₂Nₚᵢₚ), 1.27 (m, 2H, CHCH₂CH₂Nₚᵢₚ), 0.99 (d, J=6.8 Hz, 3H, Me).

¹³C NMR (100 MHz, CDCl₃) δ 154.9 (C═O), 140.2, 137.9, 134.9, 134.7, 132.4, 132.0, 129.2, 129.0, 128.8, 128.6, 128.4, 128.0, 127.8, 127.5, 126.7, 122.5, 66.6 (CH₂O), 57.2 (CHCH₂CH₂Nₚᵢₚ), 53.6 (NCH₂Ph), 51.7 (CHMe), 43.3 (CHCH₂CH₂Nₚᵢₚ), 31.8 (CHCH₂CH₂Nₚᵢₚ), 11.1 (Me).

LC/MS (ES⁺) m/z 648.3 (M+H)⁺

### Preparation of (S)-2-(dibenzylamino)propyl 4-(N-phenyl-(5-dimethylamino)naphthalen-1-ylsulfonamido)piperidine-1-carboxylate (Compound no 172)

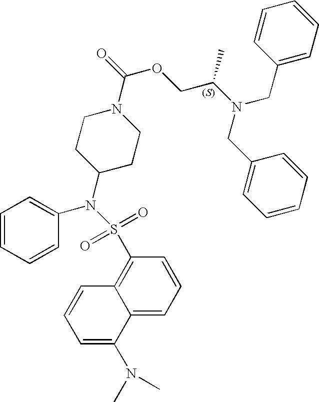

From 5-(dimethylamino)naphthalene-1-sulfonyl chloride and (S)-2-(dibenzylamino)propyl 4-(phenylamino)piperidine-1-carboxylate.

Pale green solid (33%).

Flash chromatography on silica gel (cyclohexane/ethyl acetate 9/1)

¹H NMR (400 MHz, CDCl₃) δ 8.49 (d, J=8.4 Hz, 1H, Napht.), 8.31 (d, J=8.4 Hz, 1H, Napht.), 8.03 (d, J=7.2 Hz, 1H, Ar), 7.56 (t, J=8.0 Hz, 1H, Napht.), 7.38 (t, J=7.6 Hz, 1H, Napht.), 7.27 (m, 6H, Ar), 7.19 (m, 8H, Ar), 6.93 (d, J=7.6 Hz, 2H, Ar), 4.47 (m, 1H, CHCH₂CH₂Nₚᵢₚ), 4.12 (m, 3H, CHCH₂CH₂Nₚᵢₚ, CH₂O), 3.90 (m, 1H, CH₂O), 3.65 (d, J=13.8 Hz, 2H, NCH₂Ph), 3.49 (d, J=13.8 Hz, 2H, NCH₂Ph), 3.01 (m, 1H, CHMe), 2.85 (m, 8H, N(CH₃)₂, CHCH₂CH₂Nₚᵢₚ), 1.86 (bd, 2H, CHCH₂CH₂Nₚᵢₚ), 1.27 (m, 2H, CHCH₂CH₂Nₚᵢₚ), 1.00 (d, J=6.8 Hz, 3H, Me).

¹³C NMR (100 MHz, CDCl₃) δ 154.9 (C═O), 151.8, 140.2, 134.7, 134.6, 134.5, 132.5, 131.2, 130.5, 129.8, 129.7, 128.8, 128.4, 128.0, 127.9, 126.7, 123.0, 119.4, 115.1, 66.5 (CH₂O), 56.2 (CHCH₂CH₂Nₚᵢₚ), 53.5 (NCH₂Ph), 51.7 (CHMe), 45.4 (N(CH₃)₂), 43.4 (CHCH₂CH₂Nₚᵢₚ), 31.6 (CHCH₂CH₂Nₚᵢₚ), 11.1 (Me).

LC/MS (ES⁺) m/z 691.3 (M+H)⁺

### Preparation of (S)-2-(dibenzylamino)-3-phenylpropyl 4-(N-phenyl-4-methoxyphenylsulfonamido)piperidine-1-carboxylate (Compound no 157)

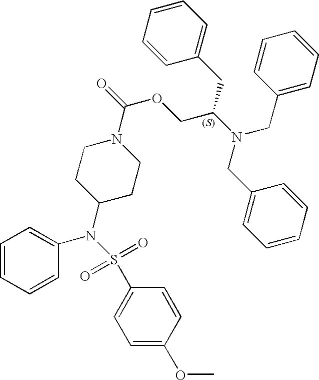

From 4-methoxyphenylsulfonyl chloride and (S)-2-(dibenzylamino)-3-phenylpropyl 4-(phenylamino)piperidine-1-carboxylate.

White solid (78%).

Flash chromatography on silica gel (cyclohexane/ethyl acetate 9/1)

¹H NMR (400 MHz, CDCl₃) δ 7.66 (d, J=8.8 Hz, 2H, Ar), 7.34 (m, 4H, Ar), 7.19 (m, 12H, Ar), 7.00 (m, 4H, Ar), 6.93 (d, J=8.8 Hz, 2H, Ar), 4.34 (m, 1H, CHCH₂CH₂Nₚᵢₚ), 4.18 (m, 2H, CHCH₂CH₂Nₚᵢₚ), 4.03 (m, 2H, CH₂O), 3.88 (s, 3H, OMe), 3.68 (d, J=14.0 Hz, 2H, NCH₂Ph), 3.67 (d, J=14.0 Hz, 2H, NCH₂Ph), 3.15 (m, 1H, CHMe), 2.97 (dd, J=13.6, 6.0 Hz, 1H, CHCH₂Ph), 2.78 (bt, 2H, CHCH₂CH₂Nₚᵢₚ), 2.58 (dd, J=13.6, 8.4 Hz, 1H, CHCH₂Ph), 1.80 (bd, 2H, CHCH₂CH₂Nₚᵢₚ), 1.27 (m, 2H, CHCH₇CH₂Nₚᵢₚ).

¹³C NMR (100 MHz, CDCl₃) δ 162.7, 154.8 (C═O), 139.8, 139.4, 135.1, 132.8, 132.4, 129.4, 129.1, 129.0, 128.9, 128.5, 128.3, 128.1, 126.8, 126.0, 114.0, 64.5 (CH₂O), 56.9 (OMe), 55.5 (CHCH₂CH₂Nₚᵢₚ), 53.7 (NCH₂Ph), 53.4 (CHBn), 43.3 (CHCH₂CH₂Nₚᵢₚ), 34.0 (CHCH₂Ph), 31.7 (CHCH₂CH₂Nₚᵢₚ).

LC/MS (ES⁺) m/z 704.3 (M+H)⁺

### Preparation of (S)-2-(dibenzylamino)-3-phenylpropyl 4-(N-phenyl-4-(trifluoromethyl)phenylsulfonamido)piperidine-1-carboxylate (Compound no 183)

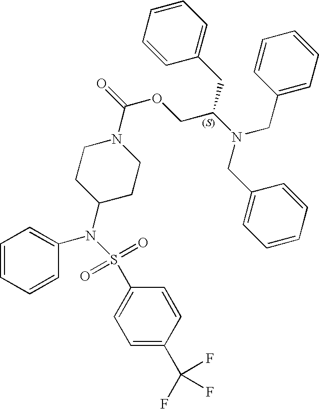

From 4-(trifluoromethyl)phenylsulfonyl chloride and (S)-2-(dibenzylamino)-3-phenylpropyl 4-(phenylamino)piperidine-1-carboxylate.

White solid (62%).

Flash chromatography on silica gel (cyclohexane/ethyl acetate 9/1)

¹H NMR (400 MHz, CDCl₃) δ 7.85 (d, J=8.0 Hz, 2H, Ar), 7.75 (d, J=8.4 Hz, 2H, Ar), 7.39-7.33 (m, 4H, Ar), 7.20 (m, 12H, Ar), 6.99 (m, 4H, Ar), 4.39 (m, 1H, CHCH₂CH₂Nₚᵢₚ), 4.17-4.19 (m, 2H, CHCH₂CH₂Nₚᵢₚ), 4.01 (m, 2H, CH₂O), 3.70 (d, J=14.0 Hz, 2H, NCH₂Ph), 3.66 (d, J=14.0 Hz, 2H, NCH₂Ph), 3.16 (m, 1H, CHBn), 2.58 (dd, J=13.6, 6.0 Hz, 1H, CHCH₂Ph), 2.83 (bt, 2H, CHCH₂CH₂Nₚᵢₚ), 2.58 (dd, J=13.6, 8.4 Hz, 1H, CHCH₂Ph), 1.79 (m, 2H, CHCH₂CH₂Nₚᵢₚ), 1.29 (m, 2H, CHCH₂CH₂Nₚᵢₚ).

¹³C NMR (100 MHz, CDCl₃) δ 154.7 (C═O), 144.6, 139.8, 139.4, 134.4, 132.2, 129.3, 129.2, 129.1, 128.5, 128.3, 128.1, 127.7, 126.8, 126.1, 126.0, 125.9, 64.5 (CH₂O), 57.4 (CHCH₂CH₂Nₚᵢₚ), 53.7 (NCH₂Ph), 53.4 (CHBn), 43.2 (CHCH₂CH₂Nₚᵢₚ), 34.0 (CHCH₂Ph), 30.4 (CHCH₂CH₂Nₚᵢₚ).

LC/MS (ES⁺) m/z 742.3 (M+H)⁺

### Preparation of (S)-2-(dibenzylamino)-3-phenylpropyl 4-(N-phenyl-naphthalen-1-ylsulfonamido)piperidine-1-carboxylate (Compound no 162)

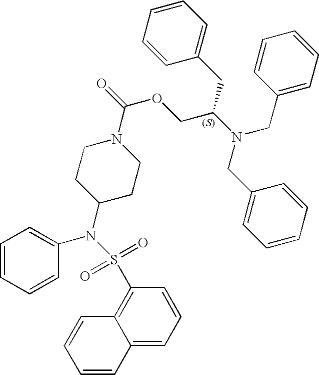

From naphthalene-1-sulfonyl chloride and (S)-2-(dibenzylamino)-3-phenylpropyl 4-(phenylamino)piperidine-1-carboxylate.

White solid (8%).

Flash chromatography on silica gel (cyclohexane/ethyl acetate 9/1)

¹H NMR (400 MHz, CDCl₃) δ 8.63 (d, J=8.0 Hz, 1H, Napht.), 8.15 (t, J=9.0 Hz, 2H, Napht.), 7.94 (d, J=8.4 Hz, 1H, Ar), 7.64 (m, 2H, Napht.), 7.40 (t, J=8.0 Hz, 2H, Napht.), 7.25-7.15 (m, 15H, Ar), 6.99 (d, J=6.8 Hz, 2H, Ar), 6.91 (m, 2H, Ar), 4.49 (m, 1H, CHCH₂CH₂Nₚᵢₚ), 4.15 (m, 2H, CHCH₂CH₂Nₚᵢₚ), 3.98 (m, 2H, CH₂O), 3.67 (d, J=13.4 Hz, 2H, NCH₂Ph), 3.64 (d, J=13.4 Hz, 2H, NCH₂Ph), 3.14 (m, 1H, CHBn), 2.96 (m, 1H, CHCH₂Ph), 2.82 (bt, 2H, CHCH₂CH₂Nₚᵢₚ), 2.58 (m, 1H, CHCH₂Ph), 1.83 (bd, 2H, CHCH₂CH₂Nₚᵢₚ), 1.26 (m, 2H, CHCH₂CH₂Nₚᵢₚ).

LC/MS (ES⁺) m/z 724.3 (M+H)⁺

### Preparation of (S)-2-(dibenzylamino)propyl diphenylthioureido)piperidine-1-carboxylate (Compound no 165)

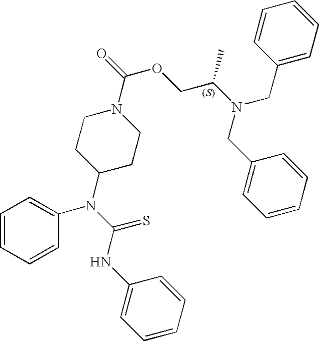

From phenylisothiocyanate and (S)-2-(dibenzylamino)propyl 4-(phenylamino)piperidine-1-carboxylate

Colorless oil (18%).

Flash chromatography on silica gel (cyclohexane/ethyl acetate 95/5)

¹H NMR (400 MHz, CDCl₃) δ 7.52 (m, 3H, Ar), 7.33-7.17 (m, 17H, Ar), 6.68 (s, 1H, NH), 5.80 (m, 1H, CHCH₂CH₂Nₚᵢₚ), 4.24-4.12 (m, 3H, CHCH₂CH₂Nₚᵢₚ, CH₂O), 3.96 (dd, J=11.2, 5.6 Hz, 1H, CH₂O), 3.68 (d, J=14.0 Hz, 2H, NCH₂Ph), 3.51 (d, J=14.0 Hz, 21-1, NCH₂Ph), 3.05 (m, 1H, CHMe), 2.89 (m, 2H, CHCH₂CH₂Nₚᵢₚ), 2.07 (bd, 2H, CHCH₂CH₂Nₚᵢₚ), 1.23 (m, 2H, CHCH₂CH₂Nₚᵢₚ), 1.03 (d, J=6.8 Hz, 3H, Me).

LC/MS (ES⁺) m/z 593.2 (M+H)⁺

### Preparation of (S)-2-(dibenzylamino)propyl 4-(3-ethyl-1-phenylureido)piperidine-1-carboxylate (Compound no 193)

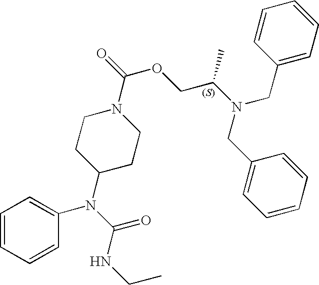

From ethylisocyanate and (S)-2-(dibenzylamino)propyl 4-(phenylamino)piperidine-1-carboxylate

Pale yellow oil (11%).

Flash chromatography on silica gel (cyclohexane/ethyl acetate 8/2)

¹H NMR (400 MHz, CDCl₃) δ 7.41 (m, 31-1, Ar), 7.31 (d, J=7.2 Hz, 4H, Bn), 7.26-7.18 (m, 6H, Ar), 7.13 (m, 2H, Ar), 4.69 (m, 1H, CHCH₂CH₂Nₚᵢₚ), 4.24-4.12 (m, 3H, CHCH₂CH₂Nₚᵢₚ, CH₂O), 3.93 (dd, J=11.2, 6.0 Hz, 1H, CH₂O), 3.86 (m, 1H, NH), 3.68 (d, J=14.0 Hz, 2H, NCH₂Ph), 3.50 (d, J=14.0 Hz, 2H, NCH₂Ph), 3.18 (m, 2H, CH₂CH₃), 3.05 (m, 1H, CHMe), 2.82 (m, 2H, CHCH₂CH₂Nₚᵢₚ), 1.85 (bd, 2H, CHCH₂CH₂Nₚᵢₚ), 1.25 (m, 2H, CHCH₂CH₂Nₚᵢₚ), 1.00 (m, 6H, CHMe, CH₂CH₃).

¹³C NMR (100 MHz, CDCl₃) δ 156.8 (C═O), 155.1 (C═O), 140.2, 137.7, 131.2, 129.8, 128.6, 128.4, 128.0, 126.7, 66.4 (CH₂O), 53.6 (NCH₂Ph), 52.4 (CHMe), 51.8 (CHCH₂CH₂Nₚᵢₚ), 43.4 (CHCH₂CH₂Nₚᵢₚ), 35.5 (CH₂CH₃), 31.1 (CHCH₂CH₂Nₚᵢₚ), 15.5 (CH₂CH₃), 11.1 (Me).

LC/MS (ES⁺) m/z 529.3 (M+H)⁺

### Preparation of (S)-2-(dibenzylamino)propyl 4-(N-phenylpivalamido)piperidine-1-carboxylate (Compound no 163)

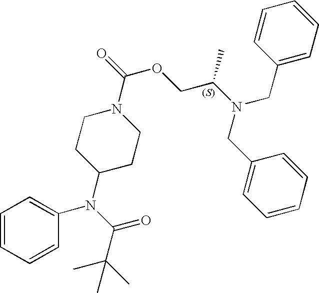

To a stirred solution of the (S)-2-(dibenzylamino)propyl 4-(phenylamino)piperidine-1-carboxylate (22 mg, 0.05 mmol) in pyridine (0.3 mL) were added DMAP (3 mg, 0.025 mmol) and pivaloyl chloride (12 μL, 0.01 mmol) under N₂ atmosphere. The reaction mixture was heated for 48 h at 85° C. The solvent was removed on vacuo and the residue was purified by flash chromatography (silica gel, cyclohexane/ethyl acetate 6/4) to afford a pale yellow solid (7 mg, 27%).

¹H NMR (400 MHz, CDCl₃) δ 7.37 (m, 4H, Ar), 7.30 (d, J=6.8 Hz, 4H, Ar), 7.26-7.19 (m, 5H, Ar), 7.11 (m, 2H, Ar), 4.78 (m, 1H, CHCH₂CH₂Nₚᵢₚ), 4.15-4.11 (m, 3H, CHCH₂CH₂Nₚᵢₚ, CH₂O), 3.92 (dd, J=11.2, 5.6 Hz, 1H, CH₂O), 3.68 (d, J=13.6 Hz, 2H, NCH₂Ph), 3.50 (d, J=13.6 Hz, 2H, NCH₂Ph), 3.03 (m, 1H, CHMe), 2.85 (m, 2H, CHCH₂CH₂Nₚᵢₚ), 1.80 (bd, 2H, CHCH₂CH₂Nₚᵢₚ), 1.24 (m, 2H, CHCH₂CH₂Nₚᵢₚ), 1.03 (d, 3H, Me), 0.98 (s, 9H, tBu).

¹³C NMR (100 MHz, CDCl₃) δ 177.5 (C═O), 155.1 (C═O), 140.2, 138.7, 131.8, 128.5, 128.4, 128.0, 126.7, 66.4 (CH₂O), 54.3 (NCH₂Ph), 53.6 (CHMe), 51.8 (CHCH₂CH₂Nₚᵢₚ), 43.5 (CHCH₇CH₂Nₚᵢₚ), 41.4 (C(Me)₃), 30.3 (CHCH₂CH₂Nₚᵢₚ), 29.5 (Me), 11.2 (Me).

LC/MS (ES⁺) m/z 542.3 (M+H)⁺

### Preparation of (S)-2-(dibenzylamino)propyl 4-(N-methyl-N-phenylamino)piperidine-1-carboxylate (Compound no 166)

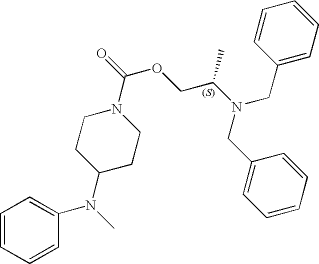

To a stirred solution of the (S)-2-(dibenzylamino)propyl 4-(phenylamino)piperidine-1-carboxylate (79 mg, 0.17 mmol) in acetic acid (0.5 mL) were added formaldehyde solution in water (82 μL, 1.87 mmol, 36.5%) and supported NaBH₃CN (127 mg, 0.56 mmol, 4.4 mmol/g) under N₂ atmosphere. The reaction mixture was stirred at room temperature for 17 h temperature and was filtered. The filtrate was concentrated on vacuo and the residue was purified by flash chromatography (silica gel, cyclohexane/ethyl acetate 8/2) to afford a colorless oil (6 mg, 8%).

¹H NMR (400 MHz, CDCl₃) δ 7.38 (d, J=7.2 Hz, 4H, Ar), 7.31-7.20 (m, 8H, Ar), 6.83 (d, J=8.4 Hz, 2H, Ar), 6.76 (t, J=7.2 Hz, 1H, Ar), 4.26-4.19 (m, 3H, CHCH₂CH₂Nₚᵢₚ, CH₂O), 4.05 (m, 1H, CH₂O), 3.76 (d, J=14.0 Hz, 2H, NCH₂Ph), 3.57 (d, J=14.0 Hz, 2H, NCH₂Ph), 3.12 (m, 1H, CHMe), 2.85 (m, 2H, CHCH₂CH₂Nₚᵢₚ), 2.77 (s, 3H, NCH₃), 1.80 (bd, 2H, CHCH₂CH₂Nₚᵢₚ), 1.24 (m, 2H, CHCH₂CH₂Nₚᵢₚ), 1.09 (d, J=6.8 Hz 3H, Me).

LC/MS (ES⁺) m/z 472.2 (M+H)⁺

### 21) Thiourea Preparation

### Preparation of N-((S)-2-(dibenzylamino)propyl)-4-(1,2-dihydro-2-oxobenzo[d]imidazol-3-yl)piperidine-1-carbothioamide (Compound no 91)

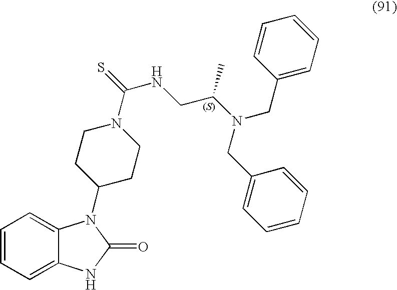

To a stirred solution of EDCI (30 mg, 0.16 mmol) in acetonitrile (0.4 mL) cooled to −10° C. under N₂ was added CS₂ (94 μL, 1.57 mmol). A solution of (S)—N,N-dibenzylpropane-1,2-diamine (40 mg, 0.16 mmol) in acetonitrile (0.4 mL) was added dropwise. The reaction mixture was stirred for 3 h at room temperature and a solution of 1-(piperidin-4-yl)-1H-benzo[d]imidazol-2(3H)-one (34 mg, 0.16 mmol) in acetonitrile (0.8 mL) was added. The mixture was heated at 55° C. overnight and the solvent was removed on vacuo. The crude material was purified by flash chromatography (silica gel, cyclohexane/ethyl acetate 6/4) to afford a pale yellow solid (32 mg, 40%).

¹H NMR (400 MHz, CDCl₃) δ 9.09 (bs, 1H, NH), 7.29-7.16 (m, 10H, Ar), 7.09 (m, 4H, Ar), 6.45 (bs, 1H, NH), 4.79-4.56 (m, 3H, CHCH₂CH₂Nₚᵢₚ, CHCH₂CH₂Nₚᵢₚ), 3.78 (m, 3H, NCH₂Ph, CH₂NH), 3.33 (m, 3H, NCH₂Ph, CH₂NH), 3.03 (m, 2H, CHMe, CHCH₂CH₂Nₚᵢₚ), 2.42-2.27 (m, 2H, CHCH₂CH₂Nₚᵢₚ), 1.92 (m, 2H, CHCH₂CH₂Nₚᵢₚ), 1.18 (d, J=6.0 Hz, 3H, Me).

¹³C NMR (100 MHz, CDCl₃) δ 180.3 (C═S), 154.9 (C═O), 139.5 (C_{Bn}), 129.2 (CH_{Bn}), 128.7 (C_{Ar}), 128.5 (CH_{Bn}), 127.9 (C_{Ar}), 127.4 (CH_{Bn}), 121.5 (CH_{Ar}), 121.3 (CH_{Ar}), 109.8 (CH_{Ar}), 109.4 (CH_{Ar}), 53.7 (NCH₂Ph), 51.9 (CHMe), 50.6 (CHCH₂CH₂Nₚᵢₚ), 47.2 (CH₂NH), 46.7 (CHCH₂CH₂Nₚᵢₚ), 28.9 (CHCH₂CH₂Nₚᵢₚ), 9.9 (Me).

LC/MS (ES⁺) m/z 514.2 (M+H)⁺

### 22) Urea Preparation

### Preparation of N—((S)-2-(dibenzylamino)propyl)-4-(1,2-dihydro-2-oxobenzo[d]imidazol-3-yl)piperidine-1-carboxamide (Compound no 92)

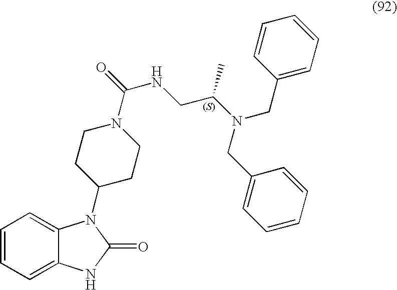

To a stirred solution of the (S)—N,N-dibenzylpropane-1,2-diamine (45 mg, 0.18 mmol) in THF (0.8 mL) at 0° C. was added CDI (29 mg, 0.18 mmol) under N₂ atmosphere. The reaction mixture was stirred for 2 h at room temperature. The 4-(2-keto-1-benzimidazolinyl)-piperidine (35 mg, 0.16 mmol) in THF (1.5 mL) was added and the reaction mixture was refluxed overnight. The solvent was removed on vacuo and the crude material was purified by flash chromatography (silica gel, ethyl acetate) to afford a white solid (30 mg, 38%).

¹H NMR (400 MHz, CDCl₃) δ 9.73 (bs, 1H, NH), 7.28-7.17 (m, 10H, Ar), 7.12 (m, 2H, Ar), 7.06 (m, 2H, Ar), 5.21 (bs, 1H, NH), 4.52 (m, 1H, CHCH₂CH₂Nₚᵢₚ), 4.02 (m, 2H, CHCH₂CH₂Nₚᵢₚ), 3.79 (d, J=13.2 Hz, 2H, NCH₂Ph), 3.33 (m, 3H, NCH₂Ph, CH₂NH), 3.12 (m, 1H, CH₂NH), 2.93-2.85 (m, 2H, CHMe, CHCH₂CH₂Nₚᵢₚ), 2.37-2.25 (m, 2H, CHCH₂CH₂Nₚᵢₚ), 1.88 (bt, 2H, CHCH₂CH₂Nₚᵢₚ), 1.12 (d, J=6.4 Hz, 3H, Me).

¹³C NMR (100 MHz, CDCl₃) δ 157.0 (C═O), 155.0 (C═O), 139.9 (CH_{Bn}), 129.0 (CH_{Bn}), 128.8 (C_{Ar}), 128.4 (CH_{Bn}), 128.0 (C_{Ar}), 127.1 (CH_{Bn}), 121.3 (CH_{Ar}), 121.1 (CH_{Ar}), 109.8 (CH_{Ar}), 109.4 (CH_{Ar}), 52.9 (NCH₂Ph), 52.4 (CHMe), 50.7 (CHCH₂CH₂Nₚᵢₚ), 43.6 (CH₂NH), 43.4 (CHCH₂CH₂Nₚᵢₚ), 29.7 (CHCH₂CH₂Nₚᵢₚ), 10.1 (Me).

LC/MS (ES⁺) m/z 498.4 (M+H)⁺

### 23) Benzimidazol-2-amine Derivative Preparation

### Preparation of N-(1-benzylpiperidin-4-yl)-1H-benzo[d]imidazol-2-amine (Intermediate)

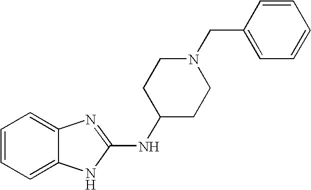

A solution of the 1H-benzo[d]imidazol-2(3H)-one (150 mg, 1.12 mmol) in POCl₃ (0.35 mL, 3.70 mmol) was refluxed overnight. Cool ice (3 mL) was introduced and 4N NaOH solution was added to pH 8. The mixture was extracted with ethyl acetate (3×5 mL). The combined organic layers were dried over anhydrous Na₂SO₄, filtered and concentrated under vacuum. To the crude material was added benzylpiperidin-4-amine (900 μL, 4.48 mmol). The reaction mixture was heated at 130° C. for 72 h. The residue was purified by flash chromatography (silica gel, ethyl acetate/MeOH/NEt₃ 89/10/1) to afford a pale yellow oil which contains 50% of initial product (270 mg of mixture, 40%).

LC/MS (ES⁺) m/z 307.2 (M+H)⁺

### Preparation of (S)-2-(Dibenzylamino)propyl 4-(1H-benzo[d]imidazol-2-ylamino)piperidine-1-carboxylate (Compound no 93)

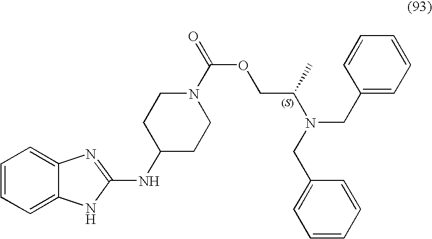

A solution of N-(1-benzylpiperidin-4-yl)-1H-benzo[d]imidazol-2-amine (270 mg, 0.86 mmol) in MeOH (10 mL/mmol) was submitted to hydrogenation in the presence of HCl (4N in dioxane, 215 μL, 0.86 mmol) and 10% Pd/C (46 mg) at room pressure and temperature for 16 h. The reaction mixture was filtered through a pad of Celite. The filtrate was concentrated under vacuo. To a stirred solution of (S)-2-(dibenzylamino)propan-1-ol (213 mg, 0.81 mmol) in THF (3.5 mL) cooled at 0° C. was added CDI (144 mg, 0.88 mmol) under N₂ atmosphere. The reaction mixture was stirred for 2 h at room temperature. Triethylamine (1.0 mL, 7.30 mmol) and the crude salt were added and the reaction mixture was stirred for 72 h at room temperature. The crude material was purified by flash chromatography (silica gel, cyclohexane/ethyl acetate 7/3) to afford a colorless oil (90 mg, 22%).

¹H NMR (400 MHz, CDCl₃) δ 7.33 (d, J=7.6 Hz, 4H, Bn), 7.27 (m, 6H. Ar), 7.19 (m, 2H, Ar), 7.04 (dd, J=6.0, 3.2 Hz, 2H, Ar), 5.12 (s, 1H, NH), 4.19 (m, 1H, CH₂O), 4.03-3.93 (m, 4H, CHCH₂CH₂Nₚᵢₚ, CHCH₂CH₂Nₚᵢₚ, CH₂O), 3.69 (d, J=14.0 Hz, 2H, NCH₂Ph), 3.51 (d, J=14.0 Hz, 2H, NCH₂Ph), 3.08 (m, 1H, CHMe), 2.86 (m, 2H, CHCH₂CH₂Nₚᵢₚ), 2.05 (m, 2H, CHCH₂CH₂Nₚᵢₚ), 1.32 (m, 2H, CHCH₂CH₂Nₚᵢₚ), 1.03 (d, J=6.4 Hz, 3H, Me).

¹³C NMR (100 MHz, CDCl₃) δ 155.4 (C═O), 153.8 (C═N), 140.1 (C_{Bn}), 137.9 (C_{Ar}), 128.3 (CH_{Bn}), 128.0 (CH_{Bn}), 126.7 (CH_{Bn}), 121.0 (CH_{Ar}), 112.1 (CH_{Ar}), 66.9 (CH₂O), 53.6 (NCH₂Ph), 51.8 (CHMe), 49.8 (CHCH₂CH₂Nₚᵢₚ), 42.6 (CHCH₂CH₂Nₚᵢₚ), 32.5 (CHCH₂CH₂Nₚᵢₚ), 11.0 (Me).

LC/MS (ES⁺) m/z 498.3 (M+H)⁺

### Preparation of (S)-2-(dibenzylamino)propyl 2-(1-(((S)-2-(dibenzylamino)propoxy)carbonyl)piperidin-4-ylamino)-1H-benzo[d]imidazole-1-carboxylate (Compound no 94)

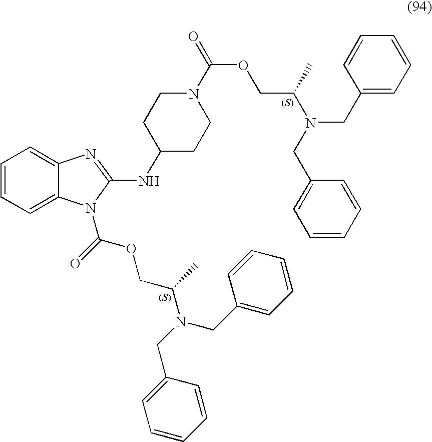

Colorless oil (4 mg, 2%).

¹H NMR (400 MHz, CDCl₃) δ 7.69 (d, J=8.0 Hz, 1H, Ar), 7.45 (m, 1H, Ar), 7.37 (d, J=7.6 Hz, 4H, Ar), 7.32-7.27 (m, 10H, Ar), 7.22 (m, 2H, Ar), 7.17-7.15 (m, 6H, Ar), 4.55 (m, 1H, CH₂O), 4.27 (m, 1H, CH₂O), 4.22 (m, 2H, CH₂O), 4.11-4.00 (m, 2H, CHCH₂CH₂Nₚᵢₚ, CHCH₂CH₂Nₚᵢₚ), 3.84 (d, J=14.0 Hz, 2H, NCH₂Ph), 3.75 (d, J=14.0 Hz, 2H, NCH₂Ph), 3.56 (d, J=14.0 Hz, 2H, NCH₂Ph), 3.49 (d, J=14.0 Hz, 2H, NCH₂Ph), 3.37 (m, 1H, CHMe), 3.12 (m, 3H, CHMe, CHCH₂CH₂Nₚᵢₚ), 2.16 (m, 2H, CHCH₂CH₂Nₚᵢₚ), 1.59 (m, 2H, CHCH₂CH₂Nₚᵢₚ), 1.17 (d, J=6.8 Hz, 3H, Me), 1.09 (d, J=6.8 Hz, 3H, Me).

¹³C NMR (100 MHz, CDCl₃) δ 155.3 (2×C═O). 153.2 (C═N), 140.3, 139.5, 128.6, 128.5, 128.3, 128.2, 128.1, 127.1, 126.9, 126.8, 124.7, 120.7, 116.3, 114.4, 67.8 (CH₂O), 66.2 (CH₂O), 53.3 (NCH₂Ph), 53.2 (NCH₂Ph), 51.4 (CHMe), 51.2 (CHMe), 48.9 (CHCH₂CH₂Nₚᵢₚ), 42.1 (CHCH₂CH₂Nₚᵢₚ), 31.5 (CHCH₂CH₂Nₚᵢₚ), 10.8 (Me), 9.3 (Me).

LC/MS (ES⁺) m/z 779.5 (M+H)⁺

### 24) Guanidine Derivative Preparation

### Preparation of N—((S)-2-(dibenzylamino)propyl)-4-(1,2-dihydro-2-oxobenzo[d]imidazol-3-yl)piperidine-1-carboxamidine (Compound no 95)

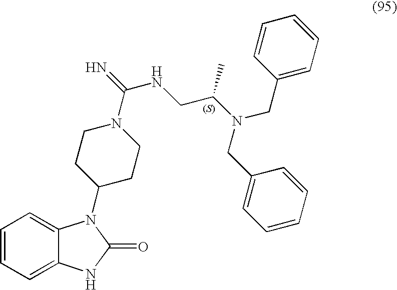

To a stirred solution of the 4-(2-keto-1-benzimidazolinyl)-piperidine (48 mg, 0.22 mmol) in THF/DMF 4:1 (0.6 mL) was added di(1H-imidazol-1-yl)methanimine (58 mg, 0.36 mmol) under N₂ atmosphere. The reaction mixture was stirred for 72 h at room temperature. Water (1 mL) was introduced and the mixture was extracted with ethyl acetate (3×5 mL). The combined organic layers were dried over anhydrous Na₂SO₄, filtered and concentrated under vacuum. To the crude material was added a solution of (S)—N,N-dibenzylpropane-1,2-diamine in THF (0.4 mL). The solution was refluxed for 72 h and the solvent was removed on vacuo. The residue was purified with preparative HPLC to obtain a colorless oil (4 mg, 4%).

¹H NMR (400 MHz, CDCl₃) δ 10.88 (bs, 1H, NH), 7.29 (m, 6H, Ar), 7.15 (m, 5H, Ar), 7.00 (m, 3H, Ar), 5.97 (s, 1H, NH), 4.21 (m, 1H, CHCH₂CH₂Nₚᵢₚ), 3.86 (m, 1H, CH₂N), 3.74 (d, J=13.2 Hz, 2H, NCH₂Ph), 3.46 (m, 1H, m, CH₂N), 3.33 (d, J=13.2 Hz, 2H, NCH₂Ph), 3.29-3.05 (m, 2H, CHCH₂CH₂Nₚᵢₚ), 3.00-2.91 (m, 3H, CHMe, CHCH₂CH₂Nₚᵢₚ), 2.58 (m, 1H, CHCH₂CH₂Nₚᵢₚ), 2.25 (m, 1H, CHCH₂CH₂Nₚᵢₚ), 1.86 (bd, J=10.4 Hz, 1H, CHCH₂CH₂Nₚᵢₚ), 1.75 (bd, J=10.8 Hz, 1H, CHCH₂CH₂Nₚᵢₚ), 1.19 (d, J=5.6 Hz, 3H, Me).

LC/MS (ES⁺) m/z 497.3 (M+H)⁺

### 25) “Amide Link” Procedure

### Preparation of 1-[1-(4-Dibenzylamino-butyryl)-piperidin-4-yl]-1,3-dihydrobenzoimidazol-2-one (Compound no 96)

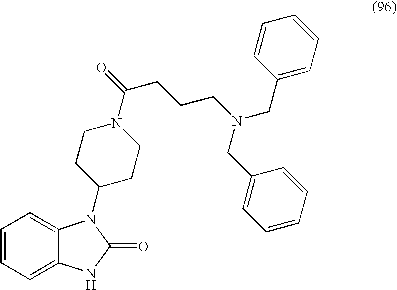

To a solution of 91 mg (0.2 mmol, 1 equiv.) of 4-dibenzylaminobutanoic acid in 1 ml of dry dichloromethane at 0° C. was added successively 83 μL (0.6 mmol, 3 eq) of triethylamine, one drop of DMF, 16 μL (0.22 mmol, 1.1 eq) of thionyl chloride. After 30′, 56 mg (0.26 mmol, 1.3 equiv.) of 4-(2-keto-1-benzimidazolinyl)-piperidine was added. The reaction was allowed to warm to room temperature overnight. The mixture was diluted by dichloromethane, washed by saturated sodium hydrogenocarbonate. The organic layer was dried over sodium sulphate, the solvent evaporated and the residue purified on silica gel.

Colorless oil (20%).

Flash chromatography on silica gel (cyclohexane/ethyl acetate 7/3)

¹H NMR (400 MHz, CDCl₃) δ 9.86 (s, 1H, NH), 7.37 (d, J=7.3 Hz, 4H, Ph), 7.31 (t, J=7.4 Hz, 4H, Ph), 7.24 (m, 2H, Ph), 7.09 (m, 4H, Ph), 4.85 (d, J=13.0 Hz, 1H, CHCH₂CH₂N), 4.53 (tt, J=12.3, 3.8 Hz, 1H, CHCH₂CH₂N), 3.94 (d, J=13.1 Hz, 1H, CHCH₂CH₂N), 3.61 (s, 4H, NCH₂Ph), 3.12 (t, J=12.5 Hz, 1H, CHCH₂CH₂N), 2.63 (t, J=12.5 Hz, 1H, CHCH₂CH₂N), 2.52 (m, 2H, COCH₂CH₂CH₂N), 2.32 (m, 4H, COCH₂CH₂CH₂N, CHCH₂CH₂N), 1.89 (m, 4H, COCH₂CH₂CH₂N, CHCH₂CH₂N)

¹³C NMR (100 MHz, CDCl₃) δ 171.4, 154.8 (C═O), 128.8, 128.1, 127.9, 126.9, 121.4, 121.2, 109.8, 109.2 (C_{Ph}), 58.4 (NCH₂Ph), 52.8 (COCH₂CH₂CH₂N), 50.7 (CHCH₂CH₂N), 41.3 (CHCH₂CH₂N), 31.1 (COCH₂CH₂CH₂N), 29.8 (CHCH₂CH₂N), 22.6 (COCH₂CH₂CH₂N)

LC/MS (ES⁺) m/z 483.3 (M+H)⁺

### 26) Preparation of 2-thiobenzimidazolone

### Preparation of (S)-2-(dibenzylamino)propyl 4-(1,2-dihydro-2-thioxobenzo[d]imidazol-3-yl)piperidine-1-carboxylate (Compound no 97)

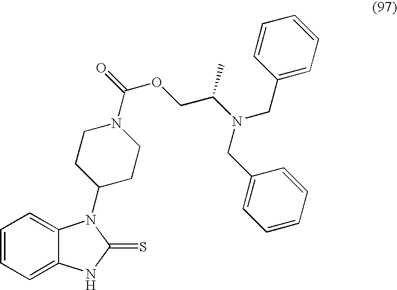

To a solution of 147 mg (0.295 mmol, 1 equiv.) of (S)-2-(dibenzylamino)propyl 4-(1,2-dihydro-2-oxobenzo[d]imidazol-3-yl)piperidine-1-carboxylate in 3 ml of toluene was added 89 mg (0.221 mmol, 0.221 equiv.) of Lawesson reagent. The suspension was refluxed overnight. The solid was filtered and the solvent evaporated in vacuum. The residue was purified on silica gel.

Colorless oil (20%).

Preparative LCMS

¹H NMR (400 MHz, CDCl₃) δ 11.20 (s, 1H, NH), 7.39 (d, J=7.3 Hz, 4H, Ph), 7.28 (t, J=7.4 Hz, 6H, Ph), 7.21 (t, J=7.3 Hz, 3H, Ph), 7.15 (t, J=7.6 Hz, 1H, Ph), 5.46 (m, 1H, CHCH₂CH₂N), 4.45 (m, 1H), 4.30 (dd, J=10.9, 7.6 Hz, 1H, OCH₂), 4.10 (dd, J=12.2, 6.4 Hz, 1H, OCH₂), 3.78 (d, J=13.9 Hz, 2H, NCH₂Ph), 3.57 (d, J=13.9 Hz, 2H, NCH₂Ph), 3.17 (seat., J=6.8 Hz, 1H, CHCH₃), 3.02 (m, 2H, CHCH₂CH₂N), 2.37 (dd, J=12.3, 3.6 Hz, 2H, CHCH₂CH₂N), 1.96 (d, J=8.1 Hz, 2H, CHCH₂CH₂N), 1.12 (d, J=6.8 Hz, 3H, CHCH₃)

LC/MS (ES⁺) m/z 515.1 (M+H)⁺

### 27) Amine Preparation

### Preparation of 2-((S)-2-(dibenzylamino)propyl)isoindoline-1,3-dione (Intermediate)

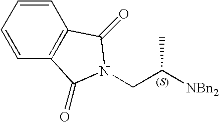

To a stirred suspension of phtalimide (824 mg, 5.60 mmol), (S)-2-(dibenzylamino)propan-1-ol (1.02 g, 4.00 mmol) and PPh₃ (1.47 g, 5.60 mmol) in THF (30 mL) cooled to 0° C. under N₂ was added dropwise DEAD (1.2 mL, 7.60 mmol). The resulting mixture was allowed to warm to room temperature and stirred overnight. The solvent was evaporated. The residue was purified by flash chromatography (silica gel, cyclohexane/methylene chloride 5/5) to afford a white solid (1.08 g, 70%).

¹H NMR (400 MHz, CDCl₃) δ 7.81 (dd, J=5.6, 2.8 Hz, 2H, Ar), 7.74 (dd, J=5.6, 3.2 Hz, 2H, Ar), 7.20 (m, 4H, Ar), 7.11 (m, 6H, Ar), 4.00 (dd, J=14.0, 9.2 Hz, 1H, CH₂N), 3.81 (d, J=13.6 Hz, 2H, NCH₂Ph), 3.38 (m, 3H, CH₂N, NCH₂Ph), 3.23 (m, 1H, CHMe), 1.13 (d, J=6.8 Hz, 3H, Me).

LC/MS (ES⁺) m/z 385.3 (M+H)⁺

### Preparation of (S)—N,N-dibenzylpropane-1,2-diamine (Intermediate)

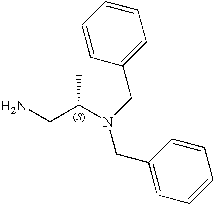

To a stirred solution of 2-((S)-2-(dibenzylamino)propyl)isoindoline-1,3-dione (1.08 g, 2.80 mmol) in ethanol (30 mL) was added hydrazine monohydrate (354 μL, 7.30 mmol) under N₂ atmosphere. The reaction mixture was refluxed for 3 h at room temperature. The residue was filtered and washed with ethanol. The filtrate was concentrated under vacuo and the crude material was purified by flash chromatography (silica gel, methylene chloride/ethanol/NEt₃ 89/10/1) to afford a colorless oil (700 mg, 98%).

¹H NMR (400 MHz, CDCl₃) δ 7.32 (m, 8H, Ar), 7.23 (m, 2H, Ar), 3.77 (d, J=13.6 Hz, 2H, NCH₂Ph), 3.37 (d, J=13.6 Hz, 2H, NCH₂Ph), 2.73 (m, 2H, CH₂N), 2.47 (m, 1H, CHMe), 0.98 (d, J=6.4 Hz, 3H, Me).

LC/MS (ES⁺) in, 255.1 (M+H)⁺

### 28) Preparation of Aminoalcohols

### a. By Dibenzylation

### Preparation of 2-Dibenzylamino-ethanol (Intermediate)

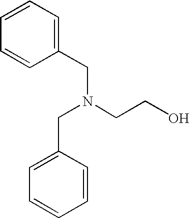

To a vigorously stirred solution of 6.7 ml (1.12 mol, 1 equiv.) ethanolamine in 120 ml of (1/1) MeOH/1-120 was added 7.2 g (1.8 mol., 1.6 equiv.) of sodium hydroxide. The suspension was refluxed for 30 minutes before addition of 24.5 ml (2.3 mol., 2 equiv.) of benzyl chloride. The mixture was refluxed overnight before cooled to room temperature and extracted with 3×160 ml of diethyl ether. The organic solution was dried over sodium sulphate, and evapored. The residue was distilled by Kugel Rohr (100° C., 1 mm

Colorless oil (54%).

¹H NMR (400 MHz, CDCl₃) δ 7.30 (m, 10H, Ph), 3.68 (s, 4H, NCH₂Ph), 3.60 (t, J=5.4 Hz, 211, OCH₂CH₂N), 2.68 (t, J=5.4 Hz, 2H, OCH₂CH₂N), 2.36 (s_{b}, 1H, OH)

LC/MS (ES⁺) m/z 242.1 (M+H)⁺

### b. By Reduction of Corresponding Acid

### Preparation of N,N-Dibenzyl-Succinamic Acid (Intermediate)

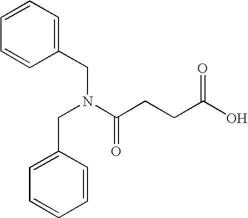

To a stirred solution of dibenzylamine (960 μL, 5 mmol, 1 equiv.) was added 1 g (5 mmol, 1 equiv.) of anhydride succinic acid and the resulting mixture heated at 50° C. overnight. The white solid obtained was dissolved in 10 ml of sodium hydroxide (1M) and was extracted twice by diethyl ether. The aqueous layer was acidified by HCl (4M) to pH 1 and was extracted by dichloromethane. The dichloromethane layer was dried over sodium sulphate, evaporated and used without further purification.

White solid (91%)

¹H NMR (400 MHz, CDCl₃) δ 10.00 (s_{b}, 1H, COOH), 7.30 (m, 10H, Ph), 4.62 (s, 2H, NCH₂Ph), 4.48 (s, 2H, NCH₂Ph), 2.77 (m, 4H, NCH₂CH₂COOH

LC/MS (ES⁺) m/z 298.1 (M+H)⁺

### Preparation of 4-Dibenzylamino-butan-1-ol (Intermediate)

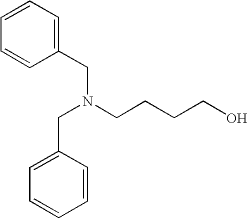

To a stirred suspension lithium aluminum hydride in 4 ml of anhydrous THF was added N,N-Dibenzyl-succinamic acid (185 mg (0.623 mmol, 1 equiv.). The resulting mixture was stirred overnight. Successively 60 μL of water, 60 μL of sodium hydride (2N) and 180 μL of water were added. The precipitate was filtered off and washed with ethyl acetate. The filtrate was dried over sodium sulphate, evaporated and used without further purification.

Yellow oil (75%)

¹H NMR (400 MHz, CDCl₃) δ 7.30 (m, 10H, Ph), 4.62 (m, 6H, NCH₂Ph, OCH₂(CH₂)₂CH₂N), 0.62 (m, 6H, NCH₂Ph, OCH₂(CH₂)₂CH₂N), 2.48 (t, J=6.1 Hz, 2H, OCH₂(CH₂)₂CH₂N), 1.65 (m, 4H, OCH₂(CH₂)₂CH₂N)

LC/MS (ES⁺) m/z 270.2 (M+H)⁺

### 29) Preparation of Bromide from Alcohol

To a stirred solution of (1 mmol., 1 equiv.) appropriate alcohol in 6 ml of anhydrous dichloromethane at 0° C. were added successively 365 mg (1.1 mmol., 1.1 equiv.) of carbon tetrabromide and 287 mg (1.1 mmol., 1.1 equiv.) of triphenylphosphine. The reaction was pursued at 0° C. for 1 h. The mixture was evaporated and purified on silica gel as below or was used without purification for “one-pot” alkylation procedure.

### Preparation of (2-Bromo-ethyl)-carbamic acid tert-butyl ester (Intermediate)

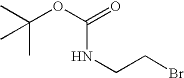

Flash chromatography on silica gel (methylene chloride/methyl alcohol 98/2)

Colorless oil (96%)

¹H NMR (400 MHz, CDCl₃) δ 4.96 (s_{b}, 1H, NH), 3.53 (m, 2H, NCH₂CH₂Br), 3.45 (m, 2H, NCH₂CH₂Br), 1.45 (s, 9H, tBu)

LC/MS (ES⁺) m/z 224.1 (M+H)⁺

### 30) Preparation of Aldehyde and Acid

### Preparation of 4-Dibenzylamino-butyric acid ethyl ester (Intermediate)

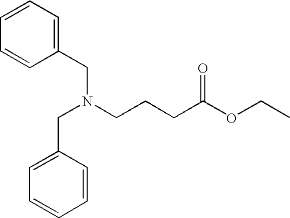

To a solution of dibenzylamine (962 μL, 5 mmol., 1 equiv.) in 2.5 ml of anhydrous DMF was added 715 μL (5 mmol., 1 equiv.) of ethyl 4-bromobutyrate, 1.38 g (10 mmol., 2 equiv.) of potassium carbonate and 83 mg (0.5 mmol., 0.1 equiv.) of potassium iodide. The resulting mixture was heated at 80° C. overnight. The slurry was partitioned with water and ethyl acetate. The aqueous layer was extracted twice with ethyl acetate. The organic layer was dried over sodium sulphate, evaporated and used without further purification.

Yellow oil (98%)

¹H NMR (400 MHz, CDCl₃) δ 7.26 (m, 10H, Ph), 4.10 (q, J=7.1 Hz, 2H, OCH₂CH₃), 3.56 (s, 4H, NCH₂Ph), 2.45 (t, J=6.8 Hz, 2H, NCH₂CH₂CH₂COOEt), 2.32 (t, J=7.5 Hz, 2H, NCH₂CH₂CH₂COOEt), 1.83 (m, 2H, NCH₂CH₂CH₂COOEt), 1.22 (t, J=7.1 Hz, 3H, OCH₂CH₃),

LC/MS (ES⁺) m/z 313.3 (M+H)⁺

### Preparation of 4-Dibenzylamino-butyraldehyde (Intermediate)

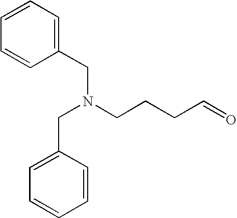

To stirred solution of 297 mg (1 mmol., 1 equiv.) of 4-Dibenzylamino-butyric acid ethyl ester in 2 ml of anhydrous dichloromethane at −78° C. was added dropwise 1.1 ml (1.1 mmol., 1.1 equiv.) of diisobutylaluminium hydride (1 M in dichloromethane). After 40 minutes, 2×40 μL of water was added, and allowed to warm to room temperature. The mixture was diluted with ethyl acetate and filtered. The organic layer was dried over sodium sulphate, evaporated and used without further purification.

Yellow oil (74%)

¹H NMR (400 MHz, CDCl₃) δ 9.70 (s, 1H, CHO), 7.26 (m, 10H, Ph), 3.54 (s, 4H, NCH₂Ph), 2.44 (m, 4H, NCH₂CH₂CH₂CHO), 1.83 (m, 2H, NCH₂CH₂CH₂CHO)

LC/MS (ES⁺) m/z 268.1 (M+H)⁺

### Preparation of 4-Dibenzylamino-butyric acid (Intermediate)

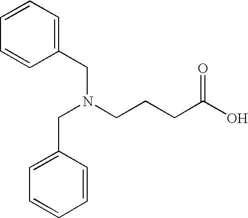

To a stirred solution of 500 mg (1.6 mmol., 1 equiv.) of 4-Dibenzylamino-butyric acid ethyl ester in 1 ml of THF was traited by 4 ml of HCl 2N for 48 hours. The aqueous layer was extracted twice by ethyl acetate. The pH was adjusted to five with sodium carbonate and extracted by dichloromethane. The dichloromethane layer was dried over sodium sulphate, evaporated and used without further purification.

Yellow oil (40%)

¹H NMR (400 MHz, CDCl₃) δ 12.00 (s_{b}, 1H, COOH), 7.30 (m, 10H, Ph), 3.75 (s, 4H, NCH₂Ph), 2.67 (t, J=6.0 Hz, 2H, NCH₂CH₂CH₂COOH), 2.25 (t, J=6.5 Hz, 2H, NCH₂CH₂CH₂COOH), 1.80 (m, 2H, NCH₂CH₂CH₂COOH)

LC/MS (ES⁺) m/z 284.1 (M+H)⁺

### 31) Preparation of N-Boc-4-hydroxypiperidine (Intermediate)

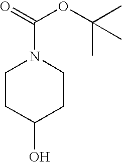

To a stirred solution of 2.5 g (12.5 mmol, 1 equiv.) of N-tert-buthyloxycarbonyl-4-piperidone in 25 ml of methyl alcohol was added 946 mg (25 mmol, 2 eq) of sodium borohydride at 0° C. The reaction was pursued at 0° C. for 2 hours and 2 hours at room temperature. The mixture was diluted by water and brine and extracted by ethyl acetate. The organic layer was dried over sodium sulphate and the solvent was evaporated in vacuum. The residue was used without further purification.

White solide (100%)

¹H NMR (400 MHz, CDCl₃) δ 3.80 (m, 3H, NCH₂CH₂CH), 3.03 (t, J=10.4 Hz, 2H, NCH₂CH₂CH), 3.75 (s, 4H, NCH₂Ph), 2.67 (t, J=6.0 Hz, 2H, NCH₂CH₂CH₂COOH), 2.25 (t, J=6.5 Hz, 2H, NCH₂CH₂CH₂COOH), 1.80 (m, 2H, NCH₂CH₂CH₂COOH)

LC/MS (ES⁺) m/z 284.1 (M+H)⁺

### 32) Preparation of Building Blocks by Alkylation

### Preparation of 4-(2-Oxo-1,2-dihydro-indol-3-ylidene)-piperidine-1-carboxylic acid tert-butyl ester (Intermediate)

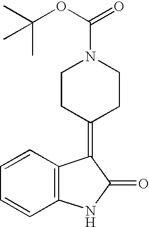

To a stirred solution of indolinone (666 mg, 5 mmol, 1 equiv.) of was solubilized in 10 ml of anhydrous THF. To this solution was added by portion 440 mg (11 mmol, 2.2 equiv.) of sodium hydride at 0° C. After 30 minutes, 996 mg (5 mmol, 1 equiv.) of N-t-butylcarbonylpiperidin-4-one was added. The reaction was allowed to warm to room temperature overnight and stopped by addition of satured solution of ammonium chloride. The mixture was extracted by ethyl acetate. The organic layer was dried over sodium sulphate, evaporated and purified on silica gel.

Flash chromatography on silica gel (cyclohexane/ethyl acetate 8/2 to 1/1)

Pale Yellow oil (50%)

¹H NMR (400 MHz, CDCl₃) δ 7.95 (s, 1H, NH), 7.51 (d, J=7.8 Hz, 1H, Ph), 7.23 (dd, J=10.5, 18.1 Hz, 1H, Ph), 7.02 (t, J=7.6 Hz, 1H, Ph), 6.87 (d, J=7.7 Hz, 1H, Ph), 3.61 (t, J=5.7 Hz, 2H, CqCH₂CH₂N), 3.56 (m, 4H, CqCH₂CH₂N), 3.01 (t, J=5.9 Hz, 2H, CqCH₂CH₂N), 1.49 (s, 9H, t-Bu)

¹³C NMR (100 MHz, CDCl₃) δ 169.6 (tBuOC═O), 156.3 (C═O), 139.7 (C_{q}), 128.1, 123.8 (CH_{Ph}), 123.5, 122.4 (C_{q}), 121.8 (CH_{Ph}), 114.9 (C_{q}), 109.7 (CH_{Ph}), 79.8 ((CH₃)₃CqO), 32.0, 30.1 (CqCE₂CH₂N), 28.4 (CqCH₂CH₂N), 28.3 ((CH₃)₃C_{q}O)

LC/MS (ES⁺) m/z 315.1 (M+H)⁺

### 33) Preparation of Hydrochloride Salt

### Preparation of (S)-2-(dibenzylamino)-3-phenylpropyl 4-(1-benzyl-1,2-dihydro-2-oxobenzo[d]imidazol-3-yl)piperidine-1-carboxylate, hydrochloride salt (Compound no 37)

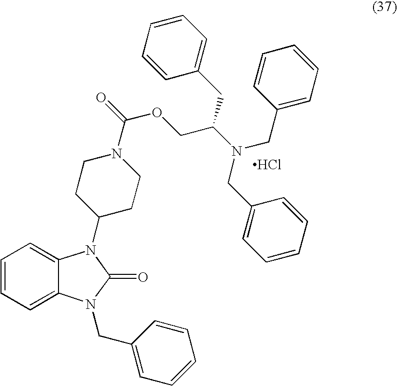

The hydrochloride salt was prepared from (S)-2-(dibenzylamino)-3-phenylpropyl 4-(1-benzyl-1,2-dihydro-2-oxobenzo[d]imidazol-3-yl)piperidine-1-carboxylate after treatment by HCl/AcOEt (1 M). The solid was purified by washing with dry diethyl ether.

White solid (100%).

LC/MS (ES⁺) m/z 665.4 (M+H)⁺

### 34) “One Pot Procedure”: Perbenzylation, Reduction of Corresponding Ester and CDI Coupling

General Protocol:

To a stirred solution of the appropriate aminoacid (1 equiv.) in MeOH (2 mL/mmol) were added BnBr (2.2 equiv.) and K₂CO₃ (2.5 equiv.) under N₂ atmosphere. The reaction mixture was stirred for 16 h at room temperature and the solvent was removed on vacuo. H₂O (2 mL/mmol) was added and the aqueous phase was extracted with dichloromethane (3×5 mL/mmol). The combined organic layers were dried (Na₂SO₄), filtered and concentrated on vacuo. To the crude material (1.5 equiv.) in THF (2 mL/mmol) at 0° C. was added LiAlH₄ (2 equiv.) and the reaction mixture was stirred for 16 h at room temperature. Successively water (40 μL/mmol LiAlH₄), aqueous sodium hydroxide 2N (40 μL/mmol LiAlH₄) and water (120 μL/mmol LiAlH₄) were added. The precipitated was filtered off and washed with ethyl acetate. The filtrate was dried over sodium sulphate, evaporated. To a stirred solution of the residue in THF (2 mL/mmol) at 0° C. was added CDI (1.1 equiv.) under N₂ atmosphere. The reaction mixture was stirred for 2 h at room temperature. The appropriate amine (1.5 equiv.) in THF (10 mL/mmol) was added and the reaction mixture was stirred for 72 h at room temperature. The solvent was removed on vacuo and the residue was purified by Clash chromatography on silica gel.

(Ref: J. Org. Chem., 1996, 3635-45; J. Org. Chem., 2003, 613-16)

### Preparation of (S)-2-(dibenzylamino)-4-methylpentyl 4-(1,2-dihydro-2-oxobenzo[d]imidazol-3-yl)piperidine-1-carboxylate (Compound no 108)

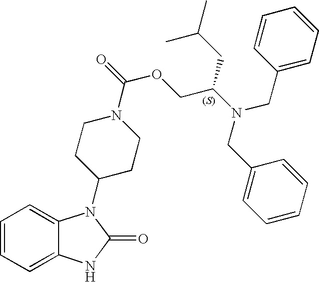

From 4-(2-keto-1-benzimidazolinyl)-piperidine and (S)-2-amino-4-methylpentanoic acid

White solid (48%).

Flash chromatography on silica gel (dichloromethane/methanol 95/5)

¹H NMR (400 MHz, CDCl₃) δ 10.26 (s, 1H, NH), 7.39 (d, J=7.2 Hz, 4H, Ph), 7.28 (t, J=7.6 Hz, 4H, Ph), 7.21 (t, J=7.3, 2H, Ph), 7.14 (m, 2H, Ph), 7.07 (m, 2H, Ph), 4.55 (m, 1H, CHCH₂CH₂N), 4.35 (m, 2H, CHCH₂CH₂N), 4.31 (dd, J=11.2, 6.8 Hz, 1H, CH₂O), 4.08 (m, 1H, CH₂O), 3.73 (d, J=13.6 Hz, 2H, NCH₂Ph), 3.66 (d, J=13.6 Hz, 2H, NCH₂Ph), 2.99 (m, 3H, CHCH₂CH₂N, CHCH₂iPr), 2.40 (m, 2H, CHCH₂CH₂N), 1.91 (m, 2H, CHCH₂CH₂N), 1.77 (m, 1H, CH(CH₃)₂), 1.56 (m, 1H, CH₂CH(CH₃)₂), 1.19 (m, 1H, CH₂CH(CH₃)₂), 0.88 (d, J=6.8 Hz, 3H, CHCH₃), 0.70 (d, J=6.8 Hz, 3H, CHCH₃)

¹³C NMR (100 MHz, CDCl₃) δ 155.3 (C═O), 155.2 (C═O), 140.2, 128.8, 128.1, 128.0, 126.8, 121.4, 121.1, 109.9, 109.3, 66.8 (CH₂O), 54.1 (NCH₂Ph), 53.9 (CHiPr), 50.7 (CHCH₂CH₂N), 43.3 (CHCH₂CH₂N), 30.8 (CH(CH₃)₂), 29.2 (CHCH₂CH₂N), 24.9 (CH₂CH(CH₃)₂), 23.0 (2×CHCH₃)

LC/MS (ES⁺) m/z 541.3 (M+H)⁺

### Preparation of (S)-2-(dibenzylamino)-3-methylbutyl 4-(1,2-dihydro-2-oxobenzo[d]imidazol-3-yl)piperidine-1-carboxylate (Compound no 107)

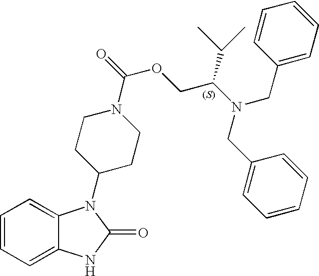

From 4-(2-keto-1-benzimidazolinyl)-piperidine and (S)-2-amino-3-methylbutanoic acid.

Colorless oil (27%).

Flash chromatography on silica gel (dichloromethane/methanol 95/5)

¹H NMR (400 MHz, CDCl₃) δ 9.85 (s, 1H, NH), 7.37 (d, J=7.6 Hz, 4H, Ph), 7.28 (t, J=7.6 Hz, 4H, Ph), 7.21 (t, J=7.3 Hz, 2H, Ph), 7.14 (m, 4H, Ph), 4.54 (m, 3H, CHCH₂CH₂N, CHCH₂CH₂N), 4.31 (m, 2H, CH₂O), 3.93 (d, J=14.0 Hz, 2H, NCH₂Ph), 3.56 (d, J=14.0 Hz, 2H, NCH₂Ph), 2.99 (m, 2H, CHCH₂CH₂N), 2.48 (m, 1H, CHiPr), 2.43 (m, 2H, CHCH₂CH₂N), 2.05 (m, CH(Me)₃), 1.89 (m, 2H, CHCH₂CH₂N), 1.06 (d, J=6.8 Hz, 3H, CHCH₃), 0.92 (d, J=6.4 Hz, 3H, CHCH₃)

¹³C NMR (100 MHz, CDCl₃) δ 155.2 (C═O), 154.7 (C═O), 140.1, 128.8, 128.1, 127.8, 126.8, 121.4, 121.2, 109.8, 109.2, 62.6 (CH₂O), 62.1 (CHiPr), 54.6 (NCH₂Ph), 50.6 (CHCH₂CH₂N), 43.7 (CHCH₂CH₂N), 29.2 (CHCH₂CH₂N), 27.8 (CH(CH₃)₂), 21.2 (CHCH₃), 20.3 (2×CHCH₃)

LC/MS (ES⁺) m/z 527.2 (M+H)⁺

### Preparation of 6-(dibenzylamino)hexyl 4-(1,2-dihydro-2-oxobenzo[d]imidazol-3-yl)piperidine-1-carboxylate (Compound no 180)

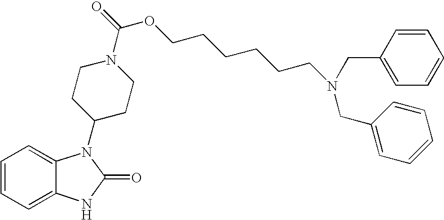

From 4-(2-keto-1-benzimidazolinyl)-piperidine and 6-aminohexanoic acid

White solid (90%).

Flash chromatography on silica gel (dichloromethane/methanol 95/5)

¹H NMR (400 MHz, CDCl₃) δ 9.52 (s, 1H, NH), 7.36-7.26 (m, 10H, Ph), 7.12 (m, 2H, Ph), 7.07 (m, 2H, Ph), 4.51-4.45 (m, 3H, CHCH₂CH₂N, CHCH₂CH₂N), 4.08 (t, J=6.4 Hz 2H, CH₂O), 3.54 (s, 4H, NC′H₂Ph), 2.92 (m, 2H, CHCH₂CH₂N), 2.43-2.33 (m, 2H, CHCH₂CH₂N, CH₂CH₂CH₂NBn₂), 1.85 (m, 2H, CHCH₂CH₂N), 1.63 (m, 2H), 1.43 (m, 2H), 1.31 (m, 4H)

¹³C NMR (100 MHz, CDCl₃) δ 155.5 (C═O), 155.2 (C═O), 140.0, 128.8, 128.5, 128.1, 127.8, 126.7, 121.4, 121.0, 109.9, 109.3, 65.8 (CH₂O), 58.3 (NCH₂Ph), 53.2 (CH₂CH₂CH₂NBn₂), 50.6 (CHCH₂CH₂N), 43.6 (CHCH₂CH₂N), 29.2 (CHCH₂CH₂N), 29.0, 26.9, 25.8

LC/MS (ES⁺) m/z 541.2 (M+H)⁺

### Preparation of (S)-2-(dibenzylamino)-2-phenylethyl 4-(1,2-dihydro-2-oxobenzo[d]imidazol-3-yl)piperidine-1-carboxylate (Compound no 112)

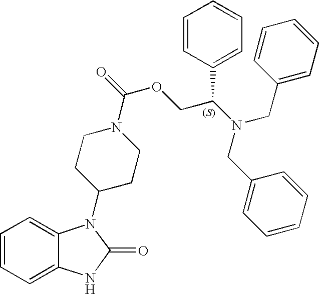

From 4-(2-keto-1-benzimidazolinyl)-piperidine and (S)-2-amino-2-phenylacetic acid.

Colorless oil (21%).

Flash chromatography on silica gel (dichloromethane/methanol 95/5)

¹H NMR (400 MHz, CDCl₃) δ 9.93 (s, 1H, NH), 7.41 (d, J=7.2 Hz, 6H, Ph), 7.30 (t, J=7.6 Hz, 6H, Ph), 7.21 (m, 3H, Ph), 7.10 (m, 2H, Ph), 7.05 (m, 2H, Ph), 4.78 (m, 1H, CHCH₂CH₂N), 4.50 (m, 3H, CH₂O, CHCH₂CH₂N), 4.13 (m, 1H, CH₂O), 3.88 (d, J=14.0 Hz, 2H, NCH₂Ph), 3.36 (d, J=14.0 Hz, 2H, NCH₂Ph), 2.91 (m, 2H, CHCH₂CH₂N), 2.33 (m, 1H, CHPh), 1.85 (m, 2H, CHCH₂CH₂N), 1.66 (m, 2H, CHCH₂CH₂N)

¹³C NMR (100 MHz, CDCl₃) δ 155.0 (C═O), 154.9 (C═O), 139.8, 136.6, 128.8, 128.7, 128.2, 128.1, 127.9, 127.5, 126.9, 121.4, 121.2, 114.9, 109.8, 109.3, 64.1 (CH₂O), 60.6 (CHPh), 54.0 (NCH₂Ph), 50.6 (CHCH₂CH₂N), 43.6 (CHCH₂CH₂N), 29.1 (CHCH₂CH₂N)

LC/MS (ES⁺) m/z 561.2 (M+H)⁺

### Preparation of 2-(dibenzylamino)-2-methylpropyl 4-(1,2-dihydro-2-oxobenzo[d]imidazol-3-yl)piperidine-1-carboxylate (Compound no 111)

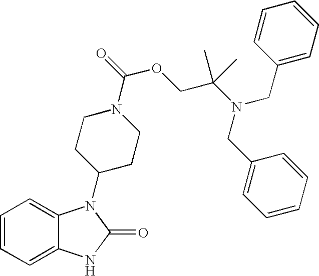

From 4-(2-keto-1-benzimidazolinyl)-piperidine and 2-amino-2-methylpropanoic acid

White solid (34%).

Flash chromatography on silica gel (dichloromethane/methanol 95/5)

¹H NMR (400 MHz, CDCl₃) δ 9.25 (s, 1H, NH), 7.30 (d, J=7.2 Hz, 4H, Ph), 7.17 (t, J=7.2 Hz, 4H, Ph), 7.05 (m, 6H, Ph), 4.54 (m, 1H, CHCH₂CH₂N), 4.40 (m, 2H, CHCH₂CH₂N), 4.17 (s, 2H, CH₂O), 3.82 (s, 4H, NCH₂Ph), 2.97 (m, 2H, CHCH₂CH₂N), 2.37 (m, 2H, CHCH₂CH₂N), 1.89 (m, 2H, CHCH₂CH₂N), 1.18 (s, 6H, Me)

¹³C NMR (100 MHz, CDCl₃) δ 155.2 (C═O), 154.5 (C═O), 142.0, 129.1, 128.2, 127.8, 127.7, 126.3, 121.4, 121.3, 109.6, 109.3, 70.8 (CH₂O), 58.1 (C(Me)₂), 54.1 (NCH₂Ph), 50.6 (CHCH₂CH₂N). 43.6 (CHCH₂CH₂N), 29.5 (CHCH₂CH₂N), 23.2 (Me)

LC/MS (ES⁺) m/z 513.2 (M+H)⁺

### Preparation of 3-(dibenzylamino)butyl 4-(1,2-dihydro-2-oxobenzo[d]imidazol-3-yl)piperidine-1-carboxylate (Compound no 110)

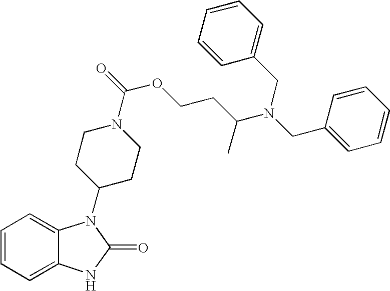

From 4-(2-keto-1-benzimidazolinyl)-piperidine and 3-aminobutanoic acid

Colorless oil (82%).

Flash chromatography on silica gel (dichloromethane/methanol 95/5)

¹H NMR (400 MHz, CDCl₃ δ 9.65 (s, 1H, NH), 7.30 (d. J=7.6 Hz, 4H, Ph), 7.17 (t, J=7.2 Hz, 4H, Ph), 7.05 (t, J=7.2 Hz, 2H, Ph), 7.14-7.06 (m, 4H, Ph), 4.48-4.24 (m, 5H, CHCH₂CH₂N, CH₂O, CHCH₂CH₂N). 3.74 (d, J=13.6 Hz, 2H, NCH₂Ph), 3.40 (d. J=13.6 Hz, 2H, NCH₂Ph), 2.92 (m, 1H, CH₂CHMe), 2.78 (m, 2H, CHCH₂CH₂N), 2.28 (m. 2H, CHCH₂CH₂N), 1.93 (m, 1H, CH₂CHMe), 1.83 (m, 2H, CHCH₂CH₂N),), 1.65 (m, 1H, CH₂CHMe), 1.18 (d, J=6.8 Hz, 3H, Me)

¹³C NMR (100 MHz, CDCl₃) δ 155.3 (C═O), 155.2 (C═O), 140.3, 128.6, 128.5, 128.1, 128.0, 126.7, 121.4, 121.1, 109.6, 109.4, 63.2 (CH₂O), 53.4 (CH₂CHMe). 53.2 (NCH₇Ph), 50.6 (CHCH₂CH₂N). 43.3 (CHCH₂CH₂N), 33.3 (CH₂CHMe), 26.9 (CHCH₂CH₂N), 12.9 (Me)

LC/MS (ES⁺) m/z 513.2 (M+H)⁺

### Preparation of 2-(dibenzylamino)-3,3-dimethylbutyl 4-(1,2-dihydro-2-oxobenzo[d]imidazol-3-yl)piperidine-1-carboxylate (Compound no 109)

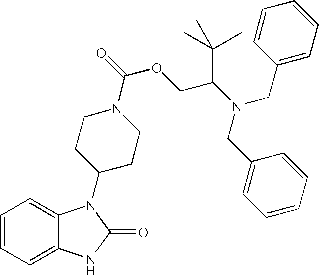

From 4-(2-keto-1-benzimidazolinyl)-piperidine and 2-amino-3,3-dimethylbutanoic acid

White solid (27%).

Flash chromatography on silica gel (dichloromethane/methanol 95/5)

¹H NMR (400 MHz, CDCl₃) δ 8.74 (s, 1H, NH), 7.38 (d, J=6.8 Hz, 4H, Ph), 7.31 (t, J=7.2 Hz, 4H, Ph), 7.18 (m, 2H, Ph), 7.07 (m, 4H, Ph), 4.54 (m, 3H, CHCH₂CH₂N, CHCH₂CH₂N), 4.40-4.17 (bs, 2H, CH₂O), 3.95 (d, J=12.8 Hz, 2H, NCH₂Ph), 3.63 (d, J=13.6 Hz, 2H, NCH₂Ph), 3.02 (m, 2H, CHCH₂CH₂N), 2.80 (m, 1H, CHtBu), 2.37 (m, 2H, CHCH₂CH₂N), 1.89 (m, 2H, CHCH₂CH₂N), 0.87 (s, 9H, tBu)

LC/MS (ES⁺) m/z 541.3 (M+H)⁺

### Preparation of (1-benzylpiperidin-4-yl)methyl 4-(1,2-dihydro-2-oxobenzo[d]imidazol-3-yl)piperidine-1-carboxylate (Compound no 192)

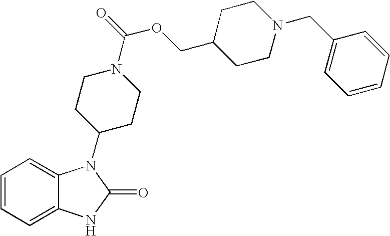

From 4-(2-keto-1-benzimidazolinyl)-piperidine and piperidine-4-carboxylic acid

White solid (65%).

Flash chromatography on silica gel (dichloromethane/methanol 95/5)

¹H NMR (400 MHz, CDCl₃) δ 9.53 (bs, 1H, NH), 7.30 (m, 4H, Bn), 7.25 (m, 1H, Bn), 7.05 (m, 4H. Ar), 4.49 (m, 1H, CHCH₂CH₂N), 4.45 (m, 2H, CHCH₂CH₂N), 3.99 (d, J=5.6 Hz, 2H, CH₂O), 3.52 (s, 2H, NCH₂Ph), 2.92 (m, 4H, CHCH₂CH₂N), 2.34 (m, 2H, CHCH₂CH₂N), 1.98 (m, 2H, CHCH₂CH₂N), 1.85 (m, 2H, CHCH₂CH₂N), 1.71 (m, 3H, CHCH₂CF₂N, CHCH₂CH₂N), 1.36 (m, 2H, CHCH₂CH₂N).

¹³C NMR (100 MHz, CDCl₃) δ 152.7 (C═O), 151.9 (C═O), 132.3, 126.5, 126.2, 125.4, 125.2, 124.2, 118.6, 118.4, 106.9, 106.5, 67.3 (CH₂O), 60.6 (NCH₂Ph), 50.4 (CHCH₂CH₂N), 47.9 (CHCH₂CH₂N), 40.8 (CHCH₂CH₂N), 32.9 (CHCH₂CH₂N), 26.2 (CHCH₂CH₂N).

LC/MS (ES⁺) m/z 449.2 (M+H)⁺

### 35) “One Pot Procedure”: Reductive Amination with Benzaldehyde and CDI Coupling

General Procedure:

To a stirred solution of the appropriate aminoalcohol (1 equiv.) dichloroethane (1 mL/mmol) were added benzaldehyde (2 equiv.) and NaBH(OAc)₃ (2 equiv.) under N₂ atmosphere. The reaction mixture was stirred for 48 h at room temperature and the solvent was removed on vacuo. Saturated aqueous NaHCO₃ solution (2 mL/mmol) was added and the aqueous phase was extracted with ethyl acetate (3×5 mL/mmol). The combined organic layers were dried (NaSO₄), filtered and concentrated on vacuo. To a stirred solution of the residue in THF (2 mL/mmol) at 0° C. was added CDI (1.1 equiv.) under N₂ atmosphere. The reaction mixture was stirred for 2 h at room temperature. The appropriate amine (1.5 equiv.) in THF (10 mL/mmol) was added and the reaction mixture was stirred for 72 h at room temperature. The solvent was removed on vacuo and the residue was purified by flash chromatography on silica gel.

### Preparation of (1-benzylpiperidin-2-yl)methyl 4-(1,2-dihydro-2-oxobenzo[d]imidazol-3-yl)piperidine-1-carboxylate (Compound no 182)

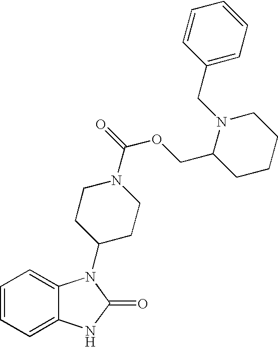

From 4-(2-keto-1-benzimidazolinyl)-piperidine and (piperidin-2-yl)methanol

White solid (13%).

Flash chromatography on silica gel (methylene chloride/methanol 95/5)

¹H NMR (400 MHz, CDCl₃) δ 10.18 (bs, 1H, NH), 7.28 (m, 4H, Bn), 7.25 (m, 1H, Bn), 7.12 (m, 2H, Ar), 7.06 (m, 2H, Ar), 4.49 (m, 1H, CHCH₂CH₂N), 4.46-4.15 (m, 2H, CHCH₂CH₂N), 4.06 (dd, J=10.8, 6.0 Hz, 1H, CH₂O), 3.96 (dd, J=10.8, 7.6 Hz, 1H, CH₂O), 3.51 (s, 2H, NCH₂Ph), 2.92 (m, 3H, CH₂CH₂N, CH₂CHN), 2.31 (m, 2H, CH₂CH₂N), 1.99 (m, 2H, CH₂CH₂N), 1.85 (m, 6H, 2×CH₂CH₂N, CH₂CHN), 1.16 (m, 2H, CH₂CH₂CH₂N).

¹³C NMR (100 MHz, CDCl₃) δ 155.3 (C═O), 155.0 (C═O), 138.2, 129.1, 128.9, 128.3, 128.1, 126.9, 121.3, 121.0, 109.8, 109.2, 68.3 (CH₂O), 63.4 (CH₂CHN), 56.8 (CH₂CH₂N), 54.1 (NCH₂Ph), 50.5 (CHCH₂CH₂N), 43.6 (CHCH₂CH₂N), 35.9 (CH₂CHN), 29.1 (CHCH₂CH₂N), 27.1 (CH₂CH₂N), 24.5 (CH₂CH₂CH₂N).

LC/MS (ES⁺) tri/z 449.2 (M+H)⁺

### 36) Solid Phase Synthesis

a. Synthesis of N-Benzyl-N-methyl polystyrene (Intermediate)

To a suspension of Merrified resin (3.1 mmol, 1 equiv., 1.97 mmol/g) in 50 ml of dry DMF was successively added 1.4 g (9.4 mmol, 3 equiv.) of sodium iodide, 1 g (4.7 mmol, 1.5 equiv.) of 1,8-bis(dimethylamine)naphtalene and 1.7 ml (15.6 mmol, 5 equiv.) of benzylamine. The suspension was heated for 30 hours at 90° C. The resin was filtered off and washed with hot DMF (2×20 ml), water (2×20 ml), and consecutive wash of methyl alcohol and dichloromethane (4×10 ml). Resin was dried in vacuo after washing with diethyl ether (1×10 ml)

White resin

Loading: 1.36 mmol/g

IR (KBr): ν=3446, 3022, 2922, 2308, 1944, 1872, 1798, 1746, 1720, 1652, 1601, 1509, 1492, 1452, 1360, 1181, 1102, 1022, 964, 903, 820, 738, 695, 522 cm

Anal. Calcd N=2.42%. Found N=1.90%

b. Synthesis of (4-(3-Benzyl-3-methylpolystyryl-1-triazenyl)-phenyl)-methanol (Intermediate)

To a solution of 893 mg (7.25 mmol, 5 equiv.) of 4-aminobenzylalcohol, 1.8 ml (14.5 mmol, 10 equiv.) of boron trifluoride diethyl ether complex in 10 ml of dry THF was added 1.7 ml (14.5 mmol, 10 equiv.) of t-butylnitrite at −10° C. The mixture was stirred 1 hours and the diazo suspension was solubilized by addition 10 ml of a solution of dried DMF/pyridine (1/1). 1 g (1.41 mmol, 1 equiv.) of N-Benzyl-N-methyl polystyrene was added and stirred for 1 hour. Resin was filtered off and washed by successively (9/1) DMF/pyridine (3×10 ml), (9/1) THF/NEt₃ (3×10 ml), (9/1) MeOH/NEt₃ (3×10 ml), MeOH (1×10 ml). This procedure was repeated for optimal loading. Resin was dried in vacuo after washing with diethyl ether (1×10 ml)

Orange resin

Loading: 0.92 mmol/g

IR (KBr): ν=3439, 3052, 3025, 2918, 1943, 1802, 1601, 1493, 1450, 1346, 1143, 1073, 1026, 904, 841, 753, 697, 537 cm⁻¹

Anal. Calcd N=5.89%. Found: N=3.85%

c. Synthesis of (4-(3-Benzyl-3-methylpolystyryl-1-triazenyl)-phenyl)-formaldehyde (Intermediate)

To a suspension of N-Benzyl-N-methyl-N-(4-methanolphenyl) polystyrene (100 mg, 0.143 mmol, 1 equiv.) in 5 ml of dry dichloromethane was added 112 mg (0.28 mmol, 2 equiv.) of Dess Martin reagent. The resin was shaken overnight, filtered off and washed by CH₂Cl₂ (4×5 ml) and consecutive wash of methyl alcohol and dichloromethane (4×10 ml). Resin was dried in vacuo after washing with diethyl ether (1×10 ml)

Orange resin

IR (KBr): ν=3056, 3025, 2918, 2860, 2732, 2339, 1945, 1879, 1800, 1693, 1597, 1492, 1448, 1402, 1338, 1138, 1109, 838, 747, 696, 535 cm

d. Typical Procedure for the Preparation of Aminoalcohol Resins, First Reductive Amination

To a suspension of 100 mg (0.14 mmol, 1 equiv.) of N-Benzyl-N-methyl-N-(4-formylphenyl)polystyrene in 2 ml of a solution of dry dichloromethane/acetic acid (2.5% v/v) was added 1.4 mmol (10 equiv.) of appropriate aminoalcohol (typically ethanolamine) and shaken for 2 hours. 424 mg (2 mmol, 15 equiv.) of sodium triacetylborohydride was added and the suspension was shaken overnight. The excess of sodium triacetylborohydride was destroyed by carefully addition of methyl alcohol, resine was filtered off and wash by MeOH (1×5 ml), (9/1) THF/NEt₃ (3×5 ml×15 minutes), (1/1) THF/water (2×5 ml) and consecutive wash of methyl alcohol and dichloromethane (4×5 ml). Resin was dried in vacuo after washing with diethyl ether (1×5 ml)

e. Typical Procedure for the Preparation of Aminoalcohol Resins, Second Reductive Amination

To a suspension of 100 mg (0.14 mmol, 1 equiv.) of N-alkylaminoalcohol resin in 2 ml of dry trimethyl orthoformate was added 22.5 mg (1.4 mmol, 10 equiv.) of the corresponding aldehyde and the suspension was shaken overnight. The resin was filtered off in N, atmosphere and washed by dry DMF (2×2 ml), and dried in vacuo. Resin was suspended in 4 ml of dry dichloromethane before addition of 315 mg (1.4 mmol, 10 equiv.) of sodium triacetylborohydride and shaken overnight. Resine was filtered off and washed by MeOH (1×5 ml), (9/1) THF/NEt₃ to (3×5 ml×15 minutes), (1/1) THF/water (2×5 ml) and consecutive wash of methyl alcohol and dichloromethane (4×5 ml). Resin was dried in vacuo after washing with diethyl ether (1×5 ml).

f. Typical “CDI Procedure” on Solid Phase

To a suspension of 95 mg (0.136 mmol, 1 equiv.) of N,N-dialkylaminoalcohol resin in 5 ml of dry THF was added 220 mg (1.36 mmol, 10 equiv.) of carbonyldiimidazole. The resin was shaken overnight, filtered off and washed by dry THF (3×5 mix 5 minutes). Resin was suspended in 10 ml of dry THF and 147 mg (0.68 mmol, 5 equiv.) of 4-(2-keto-1-benzimidazolinyl)-piperidine was added. The resin was shaken for 72 hours, filtered off, washed by DMF (3×4 ml) and consecutively by methyl alcohol and dichloromethane (4×10 ml). Resin was dried in vacuo after washing with diethyl ether (1×5 ml).

g. Typical Cleavage Procedure

To a suspension of 53 mg (0.076 mmol, 1 equiv.) of triazen resin in 2 ml of dry dichloromethane was added at room temperature 30 μL (0.30 mmol, 4 equiv.) of trichlorosilane. The suspension was shaken for 12 hours and the excess of trichlorosilane was destroyed by addition of silice until no gaz appeared and directly eluted by 5 ml of methyl alcohol. The filtrate was concentrated in vacuo and purified on aminopropyl silica gel (eluant dichloromethane/methyl alcohol 9/1) afforded desired pure product. In some case a preparative HPLC purification was needed.

### Preparation of 2-(N-(4-nitrobenzyl)-N-benzylamino)ethyl 4-(1,2-dihydro-2-oxobenzo[d]imidazol-3-yl)piperidine-1-carboxylate (Compound no 98)

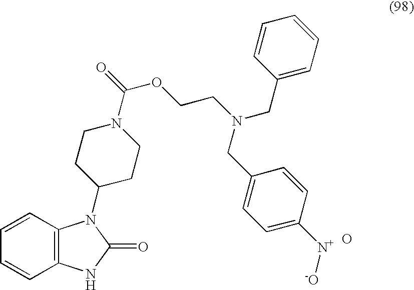

Colorless oil (16% for six steps).

Preparative HPLC

¹H NMR (400 MHz, CDCl₃) δ 9.11 (s, 1H, NH), 8.17 (d, J=8.4 Hz, 2H, Ph), 7.56 (d, J=8.4 Hz, 2H, Ph), 7.32 (t, J=7.6 Hz, 5H, Ph), 7.06 (m, 4H, Ph), 4.50 (m, 2H, CHCH₂CH₂N), 4.26 (m, 3H, CHCH₂CH₂N, OCH₂CH₂N), 3.76 (s, 2H, CH₂Ph), 3.68 (s, 2H, CH₂Ph), 2.93 (m, 21-1, CHCH₂CH₂N), 2.79 (t, J=5.6 Hz, 2H, OCH₂CH₂N), 2.35 (m, 2H, CHCH₂CH₂N), 1.86 (d, J=11.6 Hz, 2H, CHCH₂CH₂N)

LC/MS (ES⁺) m/z 530.3 (M+H)⁺

### Preparation of (S)-2-(benzyl(4-nitrobenzyl)amino)propyl 4-(2-oxo-2,3-dihydro-1H-benzo[d]imidazol-1-yl)piperidine-1-carboxylate (Compound no 113)

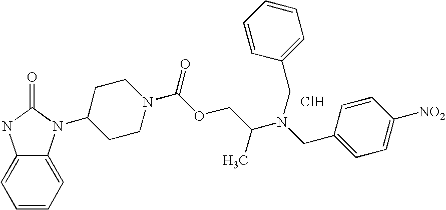

¹H NMR (400 MHz, MeOD) δ 8.16 (d, J=8.8 Hz, 2H, Ph), 7.63 (d, J=8.8 Hz, 2H, Ph), 7.37 (d, J=7.2 Hz, 2H, Ph), 7.28 (t, J=7.2 Hz, 2H, Ph), 7.19 (m, 2H, Ph), 7.05 (m, 3H, Ph), 4.47 (m, 1H, CHCH₂CH₂N), 4.33 (m, 3H, CHCH₂CH₂N, CH₂O). 3.95 (m, 1H, CH₂O), 3.90 (d, J=15.0 Hz, 1H, NCH₂Ph), 3.77 (d, J=14.0 Hz, 1H, NCH₂Ph), 3.70 (d, J=15.0 Hz, 1H, NCH₂Ph), 3.61 (d, J=14.0 Hz, 1H, NCH₂Ph), 3.12 (m, 3H, CHCH₃, CHCH₂CH₂N), 2.40 (m, 2H, CHCH₂CH₂N), 1.85 (m, 2H, CHCH₂CH₂N), 1.13 (d, J=6.8 Hz, 3H, CHCH₃)

LC/MS (ES⁺) m/z 544.2 (M+H)⁺

### Preparation of (S)-2-(benzyl(4-methoxybenzyl)amino)propyl 4-(2-oxo-2,3-dihydro-1H-benzo[d]imidazol-1-yl)piperidine-1-carboxylate hydrochloride (Compound no 194)

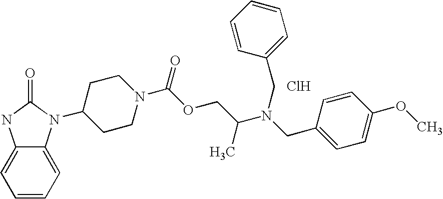

¹H NMR (400 MHz, MeOD) δ 8.30 (s, 1H, NH), 7.43 (d, J=7.2 Hz, 2H, Ar), 7.36 (m, 4H, Ar), 7.27 (m, 2H, Ar), 7.10 (m, 3H, Ar), 6.92 (d, J=8.4 Hz, 2H, Ar), 4.40 (m, 1H, CHCH₂CH₂N), 4.36 (m, 3H, CHCH₂CH₂N, CH₂O), 4.05 (m, 1H, CH₂O), 3.80 (m, 2H, NCH₂Ph), 3.62 (m, 2H, NCH₂Ph), 3.40 (s, 3H, OMe), 3.19 (m, 1H, CHCH₃), 3.05 (m, 2H, CHCH₂CH₂N), 2.40 (m, 2H, CHCH₂CH₂N), 1.88 (m, 2H, CHCH₂CH₂N), 1.17 (d, J=6.8 Hz, 3H, CHCH₃)

LC/MS (ES⁺) m/z 529.2 (M+H)⁺

### Preparation of 3-(benzyl(4-methoxybenzyl)amino)propyl 4-(2-oxo-2,3-dihydro-1H-benzo[d]imidazol-1-yl)piperidine-1-carboxylate hydrochloride (Compound no 117)

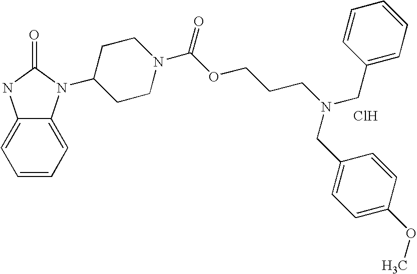

¹H NMR (400 MHz, MeOD) δ 8.26 (s, 1H, NH), 7.42-7.32 (m, 7H, Ar), 7.16 (m, 1H, Ar), 7.07 (m, 3H, Ar), 6.92 (d, J=8.4 Hz, 2H, Ar), 4.27 (m, 1H, CHCH₂CH₂N), 4.16 (m, 2H, CHCH₂CH₂N), 4.05 (t, J=5.6 Hz, 2H, CH₂O), 3.95 (s, 2H, NCH₂Ph), 3.90 (s, 2H, NCH₂Ph), 3.74 (s, 3H, OMe), 2.84 (m, 4H, CH₂CH₂N, CHCH₂CH₂N), 2.31 (m, 2H, CHCH₂CH₂N), 1.99 (m, 2H, CH₂CH₂N), 1.73 (m, 2H, CHCH₂CH₂N).

LC/MS (ES⁺) m/z 529.2 (M+H)⁺

### Preparation of 2-(benzyl(3-phenoxybenzyl)amino)ethyl 4-(2-oxo-2,3-dihydro-1H-benzo[d]imidazol-1-yl)piperidine-1-carboxylate hydrochloride (Compound no 123)

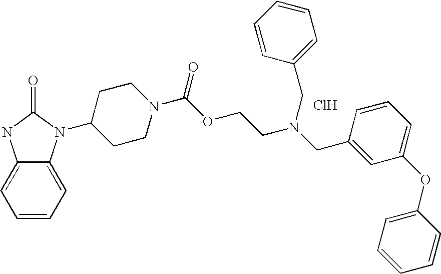

¹H NMR (400 MHz, MeOD) δ 7.31 (m, 7H, Ar), 7.27 (m, 3H, Ar), 7.04 (m, 4H, Ar), 6.93 (d, J=8.8 Hz, 2H, Ar), 6.85 (m, 1H, Ar), 4.39 (m, 1H, CHCH₂CH₂N), 4.19 (m, 4H, CHCH₂CH₂N, CH₂O), 3.66 (s, 2H, NCH₂Ph), 3.64 (s, 2H, NCH₂Ph), 2.77 (m, 2H, CHCH₂CH₂N), 2.77 (d, J=5.6 Hz, 2H, CH₂N), 2.31 (m, 2H, CHCH₂CH₂N), 1.74 (m, 2H, CHCH₂CH₂N).

LC/MS (ES⁺) m/z 577.1 (M+H)⁺

### Example 2

### In Vivo Demonstration of the Antiparasitic Activity (Anti-Malaria) of Compounds of Formula (I)

In this example, we have tested the activity of different compounds of formula (I) on the proliferation of Plasmodium falciparum.

1) Materials and Method

Plasmodium falciparum culture. Prior to the experiments, parasites were maintained in culture according to the method described by Trager and Jensen (Trager, W. et al., Science, 1976, 193, 673-675).

Labeled Hypoxanthine Incorporation Assay:

In vitro anti-malarial activity was measured on asynchronous 3D7 chloroquine-sensitive P. falciparum infected red blood cells (RBC). Suspensions of infected RBC (1.5% final hematocrite, 0.6% parasitemia) were maintained in complete medium (RPMI 1640 complemented with 0.5% type I Albumax) either in absence of the compound of formula (I) (controls) or in contact with a range of molecular concentrations of compounds of formula (I) during 48 h (length of the parasite's cycle) according to the method described by Desjardins et al., Antimicrob Agents Chemother., 1979, 16(6), 710-718.

This range consists in serial dilutions of a high concentrated (10 mM) initial preparation of the different tested compounds of formula (I) or comparative prior art compounds in DMSO, diluted in complete medium at the first expected concentration. After 48 h incubation, 0.5 μCi [3H]-hypoxanthine (radiolabelled precursor of the nucleic acids metabolism) were added to each well of the microtiter plate. After an 18 h-incubation, hypoxanthine incorporation reactions were stopped by a −80° C. freezing. Parasite macromolecules, including radiolabeled nucleic acids were collected from the cell lysates on glass-fiber filters. Amount of radiolabeled uptake was measured after addition of scintillation cocktail in a liquid scintillation counter. Radioactive background was measured from uninfected red blood cells and subtracted from each corresponding well. Parasitic viability was expressed as the ability of compounds-treated parasites to synthesize nucleic acids from the radiolabelld hypoxanthine in comparison to control parasites treated in the absence of drugs. Parasite viability is expressed as a percentage of the control. Analyses of dose-effect curves were performed with the Graphpad Prism analytical software. IC₅₀, corresponding to the drug concentration leading to 50% parasite growth inhibition, were graphically determined from two independent experiments (different cell cultures, different drug dilution stocks) performed in duplicates.

Prior art comparative molecules for inhibition of Plasmodium proliferation are chloroquine, artesunate, artemisinin and triclosan.

2 Results

The results obtained are presented in table 1 below:

TABLE 1

| | | IC₅₀ assessed | |
|---|---|---|---|
| | | on | |
| Number of the | | Plasmodium | |
| tested compound | NAME | Falciparum | |
| | Chloroquine | 0.0103 | μM |
| | Artesunate | 0.0097 | μM |
| | Artemisinine | 0.0287 | μM |
| | Triclosan | 1.55 | μM |
| 25 | (S)-2-(dibenzylamino)propyl 4-(1,2-dihydro-2- | 4.9 | μM |
| | oxobenzo[d]imidazol-3-yl)piperidine-1-carboxylate | | |
| 26 | (S)-2-(dibenzylamino)-3-phenylpropyl 4-(1,2-dihydro-2- | 1.4 | μM |
| | oxobenzo[d]imidazol-3-yl)piperidine-1-carboxylate | | |
| 30 | 2-(dibenzylamino)ethyl 4-(1,2-dihydro-2-oxobenzo[d]imidazol-3- | 6.9 | μM |
| | yl)piperidine-1-carboxylate | | |
| 29 | 3-(dibenzylamino)propyl 4-(1,2-dihydro-2-oxobenzo[d]imidazol- | 3.6 | μM |
| | 3-yl)piperidine-1-carboxylate | | |
| 28 | (S)-2-(dibenzylamino)propyl 5,6-dihydro-4-(1,2-dihydro-2- | 4.1 | μM |
| | oxobenzo[d]imidazol-3-yl)pyridine-1(2H)-carboxylate | | |
| 97 | (S)-2-(dibenzylamino)propyl 4-(1,2-dihydro-2- | 1.06 | μM |
| | thioxobenzo[d]imidazol-3-yl)piperidine-1-carboxylate | | |
| 3 | (S)-2-(dibenzylamino)propyl 4-(1H-benzo[d]imidazol-1- | 1.81 | μM |
| | yl)piperidine-1-carboxylate | | |
| 5 | (S)-2-(dibenzylamino)propyl 4(1-methyl-1H-benzo[d]imidazol-2- | 6.16 | μM |
| | yloxy)piperidine-1-carboxylate | | |
| 67 | tert-butyl 3-(1-(((S)-2- | 0.785 | μM |
| | (dibenzylamino)propoxy)carbonyl)piperidin-4-yl)-2,3-dihydro-2- | | |
| | oxobenzo[d]imidazole-1-carboxylate | | |
| 49 | 1-(1-(3-(dibenzylamino)propyl)piperidin-4-yl)-1H- | 1.27 | μM |
| | benzo[d]imidazol-2(3H)-one | | |
| 69 | (S)-2-(dibenzylamino)propyl 4-(1,2-dihydro-1-methyl-2- | 1.54 | μM |
| | oxobenzo[d]imidazol-3-yl)piperidine-1-carboxylate | | |
| 52 | 1-(1-(2-(dibenzylamino)ethyl)piperidin-4-yl)-1H- | 1.57 | μM |
| | benzo[d]imidazol-2(3H)-one | | |
| 51 | 1-(1-((S)-2-(dibenzylamino)-3-phenylpropyl)piperidin-4-yl)-1H- | 2.11 | μM |
| | benzo[d]imidazol-2(3H)-one | | |
| 50 | 1-(1-((S)-2-(dibenzylamino)propyl)piperidin-4-yl)-1H- | 3.34 | μM |
| | benzo[d]imidazol-2(3H)-one | | |
| 2 | (S)-2-(dibenzylamino)propyl 4(2-oxobenzo[d]oxazol-3(2H)- | 14.8 | μM |
| | yl)piperidine-1-carboxylate | | |
| 46 | O-2-(dibenzylamino)ethyl 4-(1,2-dihydro-2-oxobenzo[d]imidazol- | 2.84 | μM |
| | 3-yl)piperidine-1-carbothioate | | |
| 47 | O-2-(benzyloxy)ethyl 4-(1,2-dihydro-2-oxobenzo[d]imidazol-3- | 23.2 | μM |
| | yl)piperidine-1-carbothioate | | |
| 48 | O—(S)-2-(dibenzylamino)propyl 4-(1,2-dihydro-2- | 2.95 | μM |
| | oxobenzo[d]imidazol-3-yl)piperidine-1-carbothioate | | |
| 19 | (S)-2-(dibenzylamino)propyl 4-((1,2-dihydro-2- | 5.79 | μM |
| | oxobenzo[d]imidazol-3-yl)methyl)piperidine-1-carboxylate | | |
| 6 | (S)-2-(dibenzylamino)propyl 3-(1,2-dihydro-2- | 16.6 | μM |
| | oxobenzo[d]imidazol-3-yl)propylcarbamate | | |
| 11 | (S)-2-(dibenzylamino)propyl 4-(2,3-dihydro-2-oxoimidazo[4,5- | 3.84 | μM |
| | b]pyridin-1-yl)piperidine-1-carboxylate | | |
| 16 | (S)-2-(dibenzylamino)propyl 3-(1,2-dihydro-2- | 2.81 | μM |
| | oxobenzo[d]imidazol-3-yl)piperidine-1-carboxylate | | |
| 71 | (S)-2-(dibenzylamino)propyl 4-(1-benzyl-1,2-dihydro-2- | 0.77 | μM |
| | oxobenzo[d]imidazol-3-yl)piperidine-1-carboxylate | | |
| 72 | (S)-2-(dibenzylamino)propyl 4-(1-(cyclohexylmethyl)-1,2- | 1.03 | μM |
| | dihydro-2-oxobenzo[d]imidazol-3-yl)piperidine-1-carboxylate | | |
| 62 | (S)-2-(dibenzylamino)propyl 4-(2-oxo-3-methylsulfonyl-2,3- | 1.60 | μM |
| | dihydro-1H-benzo[d]imidazol-1-yl)piperidine-1-carboxylate | | |
| 15 | (S)-2-(dibenzylamino)propyl 4-(2-methyl-1H-benzo[d]imidazol-1- | 2.42 | μM |
| | yl)piperidine-1-carboxylate | | |
| 18 | 1-(S)-2-(dibenzylamino)propyl 3-ethyl 4-(1,2-dihydro-2- | 1.52 | μM |
| | oxobenzo[d]imidazol-3-yl)piperidine-1,3-dicarboxylate | | |
| 54 | (S)-2-(dibenzylamino)propyl 4-(2-oxo-3-phenylsulfonyl-2,3- | 0.23 | μM |
| | dihydro-1H-benzo[d]imidazol-1-yl)piperidine-1-carboxylate | | |
| 64 | (S)-2-(dibenzylamino)propyl 4-(1,2-dihydro-2-oxo-1- | 2.88 | μM |
| | (benzoyl)benzo[d]imidazol-3-yl)piperidine-1-carboxylate | | |
| 65 | (S)-2-(dibenzylamino)propyl 4-(1-acetyl-1,2-dihydro-2- | 2.94 | μM |
| | oxobenzo[d]imidazol-3-yl)piperidine-1-carboxylate | | |
| 38 | (S)-2-(dibenzylamino)propyl 4-(6-chloro-1,2-dihydro-2- | 4.68 | μM |
| | oxobenzo[d]imidazol-3-yl)piperidine-1-carboxylate | | |
| 8 | (S)-2-(dibenzylamino)propyl 4-(3,4-dihydroquinolin-1(2H)- | 2.81 | μM |
| | yl)piperidine-1-carboxylate | | |
| 13 | (S)-2-(dibenzylamino)propyl 4-(1,2-dihydro-2- | 10.7 | μM |
| | oxobenzo[d]imidazol-3-yl)azepane-1-carboxylate | | |
| 40 | 2-(1-benzylpiperidin-4-yl)ethyl 4-(1,2-dihydro-2- | 8.16 | μM |
| | oxobenzo[d]imidazol-3-yl)piperidine-1-carboxylate | | |
| 41 | 2-(ethyl(phenyl)amino)ethyl 4-(2-oxo-2,3-dihydro-1H- | 24.4 | μM |
| | benzo[d]imidazol-1-yl)piperidine-1-carboxylate | | |
| 42 | ((S)-1-benzylpyrrolidin-2-yl)methyl 4-(1,2-dihydro-2- | 18.8 | μM |
| | oxobenzo[d]imidazol-3-yl)piperidine-1-carboxylate | | |
| 36 | 3,3-diphenylpropyl 4-(1,2-dihydro-2-oxobenzo[d]imidazol-3- | 8.54 | μM |
| | yl)piperidine-1-carboxylate | | |
| 17 | (S)-2-(dibenzylamino)propyl 3-(1,2-dihydro-2- | 7.18 | μM |
| | oxobenzo[d]imidazol-3-yl)pyrrolidine-1-carboxylate | | |
| 23 | (S)-2-(dibenzylamino)propyl 2-((1,2-dihydro-2- | 7.27 | μM |
| | oxobenzo[d]imidazol-3-yl)methyl)pyrrolidine-1-carboxylate | | |
| 20 | (S)-2-(dibenzylamino)propyl 4-(2-(1,2-dihydro-2- | 6.84 | μM |
| | oxobenzo[d]imidazol-3-yl)ethyl)piperidine-1-carboxylate | | |
| 12 | (S)-2-(dibenzylamino)propyl 4-(1,2-dihydro-2-oxonaphtho[2,3- | 7.20 | μM |
| | d]imidazol-3-yl)piperidine-1-carboxylate | | |
| 44 | (S)-2-(dibenzylamino)propyl 4-(1H-indol-3-yl)piperidine-1- | 5.21 | μM |
| | carboxylate | | |
| 45 | (S)-2-(dibenzylamino)propyl 4-(N- | 7.20 | μM |
| | phenylpropionamido)piperidine-1-carboxylate | | |
| 96 | 1-[1-(4-dibenzylamino-butyryl)-piperidin-4-yl]-1,3-dihydro- | 11.3 | μM |
| | benzoimidazol-2-one | | |
| 75 | (S)-2-(dibenzylamino)propyl 4-(1,2-dihydro-2-oxo-1- | 2.29 | μM |
| | phenylbenzo[d]imidazol-3-yl)piperidine-1-carboxylate | | |
| 9 | 1-(1-(4-(dibenzylamino)butyl)piperidin-4-yl)-1H- | 2.95 | μM |
| | benzo[d]imidazol-2(3H)-one | | |
| 68 | (S)-2-(dibenzylamino)-3-phenylpropyl 4-(1,2-dihydro-1-methyl-2- | 0.452 | μM |
| | oxobenzo[d]imidazol-3-yl)piperidine-1-carboxylate | | |
| 53 | (S)-2-(dibenzylamino)-3-phenylpropyl 4-(1,2-dihydro-3- | 1.94 | μM |
| | phenylsulfonyl-2-oxobenzo[d]imidazol-1-yl)piperidine-1- | | |
| | carboxylate | | |
| 92 | N-((S)-2-(dibenzylamino)propyl)-4-(1,2-dihydro-2- | 3.47 | μM |
| | oxobenzo[d]imidazol-3-yl)piperidine-1-carboxamide | | |
| 91 | N-((S)-2-(dibenzylamino)propyl)-4-(1,2-dihydro-2- | 2.71 | μM |
| | oxobenzo[d]imidazol-3-yl)piperidine-1-carbothioamide | | |
| 77 | (S)-2-(dibenzylamino)propyl 4-(3-(4-ethoxy-4-oxobutyl)-2-oxo- | 0.60 | μM |
| | 2,3-dihydro-1H-benzo[d]imidazol-1-yl)piperidine-1-carboxylate | | |
| 78 | (S)-4-(3-(1-((2-(dibenzylamino)propoxy)carbonyl)piperidin-4-yl)- | 6.29 | μM |
| | 2-oxo-2,3-dihydro-1H-benzo[d]imidazol-1-yl)butanoic acid | | |
| 79 | (S)(1-((2-(dibenzylamino)propoxy)carbonyl)piperidin-4-yl)-5,6- | 3.12 | μM |
| | dihydro-imidazo[4,5,1,j,k][1]benzazepine-2,7(1H,4H)-dione | | |
| 43 | 2,2-diphenylethyl 4-(1,2-dihydro-2-oxobenzo[d]imidazol-3- | 8.33 | μM |
| | yl)piperidine-1-carboxylate | | |
| 73 | (S)-2-(dibenzylamino)propyl 4-(3-(2-tert- | 1.42 | μM |
| | butyloxycarbonylaminoethyl)-2-oxo-2,3-dihydro-1H- | | |
| | benzo[d]imidazol-1-yl)piperidine-1-carboxylate | | |
| 74 | (S)-2-(dibenzylamino)-3-phenylpropyl 4-(3-(2-tert- | 0.384 | μM |
| | butyloxycarbonylaminoethyl)-2-oxo-2,3-dihydro-1H- | | |
| | benzo[d]imidazol-1-yl)piperidine-1-carboxylate | | |
| 76 | (S)-2-(dibenzylamino)-3-phenylpropyl 4-(3-(2 aminoethyl)-2-oxo- | 0.48 | μM |
| | 2,3-dihydro-1H-benzo[d]imidazol-1-yl)piperidine-1-carboxylate | | |
| | D-biotin amid | | |
| 55 | (S)-2-(dibenzylamino)propyl 4-(2-oxo-3-(4- | 9.1 | μM |
| | propylphenylsulfonyl)-2,3-dihydro-1H-benzo[d]imidazol-1- | | |
| | yl)piperidine-1-carboxylate | | |
| 56 | (S)-2-(dibenzylamino)propyl 4-(3-(4-fluorophenylsulfonyl)-2-oxo- | 0.33 | μM |
| | 2,3-dihydro-1H-benzo[d]imidazol-1-yl)piperidine-1-carboxylate | | |
| 57 | (S)-2-(dibenzylamino)propyl 4-(3-(4-methoxyphenylsulfonyl)-2- | 0.41 | μM |
| | oxo-2,3-dihydro-1H-benzo[d]imidazol-1-yl)piperidine-1- | | |
| | carboxylate | | |
| 58 | (S)-2-(dibenzylamino)propyl 4-(2-oxo-3-(4- | 0.42 | μM |
| | (trifluoromethyl)phenylsulfonyl)-2,3-dihydro-1H- | | |
| | benzo[d]imidazol-1-yl)piperidine-1-carboxylate | | |
| 59 | (S)-2-(dibenzylamino)propyl 4-(2-oxo-3-(thiophen-2-ylsulfonyl)- | 0.47 | μM |
| | 2,3-dihydro-1H-benzo[d]imidazol-1-yl)piperidine-1-carboxylate | | |
| 60 | (S)-2-(dibenzylamino)propyl 4-(2-oxo-3-(quinolin-8-ylsulfonyl)- | 0.78 | μM |
| | 2,3-dihydro-1H-benzo[d]imidazol-1-yl)piperidine-1-carboxylate | | |
| 61 | (S)-2-(dibenzylamino)propyl 4-(3-(naphthalen-1-ylsulfonyl)-2- | 0.95 | μM |
| | oxo-2,3-dihydro-1H-benzo[d]imidazol-1-yl)piperidine-1- | | |
| | carboxylate | | |
| 94 | (S)-2-(dibenzylamino)propyl 2-(1-(((S)-2- | 2.62 | μM |
| | (dibenzylamino)propoxy)carbonyl)piperidin-4-ylamino)-1H- | | |
| | benzo[d]imidazole-1-carboxylate | | |
| 93 | (S)-2-(Dibenzylamino)propyl 4-(1H-benzo[d]imidazol-2- | 0.180 | μM |
| | ylamino)piperidine-1-carboxylate | | |
| 80 | (S)-2-(dibenzylamino)propyl 4-(phenylamino)piperidine-1- | 3.41 | μM |
| | carboxylate | | |
| 81 | (S)-2-(dibenzylamino)propyl 4-(4- | 8.42 | μM |
| | methoxyphenylamino)piperidine-1-carboxylate | | |
| 82 | (S)-2-(dibenzylamino)propyl 4-(2,4- | 2.38 | μM |
| | dimethoxyphenylamino)piperidine-1-carboxylate | | |
| 83 | (S)-2-(dibenzylamino)propyl 4-(3,4,5- | 3.09 | μM |
| | trimethoxyphenylamino)piperidine-1-carboxylate | | |
| 85 | (S)-2-(dibenzylamino)propyl 4-(4-iodophenylamino)piperidine-1- | 8.09 | μM |
| | carboxylate | | |
| 98 | 2-(N-(4-nitrobenzyl)-N-benzylamino)ethyl 4-(1,2-dihydro-2- | 3.04 | μM |
| | oxobenzo[d]imidazol-3-yl)piperidine-1-carboxylate | | |
| 63 | (S)-2-(dibenzylamino)propyl 4-(2-oxo-3-(4- | 1.54 | μM |
| | acetamidylphenylsulfonyl)-2,3-dihydro-1H-benzo[d]imidazol-1- | | |
| | yl)piperidine-1-carboxylate | | |
| 70 | (S)-2-(dibenzylamino)-3-phenylpropyl 4-(1-benzyl-1,2-dihydro-2- | 0.953 | μM |
| | oxobenzo[d]imidazol-3-yl)piperidine-1-carboxylate | | |
| 37 | (S)-2-(dibenzylamino)-3-phenylpropyl 4-(1-benzyl-1,2-dihydro-2- | 0.873 | μM |
| | oxobenzo[d]imidazol-3-yl)piperidine-1-carboxylate, | | |
| | hydrochloride salt | | |
| 95 | N-((S)-2-(dibenzylamino)propyl)-4-(1,2-dihydro-2- | 0.511 | μM |
| | oxobenzo[d]imidazol-3-yl)piperidine-1-carboxamidine | | |
| 88 | (S)-2-(dibenzylamino)propyl 4-(1,3-diphenylureido)piperidine-1- | 4.12 | μM |
| | carboxylate | | |
| 89 | (S)-2-(dibenzylamino)propyl 4-(N-phenylbenzamido)piperidine-1- | 1.46 | μM |
| | carboxylate | | |
| 90 | (S)-2-(dibenzylamino)propyl 4-(N- | 0.525 | μM |
| | phenylphenylsulfonamido)piperidine-1-carboxylate | | |
| 84 | (S)-2-(dibenzylamino)propyl 4-(3-fluorophenylamino)piperidine- | 6.74 | μM |
| | 1-carboxylate | | |
| 86 | (S)-2-(dibenzylamino)propyl 4-(3,4- | 7.25 | μM |
| | dichlorophenylamino)piperidine-1-carboxylate | | |
| 87 | (S)-2-(dibenzylamino)propyl 4-(2-aminophenylamino)piperidine- | 10.3 | μM |
| | 1-carboxylate | | |
| 99 | 2-(benzyl(2-(benzyloxy)benzyl)amino)ethyl 4-(2-oxo-2,3-dihydro- | 0.842 | μM |
| | 1H-benzo[d]imidazol-1-yl)piperidine-1-carboxylate | | |
| 100 | 2-(benzyl(4-(benzyloxy)benzyl)amino)ethyl 4-(2-oxo-2,3-dihydro- | 3.15 | μM |
| | 1H-benzo[d]imidazol-1-yl)piperidine-1-carboxylate | | |
| 101 | 2-(benzyl(2,6-difluorobenzyl)amino)ethyl 4-(2-oxo-2,3-dihydro- | 5.37 | μM |
| | 1H-benzo[d]imidazol-1-yl)piperidine-1-carboxylate | | |
| 102 | (S)-2-(benzyl(furan-2-ylmethyl)amino)propyl 4-(2-oxo-2,3- | 7.47 | μM |
| | dihydro-1H-benzo[d]imidazol-1-yl)piperidine-1-carboxylate | | |
| 103 | 2-(benzyl(3-(benzyloxy)benzyl)amino)ethyl 4-(2-oxo-2,3-dihydro- | 3.57 | μM |
| | 1H-benzo[d]imidazol-1-yl)piperidine-1-carboxylate | | |
| 104 | (E)-2-(benzyl(cinnamyl)amino)ethyl 4-(2-oxo-2,3-dihydro-1H- | 4.97 | μM |
| | benzo[d]imidazol-1-yl)piperidine-1-carboxylate | | |
| 105 | (S)-2-(benzyl(tert-butoxycarbonyl)amino)-4-methylpentyl 4-(2- | 1.42 | μM |
| | oxo-2,3-dihydro-1H-benzo[d]imidazol-1-yl)piperidine-1- | | |
| | carboxylate | | |
| 106 | (S)-2-(benzyl(tert-butoxycarbonyl)amino)-3-phenylpropyl 4-(2- | 0.828 | μM |
| | oxo-2,3-dihydro-1H-benzo[d]imidazol-1-yl)piperidine-1- | | |
| | carboxylate | | |
| 107 | (S)-2-(dibenzylamino)-3-methylbutyl 4-(1,2-dihydro-2- | 2.81 | μM |
| | oxobenzo[d]imidazol-3-yl)piperidine-1-carboxylate | | |
| 108 | (S)-2-(dibenzylamino)-4-methylpentyl 4-(1,2-dihydro-2- | 2.61 | μM |
| | oxobenzo[d]imidazol-3-yl)piperidine-1-carboxylate | | |
| 109 | 2-(dibenzylamino)-3,3-dimethylbutyl 4-(1,2-dihydro-2- | 2.4 | μM |
| | oxobenzo[d]imidazol-3-yl)piperidine-1-carboxylate | | |
| 110 | (3-(dibenzylamino)butyl 4-(1,2-dihydro-2-oxobenzo[d]imidazol- | 1.48 | μM |
| | 3-yl)piperidine-1-carboxylate | | |
| 111 | 2-(dibenzylamino)-2-methylpropyl 4-(1,2-dihydro-2- | 2.28 | μM |
| | oxobenzo[d]imidazol-3-yl)piperidine-1-carboxylate | | |
| 112 | (S)-2-(dibenzylamino)-2-phenylethyl 4-(2-oxo-2,3-dihydro-1H- | 0.5 | μM |
| | benzo[d]imidazol-1-yl)piperidine-1-carboxylate | | |
| 113 | (S)-2-(benzyl(4-nitrobenzyl)amino)propyl 4-(2-oxo-2,3-dihydro- | 2.45 | μM |
| | 1H-benzo[d]imidazol-1-yl)piperidine-1-carboxylate | | |
| 114 | 3-(benzyl(4-nitrobenzyl)amino)propyl 4-(2-oxo-2,3-dihydro-1H- | 3.17 | μM |
| | benzo[d]imidazol-1-yl)piperidine-1-carboxylate hydrochloride | | |
| 115 | 2-(benzyl(4-bromobenzyl)amino)ethyl 4-(2-oxo-2,3-dihydro-1H- | 4.41 | μM |
| | benzo[d]imidazol-1-yl)piperidine-1-carboxylate hydrochloride | | |
| 116 | S)-2-(benzyl(4-bromobenzyl)amino)propyl 4-(2-oxo-2,3-dihydro- | 2.88 | μM |
| | 1H-benzo[d]imidazol-1-yl)piperidine-1-carboxylate hydrochloride | | |
| 117 | 3-(benzyl(4-methoxybenzyl)amino)propyl 4-(2-oxo-2,3-dihydro- | 1.87 | μM |
| | 1H-benzo[d]imidazol-1-yl)piperidine-1-carboxylate hydrochloride | | |
| 118 | (R)-2-(dibenzylamino)-3-phenylpropyl 4-(2-oxo-2,3-dihydro-1H- | 1.49 | μM |
| | benzo[d]imidazol-1-yl)piperidine-1-carboxylate | | |
| 119 | 2-(benzyl(4-fluorobenzyl)amino)ethyl 4-(2-oxo-2,3-dihydro-1H- | 3.57 | μM |
| | benzo[d]imidazol-1-yl)piperidine-1-carboxylate hydrochloride | | |
| 120 | 2-(benzyl(3-phenylpropyl)amino)ethyl 4-(2-oxo-2,3-dihydro-1H- | 3.61 | μM |
| | benzo[d]imidazol-1-yl)piperidine-1-carboxylate hydrochloride | | |
| 121 | 2-(benzyl(4-methoxybenzyl)amino)ethyl 4-(2-oxo-2,3-dihydro- | 3.49 | μM |
| | 1H-benzo[d]imidazol-1-yl)piperidine-1-carboxylate hydrochloride | | |
| 122 | 2-(benzyl(3-methoxybenzyl)amino)ethyl 4-(2-oxo-2,3-dihydro- | 3.72 | μM |
| | 1H-benzo[d]imidazol-1-yl)piperidine-1-carboxylate hydrochloride | | |
| 123 | 2-(benzyl(3-phenoxybenzyl)amino)ethyl 4-(2-oxo-2,3-dihydro- | 1.42 | μM |
| | 1H-benzo[d]imidazol-1-yl)piperidine-1-carboxylate hydrochloride | | |
| 124 | 2-(benzyl(3-chlorobenzyl)amino)ethyl 4-(2-oxo-2,3-dihydro-1H- | 3.31 | μM |
| | benzo[d]imidazol-1-yl)piperidine-1-carboxylate hydrochloride | | |
| 125 | (S)-2-(benzyl(3-phenylpropyl)amino)propyl 4-(2-oxo-2,3-dihydro- | 4.2 | μM |
| | 1H-benzo[d]imidazol-1-yl)piperidine-1-carboxylate hydrochloride | | |
| 126 | (S)-2-(benzyl(3-methoxybenzyl)amino)propyl 4-(2-oxo-2,3- | 3.98 | μM |
| | dihydro-1H-benzo[d]imidazol-1-yl)piperidine-1-carboxylate | | |
| | hydrochloride | | |
| 127 | (S)-2-(benzyl(3-phenoxybenzyl)amino)propyl 4-(2-oxo-2,3- | 2.67 | μM |
| | dihydro-1H-benzo[d]imidazol-1-yl)piperidine-1-carboxylate | | |
| | hydrochloride | | |
| 128 | (S)-2-(benzyl(3-chlorobenzyl)amino)propyl 4-(2-oxo-2,3-dihydro- | 2.06 | μM |
| | 1H-benzo[d]imidazol-1-yl)piperidine-1-carboxylate hydrochloride | | |
| 129 | (S)-2-(benzyl(4-chlorobenzyl)amino)propyl 4-(2-oxo-2,3-dihydro- | 3.63 | μM |
| | 1H-benzo[d]imidazol-1-yl)piperidine-1-carboxylate hydrochloride | | |
| 130 | 2-(benzyl(naphthalen-1-ylmethyl)amino)ethyl 4-(2-oxo-2,3- | 3.87 | μM |
| | dihydro-1H-benzo[d]imidazol-1-yl)piperidine-1-carboxylate | | |
| | hydrochloride | | |
| 131 | 2-(benzyl(quinolin-4-ylmethyl)amino)ethyl 4-(2-oxo-2,3-dihydro- | 2.95 | μM |
| | 1H-benzo[d]imidazol-1-yl)piperidine-1-carboxylate hydrochloride | | |
| 132 | 2-(benzyl((1-methyl-1H-indol-2-yl)methyl)amino)ethyl 4-(2-oxo- | 4.2 | μM |
| | 2,3-dihydro-1H-benzo[d]imidazol-1-yl)piperidine-1-carboxylate | | |
| | hydrochloride | | |
| 133 | (S)-2-(benzyl(quinolin-4-ylmethyl)amino)propyl 4-(2-oxo-2,3- | 2.55 | μM |
| | dihydro-1H-benzo[d]imidazol-1-yl)piperidine-1-carboxylate | | |
| | hydrochloride | | |
| 134 | 3-(benzyl(pyridin-3-ylmethyl)amino)propyl 4-(2-oxo-2,3-dihydro- | 1.29 | μM |
| | 1H-benzo[d]imidazol-1-yl)piperidine-1-carboxylate hydrochloride | | |
| 135 | 3-(benzyl(pyridin-2-ylmethyl)amino)propyl 4-(2-oxo-2,3-dihydro- | 3.6 | μM |
| | 1H-benzo[d]imidazol-1-yl)piperidine-1-carboxylate hydrochloride | | |
| 136 | (S)-2-(benzyl(3-(benzyloxy)benzyl)amino)propyl 4-(2-oxo-2,3- | 1.60 | μM |
| | dihydro-1H-benzo[d]imidazol-1-yl)piperidine-1-carboxylate | | |
| | hydrochloride | | |
| 137 | (S)-2-(benzyl(phenanthren-9-ylmethyl)amino)propyl 4-(2-oxo- | 1.61 | μM |
| | 2,3-dihydro-1H-benzo[d]imidazol-1-yl)piperidine-1-carboxylate | | |
| | hydrochloride | | |
| 138 | (S)-2-(benzyl(naphthalen-1-ylmethyl)amino)propyl 4-(2-oxo-2,3- | 2.55 | μM |
| | dihydro-1H-benzo[d]imidazol-1-yl)piperidine-1-carboxylate | | |
| | hydrochloride | | |
| 139 | (S)-2-(benzyl(thiophen-2-ylmethyl)amino)propyl 4-(2-oxo-2,3- | 3.41 | μM |
| | dihydro-1H-benzo[d]imidazol-1-yl)piperidine-1-carboxylate | | |
| | hydrochloride | | |
| 140 | (S)-2-(benzyl(pyridin-2-ylmethyl)amino)propyl 4-(2-oxo-2,3- | 3.86 | μM |
| | dihydro-1H-benzo[d]imidazol-1-yl)piperidine-1-carboxylate | | |
| | hydrochloride | | |
| 141 | 3-(benzyl(thiophen-2-ylmethyl)amino)propyl 4-(2-oxo-2,3- | 4.17 | μM |
| | dihydro-1H-benzo[d]imidazol-1-yl)piperidine-1-carboxylate | | |
| | hydrochloride | | |
| 142 | 3-(benzyl(3-chlorobenzyl)amino)propyl 4-(2-oxo-2,3-dihydro-1H- | 2.92 | μM |
| | benzo[d]imidazol-1-yl)piperidine-1-carboxylate hydrochloride | | |
| 143 | 3-(benzyl(2-(benzyloxy)ethyl)amino)propyl 4-(2-oxo-2,3-dihydro- | 4.27 | μM |
| | 1H-benzo[d]imidazol-1-yl)piperidine-1-carboxylate hydrochloride | | |
| 144 | 3-(benzyl(naphthalen-1-ylmethyl)amino)propyl 4-(2-oxo-2,3- | 3.24 | μM |
| | dihydro-1H-benzo[d]imidazol-1-yl)piperidine-1-carboxylate | | |
| | hydrochloride | | |
| 145 | 3-(benzyl(quinolin-4-ylmethyl)amino)propyl 4-(2-oxo-2,3- | 0.731 | μM |
| | dihydro-1H-benzo[d]imidazol-1-yl)piperidine-1-carboxylate | | |
| | hydrochloride | | |
| 146 | 3-(benzyl(3-phenoxybenzyl)amino)propyl 4-(2-oxo-2,3-dihydro- | 1.96 | μM |
| | 1H-benzo[d]imidazol-1-yl)piperidine-1-carboxylate hydrochloride | | |
| 147 | 3-(benzyl(4-fluorobenzyl)amino)propyl 4-(2-oxo-2,3-dihydro-1H- | 2.22 | μM |
| | benzo[d]imidazol-1-yl)piperidine-1-carboxylate hydrochloride | | |
| 148 | 3-(benzyl(3-phenylpropyl)amino)propyl 4-(2-oxo-2,3-dihydro-1H- | 4.61 | μM |
| | benzo[d]imidazol-1-yl)piperidine-1-carboxylate hydrochloride | | |
| 149 | 3-(benzyl(3-methoxybenzyl)amino)propyl 4-(2-oxo-2,3-dihydro- | 0.588 | μM |
| | 1H-benzo[d]imidazol-1-yl)piperidine-1-carboxylate hydrochloride | | |
| 150 | 2-(benzyl(4-chlorobenzyl)amino)ethyl 4-(2-oxo-2,3-dihydro-1H- | 2.59 | μM |
| | benzo[d]imidazol-1-yl)piperidine-1-carboxylate | | |
| 151 | 2-(benzyl(2-methoxybenzyl)amino)ethyl 4-(2-oxo-2,3-dihydro- | 4.66 | μM |
| | 1H-benzo[d]imidazol-1-yl)piperidine-1-carboxylate | | |
| 152 | (S)-2-(dibenzylamino)-3-phenylpropyl 4- | 0.922 | μM |
| | (phenylamino)piperidine-1-carboxylate | | |
| 153 | (S)-2-(dibenzylamino)propyl 4-(2-phenyl-1H-benzo[d]imidazol-1- | 2.33 | μM |
| | yl)piperidine-1-carboxylate | | |
| 154 | (S)-2-(dibenzylamino)propyl 4-(N-phenyl-4- | 1.59 | μM |
| | propylphenylsulfonamido)piperidine-1-carboxylate | | |
| 155 | (S)-2-(dibenzylamino)propyl 4-(N-phenyl-4- | 0.617 | μM |
| | fluorophenylsulfonamido)piperidine-1-carboxylate | | |
| 156 | (S)-2-(dibenzylamino)propyl 4-(N-phenyl-4- | 0.783 | μM |
| | methoxyphenylsulfonamido)piperidine-1-carboxylate | | |
| 157 | (S)-2-(dibenzylamino)-3-phenylpropyl 4-(N-phenyl-4- | 0.828 | μM |
| | methoxyphenylsulfonamido)piperidine-1-carboxylate | | |
| 158 | (S)-2-(dibenzylamino)propyl 4-(N-phenyl-4- | 0.355 | μM |
| | (trifluoromethyl)phenylsulfonamido)piperidine-1-carboxylate | | |
| 159 | (S)-2-(dibenzylamino)propyl 4-(N-phenylthiophen-2- | 0.861 | μM |
| | ylsulfonamido)piperidine-1-carboxylate | | |
| 160 | (S)-2-(dibenzylamino)propyl 4-(N-phenylquinolin-8- | 1.06 | μM |
| | ylsulfonamido)piperidine-1-carboxylate | | |
| 161 | (S)-2-(dibenzylamino)propyl 4-(N-phenyl-naphthalen-1- | 0.290 | μM |
| | ylsulfonamido)piperidine-1-carboxylate | | |
| 162 | (S)-2-(dibenzylamino)-3-phenylpropyl 4-(N-phenyl-naphthalen-1- | 1.33 | μM |
| | ylsulfonamido)piperidine-1-carboxylate | | |
| 163 | (S)-2-(dibenzylamino)propyl 4-(N-phenylpivalamido)piperidine- | 2.96 | μM |
| | 1-carboxylate | | |
| 164 | (S)-2-(dibenzylamino)propyl 4-(2- | 1.26 | μM |
| | (phenylsulfonyl)phenylamino)piperidine-1-carboxylate | | |
| 165 | (S)-2-(dibenzylamino)propyl 4-(1,3- | 1.25 | μM |
| | diphenylthioureido)piperidine-1-carboxylate | | |
| 166 | (S)-2-(dibenzylamino)propyl 4-(N-methyl-N- | 3.69 | μM |
| | phenylamino)piperidine-1-carboxylate | | |
| 167 | (S)-2-(dibenzylamino)propyl 4-(N-phenyl-2- | 0.338 | μM |
| | methylphenylsulfonamido)piperidine-1-carboxylate | | |
| 168 | (S)-2-(dibenzylamino)propyl 4-(N-phenyl-3- | 0.483 | μM |
| | methylphenylsulfonamido)piperidine-1-carboxylate | | |
| 169 | (S)-2-(dibenzylamino)propyl 4-(N-phenyl-3- | 0.553 | μM |
| | (trifluoromethyl)phenylsulfonamido)piperidine-1-carboxylate | | |
| 170 | (S)-2-(dibenzylamino)propyl 4-(N-phenyl-biphenyl-4- | 2.2 | μM |
| | sulfonamido)piperidine-1-carboxylate | | |
| 171 | (S)-2-(dibenzylamino)propyl 4-(N-phenyl-naphthalen-2- | 4.43 | μM |
| | ylsulfonamido)piperidine-1-carboxylate | | |
| 172 | (S)-2-(dibenzylamino)propyl 4-(N-phenyl-(5- | 0.314 | μM |
| | dimethylamino)naphthalen-1-ylsulfonamido)piperidine-1- | | |
| | carboxylate | | |
| 173 | (S)-2-(dibenzylamino)propyl 4-(2-(4- | 2.58 | μM |
| | chlorophenylthio)phenylamino)piperidine-1-carboxylate | | |
| 174 | (S)-2-(dibenzylamino)-3-phenylpropyl 4-(2,6- | 0.913 | μM |
| | dimethylphenylamino)piperidine-1-carboxylate | | |
| 175 | (S)-2-(dibenzylamino)-3-phenylpropyl 4-(2,6- | 2.60 | μM |
| | diethylphenylamino)piperidine-1-carboxylate | | |
| 176 | (S)-2-(dibenzylamino)-3-phenylpropyl 4-(2- | 2.65 | μM |
| | (methylthio)phenylamino)piperidine-1-carboxylate | | |

### Example 3

### In Vivo Demonstration of the Antiparasitic Activity (Anti-Toxoplasma Properties) of Compounds of Formula (I)

In this example, we have tested the activity of different compounds of formula (I) on the proliferation of Toxoplasma gondii.

### 1) Materials and Method

Toxoplasma gondii culture. Toxoplasma gondii RH-β1 strain was maintained by serial passage in confluent human foreskin fibroblast (HFF) monolayer as described by Morisaki, J. H., et al., (J. Cell. Sci., 1995, 108, 2457-2464). RH-β1 strain carries the Escherichia coli lacZ ((3-galactosidase) gene under the control of SAG1 promoter (Seeber and Boothroyd, Gene169, 1996, 39-45). Cultures were maintained in Dulbecco's Modified Eagle Medium (DMEM) containing L-glutamine, supplemented with 10% fetal bovine serum (FBS) and antibiotics (10000 U/ml Penicillin and 10 mg/ml Streptomycin) (Gibco, Invitrogen Corporation, UK) at 37° C. with 5% CO₂. Toxoplasma gondii cells used in IC₅₀ assay were prepared as follows. A culture of RH-β1 which had completely lysed on HFF monolayer, was forced through a 27-gauge needle twice and was then filtered through a 3-μm-pore-size filter to remove host cell debris and parasite aggregates. The filters were rinsed with 10 ml phosphate-buffered saline (PBS). The combined flow-through was centrifuged at 250×g for 10 min to collect the parasite. Then, they were washed by re-suspension in 5 ml PBS and repeated centrifugation. The resulting parasites were re-suspended, counted, and diluted in medium to a concentration of 10⁴ per 200 μl per well.

Colorimetric microtiter assay. Microtiter plates (96 wells per plate, 32-mm² wells; Corning, N.Y.) were seeded with HFF cells and allowed to grow to confluence in DMEM containing L-glutamine, 10% fetal bovine serum (FBS) and antibiotics (10000 U/ml Penicillin and 10 mg/ml Streptomycin) (Gibco, invitrogen corporation, UK) at 37° C. with 5% CO₂. The DMEM used in this assay lacked phenol red since the readout is dependent on the generation and detection of a derivative of phenol red. HFF cell monolayers grown in the 96-well microtiter plates were infected with 10⁴ parasites per well. Invasion was allowed for 15 min at 37° C. with 5% CO₂ after 15 min of parasite sedimentation on ice. Wells were then rinsed three times with PBS. Increasing doses of molecules were added. Controls consisted of DMEM lacking phenol red, with or without DMSO. Each assay was carried out in triplicates. Plates were then incubated at 37° C. with 5% CO₂ for 48-72 hr to allow the formation of parasitophorous vacuoles (Conseil et al., Antimicrob Agents Chemother, 1999, 43, 1358-1361). β-galactosidase activity was measured as described by Seeber and Boothroyd, 1996. Briefly, plates were rinsed once with PBS and 100 μl of lysis buffer (100 mM HEPES pH8, 1 mM MgSO₄-7H₂O, 1% Triton X-100 and 5 mM DTT) was added, then plates were incubated at 50° C. for 30 min. 110 μl of β-gal buffer (100 mM β-Mercaptoethanol. 9 mM MgCl₂ in 100 mM phosphate buffer pH7.3) was added and subsequently incubated for 5 min at 37° C. A final concentration of 1 mM chlorophenol red-beta-D-galactopyranoside (CPRG), diluted in phosphate buffer, was added. The plates were incubated from 30 min to 16 hours at 37° C., depending on the magnitude of (3-galactosidase activity (i.e. color revelation). The plates were read at 570 and 630 nm on a Bio-tek microtiter plate reader. Results are presented as the mean±2 standard errors. Triplicates of the standard curve experiments were carried out in parallel, by infecting another 96-well plate with serial dilutions of the corresponding parasite strain (0-10⁷ parasite/well).

### 2) Results

The results obtained are tabulated in table 2 below:

TABLE 2

| | | IC₅₀ assessed | |
|---|---|---|---|
| Number of the | | on | |
| tested | | Toxoplama | |
| compound | NAME OF THE TESTED COMPOUND | Gondii | |
| | Triclosan | 0.320 | μM |
| 25 | (S)-2-(dibenzylamino)propyl 4-(1,2-dihydro-2-oxobenzo[d]imidazol- | 14 | μM |
| | 3-yl)piperidine-1-carboxylate | | |
| 26 | (S)-2-(dibenzylamino)-3-phenylpropyl 4-(1,2-dihydro-2- | 4.8 | μM |
| | oxobenzo[d]imidazol-3-yl)piperidine-1-carboxylate | | |
| 30 | 2-(dibenzylamino)ethyl 4-(1,2-dihydro-2-oxobenzo[d]imidazol-3- | 7.5 | μM |
| | yl)piperidine-1-carboxylate | | |
| 29 | 3-(dibenzylamino)propyl 4-(1,2-dihydro-2-oxobenzo[d]imidazol-3- | 2.5 | μM |
| | yl)piperidine-1-carboxylate | | |
| 27 | 2-(N-benzyl-N-methylamino)ethyl 4-(1,2-dihydro-2- | 2 | μM |
| | oxobenzo[d]imidazol-3-yl)piperidine-1-carboxylate | | |
| 28 | (S)-2-(dibenzylamino)propyl 5,6-dihydro-4-(1,2-dihydro-2- | 3.5 | μM |
| | oxobenzo[d]imidazol-3-yl)pyridine-1(2H)-carboxylate | | |
| 31 | 2-(benzyloxy)ethyl 4-(1,2-dihydro-2-oxobenzo[d]imidazol-3- | 0.5 | μM |
| | yl)piperidine-1-carboxylate | | |
| 97 | (S)-2-(dibenzylamino)propyl 4-(1,2-dihydro-2- | 3.5 | μM |
| | thioxobenzo[d]imidazol-3-yl)piperidine-1-carboxylate | | |
| 3 | (S)-2-(dibenzylamino)propyl 4-(1H-benzo[d]imidazol-1- | 8 | μM |
| | yl)piperidine-1-carboxylate | | |
| 5 | (S)-2-(dibenzylamino)propyl 4-(1-methyl-1H-benzo[d]imidazol-2- | 2.5 | μM |
| | yloxy)piperidine-1-carboxylate | | |
| 67 | tert-butyl 3-(1-(((S)-2-(dibenzylamino)propoxy)carbonyl)piperidin-4- | 2 | μM |
| | yl)-2,3-dihydro-2-oxobenzo[d]imidazole-1-carboxylate | | |
| 49 | 1-(1-(3-(dibenzylamino)propyl)piperidin-4-yl)-1H-benzo[d]imidazol- | 0.8 | μM |
| | 2(3H)-one | | |
| 69 | (S)-2-(dibenzylamino)propyl 4-(1,2-dihydro-1-methyl-2- | 0.2 | μM |
| | oxobenzo[d]imidazol-3-yl)piperidine-1-carboxylate | | |
| 52 | 1-(1-(2-(dibenzylamino)ethyl)piperidin-4-yl)-1H-benzo[d]imidazol- | 2 | μM |
| | 2(3H)-one | | |
| 51 | 1-(1-((S)-2-(dibenzylamino)-3-phenylpropyl)piperidin-4-yl)-1H- | 4 | μM |
| | benzo[d]imidazol-2(3H)-one | | |
| 50 | 1-(1-((S)-2-(dibenzylamino)propyl)piperidin-4-yl)-1H- | 7 | μM |
| | benzo[d]imidazol-2(3H)-one | | |
| 2 | (S)-2-(dibenzylamino)propyl 4-(2-oxobenzo[d]oxazol-3(2H)- | 0.8 | μM |
| | yl)piperidine-1-carboxylate | | |
| 46 | O-2-(dibenzylamino)ethyl 4-(1,2-dihydro-2-oxobenzo[d]imidazol-3- | 0.181 | μM |
| | yl)piperidine-1-carbothioate | | |
| 47 | O-2-(benzyloxy)ethyl 4-(1,2-dihydro-2-oxobenzo[d]imidazol-3- | 1.13 | μM |
| | yl)piperidine-1-carbothioate | | |
| 48 | O—(S)-2-(dibenzylamino)propyl 4-(1,2-dihydro-2- | 1.18 | μM |
| | oxobenzo[d]imidazol-3-yl)piperidine-1-carbothioate | | |
| 19 | (S)-2-(dibenzylamino)propyl 4-((1,2-dihydro-2- | 3.41 | μM |
| | oxobenzo[d]imidazol-3-yl)methyl)piperidine-1-carboxylate | | |
| 6 | (S)-2-(dibenzylamino)propyl 3-(1,2-dihydro-2-oxobenzo[d]imidazol- | 0.381 | μM |
| | 3-yl)propylcarbamate | | |
| 11 | (S)-2-(dibenzylamino)propyl 4-(2,3-dihydro-2-oxoimidazo[4,5- | 2.5 | μM |
| | b]pyridin-1-yl)piperidine-1-carboxylate | | |
| 33 | 1-benzylpiperidin-3-yl 4-(1,2-dihydro-2-oxobenzo[d]imidazol-3- | 0.450 | μM |
| | yl)piperidine-1-carboxylate | | |
| 16 | (S)-2-(dibenzylamino)propyl 3-(1,2-dihydro-2-oxobenzo[d]imidazol- | 2.35 | μM |
| | 3-yl)piperidine-1-carboxylate | | |
| 71 | (S)-2-(dibenzylamino)propyl 4-(1-benzyl-1,2-dihydro-2- | 1.76 | μM |
| | oxobenzo[d]imidazol-3-yl)piperidine-1-carboxylate | | |
| 72 | (S)-2-(dibenzylamino)propyl 4-(1-(cyclohexylmethyl)-1,2-dihydro-2- | 1.57 | μM |
| | oxobenzo[d]imidazol-3-yl)piperidine-1-carboxylate | | |
| 62 | (S)-2-(dibenzylamino)propyl 4-(2-oxo-3-methylsulfonyl-2,3-dihydro- | 1.98 | μM |
| | 1H-benzo[d]imidazol-1-yl)piperidine-1-carboxylate | | |
| 15 | (S)-2-(dibenzylamino)propyl 4-(2-methyl-1H-benzo[d]imidazol-1- | 3.41 | μM |
| | yl)piperidine-1-carboxylate | | |
| 18 | 1-(S)-2-(dibenzylamino)propyl 3-ethyl 4-(1,2-dihydro-2- | 2.81 | μM |
| | oxobenzo[d]imidazol-3-yl)piperidine-1,3-dicarboxylate | | |
| 54 | (S)-2-(dibenzylamino)propyl 4-(2-oxo-3-phenylsulfonyl-2,3-dihydro- | 1.68 | μM |
| | 1H-benzo[d]imidazol-1-yl)piperidine-1-carboxylate | | |
| 64 | (S)-2-(dibenzylamino)propyl 4-(1,2-dihydro-2-oxo-1- | 0.96 | μM |
| | (benzoyl)benzo[d]imidazol-3-yl)piperidine-1-carboxylate | | |
| 65 | (S)-2-(dibenzylamino)propyl 4-(1-acetyl-1,2-dihydro-2- | 0.99 | μM |
| | oxobenzo[d]imidazol-3-yl)piperidine-1-carboxylate | | |
| 38 | (S)-2-(dibenzylamino)propyl 4-(6-chloro-1,2-dihydro-2- | 1 | μM |
| | oxobenzo[d]imidazol-3-yl)piperidine-1-carboxylate | | |
| 8 | (S)-2-(dibenzylamino)propyl 4-(3,4-dihydroquinolin-1(2H)- | 25 | μM |
| | yl)piperidine-1-carboxylate | | |
| 13 | (S)-2-(dibenzylamino)propyl 4-(1,2-dihydro-2-oxobenzo[d]imidazol- | 5 | μM |
| | 3-yl)azepane-1-carboxylate | | |
| 39 | 2-(diethylamino)ethyl 4-(1,2-dihydro-2-oxobenzo[d]imidazol-3- | 12 | μM |
| | yl)piperidine-1-carboxylate | | |
| 66 | (S)-2-(dibenzylamino)propyl 4-(1,2-dihydro-2-oxo-1- | 2.80 | μM |
| | (pivaloyl)benzo[d]imidazol-3-yl)piperidine-1-carboxylate | | |
| 34 | 2-(diisopropylamino)ethyl 4-(1,2-dihydro-2-oxobenzo[d]imidazol-3- | 27 | μM |
| | yl)piperidine-1-carboxylate | | |
| 40 | 2-(1-benzylpiperidin-4-yl)ethyl 4-(1,2-dihydro-2- | 0.98 | μM |
| | oxobenzo[d]imidazol-3-yl)piperidine-1-carboxylate | | |
| 41 | 2-(ethyl(phenyl)amino)ethyl 4-(2-oxo-2,3-dihydro-1H- | 1.8 | μM |
| | benzo[d]imidazol-1-yl)piperidine-1-carboxylate | | |
| 42 | ((S)-1-benzylpyrrolidin-2-yl)methyl 4-(1,2-dihydro-2- | 5 | μM |
| | oxobenzo[d]imidazol-3-yl)piperidine-1-carboxylate | | |
| 35 | 2-(dimethylamino)ethyl 4-(1,2-dihydro-2-oxobenzo[d]imidazol-3- | 4 | μM |
| | yl)piperidine-1-carboxylate | | |
| 36 | 3,3-diphenylpropyl 4-(1,2-dihydro-2-oxobenzo[d]imidazol-3- | 1.2 | μM |
| | yl)piperidine-1-carboxylate | | |
| 17 | (S)-2-(dibenzylamino)propyl 3-(1,2-dihydro-2-oxobenzo[d]imidazol- | 3.3 | μM |
| | 3-yl)pyrrolidine-1-carboxylate | | |
| 23 | (S)-2-(dibenzylamino)propyl 2-((1,2-dihydro-2- | 3.1 | μM |
| | oxobenzo[d]imidazol-3-yl)methyl)pyrrolidine-1-carboxylate | | |
| 20 | (S)-2-(dibenzylamino)propyl 4-(2-(1,2-dihydro-2- | 4.2 | μM |
| | oxobenzo[d]imidazol-3-yl)ethyl)piperidine-1-carboxylate | | |
| 12 | (S)-2-(dibenzylamino)propyl 4-(1,2-dihydro-2-oxonaphtho[2,3- | 0.75 | μM |
| | d]imidazol-3-yl)piperidine-1-carboxylate | | |
| 44 | (S)-2-(dibenzylamino)propyl 4-(1H-indol-3-yl)piperidine-1- | 1.85 | μM |
| | carboxylate | | |
| 45 | (S)-2-(dibenzylamino)propyl 4-(N-phenylpropionamido)piperidine-1- | 1.3 | μM |
| | carboxylate | | |
| 96 | 1-[1-(4-dibenzylamino-butyryl)-piperidin-4-yl]-1,3-dihydro- | 6.3 | μM |
| | benzoimidazol-2-one | | |
| 75 | (S)-2-(dibenzylamino)propyl 4-(1,2-dihydro-2-oxo-1- | 3.5 | μM |
| | phenylbenzo[d]imidazol-3-yl)piperidine-1-carboxylate | | |
| 9 | 1-(1-(4-(dibenzylamino)butyl)piperidin-4-yl)-1H-benzo[d]imidazol- | 1.5 | μM |
| | 2(3H)-one | | |
| 68 | (S)-2-(dibenzylamino)-3-phenylpropyl 4-(1,2-dihydro-1-methyl-2- | 0.800 | μM |
| | oxobenzo[d]imidazol-3-yl)piperidine-1-carboxylate | | |
| 53 | (S)-2-(dibenzylamino)-3-phenylpropyl 4-(1,2-dihydro-3- | 22 | μM |
| | phenylsulfonyl-2-oxobenzo[d]imidazol-1-yl)piperidine-1-carboxylate | | |
| 92 | N-((S)-2-(dibenzylamino)propyl)-4-(1,2-dihydro-2- | 15 | μM |
| | oxobenzo[d]imidazol-3-yl)piperidine-1-carboxamide | | |
| 91 | N-((S)-2-(dibenzylamino)propyl)-4-(1,2-dihydro-2- | 7.5 | μM |
| | oxobenzo[d]imidazol-3-yl)piperidine-1-carbothioamide | | |
| 77 | (S)-2-(dibenzylamino)propyl 4-(3-(4-ethoxy-4-oxobutyl)-2-oxo-2,3- | 5.7 | μM |
| | dihydro-1H-benzo[d]imidazol-1-yl)piperidine-1-carboxylate | | |
| 78 | (S)-4-(3-(1-((2-(dibenzylamino)propoxy)carbonyl)piperidin-4-yl)-2- | 1.7 | μM |
| | oxo-2,3-dihydro-1H-benzo[d]imidazol-1-yl)butanoic acid | | |
| 79 | (S)(1-((2-(dibenzylamino)propoxy)carbonyl)piperidin-4-yl)-5,6- | 11.5 | μM |
| | dihydro-imidazo[4,5,1,j,k][1]benzazepine-2,7(1H,4H)-dione | | |
| 43 | 2,2-diphenylethyl 4-(1,2-dihydro-2-oxobenzo[d]imidazol-3- | 12 | μM |
| | yl)piperidine-1-carboxylate | | |
| 73 | (S)-2-(dibenzylamino)propyl 4-(3-(2-tert- | 3.0 | μM |
| | butyloxycarbonylaminoethyl)-2-oxo-2,3-dihydro-1H- | | |
| | benzo[d]imidazol-1-yl)piperidine-1-carboxylate | | |
| 74 | (S)-2-(dibenzylamino)-3-phenylpropyl 4-(3-(2-tert- | 3.2 | μM |
| | butyloxycarbonylaminoethyl)-2-oxo-2,3-dihydro-1H- | | |
| | benzo[d]imidazol-1-yl)piperidine-1-carboxylate | | |
| 76 | (S)-2-(dibenzylamino)-3-phenylpropyl 4-(3-(2 aminoethyl)-2-oxo- | 2.6 | μM |
| | 2,3-dihydro-1H-benzo[d]imidazol-1-yl)piperidine-1-carboxylate D- | | |
| | biotin amid | | |
| 55 | (S)-2-(dibenzylamino)propyl 4-(2-oxo-3-(4-propylphenylsulfonyl)- | 5.5 | μM |
| | 2,3-dihydro-1H-benzo[d]imidazol-1-yl)piperidine-1-carboxylate | | |
| 56 | (S)-2-(dibenzylamino)propyl 4-(3-(4-fluorophenylsulfonyl)-2-oxo- | 1.5 | μM |
| | 2,3-dihydro-1H-benzo[d]imidazol-1-yl)piperidine-1-carboxylate | | |
| 57 | (S)-2-(dibenzylamino)propyl 4-(3-(4-methoxyphenylsulfonyl)-2-oxo- | 1.75 | μM |
| | 2,3-dihydro-1H-benzo[d]imidazol-1-yl)piperidine-1-carboxylate | | |
| 58 | (S)-2-(dibenzylamino)propyl 4-(2-oxo-3-(4- | 8.5 | μM |
| | (trifluoromethyl)phenylsulfonyl)-2,3-dihydro-1H-benzo[d]imidazol- | | |
| | 1-yl)piperidine-1-carboxylate | | |
| 59 | (S)-2-(dibenzylamino)propyl 4-(2-oxo-3-(thiophen-2-ylsulfonyl)-2,3- | 3.8 | μM |
| | dihydro-1H-benzo[d]imidazol-1-yl)piperidine-1-carboxylate | | |
| 61 | (S)-2-(dibenzylamino)propyl 4-(3-(naphthalen-1-ylsulfonyl)-2-oxo- | 2.3 | μM |
| | 2,3-dihydro-1H-benzo[d]imidazol-1-yl)piperidine-1-carboxylate | | |
| 94 | (S)-2-(dibenzylamino)propyl 2-(1-(((S)-2- | 9 | μM |
| | (dibenzylamino)propoxy)carbonyl)piperidin-4-ylamino)-1H- | | |
| | benzo[d]imidazole-1-carboxylate | | |
| 93 | (S)-2-(Dibenzylamino)propyl 4-(1H-benzo[d]imidazol-2- | 0.98 | μM |
| | ylamino)piperidine-1-carboxylate | | |
| 80 | (S)-2-(dibenzylamino)propyl 4-(phenylamino)piperidine-1- | 9.5 | μM |
| | carboxylate | | |
| 81 | (S)-2-(dibenzylamino)propyl 4-(4-methoxyphenylamino)piperidine- | 10 | μM |
| | 1-carboxylate | | |
| 82 | (S)-2-(dibenzylamino)propyl 4-(2,4- | 7.3 | μM |
| | dimethoxyphenylamino)piperidine-1-carboxylate | | |
| 83 | (S)-2-(dibenzylamino)propyl 4-(3,4,5- | 16 | μM |
| | trimethoxyphenylamino)piperidine-1-carboxylate | | |
| 85 | (S)-2-(dibenzylamino)propyl 4-(4-iodophenylamino)piperidine-1- | 7.65 | μM |
| | carboxylate | | |
| 98 | 2-(N-(4-nitrobenzyl)-N-benzylamino)ethyl 4-(1,2-dihydro-2- | 3.33 | μM |
| | oxobenzo[d]imidazol-3-yl)piperidine-1-carboxylate | | |
| 63 | (S)-2-(dibenzylamino)propyl 4-(2-oxo-3-(4- | 3 | μM |
| | acetamidylphenylsulfonyl)-2,3-dihydro-1H-benzo[d]imidazol-1- | | |
| | yl)piperidine-1-carboxylate | | |
| 70 | (S)-2-(dibenzylamino)-3-phenylpropyl 4-(1-benzyl-1,2-dihydro-2- | 3.6 | μM |
| | oxobenzo[d]imidazol-3-yl)piperidine-1-carboxylate | | |
| 37 | (S)-2-(dibenzylamino)-3-phenylpropyl 4-(1-benzyl-1,2-dihydro-2- | 1.8 | μM |
| | oxobenzo[d]imidazol-3-yl)piperidine-1-carboxylate, hydrochloride | | |
| | salt | | |
| 95 | N-((S)-2-(dibenzylamino)propyl)-4-(1,2-dihydro-2- | 11 | μM |
| | oxobenzo[d]imidazol-3-yl)piperidine-1-carboxamidine | | |
| 88 | (S)-2-(dibenzylamino)propyl 4-(1,3-diphenylureido)piperidine-1- | 2.7 | μM |
| | carboxylate | | |
| 89 | (S)-2-(dibenzylamino)propyl 4-(N-phenylbenzamido)piperidine-1- | 2.7 | μM |
| | carboxylate | | |
| 90 | (S)-2-(dibenzylamino)propyl 4-(N- | 1.75 | μM |
| | phenylphenylsulfonamido)piperidine-1-carboxylate | | |
| 84 | (S)-2-(dibenzylamino)propyl 4-(3-fluorophenylamino)piperidine-1- | 5.5 | μM |
| | carboxylate | | |
| 86 | (S)-2-(dibenzylamino)propyl 4-(3,4-dichlorophenylamino)piperidine- | 3.75 | μM |
| | 1-carboxylate | | |
| 87 | (S)-2-(dibenzylamino)propyl 4-(2-aminophenylamino)piperidine-1- | 12.9 | μM |
| | carboxylate | | |
| 153 | (S)-2-(dibenzylamino)propyl 4-(2-phenyl-1H-benzo[d]imidazol-1- | 3.75 | μM |
| | yl)piperidine-1-carboxylate | | |
| 177 | (S)-2-(dibenzylamino)propyl 4-(2-amino-1H-benzo[d]imidazol-1- | 3.8 | μM |
| | yl)piperidine-1-carboxylate | | |
| 155 | (S)-2-(dibenzylamino)propyl 4-(4-fluoro-N- | 0.8 | μM |
| | phenylphenylsulfonamido)piperidine-1-carboxylate | | |
| 156 | (S)-2-(dibenzylamino)propyl 4-(4-methoxy-N- | 4.0 | μM |
| | phenylphenylsulfonamido)piperidine-1-carboxylate | | |
| 159 | (S)-2-(dibenzylamino)propyl 4-(N-phenylthiophene-2- | 1.0 | μM |
| | sulfonamido)piperidine-1-carboxylate | | |
| 160 | (S)-2-(dibenzylamino)propyl 4-(N-phenylquinoline-8- | 2.7 | μM |
| | sulfonamido)piperidine-1-carboxylate | | |
| 178 | 4-(dibenzylamino)phenyl 4-(1,2-dihydro-2-oxobenzo[d]imidazol-3- | 1.3 | μM |
| | yl)piperidine-1-carboxylate | | |
| 163 | (S)-2-(dibenzylamino)propyl 4-(N-phenylpivalamido)piperidine-1- | 3.7 | μM |
| | carboxylate | | |
| 179 | (S)-2-(dibenzylamino)propyl 4-(2-oxo-2,3-dihydro-1H- | 3.25 | μM |
| | benzo[d]imidazol-4-yl)piperidine-1-carboxylate | | |
| 180 | 6-(dibenzylamino)hexyl 4-(1,2-dihydro-2-oxobenzo[d]imidazol-3- | 2.0 | μM |
| | yl)piperidine-1-carboxylate | | |
| 108 | (S)-2-(dibenzylamino)-4-methylpentyl 4-(2-oxo-2,3-dihydro-1H- | 3.75 | μM |
| | benzo[d]imidazol-1-yl)piperidine-1-carboxylate | | |
| 110 | (S)-3-(dibenzylamino)butyl 4-(2-oxo-2,3-dihydro-1H- | 3.6 | μM |
| | benzo[d]imidazol-1-yl)piperidine-1-carboxylate | | |
| 112 | (S)-2-(dibenzylamino)-2-phenylethyl 4-(1,2-dihydro-2- | 3.75 | μM |
| | oxobenzo[d]imidazol-3-yl)piperidine-1-carboxylate | | |
| 181 | Benzyl 4-(1,2-dihydro-2-oxobenzo[d]imidazol-3-yl)piperidine-1- | 0.3 | μM |
| | carboxylate | | |
| 182 | (1-benzylpiperidin-2-yl)methyl 4-(1,2-dihydro-2- | 5 | μM |
| | oxobenzo[d]imidazol-3-yl)piperidine-1-carboxylate | | |
| 165 | (S)-2-(dibenzylamino)propyl 4-(1,3-diphenylthioureido)piperidine-1- | 3.0 | μM |
| | carboxylate | | |
| 114 | 3-(benzyl(4-nitrobenzyl)amino)propyl 4-(2-oxo-2,3-dihydro-1H- | 3.3 | μM |
| | benzo[d]imidazol-1-yl)piperidine-1-carboxylate hydrochloride | | |
| 115 | 2-(benzyl(4-bromobenzyl)amino)ethyl 4-(2-oxo-2,3-dihydro-1H- | 3.2 | μM |
| | benzo[d]imidazol-1-yl)piperidine-1-carboxylate hydrochloride | | |
| 152 | (S)-2-(dibenzylamino)-3-phenylpropyl 4-(phenylamino)piperidine-1- | 3.8 | μM |
| | carboxylate | | |
| 167 | (S)-2-(dibenzylamino)propyl 4-(2-methyl-N- | 3.9 | μM |
| | phenylphenylsulfonamido)piperidine-1-carboxylate | | |
| 168 | (S)-2-(dibenzylamino)propyl 4-(3-methyl-N- | 2.9 | μM |
| | phenylphenylsulfonamido)piperidine-1-carboxylate | | |
| 169 | (S)-2-(dibenzylamino)propyl 4-(N-phenyl-3- | 3.2 | μM |
| | (trifluoromethyl)phenylsulfonamido)piperidine-1-carboxylate | | |
| 172 | (S)-2-(dibenzylamino)propyl 4-(5-(dimethylamino)-N- | 0.212 | μM |
| | phenylnaphthalene-1-sulfonamido)piperidine-1-carboxylate | | |
| 118 | (R)-2-(dibenzylamino)-3-phenylpropyl 4-(1,2-dihydro-2- | 2.9 | μM |
| | oxobenzo[d]imidazol-3-yl)piperidine-1-carboxylate | | |
| 119 | 2-(benzyl(4-fluorobenzyl)amino)ethyl 4-(2-oxo-2,3-dihydro-1H- | 1.4 | μM |
| | benzo[d]imidazol-1-yl)piperidine-1-carboxylate hydrochloride | | |
| 120 | 2-(benzyl(3-phenylpropyl)amino)ethyl 4-(2-oxo-2,3-dihydro-1H- | 1.7 | μM |
| | benzo[d]imidazol-1-yl)piperidine-1-carboxylate hydrochloride | | |
| 121 | 2-(benzyl(4-methoxybenzyl)amino)ethyl 4-(2-oxo-2,3-dihydro-1H- | 5 | μM |
| | benzo[d]imidazol-1-yl)piperidine-1-carboxylate hydrochloride | | |
| 122 | 2-(benzyl(3-methoxybenzyl)amino)ethyl 4-(2-oxo-2,3-dihydro-1H- | 1.75 | μM |
| | benzo[d]imidazol-1-yl)piperidine-1-carboxylate hydrochloride | | |
| 123 | 2-(benzyl(3-phenoxybenzyl)amino)ethyl 4-(2-oxo-2,3-dihydro-1H- | 3.7 | μM |
| | benzo[d]imidazol-1-yl)piperidine-1-carboxylate hydrochloride | | |
| 183 | (S)-2-(dibenzylamino)-3-phenylpropyl 4-(N-phenyl-4- | 3.1 | μM |
| | (trifluoromethyl)phenylsulfonamido)piperidine-1-carboxylate | | |
| 184 | (S)-2-(benzyl(4-fluorobenzyl)amino)propyl 4-(2-oxo-2,3-dihydro- | 3.3 | μM |
| | 1H-benzo[d]imidazol-1-yl)piperidine-1-carboxylate hydrochloride | | |
| 125 | (S)-2-(benzyl(3-phenylpropyl)amino)propyl 4-(2-oxo-2,3-dihydro- | 4.0 | μM |
| | 1H-benzo[d]imidazol-1-yl)piperidine-1-carboxylate hydrochloride | | |
| 133 | (S)-2-(benzyl(quinolin-4-ylmethyl)amino)propyl 4-(2-oxo-2,3- | 1.0 | μM |
| | dihydro-1H-benzo[d]imidazol-1-yl)piperidine-1-carboxylate | | |
| | hydrochloride | | |
| 126 | (S)-2-(benzyl(3-methoxybenzyl)amino)propyl 4-(2-oxo-2,3-dihydro- | 3.75 | μM |
| | 1H-benzo[d]imidazol-1-yl)piperidine-1-carboxylate hydrochloride | | |
| 128 | (S)-2-(benzyl(3-chlorobenzyl)amino)propyl 4-(2-oxo-2,3-dihydro- | 5 | μM |
| | 1H-benzo[d]imidazol-1-yl)piperidine-1-carboxylate hydrochloride | | |
| 131 | 2-(benzyl(quinolin-4-ylmethyl)amino)ethyl 4-(2-oxo-2,3-dihydro- | 1.05 | μM |
| | 1H-benzo[d]imidazol-1-yl)piperidine-1-carboxylate hydrochloride | | |
| 132 | 2-(benzyl((1-methyl-1H-indol-2-yl)methyl)amino)ethyl 4-(2-oxo-2,3- | 1.2 | μM |
| | dihydro-1H-benzo[d]imidazol-1-yl)piperidine-1-carboxylate | | |
| | hydrochloride | | |
| 135 | 3-(benzyl(pyridin-2-ylmethyl)amino)propyl 4-(2-oxo-2,3-dihydro- | 2.3 | μM |
| | 1H-benzo[d]imidazol-1-yl)piperidine-1-carboxylate hydrochloride | | |
| 136 | (S)-2-(benzyl(3-(benzyloxy)benzyl)amino)propyl 4-(2-oxo-2,3- | 0.8 | μM |
| | dihydro-1H-benzo[d]imidazol-1-yl)piperidine-1-carboxylate | | |
| | hydrochloride | | |
| 137 | (S)-2-(benzyl(phenanthren-9-ylmethyl)amino)propyl 4-(2-oxo-2,3- | 0.7 | μM |
| | dihydro-1H-benzo[d]imidazol-1-yl)piperidine-1-carboxylate | | |
| | hydrochloride | | |
| 138 | (S)-2-(benzyl(naphthalen-1-ylmethyl)amino)propyl 4-(2-oxo-2,3- | 3.75 | μM |
| | dihydro-1H-benzo[d]imidazol-1-yl)piperidine-1-carboxylate | | |
| | hydrochloride | | |
| 139 | (S)-2-(benzyl(thiophen-2-ylmethyl)amino)propyl 4-(2-oxo-2,3- | 5 | μM |
| | dihydro-1H-benzo[d]imidazol-1-yl)piperidine-1-carboxylate | | |
| | hydrochloride | | |
| 140 | (S)-2-(benzyl(pyridin-2-ylmethyl)amino)propyl 4-(2-oxo-2,3- | 1.8 | μM |
| | dihydro-1H-benzo[d]imidazol-1-yl)piperidine-1-carboxylate | | |
| | hydrochloride | | |
| 185 | 2-(benzyl(tert-butoxycarbonyl)amino)ethyl 4-(2-oxo-2,3-dihydro-1H- | 2 | μM |
| | benzo[d]imidazol-1-yl)piperidine-1-carboxylate | | |
| 141 | 3-(benzyl(thiophen-2-ylmethyl)amino)propyl 4-(2-oxo-2,3-dihydro- | 4 | μM |
| | 1H-benzo[d]imidazol-1-yl)piperidine-1-carboxylate hydrochloride | | |
| 149 | 3-(benzyl(3-methoxybenzyl)amino)propyl 4-(2-oxo-2,3-dihydro-1H- | 3.5 | μM |
| | benzo[d]imidazol-1-yl)piperidine-1-carboxylate hydrochloride | | |
| 142 | 3-(benzyl(3-chlorobenzyl)amino)propyl 4-(2-oxo-2,3-dihydro-1H- | 1.2 | μM |
| | benzo[d]imidazol-1-yl)piperidine-1-carboxylate hydrochloride | | |
| 144 | 3-(benzyl(naphthalen-1-ylmethyl)amino)propyl 4-(2-oxo-2,3- | 2.7 | μM |
| | dihydro-1H-benzo[d]imidazol-1-yl)piperidine-1-carboxylate | | |
| | hydrochloride | | |
| 145 | 3-(benzyl(quinolin-4-ylmethyl)amino)propyl 4-(2-oxo-2,3-dihydro- | 3.3 | μM |
| | 1H-benzo[d]imidazol-1-yl)piperidine-1-carboxylate hydrochloride | | |
| 186 | 3-(benzyl((1-methyl-1H-indol-2-yl)methyl)amino)propyl 4-(2-oxo- | 4.8 | μM |
| | 2,3-dihydro-1H-benzo[d]imidazol-1-yl)piperidine-1-carboxylate | | |
| | hydrochloride | | |
| 146 | 3-(benzyl(3-phenoxybenzyl)amino)propyl 4-(2-oxo-2,3-dihydro-1H- | 1.5 | μM |
| | benzo[d]imidazol-1-yl)piperidine-1-carboxylate hydrochloride | | |
| 187 | 2-(benzyl(4-(methoxycarbonyl)benzyl)amino)ethyl 4-(2-oxo-2,3- | 2.2 | μM |
| | dihydro-1H-benzo[d]imidazol-1-yl)piperidine-1-carboxylate | | |
| 150 | 2-(benzyl(4-chlorobenzyl)amino)ethyl 4-(2-oxo-2,3-dihydro-1H- | 1.5 | μM |
| | benzo[d]imidazol-1-yl)piperidine-1-carboxylate | | |
| 99 | 2-(benzyl(2-(benzyloxy)benzyl)amino)ethyl 4-(2-oxo-2,3-dihydro- | 3.5 | μM |
| | 1H-benzo[d]imidazol-1-yl)piperidine-1-carboxylate | | |
| 188 | 3-(benzyl(3-(benzyloxy)benzyl)amino)propyl 4-(2-oxo-2,3-dihydro- | 3 | μM |
| | 1H-benzo[d]imidazol-1-yl)piperidine-1-carboxylate | | |
| 105 | (S)-2-(benzyl(tert-butoxycarbonyl)amino)-4-methylpentyl 4-(2-oxo- | 2.2 | μM |
| | 2,3-dihydro-1H-benzo[d]imidazol-1-yl)piperidine-1-carboxylate | | |
| 189 | (S)-2-(benzylamino)-4-methylpentyl 4-(2-oxo-2,3-dihydro-1H- | 2.3 | μM |
| | benzo[d]imidazol-1-yl)piperidine-1-carboxylate | | |
| 190 | 2-(benzylamino)-3-phenylpropyl 4-(2-oxo-2,3-dihydro-1H- | 0.8 | μM |
| | benzo[d]imidazol-1-yl)piperidine-1-carboxylate | | |

1. A compound of general formula (I) below: 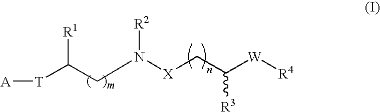
or a pharmaceutically acceptable salt thereof, wherein:
T is selected from the group consisting of —CH₂—, —CH₂CH₂—, —O—, —NH—, a simple bond and a double bond;
m and n, independently, are an integer equal to 0, 1, 2, 3 or 4;
R¹ and R² simultaneously represent an hydrogen atom or R¹ and R² form together an alkyl chain having from 2 or 3 carbon atoms with possibly on insaturation;
R³ represents hydrogen or a group selected among alkyl, alkenyl, cycloalkyl, aryl, arylalkyl, heteroalkyl, heteroaryl and heteroarylalkyl groups wherein said groups defined for R³ are optionally substituted with one or more groups independently selected from halogen, trifluoromethyl, difluoromethyl, azido, alkyl, alkoxy, cyano, nitro and carbethoxy;
R⁴ represents hydrogen or a group selected among alkyl, alkenyl, cycloalkyl, aryl, arylalkyl, heteroaryl, heteroarylalkyl and heteroalkyl, wherein said groups defined for R⁴ are optionally substituted with one or more groups independently selected from halogen, trifluoromethyl, difluoromethyl, azido, alkyl, alkoxy, cyano, nitro, carboxy and carbethoxy;
X represents a single bond, —CH₂— or a functional group chosen among the groups of formulas (X-1) to (X-6) below: 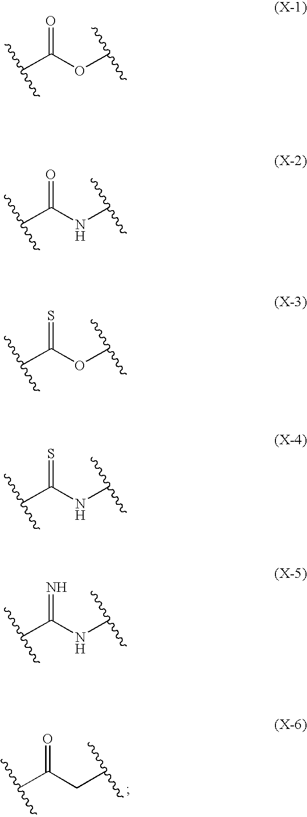
A represents a cyclic moiety selected from the group consisting of:
i) a moiety of formula (A-1) below: 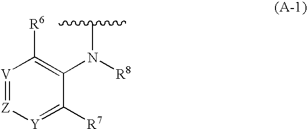
in which:
V, Y and Z, identical or different, represent —CH—, C—R⁶ or N, it being understood that V can also form, together with R⁶ or Z, a fused aryl or heteroaryl ring depending of the nature of R⁶ or Z;
Het represents an oxygen atom or a sulphur atom;
R⁶ represents hydrogen, halogen, a group selected from the group consisting in alkoxy, alkyl, nitro, alkenyl, alkynyl, cycloalkyl, aryl, arylalkyl, heteroaryl and heteroalkyl, wherein said alkoxy, alkyl, alkenyl, alkynyl cycloalkyl, aryl, arylalkyl, heteroaryl and heteroalkyl groups are optionally substituted with one or more groups independently selected from halogen, trifluoromethyl, difluoromethyl, azido, alkyl, alkoxy, amino, cyano and nitro;
R⁷ represents a hydrogen atom, a halogen atom, or a group selected among alkyl, nitro, alkoxy, aminoalkyl, aryl and heteroaryl groups;
R⁸ represents a hydrogen atom, an alkyl group, an alkenyl group, an aryl group, a heteroaryl group or a group selected from the groups having the following formulas —COOR^{S}, —C(O)NR⁹R¹⁰, —C(S)NR⁹R¹⁰, —C(O)NHR⁹, —C(O)R⁹, —SO₂R⁹ in which R⁹ and R¹⁰, which may be identical or different, represent a hydrogen atom, an alkoxy group, an amino group, an alkyl group, an alkenyl group, an aryl group and a heteroaryl group;
R⁷ and R⁸, together, can also form a moiety of formula —C(=Het)-N(R¹¹)— in which Het represents an oxygen atom or a sulphur atom and R¹¹ represents a hydrogen atom, a linear or branched alkyl or alkenyl group; a heteroaryl group, a cycloalkyl group, an aryl group and an arylalkyl group, wherein said groups are optionally substituted with one or more groups independently selected from halogen, trifluoromethyl, difluoromethyl, azido, alkyl, alkoxy, cyano, nitro; a SO₂R¹², a COR¹² group or a COOR¹² group wherein R¹² represents a group chosen among alkyl, alkenyl, cycloalkyl, aryl, heteroaryl, arylalkyl and heteroalkyl, wherein said groups mentioned for R¹² are optionally substituted with one or more groups independently selected from halogen, trifluoromethyl, difluoromethyl, azido, alkyl, alkoxy, cyano, nitro, carboxy and carbethoxy; a group of formula-alkylC(O)R¹³ in which R¹³ represents hydrogen or an alkoxy group; a group of formula alkyl-NHR¹⁴ in which R¹⁴ represents an alkoxycarbonyl group or a biotine moiety; R¹¹ may also form, together with Y when Y is a carbon atom, a saturated heterocarbonated ring comprising 3 or 4 carbon atoms in addition to the carbon atom of Y, said ring being optionally substituted with an oxo functional group;
ii) a moiety of formula (A-2) below: 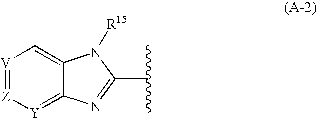
in which:
V, Z, and Y can take the same significations as the moiety of formula (A-1), and
R¹⁵ represents a hydrogen atom, an alkyl or a benzyl radical or a group of formula —NC(O)—OCH₂—CH(CH₃)—N-(Bz)₂ in which Bz stands for benzyl;
W represents C—R¹⁶, N—R¹⁶ or O—R¹⁶ wherein R¹⁶ represents a group selected among alkyl, alkenyl, cycloalkyl, aryl, arylalkyl heteroaryl and heteroalkyl, wherein said groups defined for R¹⁶ are optionally substituted with one or more groups independently selected from halogen, trifluoromethyl, difluoromethyl, azido, alkyl, alkoxy, cyano, nitro, carboxy and carbethoxy; W may also represents a N-containing ring selected among moieties having the formulas W-2, W-3 and W-4 below: 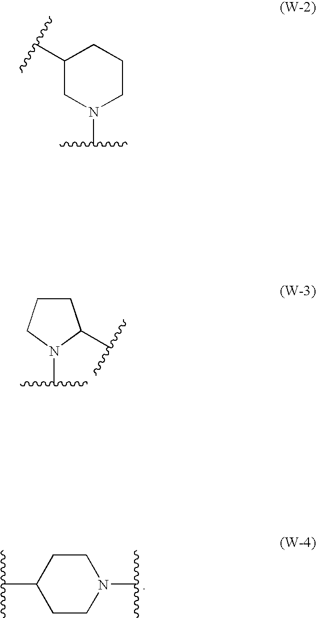
with the proviso that when A represents a moiety of formula (A-1) in which R⁸ is a group of formula —SO₂R¹² in which R¹² stands for 4-chlorophenyl, then R⁷ is different from a fluorine atom.

2. The compound of claim 1, wherein R¹ and R², together with the carbon atom (for R¹), the nitrogen atom (for R²) and the chain bonding the carbon atom bearing R¹ and the nitrogen atom bearing R² form a moiety selected from the group consisting of the following moieties: 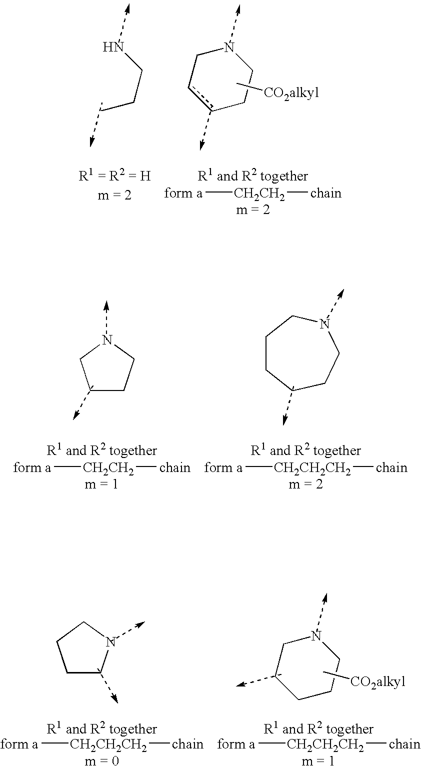
wherein the dashed arrows represent the attachment point of these moieties to T and X respectively via a covalent bond.

3. The compound of claim 1, wherein when R⁸ and R⁷ together form a moiety of formula —C(=Het)-N(R¹¹)—, (A-1) has the particular configuration (A-1-a): 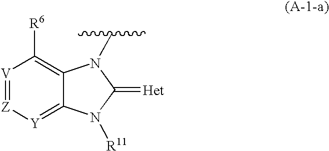
in which V, Z, Y, R⁶, Het and R¹¹ have the same significations as above-mentioned for (A-1) in claim 1.

4. The compound according to claim 1, wherein linear and branched alkyl groups are chosen among (C₁₋C₄)alkyl groups and wherein the linear and branched alkoxy groups are chosen among (C₁₋C₄)alkoxy groups.

5. The compound of claim 4, wherein (C₁₋C₄)alkyl groups are chosen among methyl, ethyl, n-propyl, isopropyl, n-butyl, tent-butyl and isobutyl radicals; whereas (C₁-C₄)alkoxy groups are chosen among methoxy, ethoxy, n-propyloxy, iso-propyloxy, n-butyloxy, tert-butyloxy and isobutyloxy radicals.

6. The compound of claim 1, wherein halogen atoms are chosen among bromine, chlorine, fluorine and iodine.

7. The compound of claim 1, wherein heteroaryl groups represents furan, pyridine, pyrrole, thiophene, imidazole, pyrazole, oxazole, isoxazole, thiazole, benzene, pyridine, pyrazine, pyrimidine, pyridazine, benzylcyclobutene, pentalene, benzofurane, isobenzofurane, indole, isoindole, benzothiophene, benzo[c]thiophene, benzimidazole, indazole, benzoxazole, benzisoxazole, benzothiazole, naphthalene, quinoline, isoquinoline, quinoxaline, quinazoline, cinnoline, purine, anthracene or acridine.

8. The compound of claim 1, wherein said compound is selected from the group consisting of:
(S)-2-(dibenzylamino)propyl 4-(1,2-dihydro-2-oxobenzo[d]imidazol-3-yl)piperidine-1-carboxylate;
(S)-2-(dibenzylamino)-3-phenylpropyl 4-(1,2-dihydro-2-oxobenzo[d]imidazol-3-yl)piperidine-1-carboxylate;
2-(dibenzylamino)ethyl 4-(1,2-dihydro-2-oxobenzo[d]imidazol-3-yl)piperidine-1-carboxylate;
3-(dibenzylamino)propyl 4-(1,2-dihydro-2-oxobenzo[d]imidazol-3-yl)piperidine-1-carboxylate;
2-(N-benzyl-N-methylamino)ethyl 4-(1,2-dihydro-2-oxobenzo[d]imidazol-3-yl)piperidine-1-carboxylate;
(S)-2-(dibenzylamino)propyl 5,6-dihydro-4-(1,2-dihydro-2-oxobenzo[d]imidazol-3-yl)pyridine-1(2H)-carboxylate;
2-(benzyloxy)ethyl 4-(1,2-dihydro-2-oxobenzo[d]imidazol-3-yl)piperidine-1-carboxylate;
(S)-2-(dibenzylamino)propyl 4-(1,2-dihydro-2-thioxobenzo[d]imidazol-3-yl)piperidine-1-carboxylate;
(S)-2-(dibenzylamino)propyl 4-(2-oxobenzo[d]oxazol-3(2H)-yl)piperidine-1-carboxylate;
(S)-2-(dibenzylamino)propyl 4-(1H-benzo[d]imidazol-1-yl)piperidine-1-carboxylate;
(S)-2-(dibenzylamino)propyl 4-(1-methyl-1H-benzo[d]imidazol-2-yloxy)piperidine-1-carboxylate;
(S)-2-(dibenzylamino)propyl 3-(1,2-dihydro-2-oxobenzo[d]imidazol-3-yl)pyrrolidine-1-carboxylate;
tert-butyl 3-(1-(((S)-2-(dibenzylamino)propoxy)carbonyl)piperidin-4-yl)-2,3-dihydro-2-oxobenzo[d]imidazole-1-carboxylate;
1-(1-(3-(dibenzylamino)propyl)piperidin-4-yl)-1H-benzo[d]-imidazol-2(3H)-one;
(S)-2-(dibenzylamino)propyl 4-(1,2-dihydro-1-methyl-2-oxobenzo-[d]imidazol-3-yl)piperidine-1-carboxylate;
1-(1-(2-(dibenzylamino)ethyl)piperidin-4-yl)-1H-benzo[d]imidazol-2(3H)-one;
1-(1-((S)-2-(dibenzylamino)-3-phenylpropyl)piperidin-4-yl)-1H-benzo[d]imidazol-2(3H)-one;
1-(1-((S)-2-(dibenzylamino)propyl)piperidin-4-yl)-1H-benzo[d]imidazol-2(3H)-one;
O-2-(dibenzylamino)ethyl 4-(1,2-dihydro-2-oxobenzo[d]imidazol-3-yl)piperidine-1-carbothioate;
O-2-(benzyloxy)ethyl 4-(1,2-dihydro-2-oxobenzo[d]imidazol-3-yl)piperidine-1-carbothioate;
O—(S)-2-(dibenzylamino)propyl 4-(1,2-dihydro-2-oxobenzo[d]imidazol-3-yl)piperidine-1-carbothioate;
(S)-2-(dibenzylamino)propyl 4-((1,2-dihydro-2-oxobenzo[d]imidazol-3-yl)methyl)piperidine-1-carboxylate;
(S)-2-(dibenzylamino)propyl 3-(1,2-dihydro-2-oxobenzo[d]imidazol-3-yl)propylcarbamate;
(S)-2-(dibenzylamino)propyl 4-(2,3-dihydro-2-oxoimidazo[4,5-b]pyridin-1-yl)piperidine-1-carboxylate;
1-benzylpiperidin-3-yl 4-(1,2-dihydro-2-oxobenzo[d]imidazol-3-yl)piperidine-1-carboxylate;
(S)-2-(dibenzylamino)propyl 3-(1,2-dihydro-2-oxobenzo[d]imidazol-3-yl)piperidine-1-carboxylate;
(S)-2-(dibenzylamino)propyl 4-(1-benzyl-1,2-dihydro-2-oxobenzo[d]imidazol-3-yl)piperidine-1-carboxylate;
(S)-2-(dibenzylamino)propyl 4-(1-(cyclohexylmethyl)-1,2-dihydro-2-oxobenzo[d]imidazol-3-yl)piperidine-1-carboxylate;
(S)-2-(dibenzylamino)propyl 4-(2-oxo-3-methylsulfonyl-2,3-dihydro-1H-benzo[d]imidazol-1-yl)piperidine-1-carboxylate;
(S)-2-(dibenzylamino)propyl 4-(2-methyl-1H-benzo[d]imidazol-1-yl)piperidine-1-carboxylate;
1-(S)-2-(dibenzylamino)propyl 3-ethyl 4-(1,2-dihydro-2-oxobenzo[d]imidazol-3-yl)piperidine-1,3-dicarboxylate;
(S)-2-(dibenzylamino)propyl 4-(2-oxo-3-phenylsulfonyl-2,3-dihydro-1H-benzo[d]imidazol-1-yl)piperidine-1-carboxylate;
(S)-2-(dibenzylamino)propyl 4-(1,2-dihydro-2-oxo-1-(benzoyl)benzo[d]imidazol-3-yl)piperidine-1-carboxylate;
(S)-2-(dibenzylamino)propyl 4-(6-chloro-1,2-dihydro-2-oxobenzo[d]imidazol-3-yl)piperidine-1-carboxylate;
(S)-2-(dibenzylamino)propyl 4(3,4-dihydroquinolin-1(2H)-yl)piperidine-1-carboxylate;
(S)-2-(dibenzylamino)propyl 4-(1,2-dihydro-2-oxobenzo[d]imidazol-3-yl)azepane-1-carboxylate;
2-(diethylamino)ethyl 4-(1,2-dihydro-2-oxobenzo[d]imidazol-3-yl)piperidine-1-carboxylate;
(S)-2-(dibenzylamino)propyl 4-(1,2-dihydro-2-oxo-1-(pivaloyl)benzo[d]imidazol-3-yl)piperidine-1-carboxylate;
2-(diisopropylamino)ethyl 4-(1,2-dihydro-2-oxobenzo[d]imidazol-3-yl)piperidine-1-carboxylate;
2-(1-benzylpiperidin-4-yl)ethyl 4-(1,2-dihydro-2-oxobenzo[d]imidazol-3-yl)piperidine-1-carboxylate;
2-(ethyl(phenyl)amino)ethyl 4-(2-oxo-2,3-dihydro-1H-benzo[d]imidazol-1-yl)piperidine-1-carboxylate;
((S)-1-benzylpyrrolidin-2-yl)methyl 4-(1,2-dihydro-2-oxobenzo[d]imidazol-3-yl)piperidine-1-carboxylate;
2-(dimethylamino)ethyl 4-(1,2-dihydro-2-oxobenzo[d]imidazol-3-yl)piperidine-1-carboxylate;
3,3-diphenylpropyl 4-(1,2-dihydro-2-oxobenzo[d]imidazol-3-yl)piperidine-1-carboxylate;
(S)-2-(dibenzylamino)propyl 2-((1,2-dihydro-2-oxobenzo[d]imidazol-3-yl)methyl)pyrrolidine-1-carboxylate;
(S)-2-(dibenzylamino)propyl 4-(2-(1,2-dihydro-2-oxobenzo[d]imidazol-3-yl)ethyl)piperidine-1-carboxylate;
(S)-2-(dibenzylamino)propyl 4-(1,2-dihydro-2-oxonaphtho[2,3-d]imidazol-3-yl)piperidine-1-carboxylate;
(S)-2-(dibenzylamino)propyl 4-(1H-indol-3-yl)piperidine-1-carboxylate;
(S)-2-(dibenzylamino)propyl 4-(N-phenylpropionamido)piperidine-1-carboxylate;
(S)-2-(dibenzylamino)propyl 4-(1,2-dihydro-2-oxo-1-phenylbenzo[d]imidazol-3-yl)piperidine-1-carboxylate;
1-(1-(4-(dibenzylamino)butyl)piperidin-4-yl)-1H-benzo[d]imidazol-2(3H)-one;
(S)-2-(dibenzylamino)-3-phenylpropyl 4-(1,2-dihydro-1-methyl-2-oxobenzo[d]imidazol-3-yl)piperidine-1-carboxylate;
(S)-2-(dibenzylamino)-3-phenylpropyl 4-(1,2-dihydro-3-phenylsulfonyl-2-oxobenzo[d]imidazol-1-yl)piperidine-1-carboxylate;
N-((S)-2-(dibenzylamino)propyl)-4-(1,2-dihydro-2-oxobenzo[d]imidazol-3-yl)piperidine-1-carboxamide;
N-((S)-2-(dibenzylamino)propyl)-4-(1,2-dihydro-2-oxobenzo[d]imidazol-3-yl)piperidine-1-carbothioamide;
(S)-2-(dibenzylamino)propyl 4-(3-(4-ethoxy-4-oxobutyl)-2-oxo-2,3-dihydro-1H-benzo[d]imidazol-1-yl)piperidine-1-carboxylate;
(S)-4-(3-(14(2-(dibenzylamino)propoxy)carbonyl)piperidin-4-O-2-oxo-2,3-dihydro-1H-benzo[d]imidazol-1-yl)butanoic acid;
2,2-diphenylethyl 4-(1,2-dihydro-2-oxobenzo[d]imidazol-3-yl)piperidine-1-carboxylate;
(S)-2-(dibenzylamino)propyl 4-(2-oxo-3-(4-propylphenylsulfonyl)-2,3-dihydro-1H-benzo[d]imidazol-1-yl)piperidine-1-carboxylate;
(S)-2-(dibenzylamino)propyl 4-(3-(4-fluorophenylsulfonyl)-2-oxo-2,3-dihydro-1H-benzo[d]imidazol-1-yl)piperidine-1-carboxylate;
(S)-2-(dibenzylamino)propyl 4-(3-(4-methoxyphenylsulfonyl)-2-oxo-2,3-dihydro-1H-benzo[d]imidazol-1-yl)piperidine-1-carboxylate;
(S)-2-(dibenzylamino)propyl 4-(2-oxo-3-(4-(trifluoromethyl)phenylsulfonyl)-2,3-dihydro-1H-benzo[d]imidazol-1-yl)piperidine-1-carboxylate;
(S)-2-(dibenzylamino)propyl 4-(2-oxo-3-(thiophen-2-ylsulfonyl)-2,3-dihydro-1H-benzo[d]imidazol-1-yl)piperidine-1-carboxylate;
(S)-2-(dibenzylamino)propyl 4-(2-oxo-3-(quinolin-8-ylsulfonyl)-2,3-dihydro-1H-benzo[d]imidazol-1-yl)piperidine-1-carboxylate;
(S)-2-(dibenzylamino)propyl 4-(3-(naphthalen-1-ylsulfonyl)-2-oxo-2,3-dihydro-1H-benzo[d]imidazol-1-yl)piperidine-1-carboxylate;
(S)-2-(dibenzylamino)propyl 2-(1-(((S)-2-(dibenzylamino)propoxy)carbonyl)piperidin-4-ylamino)-1H-benzo[d]imidazole-1-carboxylate;
(S)-2-(dibenzylamino)propyl 4-(1H-benzo[d]imidazol-2-ylamino)piperidine-1-carboxylate;
(S)-2-(dibenzylamino)propyl 4-(phenylamino)piperidine-1-carboxylate;
(S)-2-(dibenzylamino)propyl 4-(4-methoxyphenylamino)piperidine-1-carboxylate;
(S)-2-(dibenzylamino)propyl 4-(2,4-dimethoxyphenylamino)piperidine-1-carboxylate;
(S)-2-(dibenzylamino)propyl 4-(3,4,5-trimethoxyphenylamino)piperidine-1-carboxylate;
(S)-2-(dibenzylamino)propyl 4-(4-iodophenylamino)piperidine-1-carboxylate;
2-(N-(4-nitrobenzyl)-N-benzylamino)ethyl 4-(1,2-dihydro-2-oxobenzo[d]imidazol-3-yl)piperidine-1-carboxylate;
(S)-2-(dibenzylamino)propyl 4-(2-oxo-3-(4-acetamidyl-phenylsulfonyl)-2,3-dihydro-1H-benzo[d]imidazol-1-yl)piperidine-1-carboxylate;
(S)-2-(dibenzylamino)-3-phenylpropyl 4-(1-benzyl-1,2-dihydro-2-oxobenzo[d]imidazol-3-yl)piperidine-1-carboxylate;
N-((S)-2-(dibenzylamino)propyl)-4-(1,2-dihydro-2-oxobenzo[d]imidazol-3-yl)piperidine-1-carboxamidine;
(S)-2-(dibenzylamino)propyl 4-(1,3-diphenylureido)piperidine-1-carboxylate;
(S)-2-(dibenzylamino)propyl 4-(N-phenylbenzamido)piperidine-1-carboxylate;
(S)-2-(dibenzylamino)propyl 4-(N-phenylphenylsulfonamido) piperidine-1-carboxylate;
(S)-2-(dibenzylamino)propyl 4-(3-fluorophenylamino)piperidine-1-carboxylate;
(S)-2-(dibenzylamino)propyl 4-(3,4-dichlorophenylamino)piperidine-1-carboxylate;
(S)-2-(dibenzylamino)propyl 4-(2-aminophenylamino)piperidine-1-carboxylate;
(S)-2-(dibenzylamino)propyl 4-(1-acetyl-1,2-dihydro-2-oxobenzo[d]imidazol-3-yl)piperidine-1-carboxylate;
(S)(1-((2-(dibenzylamino)propoxy)carbonyl)piperidin-4-yl)-5,6-dihydro-imidazo[4,5,1,j,k][1]benzazepine-2,7(1H,4H)-dione;
(S)-2-(dibenzylamino)propyl 4-(3-(2-tert-butyloxycarbonylaminoethyl)-2-oxo-2,3-dihydro-1H-benzo[d]imidazol-1-yl)piperidine-1-carboxylate;
(S)-2-(dibenzylamino)-3-phenylpropyl 4-(3-(2-tert-butyloxy carbonylaminoethyl)-2-oxo-2,3-dihydro-1H-benzo[d]imidazol-1-yl)piperidine-1-carboxylate;
(S)-2-(dibenzylamino)-3-phenylpropyl 4-(3-(2 aminoethyl)-2-oxo-2,3-dihydro-1H-benzo[d]imidazol-1-yl)piperidine-1-carboxylate D-biotin amid;
(S)-2-(dibenzylamino)propyl 3-(1,2-dihydro-2-oxobenzo[d]imidazol-3-yl)pyrrolidine-1-carboxylate;
1-[1-(4-dibenzylamino-butyryl)-piperidin-4-yl]-1,3-dihydro-benzoimidazol-2-one;
2-(benzyl(2-(benzyloxy)benzyl)amino)ethyl 4-(2-oxo-2,3-dihydro-1H-benzo[d]imidazol-1-yl)piperidine-1-carboxylate;
2-(benzyl(4-(benzyloxy)benzyl)amino)ethyl 4-(2-oxo-2,3-dihydro-1H-benzo[d]imidazol-1-yl)piperidine-1-carboxylate;
2-(benzyl(2,6-difluorobenzyl)amino)ethyl 4-(2-oxo-2,3-dihydro-1H-benzo[d]imidazol-1-yl)piperidine-1-carboxylate;
(S)-2-(benzyl(furan-2-ylmethyl)amino)propyl 4-(2-oxo-2,3-dihydro-1H-benzo[d]imidazol-1-yl)piperidine-1-carboxylate;
2-(benzyl(3-(benzyloxy)benzyl)amino)ethyl 4-(2-oxo-2,3-dihydro-1H-benzo[d]imidazol-1-yl)piperidine-1-carboxylate;
(E)-2-(benzyl(cinnamyl)amino)ethyl 4-(2-oxo-2,3-dihydro-1H-benzo[d]imidazol-1-yl)piperidine-1-carboxylate;
(S)-2-(benzyl(tert-butoxycarbonyl)amino)-4-methylpentyl 4-(2-oxo-2,3-dihydro-1H-benzo[d]imidazol-1-yl)piperidine-1-carboxylate;
(S)-2-(benzyl(tert-butoxycarbonyl)amino)-3-phenylpropyl 4-(2-oxo-2,3-dihydro-1H-benzo[d]imidazol-1-yl)piperidine-1-carboxylate;
(S)-2-(dibenzylamino)-3-methylbutyl 4-(1,2-dihydro-2-oxobenzo[d]imidazol-3-yl)piperidine-1-carboxylate;
(S)-2-(dibenzylamino)-4-methylpentyl 4-(1,2-dihydro-2-oxobenzo[d]imidazol-3-yl)piperidine-1-carboxylate;
2-(dibenzylamino)-3,3-dimethylbutyl 4-(1,2-dihydro-2-oxobenzo[d]imidazol-3-yl)piperidine-1-carboxylate;
(3-(dibenzylamino)butyl 4-(1,2-dihydro-2-oxobenzo[d]imidazol-3-yl)piperidine-1-carboxylate;
2-(dibenzylamino)-2-methylpropyl 4-(1,2-dihydro-2-oxobenzo[d]imidazol-3-yl)piperidine-1-carboxylate;
(S)-2-(dibenzylamino)-2-phenylethyl 4-(2-oxo-2,3-dihydro-1H-benzo[d]imidazol-1-yl)piperidine-1-carboxylate;
(S)-2-(benzyl(4-nitrobenzyl)amino)propyl 4-(2-oxo-2,3-dihydro-1H-benzo[d]imidazol-1-yl)piperidine-1-carboxylate;
3-(benzyl(4-nitrobenzyl)amino)propyl 4-(2-oxo-2,3-dihydro-1H-benzo[d]imidazol-1-yl)piperidine-1-carboxylate;
2-(benzyl(4-bromobenzyl)amino)ethyl 4-(2-oxo-2,3-dihydro-1H-benzo[d]imidazol-1-yl)piperidine-1-carboxylate;
S)-2-(benzyl(4-bromobenzyl)amino)propyl 4-(2-oxo-2,3-dihydro-1H-benzo[d]imidazol-1-yl)piperidine-1-carboxylate;
3-(benzyl(4-methoxybenzyl)amino)propyl 4-(2-oxo-2,3-dihydro-1H-benzo[d]imidazol-1-yl)piperidine-1-carboxylate;
(R)-2-(dibenzylamino)-3-phenylpropyl 4-(2-oxo-2,3-dihydro-1H-benzo[d]imidazol-1-yl)piperidine-1-carboxylate;
2-(benzyl(4-fluorobenzyl)amino)ethyl 4-(2-oxo-2,3-dihydro-1H-benzo[d]imidazol-1-yl)piperidine-1-carboxylate;
2-(benzyl(3-phenylpropyl)amino)ethyl 4-(2-oxo-2,3-dihydro-1H-benzo[d]imidazol-1-yl)piperidine-1-carboxylate;
2-(benzyl(4-methoxybenzyl)amino)ethyl 4-(2-oxo-2,3-dihydro-1H-benzo[d]imidazol-1-yl)piperidine-1-carboxylate;
2-(benzyl(3-methoxybenzyl)amino)ethyl 4-(2-oxo-2,3-dihydro-1H-benzo[d]imidazol-1-yl)piperidine-1-carboxylate;
2-(benzyl(3-phenoxybenzyl)amino)ethyl 4-(2-oxo-2,3-dihydro-1H-benzo[d]imidazol-1-yl)piperidine-1-carboxylate;
2-(benzyl(3-chlorobenzyl)amino)ethyl 4-(2-oxo-2,3-dihydro-1H-benzo[d]imidazol-1-yl)piperidine-1-carboxylate;
(S)-2-(benzyl(3-phenylpropyl)amino)propyl 4-(2-oxo-2,3-dihydro-1H-benzo[d]imidazol-1-yl)piperidine-1-carboxylate;
(S)-2-(benzyl(3-methoxybenzyl)amino)propyl 4-(2-oxo-2,3-dihydro-1H-benzo[d]imidazol-1-yl)piperidine-1-carboxylate;
(S)-2-(benzyl(3-phenoxybenzyl)amino)propyl 4-(2-oxo-2,3-dihydro-1H-benzo[d]imidazol-1-yl)piperidine-1-carboxylate;
(S)-2-(benzyl(3-chlorobenzyl)amino)propyl 4-(2-oxo-2,3-dihydro-1H-benzo[d]imidazol-1-yl)piperidine-1-carboxylate;
(S)-2-(benzyl(4-chlorobenzyl)amino)propyl 4-(2-oxo-2,3-dihydro-1H-benzo[d]imidazol-1-yl)piperidine-1-carboxylate;
2-(benzyl(naphthalen-1-ylmethyl)amino)ethyl 4-(2-oxo-2,3-dihydro-1H-benzo[d]imidazol-1-yl)piperidine-1-carboxylate;
2-(benzyl(quinolin-4-ylmethyl)amino)ethyl 4-(2-oxo-2,3-dihydro-1H-benzo[d]imidazol-1-yl)piperidine-1-carboxylate;
2-(benzyl((1-methyl-1H-indol-2-yl)methyl)amino)ethyl 4-(2-oxo-2,3-dihydro-1H-benzo[d]imidazol-1-yl)piperidine-1-carboxylate;
(S)-2-(benzyl(quinolin-4-ylmethyl)amino)propyl 4-(2-oxo-2,3-dihydro-1H-benzo[d]imidazol-1-yl)piperidine-1-carboxylate;
3-(benzyl(pyridin-3-ylmethyl)amino)propyl 4-(2-oxo-2,3-dihydro-1H-benzo[d]imidazol-1-yl)piperidine-1-carboxylate;
3-(benzyl(pyridin-2-ylmethyl)amino)propyl 4-(2-oxo-2,3-dihydro-1H-benzo[d]imidazol-1-yl)piperidine-1-carboxylate;
(S)-2-(benzyl(3-(benzyloxy)benzyl)amino)propyl 4-(2-oxo-2,3-dihydro-1H-benzo[d]imidazol-1-yl)piperidine-1-carboxylate;
(S)-2-(benzyl(phenanthren-9-ylmethyl)amino)propyl 4-(2-oxo-2,3-dihydro-1H-benzo[d]imidazol-1-yl)piperidine-1-carboxylate;
(S)-2-(benzyl(naphthalen-1-ylmethyl)amino)propyl 4-(2-oxo-2,3-dihydro-1H-benzo[d]imidazol-1-yl)piperidine-1-carboxylate;
(S)-2-(benzyl(thiophen-2-ylmethyl)amino)propyl 4-(2-oxo-2,3-dihydro-1H-benzo[d]imidazol-1-yl)piperidine-1-carboxylate;
(S)-2-(benzyl(pyridin-2-ylmethyl)amino)propyl 4-(2-oxo-2,3-dihydro-1H-benzo[d]imidazol-1-yl)piperidine-1-carboxylate;
3-(benzyl(thiophen-2-ylmethyl)amino)propyl 4-(2-oxo-2,3-dihydro-1H-benzo[d]imidazol-1-yl)piperidine-1-carboxylate;
3-(benzyl(3-chlorobenzyl)amino)propyl 4-(2-oxo-2,3-dihydro-1H-benzo[d]imidazol-1-yl)piperidine-1-carboxylate;
3-(benzyl(2-(benzyloxy)ethyl)amino)propyl 4-(2-oxo-2,3-dihydro-1H-benzo[d]imidazol-1-yl)piperidine-1-carboxylate;
3-(benzyl(naphthalen-1-ylmethyl)amino)propyl 4-(2-oxo-2,3-dihydro-1H-benzo[d]imidazol-1-yl)piperidine-1-carboxylate;
3-(benzyl(quinolin-4-ylmethyl)amino)propyl 4-(2-oxo-2,3-dihydro-1H-benzo[d]imidazol-1-yl)piperidine-1-carboxylate;
3-(benzyl(3-phenoxybenzyl)amino)propyl 4-(2-oxo-2,3-dihydro-1H-benzo[d]imidazol-1-yl)piperidine-1-carboxylate;
3-(benzyl(4-fluorobenzyl)amino)propyl 4-(2-oxo-2,3-dihydro-1H-benzo[d]imidazol-1-yl)piperidine-1-carboxylate;
3-(benzyl(3-phenylpropyl)amino)propyl 4-(2-oxo-2,3-dihydro-1H-benzo[d]imidazol-1-yl)piperidine-1-carboxylate;
3-(benzyl(3-methoxybenzyl)amino)propyl 4-(2-oxo-2,3-dihydro-1H-benzo[d]imidazol-1-yl)piperidine-1-carboxylate;
2-(benzyl(4-chlorobenzyl)amino)ethyl 4-(2-oxo-2,3-dihydro-1H-benzo[d]imidazol-1-yl)piperidine-1-carboxylate;
2-(benzyl(2-methoxybenzyl)amino)ethyl 4-(2-oxo-2,3-dihydro-1H-benzo[d]imidazol-1-yl)piperidine-1-carboxylate;
(S)-2-(dibenzylamino)-3-phenylpropyl 4-(phenylamino)piperidine-1-carboxylate;
(S)-2-(dibenzylamino)propyl 4-(2-phenyl-1H-benzo[d]imidazol-1-yl)piperidine-1-carboxylate;
(S)-2-(dibenzylamino)propyl 4-(N-phenyl-4-propylphenylsulfonamido)piperidine-1-carboxylate;
(S)-2-(dibenzylamino)propyl 4-(N-phenyl-4-fluorophenylsulfonamido)piperidine-1-carboxylate;
(S)-2-(dibenzylamino)propyl 4-(N-phenyl-4-methoxyphenylsulfonamido)piperidine-1-carboxylate;
(S)-2-(dibenzylamino)-3-phenylpropyl 4-(N-phenyl-4-methoxyphenylsulfonamido)piperidine-1-carboxylate;
(S)-2-(dibenzylamino)propyl 4-(N-phenyl-4-(trifluoromethyl)phenylsulfonamido)piperidine-1-carboxylate;
(S)-2-(dibenzylamino)propyl 4-(N-phenylthiophen-2-ylsulfonamido)piperidine-1-carboxylate;
(S)-2-(dibenzylamino)propyl 4-(N-phenylquinolin-8-ylsulfonamido)piperidine-1-carboxylate;
(S)-2-(dibenzylamino)propyl 4-(N-phenyl-naphthalen-1-ylsulfonamido)piperidine-1-carboxylate;
(S)-2-(dibenzylamino)-3-phenylpropyl 4-(N-phenyl-naphthalen-1-ylsulfonamido)piperidine-1-carboxylate;
(S)-2-(dibenzylamino)propyl 4-(N-phenylpivalamido)piperidine-1-carboxylate;
(S)-2-(dibenzylamino)propyl 4-(2-(phenylsulfonyl)phenylamino)piperidine-1-carboxylate;
(S)-2-(dibenzylamino)propyl 4-(1,3-diphenylthioureido)piperidine-1-carboxylate;
(S)-2-(dibenzylamino)propyl 4-(N-methyl-N-phenylamino)piperidine-1-carboxylate;
(S)-2-(dibenzylamino)propyl 4-(N-phenyl-2-methylphenylsulfonamido)piperidine-1-carboxylate;
(S)-2-(dibenzylamino)propyl 4-(N-phenyl-3-methylphenylsulfonamido)piperidine-1-carboxylate;
(S)-2-(dibenzylamino)propyl 4-(N-phenyl-3-(trifluoromethyl)phenylsulfonamido)piperidine-1-carboxylate;
(S)-2-(dibenzylamino)propyl 4-(N-phenyl-biphenyl-4-sulfonamido)piperidine-1-carboxylate;
(S)-2-(dibenzylamino)propyl 4-(N-phenyl-naphthalen-2-ylsulfonamido)piperidine-1-carboxylate;
(S)-2-(dibenzylamino)propyl 4-(N-phenyl-(5-dimethylamino)naphthalen-1-ylsulfonamido)piperidine-1-carboxylate;
(S)-2-(dibenzylamino)propyl 4-(2-(4-chlorophenylthio)phenylamino)piperidine-1-carboxylate;
(S)-2-(dibenzylamino)-3-phenylpropyl 4-(2,6-dimethylphenylamino)piperidine-1-carboxylate;
(S)-2-(dibenzylamino)-3-phenylpropyl 4-(2,6-diethylphenylamino)piperidine-1-carboxylate;
(S)-2-(dibenzylamino)-3-phenylpropyl 4-(2-(methylthio)phenylamino)piperidine-1-carboxylate;
(S)-2-(dibenzylamino)propyl 4-(2-amino-1H-benzo[d]imidazol-1-yl)piperidine-1-carboxylate;
4-(dibenzylamino)phenyl 4-(1,2-dihydro-2-oxobenzo[d]imidazol-3-yl)piperidine-1-carboxylate;
(S)-2-(dibenzylamino)propyl 4-(2-oxo-2,3-dihydro-1H-b enzo[d]imidazol-4-yl)piperidine-1-carboxylate;
6-(dibenzylamino)hexyl 4-(1,2-dihydro-2-oxobenzo[d]imidazol-3-yl)piperidine-1-carboxylate;
Benzyl 4-(1,2-dihydro-2-oxobenzo[d]imidazol-3-yl)piperidine-1-carboxylate;
(1-benzylpiperidin-2-yl)methyl 4-(1,2-dihydro-2-oxobenzo[d]imidazol-3-yl)piperidine-1-carboxylate;
(S)-2-(dibenzylamino)-3-phenylpropyl 4-(N-phenyl-4-(trifluoromethyl)phenylsulfonamido)piperidine-1-carboxylate;
(S)-2-(benzyl(4-fluorobenzyl)amino)propyl 4-(2-oxo-2,3-dihydro-1H-benzo[d]imidazol-1-yl)piperidine-1-carboxylate;
2-(benzyl(tert-butoxycarbonyl)amino)ethyl 4-(2-oxo-2,3-dihydro-1H-benzo[d]imidazol-1-yl)piperidine-1-carboxylate;
3-(benzyl((1-methyl-1H-indol-2-yl)methyl)amino)propyl 4-(2-oxo-2,3-dihydro-1H-benzo[d]imidazol-1-yl)piperidine-1-carboxylate hydrochloride;
2-(benzyl(4-(methoxycarbonyl)benzyl)amino)ethyl 4-(2-oxo-2,3-dihydro-1H-benzo[d]imidazol-1-yl)piperidine-1-carboxylate;
3-(benzyl(3-(benzyloxy)benzyl)amino)propyl 4-(2-oxo-2,3-dihydro-1H-benzo[d]imidazol-1-yl)piperidine-1-carboxylate;
(S)-2-(benzylamino)-4-methylpentyl 4-(2-oxo-2,3-dihydro-1H-benzo[d]imidazol-1-yl)piperidine-1-carboxylate;
2-(benzylamino)-3-phenylpropyl 4-(2-oxo-2,3-dihydro-1H-benzo[d]imidazol-1-yl)piperidine-1-carboxylate and pharmaceutically acceptable salts thereof.

9. The compound of claim 1, wherein said pharmaceutically acceptable salts are acid addition salts formed with pharmaceutically acceptable acids.

10. The compound of claim 9, wherein said acid addition salts are hydrochloride, hydrobromide, sulphate or bisulphate, phosphate or hydrogenophosphate, acetate, benzoate, succinate, fumarate, maleate, lactate, citrate, tartrate, gluconate, methanesulphonate, benzene-sulphonate, paratoluene-sulphonate salts.

11. A method for the prevention of parasitic diseases involving apicomplexan parasites comprising administering the compounds of formula (I) as set forth in claim 1.

12. A method for the treatment of parasitic diseases involving apicomplexan parasites comprising administering the compounds as set forth in claim 1.

13. The method of claim 11, wherein when the parasitic disease is malaria, and wherein the compounds of formula (I) are selected in the group consisting of:
(S)-2-(dibenzylamino)propyl 4-(1H-benzo[d]imidazol-2-ylamino)piperidine-1-carboxylate;
(S)-2-(dibenzylamino)propyl 4-(2-oxo-3-phenylsulfonyl-2,3-dihydro-1H-benzo[d]imidazol-1-yl)piperidine-1-carboxylate;
(S)-2-(dibenzylamino)propyl 4-(3-(4-fluorophenylsulfonyl)-2-oxo-2,3-dihydro-1H-benzo[d]imidazol-1-yl)piperidine-1-carboxylate;
(S)-2-(dibenzylamino)propyl 4-(3-(4-methoxyphenylsulfonyl)-2-oxo-2,3-dihydro-1H-benzo[d]imidazol-1-yl)piperidine-1-carboxylate;
(S)-2-(dibenzylamino)propyl 4-(2-oxo-3-(4-(trifluoromethyl)phenylsulfonyl)-2,3-dihydro-1H-benzo[d]imidazol-1-yl)piperidine-1-carboxylate;
(S)-2-(dibenzylamino)-3-phenylpropyl 4-(1,2-dihydro-1-methyl-2-oxobenzo[d]imidazol-3-yl)piperidine-1-carboxylate;
(S)-2-(dibenzylamino)propyl 4-(2-oxo-3-(thiophen-2-ylsulfonyl)-2,3-dihydro-1H-benzo[d]imidazol-1-yl)piperidine-1-carboxylate;
N—((S)-2-(dibenzylamino)propyl)-4-(1,2-dihydro-2-oxobenzo[d]imidazol-3-yl)piperidine-1-carboxamidine;
(S)-2-(dibenzylamino)propyl 4-(N-phenylphenylsulfonamido)piperidine-1-carboxylate;
(S)-2-(dibenzylamino)propyl 4-(3-(4-ethoxy-4-oxobutyl)-2-oxo-2,3-dihydro-1H-benzo[d]imidazol-1-yl)piperidine-1-carboxylate;
(S)-2-(dibenzylamino)propyl 4-(1-benzyl-1,2-dihydro-2-oxobenzo[d]imidazol-3-yl)piperidine-1-carboxylate;
(S)-2-(dibenzylamino)propyl 4-(2-oxo-3-(quinolin-8-ylsulfonyl)-2,3-dihydro-1H-benzo[d]imidazol-1-yl)piperidine-1-carboxylate;
tert-butyl 3-(1-(4S)-2-(dibenzylamino)propoxy)carbonyl)piperidin-4-yl)-2,3-dihydro-2-oxobenzo[d]imidazole-1-carboxylate;
(S)-2-(dibenzylamino)-3-phenylpropyl 4-(1-benzyl-1,2-dihydro-2-oxobenzo[d]imidazol-3-yl)piperidine-1-carboxylate;
(S)-2-(dibenzylamino)propyl 4-(3-(naphthalen-1-ylsulfonyl)-2-oxo-2,3-dihydro-1H-benzo[d]imidazol-1-yl)piperidine-1-carboxylate;
(S)-2-(dibenzylamino)propyl 4-(1-(cyclohexylmethyl)-1,2-dihydro-2-oxobenzo[d]imidazol-3-yl)piperidine-1-carboxylate;
(S)-2-(dibenzylamino)propyl 4-(1,2-dihydro-2-thioxobenzo[d]imidazol-3-yl)piperidine-1-carboxylate;
1-(1-(3-(dibenzylamino)propyl)piperidin-4-yl)-1H-benzo[d]imidazol-2(3H)-one;
(S)-2-(dibenzylamino)-3-phenylpropyl 4-(1,2-dihydro-2-oxobenzo[d]imidazol-3-yl)piperidine-1-carboxylate;
(S)-2-(dibenzylamino)propyl 4-(N-phenylbenzamido)piperidine-1-carboxylate;
1-(S)-2-(dibenzylamino)propyl 3-ethyl 4-(1,2-dihydro-2-oxobenzo[d]imidazol-3-yl)piperidine-1,3-dicarboxylate;
(S)-2-(dibenzylamino)propyl 4-(1,2-dihydro-1-methyl-2-oxobenzo[d]imidazol-3-yl)piperidine-1-carboxylate;
(S)-2-(dibenzylamino)propyl 4-(2-oxo-3-(4-acetamidyl phenylsulfonyl)-2,3-dihydro-1H-benzo[d]imidazol-1-yl)piperidine-1-carboxylate;
1-(1-(2-(dibenzylamino)ethyl)piperidin-4-yl)-1H-benzo[d]imidazol-2(3H)-one;
(S)-2-(dibenzylamino)propyl 4-(2-oxo-3-methylsulfonyl-2,3-dihydro-1H-benzo[d]imidazol-1-yl)piperidine-1-carboxylate;
(S)-2-(dibenzylamino)propyl 4-(1H-benzo[d]imidazol-1-yl)piperidine-1-carboxylate;
(S)-2-(dibenzylamino)-3-phenylpropyl 4-(1,2-dihydro-3-phenylsulfonyl-2-oxobenzo[d]imidazol-1-yl)piperidine-1-carboxylate;
(S)-2-(dibenzylamino)propyl 4-(3-(2-tert-butyloxy carbonylaminoethyl)-2-oxo-2,3-dihydro-1H-benzo[d]imidazol-1-yl)piperidine-1-carboxylate;
(S)-2-(dibenzylamino)-3-phenylpropyl 4-(3-(2-tert-butyloxy carbonylaminoethyl)-2-oxo-2,3-dihydro-1H-benzo[d]imidazol-1-yl)piperidine-1-carboxylate;
(S)-2-(dibenzylamino)-3-phenylpropyl 4-(3-(2 aminoethyl)-2-oxo-2,3-dihydro-1H-benzo[d]imidazol-1-yl)piperidine-1-carboxylate D-biotin amid;
2-(benzyl(2-(benzyloxy)benzyl)amino)ethyl 4-(2-oxo-2,3-dihydro-1H-benzo[d]imidazol-1-yl)piperidine-1-carboxylate;
2-(benzyl(4-(benzyloxy)benzyl)amino)ethyl 4-(2-oxo-2,3-dihydro-1H-benzo[d]imidazol-1-yl)piperidine-1-carboxylate;
2-(benzyl(2,6-difluorobenzyl)amino)ethyl 4-(2-oxo-2,3-dihydro-1H-benzo[d]imidazol-1-yl)piperidine-1-carboxylate;
(S)-2-(benzyl(furan-2-ylmethyl)amino)propyl 4-(2-oxo-2,3-dihydro-1H-benzo[d]imidazol-1-yl)piperidine-1-carboxylate;
2-(benzyl(3-(benzyloxy)benzyl)amino)ethyl 4-(2-oxo-2,3-dihydro-1H-benzo[d]imidazol-1-yl)piperidine-1-carboxylate;
(E)-2-(benzyl(cinnamyl)amino)ethyl 4-(2-oxo-2,3-dihydro-1H-benzo[d]imidazol-1-yl)piperidine-1-carboxylate;
(S)-2-(benzyl(tert-butoxycarbonyl)amino)-4-methylpentyl 4-(2-oxo-2,3-dihydro-1H-benzo[d]imidazol-1-yl)piperidine-1-carboxylate;
(S)-2-(benzyl(tert-butoxycarbonyl)amino)-3-phenylpropyl 4-(2-oxo-2,3-dihydro-1H-benzo[d]imidazol-1-yl)piperidine-1-carboxylate;
(S)-2-(dibenzylamino)-3-methylbutyl 4-(1,2-dihydro-2-oxobenzo[d]imidazol-3-yl)piperidine-1-carboxylate;
(S)-2-(dibenzylamino)-4-methylpentyl 4-(1,2-dihydro-2-oxobenzo[d]imidazol-3-yl)piperidine-1-carboxylate;
2-(dibenzylamino)-3,3-dimethylbutyl 4-(1,2-dihydro-2-oxobenzo[d]imidazol-3-yl)piperidine-1-carboxylate;
(3-(dibenzylamino)butyl 4-(1,2-dihydro-2-oxobenzo[d]imidazol-3-yl)piperidine-1-carboxylate;
2-(dibenzylamino)-2-methylpropyl 4-(1,2-dihydro-2-oxobenzo[d]imidazol-3-yl)piperidine-1-carboxylate;
(S)-2-(dibenzylamino)-2-phenylethyl 4-(2-oxo-2,3-dihydro-1H-benzo[d]imidazol-1-yl)piperidine-1-carboxylate;
(S)-2-(benzyl(4-nitrobenzyl)amino)propyl 4-(2-oxo-2,3-dihydro-1H-benzo[d]imidazol-1-yl)piperidine-1-carboxylate;
3-(benzyl(4-nitrobenzyl)amino)propyl 4-(2-oxo-2,3-dihydro-1H-benzo[d]imidazol-1-yl)piperidine-1-carboxylate;
2-(benzyl(4-bromobenzyl)amino)ethyl 4-(2-oxo-2,3-dihydro-1H-benzo[d]imidazol-1-yl)piperidine-1-carboxylate;
S)-2-(benzyl(4-bromobenzyl)amino)propyl 4-(2-oxo-2,3-dihydro-1H-benzo[d]imidazol-1-yl)piperidine-1-carboxylate;
3-(benzyl(4-methoxybenzyl)amino)propyl 4-(2-oxo-2,3-dihydro-1H-benzo[d]imidazol-1-yl)piperidine-1-carboxylate;
(R)-2-(dibenzylamino)-3-phenylpropyl 4-(2-oxo-2,3-dihydro-1H-benzo[d]imidazol-1-yl)piperidine-1-carboxylate;
2-(benzyl(4-fluorobenzyl)amino)ethyl 4-(2-oxo-2,3-dihydro-1H-benzo[d]imidazol-1-yl)piperidine-1-carboxylate;
2-(benzyl(3-phenylpropyl)amino)ethyl 4-(2-oxo-2,3-dihydro-1H-benzo[d]imidazol-1-yl)piperidine-1-carboxylate;
2-(benzyl(4-methoxybenzyl)amino)ethyl 4-(2-oxo-2,3-dihydro-1H-benzo[d]imidazol-1-yl)piperidine-1-carboxylate;
2-(benzyl(3-methoxybenzyl)amino)ethyl 4-(2-oxo-2,3-dihydro-1H-benzo[d]imidazol-1-yl)piperidine-1-carboxylate;
2-(benzyl(3-phenoxybenzyl)amino)ethyl 4-(2-oxo-2,3-dihydro-1H-benzo[d]imidazol-1-yl)piperidine-1-carboxylate;
2-(benzyl(3-chlorobenzyl)amino)ethyl 4-(2-oxo-2,3-dihydro-1H-benzo[d]imidazol-1-yl)piperidine-1-carboxylate;
(S)-2-(benzyl(3-phenylpropyl)amino)propyl 4-(2-oxo-2,3-dihydro-1H-benzo[d]imidazol-1-yl)piperidine-1-carboxylate;
(S)-2-(benzyl(3-methoxybenzyl)amino)propyl 4-(2-oxo-2,3-dihydro-1H-benzo[d]imidazol-1-yl)piperidine-1-carboxylate;
(S)-2-(benzyl(3-phenoxybenzyl)amino)propyl 4-(2-oxo-2,3-dihydro-1H-benzo[d]imidazol-1-yl)piperidine-1-carboxylate;
(S)-2-(benzyl(3-chlorobenzyl)amino)propyl 4-(2-oxo-2,3-dihydro-1H-benzo[d]imidazol-1-yl)piperidine-1-carboxylate;
(S)-2-(benzyl(4-chlorobenzyl)amino)propyl 4-(2-oxo-2,3-dihydro-1H-benzo[d]imidazol-1-yl)piperidine-1-carboxylate;
2-(benzyl(naphthalen-1-ylmethyl)amino)ethyl 4-(2-oxo-2,3-dihydro-1H-benzo[d]imidazol-1-yl)piperidine-1-carboxylate;
2-(benzyl(quinolin-4-ylmethyl)amino)ethyl 4-(2-oxo-2,3-dihydro-1H-benzo[d]imidazol-1-yl)piperidine-1-carboxylate;
2-(benzyl((1-methyl-1H-indol-2-yl)methyl)amino)ethyl 4-(2-oxo-2,3-dihydro-1H-benzo[d]imidazol-1-yl)piperidine-1-carboxylate;
(S)-2-(benzyl(quinolin-4-ylmethyl)amino)propyl 4-(2-oxo-2,3-dihydro-1H-benzo[d]imidazol-1-yl)piperidine-1-carboxylate;
3-(benzyl(pyridin-3-ylmethyl)amino)propyl 4-(2-oxo-2,3-dihydro-1H-benzo[d]imidazol-1-yl)piperidine-1-carboxylate;
3-(benzyl(pyridin-2-ylmethyl)amino)propyl 4-(2-oxo-2,3-dihydro-1H-benzo[d]imidazol-1-yl)piperidine-1-carboxylate;
(S)-2-(benzyl(3-(benzyloxy)benzyl)amino)propyl 4-(2-oxo-2,3-dihydro-1H-benzo[d]imidazol-1-yl)piperidine-1-carboxylate;
(S)-2-(benzyl(phenanthren-9-ylmethyl)amino)propyl 4-(2-oxo-2,3-dihydro-1H-benzo[d]imidazol-1-yl)piperidine-1-carboxylate;
(S)-2-(benzyl(naphthalen-1-ylmethyl)amino)propyl 4-(2-oxo-2,3-dihydro-1H-benzo[d]imidazol-1-yl)piperidine-1-carboxylate;
(S)-2-(benzyl(thiophen-2-ylmethyl)amino)propyl 4-(2-oxo-2,3-dihydro-1H-benzo[d]imidazol-1-yl)piperidine-1-carboxylate;
(S)-2-(benzyl(pyridin-2-ylmethyl)amino)propyl 4-(2-oxo-2,3-dihydro-1H-benzo[d]imidazol-1-yl)piperidine-1-carboxylate;
3-(benzyl(thiophen-2-ylmethyl)amino)propyl 4-(2-oxo-2,3-dihydro-1H-benzo[d]imidazol-1-yl)piperidine-1-carboxylate;
3-(benzyl(3-chlorobenzyl)amino)propyl 4-(2-oxo-2,3-dihydro-1H-benzo[d]imidazol-1-yl)piperidine-1-carboxylate;
3-(benzyl(2-(benzyloxy)ethyl)amino)propyl 4-(2-oxo-2,3-dihydro-1H-benzo[d]imidazol-1-yl)piperidine-1-carboxylate;
3-(benzyl(naphthalen-1-ylmethyl)amino)propyl 4-(2-oxo-2,3-dihydro-1H-benzo[d]imidazol-1-yl)piperidine-1-carboxylate;
3-(benzyl(quinolin-4-ylmethyl)amino)propyl 4-(2-oxo-2,3-dihydro-1H-benzo[d]imidazol-1-yl)piperidine-1-carboxylate;
3-(benzyl(3-phenoxybenzyl)amino)propyl 4-(2-oxo-2,3-dihydro-1H-benzo[d]imidazol-1-yl)piperidine-1-carboxylate;
3-(benzyl(4-fluorobenzyl)amino)propyl 4-(2-oxo-2,3-dihydro-1H-benzo[d]imidazol-1-yl)piperidine-1-carboxylate;
3-(benzyl(3-phenylpropyl)amino)propyl 4-(2-oxo-2,3-dihydro-1H-benzo[d]imidazol-1-yl)piperidine-1-carboxylate;
3-(benzyl(3-methoxybenzyl)amino)propyl 4-(2-oxo-2,3-dihydro-1H-benzo[d]imidazol-1-yl)piperidine-1-carboxylate;
2-(benzyl(4-chlorobenzyl)amino)ethyl 4-(2-oxo-2,3-dihydro-1H-benzo[d]imidazol-1-yl)piperidine-1-carboxylate;
2-(benzyl(2-methoxybenzyl)amino)ethyl 4-(2-oxo-2,3-dihydro-1H-benzo[d]imidazol-1-yl)piperidine-1-carboxylate;
(S)-2-(dibenzylamino)-3-phenylpropyl 4-(phenylamino)piperidine-1-carboxylate;
(S)-2-(dibenzylamino)propyl 4-(2-phenyl-1H-benzo[d]imidazol-1-yl)piperidine-1-carboxylate;
(S)-2-(dibenzylamino)propyl 4-(N-phenyl-4-propylphenylsulfonamido)piperidine-1-carboxylate;
(S)-2-(dibenzylamino)propyl 4-(N-phenyl-4-fluorophenylsulfonamido)piperidine-1-carboxylate;
(S)-2-(dibenzylamino)propyl 4-(N-phenyl-4-methoxyphenylsulfonamido)piperidine-1-carboxylate;
(S)-2-(dibenzylamino)-3-phenylpropyl 4-(N-phenyl-4-methoxyphenylsulfonamido)piperidine-1-carboxylate;
(S)-2-(dibenzylamino)propyl 4-(N-phenyl-4-(trifluoromethyl)phenylsulfonamido)piperidine-1-carboxylate;
(S)-2-(dibenzylamino)propyl 4-(N-phenylthiophen-2-ylsulfonamido)piperidine-1-carboxylate;
(S)-2-(dibenzylamino)propyl 4-(N-phenylquinolin-8-ylsulfonamido)piperidine-1-carboxylate;
(S)-2-(dibenzylamino)propyl 4-(N-phenyl-naphthalen-1-ylsulfonamido)piperidine-1-carboxylate;
(S)-2-(dibenzylamino)-3-phenylpropyl 4-(N-phenyl-naphthalen-1-ylsulfonamido)piperidine-1-carboxylate;
(S)-2-(dibenzylamino)propyl 4-(N-phenylpivalamido)piperidine-1-carboxylate;
(S)-2-(dibenzylamino)propyl 4-(2-(phenylsulfonyl)phenylamino)piperidine-1-carboxylate;
(S)-2-(dibenzylamino)propyl 4-(1,3-diphenylthioureido)piperidine-1-carboxylate;
(S)-2-(dibenzylamino)propyl 4-(N-methyl-N-phenylamino)piperidine-1-carboxylate;
(S)-2-(dibenzylamino)propyl 4-(N-phenyl-2-methylphenylsulfonamido)piperidine-1-carboxylate;
(S)-2-(dibenzylamino)propyl 4-(N-phenyl-3-methylphenylsulfonamido)piperidine-1-carboxylate;
(S)-2-(dibenzylamino)propyl 4-(N-phenyl-3-(trifluoromethyl)phenylsulfonamido)piperidine-1-carboxylate;
(S)-2-(dibenzylamino)propyl 4-(N-phenyl-biphenyl-4-sulfonamido)piperidine-1-carboxylate;
(S)-2-(dibenzylamino)propyl 4-(N-phenyl-naphthalen-2-ylsulfonamido)piperidine-1-carboxylate;
(S)-2-(dibenzylamino)propyl 4-(N-phenyl-(5-dimethylamino)naphthalen-1-ylsulfonamido)piperidine-1-carboxylate;
(S)-2-(dibenzylamino)propyl 4-(2-(4-chlorophenylthio)phenylamino)piperidine-1-carboxylate;
(S)-2-(dibenzylamino)-3-phenylpropyl 4-(2,6-dimethylphenylamino)piperidine-1-carboxylate;
(S)-2-(dibenzylamino)-3-phenylpropyl 4-(2,6-diethylphenylamino)piperidine-1-carboxylate;
(S)-2-(dibenzylamino)-3-phenylpropyl 4-(2-(methylthio)phenylamino)piperidine-1-carboxylate; and pharmaceutically acceptable salts thereof.

14. The method of claim 11, wherein when the parasitic disease is toxoplasmose, and wherein the compounds of formula (I) are selected in the group consisting of:
O-2-(dibenzylamino)ethyl 4-(1,2-dihydro-2-oxobenzo[d]imidazol-3-yl)piperidine-1-carbothioate;
(S)-2-(dibenzylamino)propyl 4-(1,2-dihydro-1-methyl-2-oxobenzo[d]imidazol-3-yl)piperidine-1-carboxylate;
(S)-2-(dibenzylamino)propyl 3-(1,2-dihydro-2-oxobenzo[d]imidazol-3-yl)propylcarbamate;
1-benzylpiperidin-3-yl 4-(1,2-dihydro-2-oxobenzo[d]imidazol-3-yl)piperidine-1-carboxylate;
2-(benzyloxy)ethyl 4-(1,2-dihydro-2-oxobenzo[d]imidazol-3-yl)piperidine-1-carboxylate;
(S)-2-(dibenzylamino)propyl 4-(1,2-dihydro-2-oxonaphtho[2,3-d]imidazol-3-yl)piperidine-1-carboxylate;
1-(1-(3-(dibenzylamino)propyl)piperidin-4-yl)-1H-benzo[d]imidazol-2(3H)-one;
(S)-2-(dibenzylamino)propyl 4-(2-oxobenzo[d]oxazol-3(2H)-yl)piperidine-1-carboxylate;
(S)-2-(dibenzylamino)-3-phenylpropyl 4-(1,2-dihydro-1-methyl-2-oxobenzo[d]imidazol-3-yl)piperidine-1-carboxylate;
(S)-2-(dibenzylamino)propyl 4-(1,2-dihydro-2-oxo-1-(benzoyl)benzo[d]imidazol-3-yl)piperidine-1-carboxylate;
2-(1-benzylpiperidin-4-yl)ethyl 4-(1,2-dihydro-2-oxobenzo[d]imidazol-3-yl)piperidine-1-carboxylate;
(S)-2-(dibenzylamino)propyl 4-(1H-benzo[d]imidazol-2-ylamino)piperidine-1-carboxylate;
(S)-2-(dibenzylamino)propyl 4-(1-acetyl-1,2-dihydro-2-oxobenzo[d]imidazol-3-yl)piperidine-1-carboxylate;
(S)-2-(dibenzylamino)propyl 4-(6-chloro-1,2-dihydro-2-oxobenzo[d]imidazol-3-yl)piperidine-1-carboxylate;
O-2-(benzyloxy)ethyl 4-(1,2-dihydro-2-oxobenzo[d]imidazol-3-yl)piperidine-1-carbothioate;
O—(S)-2-(dibenzylamino)propyl 4-(1,2-dihydro-2-oxobenzo[d]imidazol-3-yl)piperidine-1-carbothioate;
3,3-diphenylpropyl 4-(1,2-dihydro-2-oxobenzo[d]imidazol-3-yl)piperidine-1-carboxylate;
(S)-2-(dibenzylamino)propyl 4-(N-phenylpropionamido)piperidine-1-carboxylate;
1-(1-(4-(dibenzylamino)butyl)piperidin-4-yl)-1H-benzo[d]imidazol-2(3H)-one;
(S)-2-(dibenzylamino)propyl 4-(3-(4-fluorophenylsulfonyl)-2-oxo-2,3-dihydro-1H-benzo[d]imidazol-1-yl)piperidine-1-carboxylate;
(S)-2-(dibenzylamino)propyl 4-(1-(cyclohexylmethyl)-1,2-dihydro-2-oxobenzo[d]imidazol-3-yl)piperidine-1-carboxylate;
(S)-2-(dibenzylamino)propyl 4-(2-oxo-3-phenylsulfonyl-2,3-dihydro-1H-benzo[d]imidazol-1-yl)piperidine-1-carboxylate;
(S)-4-(3-(1-42-(dibenzylamino)propoxy)carbonyl)piperidin-4-yl)-2-oxo-2,3-dihydro-1H-benzo[d]imidazol-1-yl)butanoic acid;
(S)-2-(dibenzylamino)propyl 4-(3-(4-methoxyphenylsulfonyl)-2-oxo-2,3-dihydro-1H-benzo[d]imidazol-1-yl)piperidine-1-carboxylate;
(S)-2-(dibenzylamino)propyl 4-(N-phenylphenylsulfonamido)piperidine-1-carboxylate;
(S)-2-(dibenzylamino)propyl 4-(1-benzyl-1,2-dihydro-2-oxobenzo[d]imidazol-3-yl)piperidine-1-carboxylate;
2-(ethyl(phenyl)amino)ethyl 4-(2-oxo-2,3-dihydro-1H-benzo[d]imidazol-1-yl)piperidine-1-carboxylate;
(S)-2-(dibenzylamino)-3-phenylpropyl 4-(1-benzyl-1,2-dihydro-2-oxobenzo[d]imidazol-3-yl)piperidine-1-carboxylate;
(S)-2-(dibenzylamino)propyl 4-(1H-indol-3-yl)piperidine-1-carboxylate;
(S)-2-(dibenzylamino)propyl 4-(2-oxo-3-methylsulfonyl-2,3-dihydro-1H-benzo[d]imidazol-1-yl)piperidine-1-carboxylate;
(S)-2-(dibenzylamino)propyl 4-(1H-benzo[d]imidazol-2-ylamino)piperidine-1-carboxylate;
(S)-2-(dibenzylamino)propyl 4-(2-oxo-3-phenylsulfonyl-2,3-dihydro-1H-benzo[d]imidazol-1-yl)piperidine-1-carboxylate;
(S)-2-(dibenzylamino)propyl 4-(3-(4-fluorophenylsulfonyl)-2-oxo-2,3-dihydro-1H-benzo[d]imidazol-1-yl)piperidine-1-carboxylate;
(S)-2-(dibenzylamino)propyl 4-(3-(4-methoxyphenylsulfonyl)-2-oxo-2,3-dihydro-1H-benzo[d]imidazol-1-yl)piperidine-1-carboxylate;
(S)-2-(dibenzylamino)propyl 4-(2-oxo-3-(4-(trifluoromethyl)phenylsulfonyl)-2,3-dihydro-1H-benzo[d]imidazol-1-yl)piperidine-1-carboxylate;
(S)-2-(dibenzylamino)-3-phenylpropyl 4-(1,2-dihydro-1-methyl-2-oxobenzo[d]imidazol-3-yl)piperidine-1-carboxylate;
(S)-2-(dibenzylamino)propyl 4-(2-oxo-3-(thiophen-2-ylsulfonyl)-2,3-dihydro-1H-benzo[d]imidazol-1-yl)piperidine-1-carboxylate;
N-((S)-2-(dibenzylamino)propyl)-4-(1,2-dihydro-2-oxobenzo[d]imidazol-3-yl)piperidine-1-carboxamidine;
(S)-2-(dibenzylamino)propyl 4-(N-phenylphenylsulfonamido)piperidine-1-carboxylate;
(S)-2-(dibenzylamino)propyl 4-(3-(4-ethoxy-4-oxobutyl)-2-oxo-2,3-dihydro-1H-benzo[d]imidazol-1-yl)piperidine-1-carboxylate;
(S)-2-(dibenzylamino)propyl 4-(1-benzyl-1,2-dihydro-2-oxobenzo[d]imidazol-3-yl)piperidine-1-carboxylate;
(S)-2-(dibenzylamino)propyl 4-(2-oxo-3-(quinolin-8-ylsulfonyl)-2,3-dihydro-1H-benzo[d]imidazol-1-yl)piperidine-1-carboxylate;
tert-butyl 3-(1-(4S)-2-(dibenzylamino)propoxy)carbonyl)piperidin-4-yl)-2,3-dihydro-2-oxobenzo[d]imidazole-1-carboxylate;
(S)-2-(dibenzylamino)-3-phenylpropyl 4-(1-benzyl-1,2-dihydro-2-oxobenzo[d]imidazol-3-yl)piperidine-1-carboxylate;
(S)-2-(dibenzylamino)propyl 4-(3-(naphthalen-1-ylsulfonyl)-2-oxo-2,3-dihydro-1H-benzo[d]imidazol-1-yl)piperidine-1-carboxylate;
(S)-2-(dibenzylamino)propyl 4-(1-(cyclohexylmethyl)-1,2-dihydro-2-oxobenzo[d]imidazol-3-yl)piperidine-1-carboxylate;
(S)-2-(dibenzylamino)propyl 4-(1,2-dihydro-2-thioxobenzo[d]imidazol-3-yl)piperidine-1-carboxylate;
1-(1-(3-(dibenzylamino)propyl)piperidin-4-yl)-1H-benzo[d]imidazol-2(3H)-one;
(S)-2-(dibenzylamino)-3-phenylpropyl 4-(1,2-dihydro-2-oxobenzo[d]imidazol-3-yl)piperidine-1-carboxylate;
(S)-2-(dibenzylamino)propyl 4-(N-phenylbenzamido)piperidine-1-carboxylate;
1-(S)-2-(dibenzylamino)propyl 3-ethyl 4-(1,2-dihydro-2-oxobenzo[d]imidazol-3-yl)piperidine-1,3-dicarboxylate;
(S)-2-(dibenzylamino)propyl 4-(1,2-dihydro-1-methyl-2-oxobenzo[d]imidazol-3-yl)piperidine-1-carboxylate;
(S)-2-(dibenzylamino)propyl 4-(2-oxo-3-(4-acetamidyl phenylsulfonyl)-2,3-dihydro-1H-benzo[d]imidazol-1-yl)piperidine-1-carboxylate;
1-(1-(2-(dibenzylamino)ethyl)piperidin-4-yl)-1H-benzo[d]imidazol-2(3H)-one;
(S)-2-(dibenzylamino)propyl 4-(2-oxo-3-methylsulfonyl-2,3-dihydro-1H-benzo[d]imidazol-1-yl)piperidine-1-carboxylate;
(S)-2-(dibenzylamino)propyl 4-(1H-benzo[d]imidazol-1-yl)piperidine-1-carboxylate;
(S)-2-(dibenzylamino)-3-phenylpropyl 4-(1,2-dihydro-3-phenylsulfonyl-2-oxobenzo[d]imidazol-1-yl)piperidine-1-carboxylate;
(S)-2-(dibenzylamino)propyl 4(3(2-tert-butyloxy carbonylaminoethyl)-2-oxo-2,3-dihydro-1H-benzo[d]imidazol-1-yl)piperidine-1-carboxylate;
(S)-2-(dibenzylamino)-3-phenylpropyl 4(3(2-tert-butyloxy carbonylaminoethyl)-2-oxo-2,3-dihydro-1H-benzo[d]imidazol-1-yl)piperidine-1-carboxylate;
(S)-2-(dibenzylamino)-3-phenylpropyl 4-(3-(2 aminoethyl)-2-oxo-2,3-dihydro-1H-benzo[d]imidazol-1-yl)piperidine-1-carboxylate D-biotin amid;
(S)-2-(dibenzylamino)propyl 4-(2-phenyl-1H-benzo[d]imidazol-1-yl)piperidine-1-carboxylate;
(S)-2-(dibenzylamino)propyl 4-(2-amino-1H-benzo[d]imidazol-1-yl)piperidine-1-carboxylate;
(S)-2-(dibenzylamino)propyl 4-(4-fluoro-N-phenylphenylsulfonamido)piperidine-1-carboxylate;
(S)-2-(dibenzylamino)propyl 4-(4-methoxy-N-phenylphenylsulfonamido)piperidine-1-carboxylate;
(S)-2-(dibenzylamino)propyl 4(N-phenylthiophene-2-sulfonamido)piperidine-1-carboxylate;
(S)-2-(dibenzylamino)propyl 4(N-phenylquinoline-8-sulfonamido)piperidine-1-carboxylate;
4-(dibenzylamino)phenyl 4-(1,2-dihydro-2-oxobenzo[d]imidazol-3-yl)piperidine-1-carboxylate;
(S)-2-(dibenzylamino)propyl 4(N-phenylpivalamido)piperidine-1-carboxylate;
(S)-2-(dibenzylamino)propyl 4-(2-oxo-2,3-dihydro-1H-benzo[d]imidazol-4-yl)piperidine-1-carboxylate;
6-(dibenzylamino)hexyl 4-(1,2-dihydro-2-oxobenzo[d]imidazol-3-yl)piperidine-1-carboxylate;
(S)-2-(dibenzylamino)-4-methylpentyl 4-(2-oxo-2,3-dihydro-1H-benzo[d]imidazol-1-yl)piperidine-1-carboxylate;
(S)-3-(dibenzylamino)butyl 4-(2-oxo-2,3-dihydro-1H-benzo[d]imidazol-1-yl)piperidine-1-carboxylate;
(S)-2-(dibenzylamino)-2-phenylethyl 4-(1,2-dihydro-2-oxobenzo[d]imidazol-3-yl)piperidine-1-carboxylate;
Benzyl 4-(1,2-dihydro-2-oxobenzo[d]imidazol-3-yl)piperidine-1-carboxylate;
(1-benzylpiperidin-2-yl)methyl 4-(1,2-dihydro-2-oxobenzo[d]imidazol-3-yl)piperidine-1-carboxylate;
(S)-2-(dibenzylamino)propyl 4-(1,3-diphenylthioureido)piperidine-1-carboxylate;
3-(benzyl(4-nitrobenzyl)amino)propyl 4-(2-oxo-2,3-dihydro-1H-benzo[d]imidazol-1-yl)piperidine-1-carboxylate;
2-(benzyl(4-bromobenzyl)amino)ethyl 4-(2-oxo-2,3-dihydro-1H-benzo[d]imidazol-1-yl)piperidine-1-carboxylate;
(S)-2-(dibenzylamino)-3-phenylpropyl 4-(phenylamino)piperidine-1-carboxylate;
(S)-2-(dibenzylamino)propyl 4-(2-methyl-N-phenylphenylsulfonamido)piperidine-1-carboxylate;
(S)-2-(dibenzylamino)propyl 4-(3-methyl-N-phenylphenylsulfonamido)piperidine-1-carboxylate;
(S)-2-(dibenzylamino)propyl 4-(N-phenyl-3-(trifluoromethyl)phenylsulfonamido)piperidine-1-carboxylate;
(S)-2-(dibenzylamino)propyl 4-(5-(dimethylamino)-N-phenylnaphthalene-1-sulfonamido)piperidine-1-carboxylate;
(R)-2-(dibenzylamino)-3-phenylpropyl 4-(1,2-dihydro-2-oxobenzo[d]imidazol-3-yl)piperidine-1-carboxylate;
2-(benzyl(4-fluorobenzyl)amino)ethyl 4-(2-oxo-2,3-dihydro-1H-benzo[d]imidazol-1-yl)piperidine-1-carboxylate;
2-(benzyl(3-phenylpropyl)amino)ethyl 4-(2-oxo-2,3-dihydro-1H-benzo[d]imidazol-1-yl)piperidine-1-carboxylate;
2-(benzyl(4-methoxybenzyl)amino)ethyl 4-(2-oxo-2,3-dihydro-1H-benzo[d]imidazol-1-yl)piperidine-1-carboxylate;
2-(benzyl(3-methoxybenzyl)amino)ethyl 4-(2-oxo-2,3-dihydro-1H-benzo[d]imidazol-1-yl)piperidine-1-carboxylate;
2-(benzyl(3-phenoxybenzyl)amino)ethyl 4-(2-oxo-2,3-dihydro-1H-benzo[d]imidazol-1-yl)piperidine-1-carboxylate;
(S)-2-(dibenzylamino)-3-phenylpropyl 4-(N-phenyl-4-(trifluoromethyl)phenylsulfonamido)piperidine-1-carboxylate;
(S)-2-(benzyl(4-fluorobenzyl)amino)propyl 4-(2-oxo-2,3-dihydro-1H-benzo[d]imidazol-1-yl)piperidine-1-carboxylate;
(S)-2-(benzyl(3-phenylpropyl)amino)propyl 4-(2-oxo-2,3-dihydro-1H-benzo[d]imidazol-1-yl)piperidine-1-carboxylate;
(S)-2-(benzyl(quinolin-4-ylmethyl)amino)propyl 4-(2-oxo-2,3-dihydro-1H-benzo[d]imidazol-1-yl)piperidine-1-carboxylate;
(S)-2-(benzyl(3-methoxybenzyl)amino)propyl 4-(2-oxo-2,3-dihydro-1H-benzo[d]imidazol-1-yl)piperidine-1-carboxylate;
(S)-2-(benzyl(3-chlorobenzyl)amino)propyl 4-(2-oxo-2,3-dihydro-1H-benzo[d]imidazol-1-yl)piperidine-1-carboxylate;
2-(benzyl(quinolin-4-ylmethyl)amino)ethyl 4-(2-oxo-2,3-dihydro-1H-benzo[d]imidazol-1-yl)piperidine-1-carboxylate;
2-(benzyl((1-methyl-1H-indol-2-yl)methyl)amino)ethyl 4-(2-oxo-2,3-dihydro-1H-benzo[d]imidazol-1-yl)piperidine-1-carboxylate;
3-(benzyl(pyridin-2-ylmethyl)amino)propyl 4-(2-oxo-2,3-dihydro-1H-benzo[d]imidazol-1-yl)piperidine-1-carboxylate;
(S)-2-(benzyl(3-(benzyloxy)benzyl)amino)propyl 4-(2-oxo-2,3-dihydro-1H-benzo[d]imidazol-1-yl)piperidine-1-carboxylate;
(S)-2-(benzyl(phenanthren-9-ylmethyl)amino)propyl 4-(2-oxo-2,3-dihydro-1H-benzo[d]imidazol-1-yl)piperidine-1-carboxylate;
(S)-2-(benzyl(naphthalen-1-ylmethyl)amino)propyl 4-(2-oxo-2,3-dihydro-1H-benzo[d]imidazol-1-yl)piperidine-1-carboxylate;
(S)-2-(benzyl(thiophen-2-ylmethyl)amino)propyl 4-(2-oxo-2,3-dihydro-1H-benzo[d]imidazol-1-yl)piperidine-1-carboxylate;
(S)-2-(benzyl(pyridin-2-ylmethyl)amino)propyl 4-(2-oxo-2,3-dihydro-1H-benzo[d]imidazol-1-yl)piperidine-1-carboxylate;
2-(benzyl(tert-butoxycarbonyl)amino)ethyl 4-(2-oxo-2,3-dihydro-1H-benzo[d]imidazol-1-yl)piperidine-1-carboxylate;
3-(benzyl(thiophen-2-ylmethyl)amino)propyl 4-(2-oxo-2,3-dihydro-1H-benzo[d]imidazol-1-yl)piperidine-1-carboxylate;
3-(benzyl(3-methoxybenzyl)amino)propyl 4-(2-oxo-2,3-dihydro-1H-benzo[d]imidazol-1-yl)piperidine-1-carboxylate;
3-(benzyl(3-chlorobenzyl)amino)propyl 4-(2-oxo-2,3-dihydro-1H-benzo[d]imidazol-1-yl)piperidine-1-carboxylate;
3-(benzyl(naphthalen-1-ylmethyl)amino)propyl 4-(2-oxo-2,3-dihydro-1H-benzo[d]imidazol-1-yl)piperidine-1-carboxylate;
3-(benzyl(quinolin-4-ylmethyl)amino)propyl 4-(2-oxo-2,3-dihydro-1H-benzo[d]imidazol-1-yl)piperidine-1-carboxylate;
3-(benzyl((1-methyl-1H-indol-2-yl)methyl)amino)propyl 4-(2-oxo-2,3-dihydro-1H-benzo[d]imidazol-1-yl)piperidine-1-carboxylate;
3-(benzyl(3-phenoxybenzyl)amino)propyl 4-(2-oxo-2,3-dihydro-1H-benzo[d]imidazol-1-yl)piperidine-1-carboxylate;
2-(benzyl(4-(methoxycarbonyl)benzyl)amino)ethyl 4-(2-oxo-2,3-dihydro-1H-benzo[d]imidazol-1-yl)piperidine-1-carboxylate;
2-(benzyl(4-chlorobenzyl)amino)ethyl 4-(2-oxo-2,3-dihydro-1H-benzo[d]imidazol-1-yl)piperidine-1-carboxylate;
2-(benzyl(2-(benzyloxy)benzyl)amino)ethyl 4-(2-oxo-2,3-dihydro-1H-benzo[d]imidazol-1-yl)piperidine-1-carboxylate;
3-(benzyl(3-(benzyloxy)benzyl)amino)propyl 4-(2-oxo-2,3-dihydro-1H-benzo[d]imidazol-1-yl)piperidine-1-carboxylate;
(S)-2-(benzyl(tert-butoxycarbonyl)amino)-4-methylpentyl 4-(2-oxo-2,3-dihydro-1H-benzo[d]imidazol-1-yl)piperidine-1-carboxylate;
(S)-2-(benzylamino)-4-methylpentyl 4-(2-oxo-2,3-dihydro-1H-benzo[d]imidazol-1-yl)piperidine-1-carboxylate;
2-(benzylamino)-3-phenylpropyl 4-(2-oxo-2,3-dihydro-1H-benzo[d]imidazol-1-yl)piperidine-1-carboxylate and pharmaceutically acceptable salts thereof.

15. A pharmaceutical composition comprising, as an active principle, at least one compound of formula (I) as defined in claim 1, and at least one pharmaceutically acceptable excipient.

16. The pharmaceutical composition of claim 15, wherein it comprises, in addition to the at least one compound of formula (I), one or more additional antiparasitic active principle.
